(19) United States
(12) Patent Application Publication
(10) Pub. No.: US 2025/0174366 A1
(43) Pub. Date: May 29, 2025

(54) Methods and Compositions for Assessing and Treating Lupus

(71) Applicant: AMPEL BioSolutions, LLC, Charlottesville, VA (US)

(72) Inventors: Amrie C. GRAMMER, Charlottesville, VA (US); Peter E. LIPSKY, Charlottesville, VA (US); Prathyusha BACHALI, Charlottesville, VA (US); Erika HUBBARD, Charlottesville, VA (US); Kathryn K. ALLISON, Charlottesville, VA (US); Andrea DAAMEN, Charlottesville, VA (US)

(21) Appl. No.: 18/934,801

(22) Filed: Nov. 1, 2024

Related U.S. Application Data
(63) Continuation of application No. PCT/US2023/020752, filed on May 2, 2023.
(60) Provisional application No. 63/449,882, filed on Mar. 3, 2023.
(60) Provisional application No. 63/424,090, filed on Nov. 9, 2022.
(60) Provisional application No. 63/407,556, filed on Sep. 16, 2022.
(60) Provisional application No. 63/338,003, filed on May 3, 2022.

(51) Int. Cl. G16H 70/60; G16B 25/10; G16B 40/30; G16H 10/40

(52) U.S. Cl. CPC G16H 70/60; G16B 25/10; G16B 40/30; G16H 10/40

(57) ABSTRACT

The present disclosure provides a method for assessing a lupus state of a patient, the method comprising: analyzing a data set comprising and/or derived from gene expression measurements of at least 2 genes selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32, to classify the lupus state of the patient, wherein the gene expression measurements are obtained from a biological sample obtained or derived from the patient.

### CROSS-REFERENCE

This application is a continuation of PCT/US2023/020752, filed May 2, 2023, which claims priority to U.S. Provisional Patent Application Nos.: 63/338,003 filed May 3, 2022; 63/407,556 filed Sep. 16, 2022; 63/424,090 filed Nov. 9, 2022 and 63/449,882 filed Mar. 3, 2023, all of which are incorporated in full herein by reference.

### BACKGROUND

Many diseases, including Systemic Lupus Erythematosus (SLE), are heterogeneous in nature, and have variable causation, course and responsiveness to therapy. There is a need for understanding biological pathways involved in the pathogenesis of these conditions to allow identification and optimization of therapies.

### SUMMARY

One aspect of the present disclosure is directed to a method for classifying a lupus disease state of a patient. The method can include analyzing a data set comprising or derived from gene expression measurements of at least 2 genes selected from genes listed in each of one or more Tables selected from Tables: 1 to 32, to classify the lupus disease state of the patient.

The number of genes selected from different selected Tables may be the same or different. The gene expression measurements can be obtained from a biological sample obtained or derived from the patient. The lupus disease state of the patient can be classified as group A lupus disease state, group B lupus disease state, group C lupus disease state, group D lupus disease state, group E lupus disease state, group F lupus disease state, group G lupus disease state, or group H lupus disease state. In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, or all genes selected from genes listed in each of the one or more Tables selected from Tables: 1 to 32, wherein the number of genes selected from different selected Tables may be the same or different. In certain embodiments, the data set comprises or is derived from gene expression measurements of an effective number of genes selected from genes listed in each of the one or more Tables selected from Tables: 1 to 32, wherein the number of gene selected from different selected Tables may be the same or different. In certain embodiments, the data set comprises or is derived from gene expression measurements of all the genes listed in each of the one or more Tables selected from Tables: 1 to 32. In certain embodiments, at least 23 Tables are selected from Tables: 1 to 32, i.e., the one or more Tables selected from Tables: 1 to 32 comprises at least 23 Tables. In certain embodiments, at least 23 Tables are selected from Tables: 1 to 32, wherein the selected Tables comprises Tables: 2; 4; 5; 6; 7; 8; 10; 11; 12; 13; 14; 15; 16; 17; 18; 19; 20; 22; 23; 24; 25; 31; and 32. In certain embodiments, at least 24 Tables are selected from Tables: 1 to 32. In certain embodiments, the one or more Tables comprise at least 24 Tables, wherein at least Tables: 2; 3; 4; 5; 6; 7; 8; 10; 11; 12; 13; 14; 15; 16; 17; 18; 19; 20; 22; 23; 24; 25; 31; and 32, are selected. In certain embodiments, at least 25 Tables are selected from Tables: 1 to 32. In certain embodiments, the one or more Tables comprise at least 25 Tables, wherein at least Tables: 1; 2; 3; 4; 5; 6; 7; 8; 10; 11; 12; 13; 14; 15; 16; 17; 18; 19; 20; 22; 23; 24; 25; 31; and 32, are selected. In certain embodiments, at least 26 Tables are selected from Tables: 1 to 32. In certain embodiments, at least 26 Tables are selected from Tables: 1 to 32, wherein the selected Tables comprises Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, Tables: 1 to 32 are selected. In certain embodiments, the data set comprises or is derived from gene expression measurements of the genes listed in the Tables selected.

The method can classify the lupus disease state of the patient with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lupus disease state of the patient with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lupus disease state of the patient with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify lupus disease state of the patient with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lupus disease state of the patient with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the data set is derived from the gene expression measurements using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof. In certain embodiments, the data set is derived from the gene expression measurements using GSVA. In certain embodiments, the data set comprises one or more GSVA scores of the patient, wherein the one or more GSVA scores are generated based on the one or more Tables selected from Tables 1 to 32, wherein for each selected Table the at least 2 genes selected from the selected Table forms an input gene set for generating a GSVA score based on the selected Table using GSVA, and wherein the one or more GSVA scores comprise the generated GSVA scores. In certain embodiments, for each selected Table the effective number of genes selected from the selected Table forms the input gene set for generating the GSVA score based on the selected Table, using GSVA. In certain embodiments, for each selected Table the genes listed in the Table forms the input gene set for generating the GSVA score based on the selected Table, using GSVA. For each selected Table, the GSVA score is generated based on enrichment of the input gene set (e.g., containing at least 2 genes, effective number of genes, or all genes selected from the Table) in the biological sample obtained or derived from the patient. Enrichment can be measured with respect to a reference dataset, as described herein.

In certain embodiments, analyzing the data set comprises providing the data set as an input to a trained machine-learning model trained to generate an inference of whether the data set is indicative of the patient having group A lupus disease state, group B lupus disease state, group C lupus disease state, group D lupus disease state, group E lupus disease state, group F lupus disease state, group G lupus disease state, or group H lupus disease state, wherein the method classify the lupus disease state of the patient based on the inference of the trained machine-learning model. In certain embodiments, the method further comprises receiving, as an output of the trained machine-learning model, the inference; and/or electronically outputting a report classifying the lupus disease state of a patient.

The trained machine-learning model can be trained using linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

In certain embodiments, the inference comprises a confidence value between 0 and 1 that the patient has the group A lupus disease state, the group B lupus disease state, the group C lupus disease state, the group D lupus disease state, the group E lupus disease state, the group F lupus disease state, the group G lupus disease state, or the group H lupus disease state.

In certain embodiments, the trained machine-learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The ROC curve can be for lupus disease state classification of the patient.

In certain embodiments, analyzing the data set comprises generating a risk score of the patient based on the data set, wherein the method classify the lupus disease state of the patient based on the risk score. In certain embodiments, the risk score of the patient is generated based on the one or more GSVA scores of the patient.

In certain embodiments, the method comprises performing Shapley Additive Explanations (SHAP) analysis on the data set to determine contribution of one or more gene features to the lupus disease state classification of the patient. The SHAP analysis can be performed on the trained machine learning model and on the dataset. The genes selected (e.g., at least 2 genes, effective number of genes or all genes) from each selected Table (e.g., the one or more Tables selected from Tables 1 to 32) can form a gene feature. Genes selected from different selected Tables can form different gene features. The Tables selected and the genes selected from the selected Tables can be as described above or elsewhere herein. The one or more gene features comprise the gene features formed based on the Tables selected. The contribution of the one or more gene features to the lupus disease state classification of the patient can be determined based on the SHAP values obtained from the SHAP analysis. The one or more gene features can be the features of the trained machine learning model, and GSVA scores of the patient generated based the one or more gene features, can be feature values for the dataset. Gene features having higher contribution to the lupus disease state classification of the patient can have higher absolute SHAP values, among the absolute SHAP values of the one or more gene features, determined based on the SHAP analysis on the dataset.

The biological sample can comprise a blood sample, a tissue biopsy, isolated peripheral blood mononuclear cells (PBMCs), or any derivative thereof. In certain embodiments, the biological sample comprises a blood sample or any derivative thereof. In certain embodiments, the biological sample comprises isolated PBMCs or any derivative thereof. In certain embodiments, the biological sample comprises a tissue biopsy sample or any derivative thereof.

In certain embodiments, the tissue is skin tissue. In certain embodiments, the tissue is kidney tissue. The patient can be a human.

In certain embodiments, the patient has lupus. In certain embodiments, the patient is at elevated risk of having lupus. In certain embodiments, the patient is asymptomatic for lupus.

In certain embodiments, the method comprises selecting, recommending, and/or administering a treatment to the patient based on the classification of the lupus disease state of the patient. In certain embodiments, the method comprises administering a treatment to the patient based on the classification of the lupus disease state of the patient. The treatment can be configured to treat, reduce severity of, and/or reduce risk of having lupus. In certain embodiments, the treatment is configured to treat lupus. In certain embodiments, the treatment is configured to treat reduce severity of lupus. In certain embodiments, the treatment is configured to reduce risk of having lupus. The treatment can comprises one or more pharmaceutical compositions. In certain embodiments, the treatment is based on the contribution of the one or more gene features to the lupus disease state classification of the patient. The contribution of one or more gene features to the lupus disease state classification of the patient, can be determined by the SHAP analysis on the data set, as described above or elsewhere herein. In certain embodiments, the treatment targets at least one gene feature out of the gene features having top 10, top 9, top 8, top 7, top 6, top 5, top 4, top 3 or top 2 absolute SHAP values among the absolute SHAP values of the one or more gene features determined by the SHAP analysis, on the data set. In certain embodiments, the treatment targets at least one gene feature out of the gene features having top 10 absolute SHAP values among the absolute SHAP values of the one or more gene features determined by the SHAP analysis. In certain embodiments, the treatment targets at least one gene feature out of the gene features having top 5 absolute SHAP values among the absolute SHAP values of the one or more gene features determined by the SHAP analysis. In certain embodiments, the treatment targets at least one gene feature out of the gene features having top 3 absolute SHAP values among the absolute SHAP values of the one or more gene features determined by the SHAP analysis. In certain embodiments, the treatment targets the gene feature having the top absolute SHAP value among the absolute SHAP values of the one or more gene features determined by the SHAP analysis. Treatment targeting a gene feature formed based on Table 8, (e.g., a gene feature containing at least 2 genes, effective number of genes, or all genes selected from the genes listed in Table 8) can comprise an IFN inhibitor such as Anifrolumab. Treatment targeting a gene feature formed based on Table 23, (e.g., a gene feature containing at least 2 genes, effective number of genes, or all genes selected from the genes listed in Table 23), can comprise a Plasma cell inhibitor such as belimumab, mycophenolate, Bortezomib, Carfilzomib, Ixazomib, isatuximab, daratumumab, elotuzumab, or any combination thereof. Treatment targeting a gene feature formed based on Table 10, (e.g., a gene feature containing at least 2 genes, effective number of genes, or all genes selected from the genes listed in Table 10) can comprise an IL1 inhibitor such as Anakinra, and/or Canakinumab. Treatment targeting a gene feature formed based on Table 31, (e.g., a gene feature containing at least 2 genes, effective number of genes, or all genes selected from the genes listed in Table 31) can comprise a TNF inhibitor such as Adalimumab, Certolizumab pegol, Etanercept, Golimumab, Infliximab, or any combination thereof. Treatment targeting a gene feature formed based on Table 19, (e.g., a gene feature containing at least 2 genes, effective number of genes, or all genes selected from the genes listed in Table 19) can comprise a Neutrophil function inhibitor such as Dasatinib, Apremilast, Roflumilast, or any combination thereof. Treatment targeting a gene feature formed based on Table 20, (e.g., a gene feature containing at least 2 genes, effective number of genes, or all genes selected from the genes listed in Table 20) can comprise a NK cell inhibitor such as Azathioprine (AZA). Treatment targeting a gene feature formed based on Table 3, (e.g., a gene feature containing at least 2 genes, effective number of genes, or all genes selected from the genes listed in Table 3) can comprise a B cell inhibitor such as Belimumab, Rituximab, Obinutuzumab, Ocrelizumab, Ofatumumab, Inebilizumab, or any combination thereof. The genes selected from the one or more selected Tables (e.g., from Tables 1 to 32) can form the one or more gene features, wherein genes selected from each selected Table can form a gene feature, and genes selected from different selected Tables form different gene features. The Tables selected and the genes selected from a selected Table can be as described above or elsewhere herein. The one or more gene features comprises the gene features formed based on the Tables selected. In certain embodiments, the treatment can target a gene feature significantly enriched in the biological sample obtained or derived from the patient. In certain embodiments, the gene feature significantly enriched in the biological sample obtained or derived from the patient can be determined based on a Z-score method, as described above or elsewhere herein. In certain embodiments, the IFN module is significantly enriched in the biological sample obtained or derived from the patient, such as a gene feature containing at least 2 genes, effective number of genes, and/or all genes selected from the genes listed in Table 8 has a Z-score greater than 2, and the treatment can comprise a IFN inhibitor such as Anifrolumab. In certain embodiments, the plasma cells module is significantly enriched in the biological sample obtained or derived from the patient, such as a gene feature containing at least 2 genes, effective number of genes, and/or all genes selected from the genes listed in Table 23 has a Z-score greater than 2, and the treatment can comprise a Plasma cell inhibitor such as belimumab, mycophenolate, Bortezomib, Carfilzomib, Ixazomib, isatuximab, daratumumab, elotuzumab, or any combination thereof. In certain embodiments, the IL1 pathway module is significantly enriched in the biological sample obtained or derived from the patient, such as a gene feature containing at least 2 genes, effective number of genes, and/or all genes selected from the genes listed in Table 10 has a Z-score greater than 2, and the treatment can comprise a IL1 inhibitor such as Anakinra, and/or Canakinumab. In certain embodiments, the TNF Waddel Up module is significantly enriched in the biological sample obtained or derived from the patient, such as a gene feature containing at least 2 genes, effective number of genes, and/or all genes selected from the genes listed in Table 31 has a Z-score greater than 2, and the treatment can comprise a TNF inhibitor such as Adalimumab, Certolizumab pegol, Etanercept, Golimumab, Infliximab, or any combination thereof. In certain embodiments, the Neutrophil module is significantly enriched in the biological sample obtained or derived from the patient, such as a gene feature containing at least 2 genes, effective number of genes, and/or all genes selected from the genes listed in Table 19 has a Z-score greater than 2, and the treatment can comprise a Neutrophil function inhibitor such as Dasatinib, Apremilast, Roflumilast, or any combination thereof. In certain embodiments, the NK cell module is significantly enriched in the biological sample obtained or derived from the patient, such as a gene feature containing at least 2 genes, effective number of genes, and/or all genes selected from the genes listed in Table 20 has a Z-score greater than 2, and the treatment can comprise a NK cell inhibitor such as Azathioprine (AZA). In certain embodiments, the B cells module is significantly enriched in the biological sample obtained or derived from the patient, such as a gene feature containing at least 2 genes, effective number of genes, and/or all genes selected from the genes listed in Table 3 has a Z-score greater than 2, and the treatment can comprise a B cell inhibitor such as Belimumab, Rituximab, Obinutuzumab, Ocrelizumab, Ofatumumab, Inebilizumab, or any combination thereof. The treatment may or may not target every gene feature that is enriched in the biological sample obtained or derived from the patient. The genes selected from the one or more selected Tables (e.g., from Tables 1 to 32) can form one or more gene features, wherein genes selected from each selected Table can form a gene feature, and genes selected from different selected Tables form different gene features. The Tables selected and the genes selected from a selected Table can be as described above or elsewhere herein. The one or more gene features comprises the gene features formed based on the Tables selected.

In certain embodiments, the treatment comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab.

Mycophenolate can be Mycophenolate Mofetil. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast.

Non-limiting examples of a NK cell inhibitor include Azathioprine. Non-limiting examples of a B cell inhibitor include Belimumab, Rituximab, Obinutuzumab, Ocrelizumab, Ofatumumab, and Inebilizumab. In certain embodiments, the treatment comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof. In certain embodiments, the treatment for, group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, NK cell inhibitor, a neutrophil function inhibitor, a TNF inhibitor, an IL1 inhibitor, a Plasma cell inhibitor or any combination thereof; group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof; and/or group H lupus disease state comprises an IFN inhibitor, a neutrophil function inhibitor, a TNF inhibitor, an IL1 inhibitor, a Plasma cell inhibitor or any combination thereof. In certain embodiments, the treatment for, group B lupus disease state comprises Belimumab, Dasatinib, Roflumilast and/or Apremilast; group C lupus disease state comprises Anifrolumab, Anakinra, Canakinumab, Adalimumab, Certolizumab pegol, Etanercept, Golimumab, Infiximab, Dasatinib, Roflumilast, Apremilast, or any combination thereof; group D lupus disease state comprises Belimumab, Rituximab, Obinutuzumab, Ocrelizumab, Ofatumumab, Inebilizumab, Anifrolumab, Mycophenolate, AZA, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast or any combination thereof; group E lupus disease state comprises Anifrolumab, Mycophenolate, Bortezomib, Isatuximab, Elotuzumab, Carfilzomib, Ixazomib, Daratumumab, Adalimumab, Certolizumab pegol, Etanercept, Golimumab, Infiximab, Dasatinib, Roflumilast, Apremilast or any combination thereof; group F lupus disease state comprises Anifrolumab, Anakinra, Canakinumab, Adalimumab, Certolizumab pegol, Etanercept, Golimumab, Infiximab, Dasatinib, Roflumilast, Apremilast, Belimumab or any combination thereof; group G lupus disease state comprises Belimumab, Rituximab, Obinutuzumab, Ocrelizumab, Ofatumumab, Inebilizumab, Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Infiximab, Dasatinib, Roflumilast, Apremilast or any combination thereof; and/or group H lupus disease state comprises Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast, Belimumab or any combination thereof.

Another aspect of the present disclosure is directed to a use of a data set described above and elsewhere herein.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

The current disclosure includes the following aspects.

Aspect 1 is directed to a method for assessing a lupus state of a patient, wherein the method comprises, analyzing a data set comprising gene expression measurements of at least 2 genes selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32; to generate an inference indicating the lupus state of the patient; wherein the gene expression measurements are obtained from a biological sample of the patient.

Aspect 2 is directed to the method of aspect 1, wherein the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980 or 989 genes.

Aspect 3 is directed to the method of aspect 1 or 2, wherein the at least 2 genes comprise at least 1 gene from each of Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32.

Aspect 4 is directed to method of any one of aspects 1 to 3, wherein the gene expression measurements comprise an enrichment score.

Aspect 5 is directed to the method of aspect 4, wherein the enrichment score is generated using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof.

Aspect 6 is directed to the method of aspect 5, wherein the enrichment score is generated using GSVA.

Aspect 7 is directed to the method of aspect 6, wherein the enrichment score comprises at least one GSVA score from (e.g., generated based on) each of the Tables selected from Table: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32, wherein for a respective Table the at least one GSVA score is generated for enrichment (e.g., in the biological sample obtained or derived from the patient) of at least one gene listed in the respective table.

Aspect 8 is directed to the method of any one of aspects 1 to 7, wherein the analyzing comprises providing the data set as an input to a trained machine-learning model trained to generate the inference.

Aspect 9 is directed to the method of aspect 8, wherein the trained machine-learning model is developed (e.g., trained) using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 10 is directed to the method of any one of aspects 1 to 9, wherein the inference comprises classification whether the patient has lupus.

Aspect 11 is directed to the method of aspect 10, wherein the inference comprises a confidence value between 0 and 1 that the patient has lupus.

Aspect 12 is directed to the method of any one of aspects 1 to 9, wherein the inference comprises classification whether the patient has active lupus or inactive lupus.

Aspect 13 is directed to the method of aspect 12, wherein the inference comprises a confidence value between 0 and 1 that the patient has active lupus.

Aspect 14 is directed to the method of any one of aspects 1 to 9, wherein the inference comprises classification of the patient to an endotype group shown in FIG. 7, or FIG. 32A.

Aspect 15 is directed to the method of any one of aspects 1 to 14, wherein the classification of the lupus state of the patient has, an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 16 is directed to the method of any one of aspects 1 to 15, wherein the classification of the lupus state of the patient has, a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 17 is directed to the method of any one of aspects 1 to 16, wherein the classification of the lupus state of the patient has, a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 18 is directed to the method of any one of aspects 1 to 17, wherein the classification of the lupus state of the patient has, a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 19 is directed to the method of any one of aspects 1 to 18, wherein the classification of the lupus state of the patient has, a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 20 is directed to the method of any one of aspects 1 to 19, comprising classifying the lupus state of the patient with a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The ROC curve of the trained machine learning model of any one of aspects 8 to 19 can have the AUC values (e.g., of aspect 20).

Aspect 21 is directed to the method of any one of aspects 1 to 20, wherein the analyzing comprises calculating a risk score for the patient based at least on the gene expression measurements of the at least 2 genes, and generating the inference at least on the risk score of the patient.

Aspect 22 is directed to the method of any one of aspects 1 to 21, wherein the biological sample is a blood sample, isolated peripheral blood mononuclear cells (PBMCs), tissue biopsy sample, nasal fluid, saliva, urine, stool, or any derivative thereof.

Aspect 23 is directed to the method of any one of aspects 1 to 21, wherein the biological sample is a blood sample, isolated peripheral blood mononuclear cells (PBMCs), or any derivative thereof.

Aspect 24 is directed to the method of any one of aspects 1 to 23, further comprising administering a treatment for lupus to the patient based on the inference.

Aspect 25 is directed to the method of aspect 24, wherein the treatment is configured to treat lupus, in the patient.

Aspect 26 is directed to the method of aspect 24, wherein the treatment is configured to reduce severity of lupus, in the patient.

Aspect 27 is directed to the method of aspect 24, wherein the treatment is configured to reduce the patient's risk of developing lupus.

Aspect 28 is directed to the method of any one of aspects 24 to 27, wherein the treatment comprises a pharmaceutical composition.

Aspect 29 is directed to the method of any one of aspects 24 to 28, wherein the treatment comprises Belimumab, Prednisone, Mycophenolate such as Mycophenolate mofetil, Azathioprine, Voclosporin, Cyclophosphamide, Methylprednisolone, Anifrolumab, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Rituximab, Obinutuzumab, Ocrelizumab, Ofatumumab, Inebilizumab, or any combination thereof.

Aspect 30 is directed to the method of any one of aspects 24 to 29, wherein the treatment comprises Belimumab.

Aspect 31 is directed to a method for identifying a patient as a candidate for treatment with a lupus drug, the method comprising: analyzing a data set comprising gene expression measurements of at least 2 genes selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32; to generate an inference on whether the patient is a candidate for treatment with the lupus drug, wherein the gene expression measurements are obtained from a biological sample of the patient.

Aspect 32 is directed to the method of aspect 31, wherein the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, or 989 genes.

Aspect 33 is directed to the method of any one of aspects 31 or 32, wherein the at least 2 genes comprise at least 1 gene from each of Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32.

Aspect 34 is directed to the method of any one of aspects 31 to 33, wherein the gene expression measurements comprise an enrichment score.

Aspect 35 is directed to the method of aspect 34, wherein the enrichment score is generated using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof.

Aspect 36 is directed to the method of aspect 35, wherein the enrichment score is generated using GSVA.

Aspect 37 is directed to the method of aspect 36, wherein the enrichment score comprises at least one GSVA score from (e.g., generated based on) each of the Tables selected from Table: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32; wherein for a respective Table the at least one GSVA score is generated for enrichment (e.g., in the biological sample obtained or derived from the patient) of at least one gene listed in the respective table.

Aspect 38 is directed to method of any one of aspects 31 to 37, wherein the analyzing comprises providing the data set as an input to a trained machine-learning model trained to generate the inference.

Aspect 39 is directed to the method of aspect 38, wherein the trained machine-learning model is developed (e.g., trained) using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 40 is directed to the method of any one of aspects 31 to 39, wherein the inference comprises classification that the patient is a candidate for treatment with the lupus drug.

Aspect 41 is directed to the method of any one of aspects 31 to 40, wherein the inference comprises a confidence value between 0 and 1 that the patient is a candidate for treatment with the lupus drug.

Aspect 42 is directed to the method of any one of aspects 31 to 41, wherein the classifying that the patient is a candidate for treatment with the lupus drug has an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 43 is directed to the method of any one of aspects 31 to 42, wherein the classifying that the patient is a candidate for treatment with the lupus drug has a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 44 is directed to the method of any one of aspects 31 to 43, wherein the classifying that the patient is a candidate for treatment with the lupus drug has a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 45 is directed to the method of any one of aspects 31 to 44, wherein the classifying that the patient is a candidate for treatment with the lupus drug has a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 46 is directed to the method of any one of aspects 31 to 45, wherein the classifying that the patient is a candidate for treatment with the lupus drug has a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 47 is directed to the method of any one of aspects 31 to 46, wherein the trained machine learning model classify that the patient is a candidate for treatment with the lupus drug with a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The ROC curve of the trained machine learning model of any one of aspects 38 to 46 can have the AUC values (e.g., of aspect 20).

Aspect 48 is directed to the method of any one of aspects 31 to 47, wherein the analyzing comprises calculating a risk score for the patient based at least on the gene expression measurements of the at least 2 genes, and the inference is generated based at least on the risk score of the patient.

Aspect 49 is directed to the method of any one of aspects 31 to 48, wherein the biological sample is a blood sample, isolated peripheral blood mononuclear cells (PBMCs), tissue biopsy sample, nasal fluid, saliva, urine, stool, or any derivative thereof.

Aspect 50 is directed to the method of any one of aspects 31 to 49, wherein the biological sample is a blood sample, isolated peripheral blood mononuclear cells (PBMCs), or any derivative thereof.

Aspect 51 is directed to the method of any one of aspects 31 to 50, further comprising administering to the patient the lupus drug based on the inference that the patient is a candidate for treatment with the lupus drug.

Aspect 52 is directed to the method of any one of aspects 31 to 51, wherein the lupus drug comprises Belimumab, Prednisone, Mycophenolate such as Mycophenolate mofetil, Azathioprine, Voclosporin, Cyclophosphamide, Methylprednisolone, Anifrolumab, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Rituximab, Obinutuzumab, Ocrelizumab, Ofatumumab, Inebilizumab, or any combination thereof.

Aspect 53 is directed to the method of any one of aspects 31 to 51, wherein the lupus drug comprises belimumab.

Aspect 54 is directed to a method for developing a biomarker assay for identifying a treatment candidate for a lupus drug, the method comprising:
(a) obtaining a reference data set comprising a plurality of individual reference data sets, wherein a respective individual reference data set of the plurality of individual reference data sets comprises i) gene expression measurements of at least 2 genes selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32, of a reference patient, and ii) data regarding the reference patient's one or more lupus disease index, at a time point before administering, and at least one time point after administering the lupus drug to the reference patient;
(b) training a machine learning model using the reference data set, wherein the machine learning model is trained to infer a training patient's response to the lupus drug based on gene expression measurements of the at least 2 genes of step (a) of training patient, at a time point before administering, and at least one time point after administering the lupus drug to the training patient;
(c) determining feature importance values of one or more predictors of the machine learning model, wherein the one or more predictors comprises the at least 2 genes of step (a);
(d) selecting 2 to 30 gene predictors of the machine learning model based at least on the feature importance values determined in step (c); and
(e) developing an assay capable of measuring expression and/or encoding of the 2 to 30 genes selected in step (d) in a biological sample, to obtain the biomarker assay.

Aspect 55 is directed to the method of aspect 54, wherein the 2 to 30 gene predictors of the machine learning model selected in step (d) has top 2 to 30 feature importance values determined in step (c).

Aspect 56 is directed to the method of aspect 54 or 55, wherein the one or more lupus disease index, comprises blood anti-double-stranded DNA antibody level, blood anti-ribonucleoprotein (RNP) antibody level, blood complement component 3 (C3) protein level, blood complement component 4 (C4) protein level, SLEDAI score, LuMOS score, or any combination thereof.

Aspect 57 is directed to the method of any one aspects 55 to 56, wherein the training patient's response to the lupus drug comprises a measurement of change of the training patient's blood anti-double-stranded DNA antibody level, blood anti-ribonucleoprotein (RNP) antibody level, blood complement component 3 (C3) protein level, blood complement component 4 (C4) protein level, SLEDAI score, LuMOS score, or any combination thereof, between the time point before administration, and the at least one time point after administration of the lupus drug to the training patient.

Aspect 58 is directed to the method of any one aspects 55 to 57, wherein the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, or 989 genes.

Aspect 59 is directed to the method of any one aspects 55 to 58, wherein the at least 2 genes comprise at least 1 gene from each of Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; or 32.

Aspect 60 is directed to the method of any one aspects 55 to 59, wherein the trained machine-learning model is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 61 is directed to the method of any one of aspects 54 to 60, wherein the lupus drug comprises at least one drug approved for treatment of lupus, at least one experimental lupus drug, or a combination thereof.

Aspect 62 is directed to the method of any one aspects 54 to 61, wherein the lupus drug comprises Belimumab, Prednisone, Mycophenolate such as Mycophenolate mofetil, Azathioprine, Voclosporin, Cyclophosphamide, Methylprednisolone, Anifrolumab, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Rituximab, Obinutuzumab, Ocrelizumab, Ofatumumab, Inebilizumab, or any combination thereof.

Aspect 63 is directed to the method of any one aspects 54 to 62, wherein the lupus drug comprises belimumab.

Aspect 64 is directed to the method of any one aspects 54 to 63, wherein the biological sample is a blood sample, isolated peripheral blood mononuclear cells (PBMCs), or any derivative thereof.

Aspect 65 is directed a biomarker assay developed according to the method of any one of aspects 54 to 64.

Aspect 66 is directed to a kit comprising a biomarker assay developed according to the method of any one of aspects 54 to 64, and/or a biomarker assay of aspect 65.

Aspect 67 is directed to use of a method of any one of aspects 1-63, a biomarker assay of aspect 65, or a kit of aspect 66, to assess a lupus state of a patient, identify a treatment for a patient having lupus, identify a treatment for a patient at risk of developing lupus, or both.

Aspect 68 is directed to a method for treating lupus in a patient, the method comprising:
a) providing a data set comprising or derived from gene expression measurements of effective number of genes selected from genes listed in each of 23 or more Tables selected from Tables: 1 to 32, as an input to a trained machine-learning model trained to generate an inference of whether the data set is indicative of the patient having group A lupus disease state, group B lupus disease state, group C lupus disease state, group D lupus disease state, group E lupus disease state, group F lupus disease state, group G lupus disease state, or group H lupus disease state;
b) receiving, as an output of the trained machine-learning model, the inference;
c) electronically outputting a report classifying the lupus disease state of the patient based on the inference; and
d) administering a treatment to the patient based on the inference,
   wherein the gene expression measurements are obtained from a biological sample obtained or derived from the patient, and wherein the lupus disease state of the patient is classified with an accuracy of at least about 90%, a sensitivity of at least about 90%, a specificity of at least about 90%, a positive predictive value of at least about 90%, and a negative predictive value of at least about 90%.

Aspect 69 is directed to the method of aspect 68, wherein the lupus disease state of the patient is classified with an accuracy of at least about 95%, a sensitivity of at least about 95%, a specificity of at least about 95%, a positive predictive value of at least about 95%, a negative predictive value of at least about 95%, or any combination thereof.

Aspect 70 is directed to the method of aspect 68 or 69, wherein the lupus disease state of the patient is classified with an accuracy of at least about 98%, a sensitivity of at least about 98%, a specificity of at least about 98%, a positive predictive value of at least about 98%, a negative predictive value of at least about 98%, or any combination thereof.

Aspect 71 is directed to any one of aspects 68 to 70, wherein the 23 or more Tables selected comprises Tables: 2; 4; 5; 6; 7; 8; 10; 11; 12; 13; 14; 15; 16; 17; 18; 19; 20; 22; 23; 24; 25; 31; and 32.

Aspect 72 is directed to any one of aspects 68 to 70, wherein the 23 or more Tables selected comprises Tables: 1; 2; 3; 4; 5; 6; 7; 8; 10; 11; 12; 13; 14; 15; 16; 17; 18; 19; 20; 21; 22; 23; 24; 25; 31; and 32.

Aspect 73 is directed to any one of aspects 68 to 72, wherein each of Tables: 1 to 32, are selected.

Aspect 74 is directed to any one of aspects 68 to 73, wherein the data set comprises or is derived from gene expression measurements of all the genes listed in the Tables selected.

Aspect 75 is directed to any one of aspects 68 to 74, wherein the data set is derived from the gene expression measurements using GSVA, gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

Aspect 76 is directed to any one of aspects 68 to 74, wherein the data set is derived from the gene expression measurements using GSVA.

Aspect 77 is directed to aspect 76, wherein the data set comprises 23 or more GSVA scores of the patient, each generated based on one of the 23 or more selected Tables, wherein for each selected Table, the effective number of genes selected from the selected Table forms an input gene set for generating the GSVA score based on the selected Table using GSVA. Enrichment of the input gene set in the biological sample is measured to generate the GSVA score. Enrichment can be measured with respect to a reference data set, as described herein.

Aspect 78 is directed to any one of aspects 68 to 77, wherein the trained machine-learning model is trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 79 is directed to any one of aspects 68 to 78, wherein the inference comprises a confidence value between 0 and 1 that the patient has the group A lupus disease state, the group B lupus disease state, the group C lupus disease state, the group D lupus disease state, the group E lupus disease state, the group F lupus disease state, the group G lupus disease state, or the group H lupus disease state.

Aspect 80 is directed to any one of aspects 68 to 79, wherein the trained machine-learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 81 is directed to any one of aspects 68 to 80, wherein the biological sample is a blood sample, isolated peripheral blood mononuclear cells (PBMCs), or any derivative thereof.

Aspect 82 is directed to any one of aspects 68 to 81, wherein method further comprises performing Shapley Additive Explanations (SHAP) on the data set to determine contribution of one or more gene features to the inference.

Aspect 83 is directed to aspect 82, wherein the treatment administered is selected based on the contribution of the one or more gene features to the inference.

Aspect 84 is directed to any one of aspects 68 to 83, wherein the treatment administered comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof.

Aspect 85 is directed to any one of aspects 68 to 83, wherein the treatment administered comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof.

Aspect 86 is directed to any one of aspects, 68 to 83, wherein the treatment for, group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, NK cell inhibitor, a neutrophil function inhibitor, a TNF inhibitor, an IL1 inhibitor, a Plasma cell inhibitor or any combination thereof; group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof and/or group H lupus disease state comprises an IFN inhibitor, a neutrophil function inhibitor, a TNF inhibitor, an IL1 inhibitor, a Plasma cell inhibitor or any combination thereof.

Aspect 87 is directed to any one of aspects 68 to 83, wherein the treatment for, group B lupus disease state, Dasatinib, and/or Apremilast; group C lupus disease state comprises Anifrolumab, Anakinra, Adalimumab, Certolizumab pegol, Etanercept, Golimumab, Inflximab, Dasatinib, Apremilast, or any combination thereof; group D lupus disease state comprises Belimumab, Anifrolumab, Mycophenolate, AZA, Bortezomib, Isatuximab, Elotuzumab, Anakinra, Adalimumab, Certolizumab pegol, Etanercept, Golimumab, Infiximab, Dasatinib, Apremilast or any combination thereof; group E lupus disease state comprises Anifrolumab, Mycophenolate, Bortezomib, Isatuximab, Elotuzumab, Adalimumab, Certolizumab pegol, Etanercept, Golimumab, Inflximab, Dasatinib, Apremilast or any combination thereof; group F lupus disease state comprises Anifrolumab, Anakinra, Adalimumab, Certolizumab pegol, Etanercept, Golimumab, Inflximab, Dasatinib, Apremilast or any combination thereof; group G lupus disease state comprises Belimumab, Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast or any combination thereof; and/or group H lupus disease state comprises Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast or any combination thereof.

Aspect 88 is directed to any one of aspects 68 to 87, wherein the patient has lupus, is at elevated risk of having lupus, is suspected of having lupus, and/or is asymptomatic for lupus.

Aspect 89 is directed to any one of aspects 68 to 88, wherein the trained machine learning model is trained by at least:
a. determining gene set variation analysis (GSVA) scores for a reference data set comprising lupus samples and healthy samples, the reference data set comprising gene expression measurements of the 62 gene signatures shown in FIG. 14,
b. training a first machine-learning model based on the GSVA scores for the reference data set to generate first inferences of whether the samples of the reference data set are indicative of having lupus or not having lupus,
c. determining a first set of features of the first machine-learning model based on importance of the first set of features to the first inferences,
d. training a second machine-learning model based on the GSVA scores of the lupus samples of the reference data set to generate second inferences of whether the lupus samples of the reference data set are indicative of having active lupus or inactive lupus,
e. determining second set of features of the second machine-learning model based on importance of the second set of features to the second inferences,
f. determining a third set of features based on overlap of the first set of features and the second set of features, and
g. determining the trained machine-learning model based on GSVA scores of the third set of features to generate third inferences of whether the samples of the reference data set are indicative having group A lupus disease state, group B lupus disease state, group C lupus disease state, group D lupus disease state, group E lupus disease state, group F lupus disease state, group G lupus disease state, or group H lupus disease state.

Aspect 90 is directed to a method for classifying a lupus disease state of a patient, the method comprising: analyzing a data set comprising or derived from gene expression measurements of at least 2 genes selected from genes listed in each of one or more Tables selected from Tables: 1 to 32, to classify the lupus disease state of the patient, wherein the gene expression measurements are obtained from a biological sample obtained or derived from the patient.

Aspect 91 is directed to the method of aspect 90, wherein the lupus disease state of the patient is classified as group A lupus disease state, group B lupus disease state, group C lupus disease state, group D lupus disease state, group E lupus disease state, group F lupus disease state, group G lupus disease state, or group H lupus disease state.

Aspect 92 is directed to the method of aspect 90 or 91, wherein the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, or all genes selected from genes listed in each of the one or more Tables selected from Tables: 1 to 32, wherein the number of genes selected from different selected Tables may be the same or different.

Aspect 93 is directed to the method of aspect 90 or 91, wherein the data set comprises or is derived from gene expression measurements of an effective number of genes selected from genes listed in each of the one or more Tables selected from Tables: 1 to 32, wherein the number of genes selected from different selected Tables may be the same or different.

Aspect 94 is directed to the method of any one of aspects 90 to 93, wherein at least 23 Tables are selected from Tables: 1 to 32.

Aspect 95 is directed to the method of any one of aspects 90 to 94, wherein at least 28 Tables are selected from Tables: 1 to 32.

Aspect 96 is directed to the method of any one of aspects 90 to 95, wherein Tables: 1 to 32 are selected.

Aspect 97 is directed to the method of any one of aspects 90 to 96, wherein the data set comprises or is derived from gene expression measurements of all the genes listed in the Tables selected.

Aspect 98 is directed to the method of any one of aspects 90 to 97, wherein the method classify the lupus disease state of the patient with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 99 is directed to the method of any one of aspects 90 to 98, wherein the method classify the lupus disease state of the patient with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 100 is directed to the method of any one of aspects 90 to 99, wherein the method classify the lupus disease state of the patient with specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 101 is directed to the method of any one of aspects 90 to 100, wherein the method classify the lupus disease state of the patient with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 102 is directed to the method of any one of aspects 90 to 101, wherein the method classify the lupus disease state of the patient with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 103 is directed to the method of any one of aspects 90 to 102, wherein the data set is derived from the gene expression measurements using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

Aspect 104 is directed to the method of any one of aspects 90 to 102, wherein the data set is derived from the gene expression measurements using GSVA.

Aspect 105 is directed to the method of aspect 104, wherein the data set comprises one or more GSVA scores of the patient, wherein the one or more GSVA scores are generated based on the one or more Tables selected from Tables 1 to 32, wherein for each selected Table the at least 2 genes selected from the selected Table forms an input gene set for generating a GSVA score based on the selected Table using GSVA, and wherein the one or more GSVA scores comprise the generated GSVA scores. Enrichment of the input gene set in the biological sample is measured to generate the GSVA score. Enrichment can be measured with respect to a reference data set, as described herein.

Aspect 106 is directed to the method of any one of aspects 104 to 105, wherein for each selected Table the effective number of genes selected from the selected Table forms the input gene set for generating the GSVA score based on the selected Table.

Aspect 107 is directed to the method of any one of aspects 90 to 106, wherein analyzing the data set comprises providing the data set as an input to a trained machine-learning model trained to generate an inference of whether the data set is indicative of the patient having group A lupus disease state, group B lupus disease state, group C lupus disease state, group D lupus disease state, group E lupus disease state, group F lupus disease state, group G lupus disease state, or group H lupus disease state, wherein the method classify the lupus disease state of the patient based on the inference of the trained machine-learning model.

Aspect 108 is directed to the method of aspect 107, further comprising: a) receiving, as an output of the trained machine-learning model, the inference; and b) electronically outputting a report classifying the lupus disease state of a patient.

Aspect 109 is directed to the method of any one of aspects 107 or 108, wherein the trained machine-learning model is trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), Classification and Regression Tree (CART), hierarchical clustering, or any combination thereof.

Aspect 110 is directed to the method of any one of aspects 107 to 109, wherein the inference comprises a confidence value between 0 and 1 that the patient has the group A lupus disease state, the group B lupus disease state, the group C lupus disease state, the group D lupus disease state, the group E lupus disease state, the group F lupus disease state, group G lupus disease state, or the group H lupus disease state.

Aspect 111 is directed to the method of any one of aspects 107 to 110, wherein the trained machine-learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 112 is directed to the method of any one of aspects 90 to 106, wherein analyzing the data set comprises generating a risk score of the patient based on the data set, wherein the method classify the lupus disease state of the patient based on the risk score.

Aspect 113 is directed to the method of aspect 112, wherein the risk score of the patient is based on the one or more GSVA scores of the patient.

Aspect 114 is directed to the method of any one of aspects 90 to 113, wherein the method further comprises performing Shapley Additive Explanations (SHAP) analysis on the data set to determine contribution of one or more gene features to the lupus disease state classification of the patient.

Aspect 115 is directed to the method of any one of aspects 90 to 114, wherein the biological sample comprises a blood sample, isolated peripheral blood mononuclear cells (PBMCs), a tissue biopsy sample, or any derivative thereof.

Aspect 116 is directed to the method of any one of aspects 90 to 115, wherein the patient has lupus.

Aspect 117 is directed to the method of any one of aspects 90 to 115, wherein the patient is at elevated risk of having lupus.

Aspect 118 is directed to the method of any one of aspects 90 to 116, wherein the patient is asymptomatic for lupus.

Aspect 119 is directed to the method of any one of aspects 90 to 118, further comprising selecting, recommending and/or administering a treatment to the patient based on the classification of the lupus disease state of the patient.

Aspect 120 is directed to the method of aspect 119, wherein the treatment is configured to treat lupus.

Aspect 121 is directed to the method of aspect 119, wherein the treatment is configured to treat reduce severity of lupus.

Aspect 122 is directed to the method of aspect 119, wherein the treatment is configured to reduce risk of having lupus.

Aspect 123 is directed to the method of any one of aspects 119 to 122, wherein the treatment comprises one or more pharmaceutical compositions.

Aspect 124 is directed to the method of any one of aspects 119 to 123, wherein the treatment is based on the contribution of the one or more gene features to the lupus disease state classification of the patient.

Aspect 125 is directed to the method of any one of aspects 119 to 123, wherein the treatment targets one or more gene features significantly enriched in the biological sample.

Aspect 126 is directed to the method of any one of aspects 119 to 125, wherein the treatment comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof.

Aspect 127 is directed to the method of any one of aspects 119 to 126, wherein the treatment comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof.

Aspect 128 is directed to the method of any one of aspects 119 to 127, wherein the treatment for, group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, a NK cell inhibitor, a neutrophil function inhibitor, a TNF inhibitor, an IL1 inhibitor, a Plasma cell inhibitor or any combination thereof; group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof; and/or group H lupus disease state comprises an IFN inhibitor, a neutrophil function inhibitor, a TNF inhibitor, an IL1 inhibitor, a Plasma cell inhibitor or any combination thereof.

Aspect 129 is directed to the method of any one of aspects 119 to 128, wherein the treatment for, group B lupus disease state comprises Belimumab, Dasatinib, and/or Apremilast; group C lupus disease state comprises Anifrolumab, Anakinra, Adalimumab, Certolizumab pegol, Etanercept, Golimumab, Inflximab, Dasatinib, Apremilast, or any combination thereof; group D lupus disease state comprises Belimumab, Anifrolumab, Mycophenolate, AZA, Bortezomib, Isatuximab, Elotuzumab, Anakinra, Adalimumab, Certolizumab pegol, Etanercept, Golimumab, Infiximab, Dasatinib, Apremilast or any combination thereof; group E lupus disease state comprises Anifrolumab, Mycophenolate, Bortezomib, Isatuximab, Elotuzumab, Adalimumab, Certolizumab pegol, Etanercept, Golimumab, Inflximab, Dasatinib, Apremilast or any combination thereof; group F lupus disease state comprises Anifrolumab, Anakinra, Adalimumab, Certolizumab pegol, Etanercept, Golimumab, Infiximab, Dasatinib, Apremilast, Belimumab, or any combination thereof; group G lupus disease state comprises Belimumab, Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast or any combination thereof and group H lupus disease state comprises Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast, Belimumab, or any combination thereof.

Aspect 130 is directed to a method for assessing a SSc disease state of a patient, the method comprising: analyzing a data set comprising or derived from gene expression measurements data of at least 2 genes selected from the genes listed in Tables 1 to 32, in a biological sample obtained or derived from the patient, to classify the SSc disease state of the patient.

Aspect 131 is directed to a method of aspect 130, wherein the SSc disease state of the patient is classified as group 1, group 2, group 3 or group 4 SSc disease state.

Aspect 132 is directed to the method of aspect 130 or 131, wherein the data set comprises or is derived from gene expression measurements data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes listed in Tables 1-32, in the biological sample obtained or derived from the patient.

Aspect 133 is directed to the method of any one of aspects 130 to 132, wherein the data set comprises or is derived from gene expression measurements data of at least 2 to all, or any value or range there between, genes selected from the genes listed in each of one or more Tables selected from Tables 1 to 32, in the biological sample obtained or derived from the patient, wherein number of genes selected from the genes in each selected table may be different or same.

Aspect 134 is directed to the method of aspect 133, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, Tables selected from Tables 1 to 32.

Aspect 135 is directed to the method of any one of aspects 133 to 134, wherein the selected Tables are Tables 1 to 32.

Aspect 136 is directed to the method of any one of aspects 130 to 135, wherein the SSc disease state of the patient is classified with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 137 is directed to the method of any one of aspects 130 to 136, wherein the SSc disease state of the patient is classified with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 138 is directed to the method of any one of aspects 130 to 137, wherein the SSc disease state of the patient is classified with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 139 is directed to the method of any one of aspects 130 to 138, wherein the SSc disease state of the patient is classified with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 140 is directed to the method of any one of aspects 130 to 139, wherein the SSc disease state of the patient is classified with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 141 is directed to the method of any one of aspects 130 to 140, wherein the SSc disease state of the patient is classified with a Receiver operating characteristic (ROC) curve having an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 142 is directed to the method of any one of aspects 130 to 141, wherein the data set is derived from the gene expression measurements data using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

Aspect 143 is directed to the method of any one of aspects 130 to 141, wherein the data set is derived from the gene expression measurements data using GSVA.

Aspect 144 is directed to the method of aspect 143, wherein the data set comprises one or more GSVA scores of the patient, wherein the one or more GSVA scores are generated based on one or more Tables selected from Tables 1 to 32, wherein for each selected Table, at least one GSVA score of the patient is generated based on enrichment of expression of at least 2 genes thereof listed in the selected Table in the biological sample, and wherein the one or more GSVA scores comprise each generated GSVA score. Enrichment can be measured with respect to a reference data set.

Aspect 145 is directed to the method of aspect 144, wherein for each selected Table, the at least one GSVA score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 genes listed in the respective Table, wherein number of genes selected from different selected Tables can be same or different.

Aspect 146 is directed to the method of any one of aspects 130 to 145, wherein the analyzing the data set comprises providing the data set as an input to a trained machine-learning model to classify the SSc disease state of the patient, wherein the trained machine-learning model generate an inference indicative of the SSc disease state of the patient based at least on the data set.

Aspect 147 is directed to the method of aspect 146, wherein the data set comprises the one or more GSVA scores of the patient, and the trained machine-learning model generate the inference based at least on the one or more GSVA scores.

Aspect 148 is directed to the method of any one of aspects 146 to 147, wherein the method further comprises receiving, as an output of the trained machine-learning model, the inference; and/or electronically outputting a report indicating the SSc disease state of the patient.

Aspect 149 is directed to the method of any one of aspects 146 to 148, wherein the machine-learning model is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

Aspect 150 is directed to the method of any one of aspects 130 to 149, the patient is at elevated risk of having, is suspected of having, is asymptomatic for, and/or has SSc.

Aspect 151 is directed to the method of any one of aspects 130 to 150, further comprising selecting, recommending and/or administering a treatment to the patient based at least in part on the classification of the SSc disease state of the patient.

Aspect 152 is directed to the method of aspect 151, wherein the treatment is configured to treat, reduce a severity of lupus nephritis, and/or reduce a risk of having SSc.

Aspect 153 is directed to the method of any one of aspects 151 to 152, wherein the treatment comprises a pharmaceutical composition.

Aspect 154 is directed to the method of any one of aspects 151 to 152, wherein the treatment for SSc comprises an agent that targets TGFB fibroblasts (e.g., nintedanib, pirfenidone), and/or dendritic cells (e.g., BIIB059, Daxdilmab).

Aspect 155 is directed to the method of any one of aspects 130 to 154, wherein the biological sample comprises a blood sample, isolated peripheral blood mononuclear cells (PBMCs), skin biopsy sample, or any derivative thereof.

Aspect 156 is directed to a method for classifying a lupus disease state of a patient, the method comprising: analyzing a data set comprising gene expression measurements of at least 2 genes selected from the group of genes ATP5A1, CD247, COX15, COX6B2, NDUFA9, NDUFB2-AS1, NDUFC2, NDUFS1, NDUFS7, and SH2D1A to classify the lupus disease state of the patient, wherein the gene expression measurements are obtained from a biological sample obtained or derived from the patient, and wherein classifying the lupus disease state of the patient include classifying whether the patient has group A lupus disease state, or group B lupus disease state.

Aspect 157 is directed to the method of aspect 156, wherein the data set comprises or is derived from gene expression measurements of at least 3, 4, 5, 6, 7, 8, 9, or 10 genes selected from the group of genes ATP5A1, CD247, COX15, COX6B2, NDUFA9, NDUFB2-AS1, NDUFC2, NDUFS1, NDUFS7, and SH2D1A.

Aspect 158 is directed to the method of aspect 156 or 157, wherein the data set comprises or is derived from gene expression measurements of ATP5A1, CD247, COX15, COX6B2, NDUFA9, NDUFB2-AS1, NDUFC2, NDUFS1, NDUFS7, and SH2D1A.

Aspect 159 is directed to the method of any one of aspects 156 to 158, wherein analyzing the data set comprises providing the data set as an input to a trained machine-learning model trained to generate an inference of whether the data set is indicative of the patient having group A lupus disease state, or group B lupus disease state, wherein the method classify the lupus disease state of the patient based on the inference of the trained machine-learning model.

Aspect 160 is directed to the method aspect 159, wherein the inference comprises a confidence value between 0 and 1 that the patient has the group A lupus disease state, or the group B lupus disease state.

Aspect 161 is directed to a method for classifying a lupus disease state of a patient, the method comprising: analyzing a data set comprising gene expression measurements of at least 2 genes selected from the group of genes ADGRE2, AOAH, BACH1, CLEC4D, CLEC7A, FFAR2, LILRA6, LMNB1, TLR2, and TNFRSF1B, to classify the lupus disease state of the patient, wherein the gene expression measurements are obtained from a biological sample obtained or derived from the patient, and wherein classifying the lupus disease state of the patient include classifying whether the patient has group A lupus disease state, or group C lupus disease state.

Aspect 162 is directed to the method of aspect 161, wherein the data set comprises or is derived from gene expression measurements of at least 3, 4, 5, 6, 7, 8, 9, or 10 genes selected from the group of genes ADGRE2, AOAH, BACH1, CLEC4D, CLEC7A, FFAR2, LILRA6, LMNB1, TLR2, and TNFRSF1B.

Aspect 163 is directed to the method of aspect 161 or 162, wherein the data set comprises or is derived from gene expression measurements of ADGRE2, AOAH, BACH1, CLEC4D, CLEC7A, FFAR2, LILRA6, LMNB1, TLR2, and TNFRSF1B.

Aspect 164 is directed to the method of any one of aspects 161 to 163, wherein analyzing the data set comprises providing the data set as an input to a trained machine-learning model trained to generate an inference of whether the data set is indicative of the patient having group A lupus disease state, or group C lupus disease state, wherein the method classify the lupus disease state of the patient based on the inference of the trained machine-learning model.

Aspect 165 is directed to the method aspect 164, wherein the inference comprises a confidence value between 0 and 1 that the patient has the group A lupus disease state, or the group C lupus disease state.

Aspect 166 is directed to a method for classifying a lupus disease state of a patient, the method comprising: analyzing a data set comprising gene expression measurements of at least 2 genes selected from the group of genes ACLY, ARSE, CASP1, CASP10, CTNND2, EIF2AK2, GBP1, IFI30, IL1RN and PSMB8, to classify the lupus disease state of the patient, wherein the gene expression measurements are obtained from a biological sample obtained or derived from the patient, and wherein classifying the lupus disease state of the patient include classifying whether the patient has group A lupus disease state, or group D lupus disease state.

Aspect 167 is directed to the method of aspect 166, wherein the data set comprises or is derived from gene expression measurements of at least 3, 4, 5, 6, 7, 8, 9, or 10 genes selected from the group of genes ACLY, ARSE, CASP1, CASP10, CTNND2, EIF2AK2, GBP1, IFI30, IL1RN and PSMB8.

Aspect 168 is directed to the method of aspect 166 or 167, wherein the data set comprises or is derived from gene expression measurements of ACLY, ARSE, CASP1, CASP10, CTNND2, EIF2AK2, GBP1, IF130, IL1RN and PSMB8.

Aspect 169 is directed to the method of any one of aspects 166 to 168, wherein analyzing the data set comprises providing the data set as an input to a trained machine-learning model trained to generate an inference of whether the data set is indicative of the patient having group A lupus disease state, or group D lupus disease state, wherein the method classify the lupus disease state of the patient based on the inference of the trained machine-learning model.

Aspect 170 is directed to the method aspect 169, wherein the inference comprises a confidence value between 0 and 1 that the patient has the group A lupus disease state, or the group D lupus disease state.

Aspect 171 is directed to a method for classifying a lupus disease state of a patient, the method comprising: analyzing a data set comprising gene expression measurements of at least 2 genes selected from the group of genes AURKB, CCNE1, EIF2AK2, GBP2, IFITM3, IGHG1, IGLV4-60, IGLV5-45, ISG20, and PTTG1, to classify the lupus disease state of the patient, wherein the gene expression measurements are obtained from a biological sample obtained or derived from the patient, and wherein classifying the lupus disease state of the patient include classifying whether the patient has group A lupus disease state, or group E lupus disease state.

Aspect 172 is directed to the method of aspect 171, wherein the data set comprises or is derived from gene expression measurements of at least 3, 4, 5, 6, 7, 8, 9, or 10 genes selected from the group of genes AURKB, CCNE1, EIF2AK2, GBP2, IFITM3, IGHG1, IGLV4-60, IGLV5-45, ISG20, and PTTG1.

Aspect 173 is directed to the method of aspect 171 or 172, wherein the data set comprises or is derived from gene expression measurements of AURKB, CCNE1, EIF2AK2, GBP2, IFITM3, IGHG1, IGLV4-60, IGLV5-45, ISG20, and PTTG1.

Aspect 174 is directed to the method of any one of aspects 171 to 173, wherein analyzing the data set comprises providing the data set as an input to a trained machine-learning model trained to generate an inference of whether the data set is indicative of the patient having group A lupus disease state, or group E lupus disease state, wherein the method classify the lupus disease state of the patient based on the inference of the trained machine-learning model.

Aspect 175 is directed to the method aspect 174, wherein the inference comprises a confidence value between 0 and 1 that the patient has the group A lupus disease state, or the group E lupus disease state.

Aspect 176 is directed to a method for classifying a lupus disease state of a patient, the method comprising: analyzing a data set comprising gene expression measurements of at least 2 genes selected from the group of genes CCL28, CD247, CHIT1, CXCL1, FFAR2, LILRB5, LMNB1, PYHIN1, SECTM1, and SIGLEC5, to classify the lupus disease state of the patient, wherein the gene expression measurements are obtained from a biological sample obtained or derived from the patient, and wherein classifying the lupus disease state of the patient include classifying whether the patient has group A lupus disease state, or group F lupus disease state.

Aspect 177 is directed to the method of aspect 176, wherein the data set comprises or is derived from gene expression measurements of at least 3, 4, 5, 6, 7, 8, 9, or 10 genes selected from the group of genes CCL28, CD247, CHIT1, CXCL1, FFAR2, LILRB5, LMNB1, PYHIN1, SECTM1, and SIGLEC5.

Aspect 178 is directed to the method of aspect 176 or 177, wherein the data set comprises or is derived from gene expression measurements of CCL28, CD247, CHIT1, CXCL1, FFAR2, LILRB5, LMNB1, PYHIN1, SECTM1, and SIGLEC5.

Aspect 179 is directed to the method of any one of aspects 176 to 178, wherein analyzing the data set comprises providing the data set as an input to a trained machine-learning model trained to generate an inference of whether the data set is indicative of the patient having group A lupus disease state, or group F lupus disease state, wherein the method classify the lupus disease state of the patient based on the inference of the trained machine-learning model.

Aspect 180 is directed to the method aspect 179, wherein the inference comprises a confidence value between 0 and 1 that the patient has the group A lupus disease state, or the group F lupus disease state.

Aspect 181 is directed to a method for classifying a lupus disease state of a patient, the method comprising: analyzing a data set comprising gene expression measurements of at least 2 genes selected from the group of genes APOBR, CASP1, CASP10, FFAR2, MS4A4A, MTF1, SECTM1, SEMA4A, TLR8, and TNFRSF1B, to classify the lupus disease state of the patient, wherein the gene expression measurements are obtained from a biological sample obtained or derived from the patient, and wherein classifying the lupus disease state of the patient include classifying whether the patient has group A lupus disease state, or group G lupus disease state.

Aspect 182 is directed to the method of aspect 181, wherein the data set comprises or is derived from gene expression measurements of at least 3, 4, 5, 6, 7, 8, 9, or 10 genes selected from the group of genes APOBR, CASP1, CASP10, FFAR2, MS4A4A, MTF1, SECTM1, SEMA4A, TLR8, and TNFRSF1B.

Aspect 183 is directed to the method of aspect 181 or 182, wherein the data set comprises or is derived from gene expression measurements of APOBR, CASP1, CASP10, FFAR2, MS4A4A, MTF1, SECTM1, SEMA4A, TLR8, and TNFRSF1B.

Aspect 184 is directed to the method of any one of aspects 181 to 183, wherein analyzing the data set comprises providing the data set as an input to a trained machine-learning model trained to generate an inference of whether the data set is indicative of the patient having group A lupus disease state, or group G lupus disease state, wherein the method classify the lupus disease state of the patient based on the inference of the trained machine-learning model.

Aspect 185 is directed to the method aspect 184, wherein the inference comprises a confidence value between 0 and 1 that the patient has the group A lupus disease state, or the group G lupus disease state.

Aspect 186 is directed to a method for classifying a lupus disease state of a patient, the method comprising: analyzing a data set comprising gene expression measurements of at least 2 genes selected from the group of genes ADAM8, APOBEC3B, CCL28, CD177, CXCL1, EIF2AK2, FCGR3B, IL10RA, LILRA5, and OSCAR, to classify the lupus disease state of the patient, wherein the gene expression measurements are obtained from a biological sample obtained or derived from the patient, and wherein classifying the lupus disease state of the patient include classifying whether the patient has group A lupus disease state, or group H lupus disease state.

Aspect 187 is directed to the method of aspect 186, wherein the data set comprises or is derived from gene expression measurements of at least 3, 4, 5, 6, 7, 8, 9, or 10 genes selected from the group of genes ADAM8, APOBEC3B, CCL28, CD177, CXCL1, EIF2AK2, FCGR3B, IL10RA, LILRA5, and OSCAR.

Aspect 188 is directed to the method of aspect 186 or 187, wherein the data set comprises or is derived from gene expression measurements of ADAM8, APOBEC3B, CCL28, CD177, CXCL1, EIF2AK2, FCGR3B, IL10RA, LILRA5, and OSCAR.

Aspect 189 is directed to the method of any one of aspects 186 to 188, wherein analyzing the data set comprises providing the data set as an input to a trained machine-learning model trained to generate an inference of whether the data set is indicative of the patient having group A lupus disease state, or group H lupus disease state, wherein the method classify the lupus disease state of the patient based on the inference of the trained machine-learning model.

Aspect 190 is directed to the method aspect Aspect 190 is directed to the method aspect 189, wherein the inference comprises a confidence value between 0 and 1 that the patient has the group A lupus disease state, or the group H lupus disease state.

Aspect 191 is directed to a method for classifying a lupus disease state of a patient, the method comprising: analyzing a data set comprising gene expression measurements of at least 2 genes selected from the group of genes CANX, CASP1, CHUK, DERL2, ERGIC2, HERPUD1, IRAK1, IRAK4, RIPK1, and SEC24D, to classify the lupus disease state of the patient, wherein the gene expression measurements are obtained from a biological sample obtained or derived from the patient, and wherein classifying the lupus disease state of the patient include classifying whether the patient has group B lupus disease state, or group C lupus disease state.

Aspect 192 is directed to the method of aspect 191, wherein the data set comprises or is derived from gene expression measurements of at least 3, 4, 5, 6, 7, 8, 9, or 10 genes selected from the group of genes CANX, CASP1, CHUK, DERL2, ERGIC2, HERPUD1, IRAK1, IRAK4, RIPK1, and SEC24D.

Aspect 193 is directed to the method of aspect 191 or 192, wherein the data set comprises or is derived from gene expression measurements of CANX, CASP1, CHUK, DERL2, ERGIC2, HERPUD1, IRAK1, IRAK4, RIPK1, and SEC24D.

Aspect 194 is directed to the method of any one of aspects 191 to 193, wherein analyzing the data set comprises providing the data set as an input to a trained machine-learning model trained to generate an inference of whether the data set is indicative of the patient having group B lupus disease state, or group C lupus disease state, wherein the method classify the lupus disease state of the patient based on the inference of the trained machine-learning model.

Aspect 195 is directed to the method aspect 194, wherein the inference comprises a confidence value between 0 and 1 that the patient has the group B lupus disease state, or the group C lupus disease state.

Aspect 196 is directed to a method for classifying a lupus disease state of a patient, the method comprising: analyzing a data set comprising gene expression measurements of at least 2 genes selected from the group of genes CALR, EDEM2, EMC9, ERAP1, KDELC1, MANF, NUCB2, PSMB8, SEC24D, and TRDC, to classify the lupus disease state of the patient, wherein the gene expression measurements are obtained from a biological sample obtained or derived from the patient, and wherein classifying the lupus disease state of the patient include classifying whether the patient has as group B lupus disease state, or group D lupus disease state.

Aspect 197 is directed to the method of aspect 196, wherein the data set comprises or is derived from gene expression measurements of at least 3, 4, 5, 6, 7, 8, 9, or 10 genes selected from the group of genes CALR, EDEM2, EMC9, ERAP1, KDELC1, MANF, NUCB2, PSMB8, SEC24D, and TRDC.

Aspect 198 is directed to the method of aspect 196 or 197, wherein the data set comprises or is derived from gene expression measurements of CALR, EDEM2, EMC9, ERAP1, KDELC1, MANF, NUCB2, PSMB8, SEC24D, and TRDC.

Aspect 199 is directed to the method of any one of aspects 196 to 198, wherein analyzing the data set comprises providing the data set as an input to a trained machine-learning model trained to generate an inference of whether the data set is indicative of the patient having group B lupus disease state, or group D lupus disease state, wherein the method classify the lupus disease state of the patient based on the inference of the trained machine-learning model.

Aspect 200 is directed to the method aspect 199, wherein the inference comprises a confidence value between 0 and 1 that the patient has the group B lupus disease state, or the group D lupus disease state.

Aspect 201 is directed to a method for classifying a lupus disease state of a patient, the method comprising: analyzing a data set comprising gene expression measurements of at least 2 genes selected from the group of genes ACLY, ARSE, CD38, DERL1, DERL2, EDEM3, EIF2AK2, MANF, NFKB1 and SEC24D, to classify the lupus disease state of the patient, wherein the gene expression measurements are obtained from a biological sample obtained or derived from the patient, and wherein classifying the lupus disease state of the patient include classifying whether the patient has group B lupus disease state, or group E lupus disease state.

Aspect 202 is directed to the method of aspect 201, wherein the data set comprises or is derived from gene expression measurements of at least 3, 4, 5, 6, 7, 8, 9, or 10 genes selected from the group of genes ACLY, ARSE, CD38, DERL1, DERL2, EDEM3, EIF2AK2, MANF, NFKB1 and SEC24D.

Aspect 203 is directed to the method of aspect 201 or 202, wherein the data set comprises or is derived from gene expression measurements of ACLY, ARSE, CD38, DERL1, DERL2, EDEM3, EIF2AK2, MANF, NFKB1 and SEC24D.

Aspect 204 is directed to the method of any one of aspects 201 to 203, wherein analyzing the data set comprises providing the data set as an input to a trained machine-learning model trained to generate an inference of whether the data set is indicative of the patient having group B lupus disease state, or group E lupus disease state, wherein the method classify the lupus disease state of the patient based on the inference of the trained machine-learning model.

Aspect 205 is directed to the method aspect 204, wherein the inference comprises a confidence value between 0 and 1 that the patient has the group B lupus disease state, or the group E lupus disease state.

Aspect 206 is directed to a method for classifying a lupus disease state of a patient, the method comprising: analyzing a data set comprising gene expression measurements of at least 2 genes selected from the group of genes ACSL1, AIM2, ASAP1, CASP1, IL18, IL1B, IL1RN, MTF1, RIPK1, and SPI1, to classify the lupus disease state of the patient, wherein the gene expression measurements are obtained from a biological sample obtained or derived from the patient, and wherein classifying the lupus disease state of the patient include classifying whether the patient has group B lupus disease state, or group F lupus disease state.

Aspect 207 is directed to the method of aspect 206, wherein the data set comprises or is derived from gene expression measurements of at least 3, 4, 5, 6, 7, 8, 9, or 10 genes selected from the group of genes ACSL1, AIM2, ASAP1, CASP1, IL18, IL1B, IL1RN, MTF1, RIPK1, and SPI1.

Aspect 208 is directed to the method of aspect 206 or 207, wherein the data set comprises or is derived from gene expression measurements of ACSL1, AIM2, ASAP1, CASP1, IL18, IL1B, IL1RN, MTF1, RIPK1, and SPI1.

Aspect 209 is directed to the method of any one of aspects 206 to 208, wherein analyzing the data set comprises providing the data set as an input to a trained machine-learning model trained to generate an inference of whether the data set is indicative of the patient having group B lupus disease state, or group F lupus disease state, wherein the method classify the lupus disease state of the patient based on the inference of the trained machine-learning model.

Aspect 210 is directed to the method aspect 209, wherein the inference comprises a confidence value between 0 and 1 that the patient has the group B lupus disease state, or the group F lupus disease state.

Aspect 211 is directed to a method for classifying a lupus disease state of a patient, the method comprising: analyzing a data set comprising gene expression measurements of at least 2 genes selected from the group of genes ACLY, ARSE, BHMT, CASP10, CD37, EDEM2, GLS, HOMER2, IL1A, and TNFAIP3, to classify the lupus disease state of the patient, wherein the gene expression measurements are obtained from a biological sample obtained or derived from the patient, and wherein classifying the lupus disease state of the patient include classifying whether the patient has group B lupus disease state, or group G lupus disease state.

Aspect 212 is directed to the method of aspect 211, wherein the data set comprises or is derived from gene expression measurements of at least 3, 4, 5, 6, 7, 8, 9, or 10 genes selected from the group of genes ACLY, ARSE, BHMT, CASP10, CD37, EDEM2, GLS, HOMER2, IL1A, and TNFAIP3.

Aspect 213 is directed to the method of aspect 211 or 212, wherein the data set comprises or is derived from gene expression measurements of ACLY, ARSE, BHMT, CASP10, CD37, EDEM2, GLS, HOMER2, IL1A, and TNFAIP3.

Aspect 214 is directed to the method of any one of aspects 211 to 213, wherein analyzing the data set comprises providing the data set as an input to a trained machine-learning model trained to generate an inference of whether the data set is indicative of the patient having group B lupus disease state, or group G lupus disease state, wherein the method classify the lupus disease state of the patient based on the inference of the trained machine-learning model.

Aspect 215 is directed to the method aspect 214, wherein the inference comprises a confidence value between 0 and 1 that the patient has the group B lupus disease state, or the group G lupus disease state.

Aspect 216 is directed to a method for classifying a lupus disease state of a patient, the method comprising: analyzing a data set comprising gene expression measurements of at least 2 genes selected from the group of genes the group of genes ACSL1, AIM2, AKAP10, CASP10, CD38, CKB, IL18, NAIP, NFKB1, and TYROBP, to classify the lupus disease state of the patient, wherein the gene expression measurements are obtained from a biological sample obtained or derived from the patient, and wherein classifying the lupus disease state of the patient include classifying whether the patient has group B lupus disease state, or group H lupus disease state.

Aspect 217 is directed to the method of aspect 216, wherein the data set comprises or is derived from gene expression measurements of at least 3, 4, 5, 6, 7, 8, 9, or 10 genes selected from ACSL1, AIM2, AKAP10, CASP10, CD38, CKB, IL18, NAIP, NFKB1, and TYROBP.

Aspect 218 is directed to the method of aspect 216 or 217, wherein the data set comprises or is derived from gene expression measurements of ACSL1, AIM2, AKAP10, CASP10, CD38, CKB, IL18, NAIP, NFKB1, and TYROBP.

Aspect 219 is directed to the method of any one of aspects 216 to 218, wherein analyzing the data set comprises providing the data set as an input to a trained machine-learning model trained to generate an inference of whether the data set is indicative of the patient having group B lupus disease state, or group H lupus disease state, wherein the method classify the lupus disease state of the patient based on the inference of the trained machine-learning model.

Aspect 220 is directed to the method aspect 219, wherein the inference comprises a confidence value between 0 and 1 that the patient has the group B lupus disease state, or the group H lupus disease state.

Aspect 221 is directed to a method for classifying a lupus disease state of a patient, the method comprising: analyzing a data set comprising gene expression measurements of at least 2 genes selected from the group of genes BLK, CD247, CD3D, CD8A, IGHG1, IGHV3-20, SH2D1A, THEMIS2, TRDC, and TRG-AS1, to classify the lupus disease state of the patient, wherein the gene expression measurements are obtained from a biological sample obtained or derived from the patient, and wherein classifying the lupus disease state of the patient include classifying whether the patient has group C lupus disease state, or group D lupus disease state.

Aspect 222 is directed to the method of aspect 221, wherein the data set comprises or is derived from gene expression measurements of at least 3, 4, 5, 6, 7, 8, 9, or 10 genes selected from the group of genes BLK, CD247, CD3D, CD8A, IGHG1, IGHV3-20, SH2D1A, THEMIS2, TRDC, and TRG-AS1.

Aspect 223 is directed to the method of aspect 221 or 222, wherein the data set comprises or is derived from gene expression measurements of BLK, CD247, CD3D, CD8A, IGHG1, IGHV3-20, SH2D1A, THEMIS2, TRDC, and TRG-AS1.

Aspect 224 is directed to the method of any one of aspects 221 to 223, wherein analyzing the data set comprises providing the data set as an input to a trained machine-learning model trained to generate an inference of whether the data set is indicative of the patient having group C lupus disease state, or group D lupus disease state, wherein the method classify the lupus disease state of the patient based on the inference of the trained machine-learning model.

Aspect 225 is directed to the method aspect 224, wherein the inference comprises a confidence value between 0 and 1 that the patient has the group C lupus disease state, or the group D lupus disease state.

Aspect 226 is directed to a method for classifying a lupus disease state of a patient, the method comprising: analyzing a data set comprising gene expression measurements of at least 2 genes selected from the group of genes AURKB, CCNB1, CCNE1, EIF2AK2, IGHG1, IGHV3-20, IGLL1, IGLV4-3, IGLVI-70, and PTTG1, to classify the lupus disease state of the patient, wherein the gene expression measurements are obtained from a biological sample obtained or derived from the patient, and wherein classifying the lupus disease state of the patient include classifying whether the patient has group C lupus disease state, or group E lupus disease state.

Aspect 227 is directed to the method of aspect 226, wherein the data set comprises or is derived from gene expression measurements of at least 3, 4, 5, 6, 7, 8, 9, or 10 genes selected from the group of genes AURKB, CCNB1, CCNE1, EIF2AK2, IGHG1, IGHV3-20, IGLL1, IGLV4-3, IGLVI-70, and PTTG1.

Aspect 228 is directed to the method of aspect 226 or 227, wherein the data set comprises or is derived from gene expression measurements of AURKB, CCNB1, CCNE1, EIF2AK2, IGHG1, IGHV3-20, IGLL1, IGLV4-3, IGLVI-70, and PTTG1.

Aspect 229 is directed to the method of any one of aspects 226 to 228, wherein analyzing the data set comprises providing the data set as an input to a trained machine-learning model trained to generate an inference of whether the data set is indicative of the patient having group C lupus disease state, or group E lupus disease state, wherein the method classify the lupus disease state of the patient based on the inference of the trained machine-learning model.

Aspect 230 is directed to the method aspect 229, wherein the inference comprises a confidence value between 0 and 1 that the patient has the group C lupus disease state, or the group E lupus disease state.

Aspect 231 is directed to a method for classifying a lupus disease state of a patient, the method comprising: analyzing a data set comprising gene expression measurements of at least 2 genes selected from the group of genes CD226, CD247, CD28, CD4, CLEC1OA, HLA-DRB1, HLA-DRB6, IGIP, LY75, and TRDC, to classify the lupus disease state of the patient, wherein the gene expression measurements are obtained from a biological sample obtained or derived from the patient, and wherein classifying the lupus disease state of the patient include classifying whether the patient has as group C lupus disease state, or group F lupus disease state.

Aspect 232 is directed to the method of aspect 231, wherein the data set comprises or is derived from gene expression measurements of at least 3, 4, 5, 6, 7, 8, 9, or 10 genes selected from the group of genes CD226, CD247, CD28, CD4, CLEC10A, HLA-DRB1, HLA-DRB6, IGIP, LY75, and TRDC.

Aspect 233 is directed to the method of aspect 231or 232, wherein the data set comprises or is derived from gene expression measurements of CD226, CD247, CD28, CD4, CLEC1OA, HLA-DRB1, HLA-DRB6, IGIP, LY75, and TRDC.

Aspect 234 is directed to the method of any one of aspects 231 to 233, wherein analyzing the data set comprises providing the data set as an input to a trained machine-learning model trained to generate an inference of whether the data set is indicative of the patient having group C lupus disease state, or group F lupus disease state, wherein the method classify the lupus disease state of the patient based on the inference of the trained machine-learning model.

Aspect 235 is directed to the method aspect 234, wherein the inference comprises a confidence value between 0 and 1 that the patient has the group C lupus disease state, or the group F lupus disease state.

Aspect 236 is directed to a method for classifying a lupus disease state of a patient, the method comprising: analyzing a data set comprising gene expression measurements of at least 2 genes selected from the group of genes ACLY, CASP10, CD37, CD38, DERL1, DERL2, EDEM2, EIF2AK2, IFI30, and MANF, to classify the lupus disease state of the patient, wherein the gene expression measurements are obtained from a biological sample obtained or derived from the patient, and wherein classifying the lupus disease state of the patient include classifying whether the patient has group C lupus disease state, or group G lupus disease state.

Aspect 237 is directed to the method of aspect 236, wherein the data set comprises or is derived from gene expression measurements of at least 3, 4, 5, 6, 7, 8, 9, or 10 genes selected from the group of genes ACLY, CASP10, CD37, CD38, DERL1, DERL2, EDEM2, EIF2AK2, IF130, and MANF.

Aspect 238 is directed to the method of aspect 236 or 237, wherein the data set comprises or is derived from gene expression measurements of ACLY, CASP10, CD37, CD38, DERL1, DERL2, EDEM2, EIF2AK2, IF130, and MANF.

Aspect 239 is directed to the method of any one of aspects 236 to 238, wherein analyzing the data set comprises providing the data set as an input to a trained machine-learning model trained to generate an inference of whether the data set is indicative of the patient having group C lupus disease state, or group G lupus disease state, wherein the method classify the lupus disease state of the patient based on the inference of the trained machine-learning model.

Aspect 240 is directed to the method aspect 239, wherein the inference comprises a confidence value between 0 and 1 that the patient has the group C lupus disease state, or the group G lupus disease state.

Aspect 241 is directed to a method for classifying a lupus disease state of a patient, the method comprising: analyzing a data set comprising gene expression measurements of at least 2 genes selected from the group of genes AURKB, BRCA1, E2F3, EIF2AK2, IFITM3, MCM10, NDC80, PTTG1, SOCS3, and TNFAIP3, to classify the lupus disease state of the patient, wherein the gene expression measurements are obtained from a biological sample obtained or derived from the patient, and wherein classifying the lupus disease state of the patient include classifying whether the patient has group C lupus disease state, or group H lupus disease state.

Aspect 242 is directed to the method of aspect 241, wherein the data set comprises or is derived from gene expression measurements of at least 3, 4, 5, 6, 7, 8, 9, or 10 genes selected from the group of genes AURKB, BRCA1, E2F3, EIF2AK2, IFITM3, MCM10, NDC80, PTTG1, SOCS3, and TNFAIP3.

Aspect 243 is directed to the method of aspect 241 or 242, wherein the data set comprises or is derived from gene expression measurements of AURKB, BRCA1, E2F3, EIF2AK2, IFITM3, MCM10, NDC80, PTTG1, SOCS3, and TNFAIP3.

Aspect 244 is directed to the method of any one of aspects 241 to 243, wherein analyzing the data set comprises providing the data set as an input to a trained machine-learning model trained to generate an inference of whether the data set is indicative of the patient having group C lupus disease state, or group H lupus disease state, wherein the method classify the lupus disease state of the patient based on the inference of the trained machine-learning model.

Aspect 245 is directed to the method aspect 244, wherein the inference comprises a confidence value between 0 and 1 that the patient has the group C lupus disease state, or the group H lupus disease state.

Aspect 246 is directed to a method for classifying a lupus disease state of a patient, the method comprising: analyzing a data set comprising gene expression measurements of at least 2 genes selected from the group of genes CD3E, HLA-DMA, HLA-DPA1, HLA-DPB2, HLA-DQA2, HLA-DRB1, HLA-DRB6, KLRF1, NCAM1, and TRDC, to classify the lupus disease state of the patient, wherein the gene expression measurements are obtained from a biological sample obtained or derived from the patient, and wherein classifying the lupus disease state of the patient include classifying whether the patient has group D lupus disease state, or group E lupus disease state.

Aspect 247 is directed to the method of aspect 246, wherein the data set comprises or is derived from gene expression measurements of at least 3, 4, 5, 6, 7, 8, 9, or 10 genes selected from the group of genes CD3E, HLA-DMA, HLA-DPA1, HLA-DPB2, HLA-DQA2, HLA-DRB1, HLA-DRB6, KLRF1, NCAM1, and TRDC.

Aspect 248 is directed to the method of aspect 246 or 247, wherein the data set comprises or is derived from gene expression measurements of CD3E, HLA-DMA, HLA-DPA1, HLA-DPB2, HLA-DQA2, HLA-DRB1, HLA-DRB6, KLRF1, NCAM1, and TRDC.

Aspect 249 is directed to the method of any one of aspects 246 to 248, wherein analyzing the data set comprises providing the data set as an input to a trained machine-learning model trained to generate an inference of whether the data set is indicative of the patient having group D lupus disease state, or group E lupus disease state, wherein the method classify the lupus disease state of the patient based on the inference of the trained machine-learning model.

Aspect 250 is directed to the method aspect 249, wherein the inference comprises a confidence value between 0 and 1 that the patient has the group D lupus disease state, or the group E lupus disease state.

Aspect 251 is directed to a method for classifying a lupus disease state of a patient, the method comprising: analyzing a data set comprising gene expression measurements of at least 2 genes selected from the group of genes BLK, CD226, CD247, CD8A, HLA-DQA1, HLA-DQA2, HLA-DRB5, HLA-DRB6, TARP, and TRG-AS1, to classify the lupus disease state of the patient, wherein the gene expression measurements are obtained from a biological sample obtained or derived from the patient, and wherein classifying the lupus disease state of the patient include classifying whether the patient has group D lupus disease state, or group F lupus disease state.

Aspect 252 is directed to the method of aspect 251, wherein the data set comprises or is derived from gene expression measurements of at least 3, 4, 5, 6, 7, 8, 9, or 10 genes selected from the group of genes BLK, CD226, CD247, CD8A, HLA-DQA1, HLA-DQA2, HLA-DRB5, HLA-DRB6, TARP, and TRG-AS1.

Aspect 253 is directed to the method of aspect 251or 252, wherein the data set comprises or is derived from gene expression measurements of BLK, CD226, CD247, CD8A, HLA-DQA1, HLA-DQA2, HLA-DRB5, HLA-DRB6, TARP, and TRG-AS1.

Aspect 254 is directed to the method of any one of aspects 251 to 253, wherein analyzing the data set comprises providing the data set as an input to a trained machine-learning model trained to generate an inference of whether the data set is indicative of the patient having group D lupus disease state, or group F lupus disease state, wherein the method classify the lupus disease state of the patient based on the inference of the trained machine-learning model.

Aspect 255 is directed to the method aspect 254, wherein the inference comprises a confidence value between 0 and 1 that the patient has the group D lupus disease state, or the group F lupus disease state.

Aspect 256 is directed to a method for classifying a lupus disease state of a patient, the method comprising: analyzing a data set comprising gene expression measurements of at least 2 genes selected from the group of genes CD14, CLEC5A, LILRA2, LILRA5, LILRA6, LMNB1, NLRC4, OLR1, OSCAR, and SEMA4A, to classify the lupus disease state of the patient, wherein the gene expression measurements are obtained from a biological sample obtained or derived from the patient, and wherein classifying the lupus disease state of the patient include classifying whether the patient has group D lupus disease state, or group G lupus disease state.

Aspect 257 is directed to the method of aspect 256, wherein the data set comprises or is derived from gene expression measurements of at least 3, 4, 5, 6, 7, 8, 9, or 10 genes selected from the group of genes CD14, CLEC5A, LILRA2, LILRA5, LILRA6, LMNB1, NLRC4, OLR1, OSCAR, and SEMA4A.

Aspect 258 is directed to the method of aspect 256 or 257, wherein the data set comprises or is derived from gene expression measurements of CD14, CLEC5A, LILRA2, LILRA5, LILRA6, LMNB1, NLRC4, OLR1, OSCAR, and SEMA4A.

Aspect 259 is directed to the method of any one of aspects 256 to 258, wherein analyzing the data set comprises providing the data set as an input to a trained machine-learning model trained to generate an inference of whether the data set is indicative of the patient having group D lupus disease state, or group G lupus disease state, wherein the method classify the lupus disease state of the patient based on the inference of the trained machine-learning model.

Aspect 260 is directed to the method aspect 259, wherein the inference comprises a confidence value between 0 and 1 that the patient has the group D lupus disease state, or the group G lupus disease state.

Aspect 261 is directed to a method for classifying a lupus disease state of a patient, the method comprising: analyzing a data set comprising gene expression measurements of at least 2 genes selected from the group of genes BLK, CD177, CD247, CXCR2, FUT7, LTB4R, OSM, TARP, TRDC, and TRG-AS1, to classify the lupus disease state of the patient, wherein the gene expression measurements are obtained from a biological sample obtained or derived from the patient, and wherein classifying the lupus disease state of the patient include classifying whether the patient has group D lupus disease state, or group H lupus disease state.

Aspect 262 is directed to the method of aspect 261, wherein the data set comprises or is derived from gene expression measurements of at least 3, 4, 5, 6, 7, 8, 9, or 10 genes selected from the group of genes BLK, CD177, CD247, CXCR2, FUT7, LTB4R, OSM, TARP, TRDC, and TRG-AS1.

Aspect 263 is directed to the method of aspect 261 or 262, wherein the data set comprises or is derived from gene expression measurements of BLK, CD177, CD247, CXCR2, FUT7, LTB4R, OSM, TARP, TRDC, and TRG-AS1.

Aspect 264 is directed to the method of any one of aspects 261 to 263, wherein analyzing the data set comprises providing the data set as an input to a trained machine-learning model trained to generate an inference of whether the data set is indicative of the patient having group D lupus disease state, or group H lupus disease state, wherein the method classify the lupus disease state of the patient based on the inference of the trained machine-learning model.

Aspect 265 is directed to the method aspect 264, wherein the inference comprises a confidence value between 0 and 1 that the patient has the group D lupus disease state, or the group H lupus disease state.

Aspect 266 is directed to a method for classifying a lupus disease state of a patient, the method comprising: analyzing a data set comprising gene expression measurements of at least 2 genes selected from the group of genes BLK, CCR3, CD247, CD28, CD4, CD8A, CTLA4, HAVCR2, KLRG1, and TRG-AS1, to classify the lupus disease state of the patient, wherein the gene expression measurements are obtained from a biological sample obtained or derived from the patient, and wherein classifying the lupus disease state of the patient include classifying whether the patient has group E lupus disease state, or group F lupus disease state.

Aspect 267 is directed to the method of aspect 266, wherein the data set comprises or is derived from gene expression measurements of at least 3, 4, 5, 6, 7, 8, 9, or 10 genes selected from the group of genes BLK, CCR3, CD247, CD28, CD4, CD8A, CTLA4, HAVCR2, KLRG1, and TRG-AS1.

Aspect 268 is directed to the method of aspect 266 or 267, wherein the data set comprises or is derived from gene expression measurements of BLK, CCR3, CD247, CD28, CD4, CD8A, CTLA4, HAVCR2, KLRG1, and TRG-AS1.

Aspect 269 is directed to the method of any one of aspects 266 to 268, wherein analyzing the data set comprises providing the data set as an input to a trained machine-learning model trained to generate an inference of whether the data set is indicative of the patient having group E lupus disease state, or group F lupus disease state, wherein the method classify the lupus disease state of the patient based on the inference of the trained machine-learning model.

Aspect 270 is directed to the method aspect 269, wherein the inference comprises a confidence value between 0 and 1 that the patient has the group E lupus disease state, or the group F lupus disease state.

Aspect 271 is directed to a method for classifying a lupus disease state of a patient, the method comprising: analyzing a data set comprising gene expression measurements of at least 2 genes selected from the group of genes CALR, CD244, CTLA4, DERL2, EMC9, ERAP1, GALNT2, HAVCR2, ICOS, and PSMB8, to classify the lupus disease state of the patient, wherein the gene expression measurements are obtained from a biological sample obtained or derived from the patient, and wherein classifying the lupus disease state of the patient include classifying whether the patient has group E lupus disease state, or group G lupus disease state.

Aspect 272 is directed to the method of aspect 271, wherein the data set comprises or is derived from gene expression measurements of at least 3, 4, 5, 6, 7, 8, 9, or 10 genes selected from the group of genes CALR, CD244, CTLA4, DERL2, EMC9, ERAP1, GALNT2, HAVCR2, ICOS, and PSMB8.

Aspect 273 is directed to the method of aspect 271 or 272, wherein the data set comprises or is derived from gene expression measurements of CALR, CD244, CTLA4, DERL2, EMC9, ERAP1, GALNT2, HAVCR2, ICOS, and PSMB8.

Aspect 274 is directed to the method of any one of aspects 271 to 273, wherein analyzing the data set comprises providing the data set as an input to a trained machine-learning model trained to generate an inference of whether the data set is indicative of the patient having group E lupus disease state, or group G lupus disease state, wherein the method classify the lupus disease state of the patient based on the inference of the trained machine-learning model.

Aspect 275 is directed to the method aspect 274, wherein the inference comprises a confidence value between 0 and 1 that the patient has the group E lupus disease state, or the group G lupus disease state.

Aspect 276 is directed to a method for classifying a lupus disease state of a patient, the method comprising: analyzing a data set comprising gene expression measurements of at least 2 genes selected from the group of genes CASP1, EIF2AK2, GBP1, IFI30, IFITM3, NEK7, NLRC4, PSMB10, PSMB8, and RIPK1, to classify the lupus disease state of the patient, wherein the gene expression measurements are obtained from a biological sample obtained or derived from the patient, and wherein classifying the lupus disease state of the patient include classifying whether the patient has group E lupus disease state, or group H lupus disease state.

Aspect 277 is directed to the method of aspect 276, wherein the data set comprises or is derived from gene expression measurements of at least 3, 4, 5, 6, 7, 8, 9, or 10 genes selected from the group of genes CASP1, EIF2AK2, GBP1, IFI30, IFITM3, NEK7, NLRC4, PSMB10, PSMB8, and RIPK1.

Aspect 278 is directed to the method of aspect 276 or 277, wherein the data set comprises or is derived from gene expression measurements of CASP1, EIF2AK2, GBP1, IFI30, IFITM3, NEK7, NLRC4, PSMB10, PSMB8, and RIPK1.

Aspect 279 is directed to the method of any one of aspects 276 to 278, wherein analyzing the data set comprises providing the data set as an input to a trained machine-learning model trained to generate an inference of whether the data set is indicative of the patient having group E lupus disease state, or group H lupus disease state, wherein the method classify the lupus disease state of the patient based on the inference of the trained machine-learning model.

Aspect 280 is directed to the method aspect 279, wherein the inference comprises a confidence value between 0 and 1 that the patient has the group E lupus disease state, or the group H lupus disease state.

Aspect 281 is directed to a method for classifying a lupus disease state of a patient, the method comprising: analyzing a data set comprising gene expression measurements of at least 2 genes selected from the group of genes CALR, CD244, CTLA4, DERL2, EMC9, ERAP1, GALNT2, HAVCR2, ICOS, and PSMB8, to classify the lupus disease state of the patient, wherein the gene expression measurements are obtained from a biological sample obtained or derived from the patient, and wherein classifying the lupus disease state of the patient include classifying whether the patient has group F lupus disease state, or group G lupus disease state.

Aspect 282 is directed to the method of aspect 281, wherein the data set comprises or is derived from gene expression measurements of at least 3, 4, 5, 6, 7, 8, 9, or 10 genes selected from the group of genes CALR, CD244, CTLA4, DERL2, EMC9, ERAP1, GALNT2, HAVCR2, ICOS, and PSMB8.

Aspect 283 is directed to the method of aspect 281 or 282, wherein the data set comprises or is derived from gene expression measurements of CALR, CD244, CTLA4, DERL2, EMC9, ERAP1, GALNT2, HAVCR2, ICOS, and PSMB8.

Aspect 284 is directed to the method of any one of aspects 281 to 283, wherein analyzing the data set comprises providing the data set as an input to a trained machine-learning model trained to generate an inference of whether the data set is indicative of the patient having group F lupus disease state, or group G lupus disease state, wherein the method classify the lupus disease state of the patient based on the inference of the trained machine-learning model.

Aspect 285 is directed to the method aspect 284, wherein the inference comprises a confidence value between 0 and 1 that the patient has the group F lupus disease state, or the group G lupus disease state.

Aspect 286 is directed to a method for classifying a lupus disease state of a patient, the method comprising: analyzing a data set comprising gene expression measurements of at least 2 genes selected from the group of genes CASP1, EIF2AK2, GBP1, IFI30, IFITM3, NEK7, NLRC4, PSMB10, PSMB8, and RIPK1, to classify the lupus disease state of the patient, wherein the gene expression measurements are obtained from a biological sample obtained or derived from the patient, and wherein classifying the lupus disease state of the patient include classifying whether the patient has group F lupus disease state, or group H lupus disease state.

Aspect 287 is directed to the method of aspect 286, wherein the data set comprises or is derived from gene expression measurements of at least 3, 4, 5, 6, 7, 8, 9, or 10 genes selected from the group of genes CASP1, EIF2AK2, GBP1, IFI30, IFITM3, NEK7, NLRC4, PSMB10, PSMB8, and RIPK1.

Aspect 288 is directed to the method of aspect 286 or 287, wherein the data set comprises or is derived from gene expression measurements of CASP1, EIF2AK2, GBP1, IFI30, IFITM3, NEK7, NLRC4, PSMB10, PSMB8, and RIPK1.

Aspect 289 is directed to the method of any one of aspects 286 to 288, wherein analyzing the data set comprises providing the data set as an input to a trained machine-learning model trained to generate an inference of whether the data set is indicative of the patient having group F lupus disease state, or group H lupus disease state, wherein the method classify the lupus disease state of the patient based on the inference of the trained machine-learning model.

Aspect 290 is directed to the method aspect 289, wherein the inference comprises a confidence value between 0 and 1 that the patient has the group F lupus disease state, or the group H lupus disease state.

Aspect 291 is directed to a method for classifying a lupus disease state of a patient, the method comprising: analyzing a data set comprising gene expression measurements of at least 2 genes selected from the group of genes ATP5A1, CD160, CD244, CD3E, COX16, COX18, NDUFAF4, NDUFS1, TIMMDC1, and TTC19, to classify the lupus disease state of the patient, wherein the gene expression measurements are obtained from a biological sample obtained or derived from the patient, and wherein classifying the lupus disease state of the patient include classifying whether the patient has group G lupus disease state, or group H lupus disease state.

Aspect 292 is directed to the method of aspect 291, wherein the data set comprises or is derived from gene expression measurements of at least 3, 4, 5, 6, 7, 8, 9, or 10 genes selected from the group of genes ATP5A1, CD160, CD244, CD3E, COX16, COX18, NDUFAF4, NDUFS1, TIMMDC1, and TTC19.

Aspect 293 is directed to the method of aspect 291 or 292, wherein the data set comprises or is derived from gene expression measurements of ATP5A1, CD160, CD244, CD3E, COX16, COX18, NDUFAF4, NDUFS1, TIMMDC1, and TTC19.

Aspect 294 is directed to the method of any one of aspects 291 to 293, wherein analyzing the data set comprises providing the data set as an input to a trained machine-learning model trained to generate an inference of whether the data set is indicative of the patient having group G lupus disease state, or group H lupus disease state, wherein the method classify the lupus disease state of the patient based on the inference of the trained machine-learning model.

Aspect 295 is directed to the method aspect 294, wherein the inference comprises a confidence value between 0 and 1 that the patient has the group G lupus disease state, or the group H lupus disease state.

Aspect 296 is directed to the method of any one of aspects 156 to 295, further comprising: a) receiving, as an output of the trained machine-learning model, the inference; and b) electronically outputting a report classifying the lupus disease state of a patient.

Aspect 297 is directed to the method of any one of aspects 156 or 296, wherein the trained machine-learning model is trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), Classification and Regression Tree (CART), hierarchical clustering, or any combination thereof.

Aspect 298 is directed to the method of any one of aspects 156 to 297, wherein the trained machine-learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 299 is directed to the method of any one of aspects 156 to 298, wherein the biological sample comprises a blood sample, isolated peripheral blood mononuclear cells (PBMCs), a tissue biopsy sample, or any derivative thereof.

Aspect 300 is directed to the method of any one of aspects 156 to 299, wherein the method classify the lupus disease state of the patient with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 301 is directed to the method of any one of aspects 156 to 300, wherein the method classify the lupus disease state of the patient with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 302 is directed to the method of any one of aspects 156 to 301, wherein the method classify the lupus disease state of the patient with specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 303 is directed to the method of any one of aspects 156 to 302, wherein the method classify the lupus disease state of the patient with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 304 is directed to the method of any one of aspects 156 to 303, wherein the method classify the lupus disease state of the patient with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 305 is directed to the method of any one of aspects 156 to 304, wherein the patient has lupus.

Aspect 306 is directed to the method of any one of aspects 156 to 305, wherein the patient is at elevated risk of having lupus.

Aspect 307 is directed to the method of any one of aspects 156 to 306, wherein the patient is asymptomatic for lupus.

Aspect 308 is directed to the method of any one of aspects 156 to 307, further comprising selecting, recommending and/or administering a treatment to the patient based on the classification of the lupus disease state of the patient.

Aspect 309 is directed to the method of any one of aspects 156 to 308, further comprising administering a treatment to the patient based on the classification of the lupus disease state of the patient.

Aspect 310 is directed to the method of any one of aspects 308 to 309, wherein the treatment is configured to treat lupus.

Aspect 311 is directed to the method of any one of aspects 308 to 310, wherein the treatment is configured to treat reduce severity of lupus.

Aspect 312 is directed to the method of any one of aspects 308 to 311, wherein the treatment is configured to reduce risk of having lupus.

Aspect 313 is directed to the method of any one of aspects 308 to 312, wherein the treatment comprises one or more pharmaceutical compositions.

Aspect 314 is directed to the method of any one of aspects 308 to 313, wherein the treatment comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof.

Aspect 315 is directed to the method of any one of aspects 308 to 314, wherein the treatment comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof.

Aspect 316 is directed to the method of any one of aspects 308 to 315, wherein the treatment for, group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, a NK cell inhibitor, a neutrophil function inhibitor, a TNF inhibitor, an IL1 inhibitor, a Plasma cell inhibitor or any combination thereof; group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof; and/or group H lupus disease state comprises an IFN inhibitor, a neutrophil function inhibitor, a TNF inhibitor, an IL1 inhibitor, a Plasma cell inhibitor or any combination thereof

Aspect 317 is directed to the method of any one of aspects 308 to 316, wherein the treatment for, group B lupus disease state comprises Belimumab, Dasatinib, and/or Apremilast; group C lupus disease state comprises Anifrolumab, Anakinra, Adalimumab, Certolizumab pegol, Etanercept, Golimumab, Inflximab, Dasatinib, Apremilast, or any combination thereof; group D lupus disease state comprises Belimumab, Anifrolumab, Mycophenolate, AZA, Bortezomib, Isatuximab, Elotuzumab, Anakinra, Adalimumab, Certolizumab pegol, Etanercept, Golimumab, Infiximab, Dasatinib, Apremilast or any combination thereof; group E lupus disease state comprises Anifrolumab, Mycophenolate, Bortezomib, Isatuximab, Elotuzumab, Adalimumab, Certolizumab pegol, Etanercept, Golimumab, Inflximab, Dasatinib, Apremilast or any combination thereof; group F lupus disease state comprises Anifrolumab, Anakinra, Adalimumab, Certolizumab pegol, Etanercept, Golimumab, Infiximab, Dasatinib, Apremilast, Belimumab, or any combination thereof; group G lupus disease state comprises Belimumab, Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast or any combination thereof; and group H lupus disease state comprises Anifrolumab, Mycophenolate, Bortezomib, Isatuximab, Elotuzumab, Anakinra, Adalimumab, Certolizumab pegol, Etanercept, Golimumab, Inflximab, Dasatinib, Apremilast, Belimumab, or any combination thereof.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: Identification of six adult lupus endotypes. k-means clustering of GSVA scores of the 32 features in 1620 adult lupus patients from GSE88884 (Illuminate 1 & 2) yielded six clusters using baseline gene expression. Color labels above the heatmap indicate patient clusters and colors were randomly generated in R. Color labels below the heatmap indicate patient ancestry.

FIGS. 2A-E: Clinical characteristics (FIG. 2A: SLEDAI score; FIG. 2B: blood anti-double-stranded DNA antibody level; FIG. 2C: blood anti-ribonucleoprotein (RNP) antibody level; FIG. 2D: blood complement component 3 (C3) protein level; FIG. 2E: blood complement component 4 (C4) protein level) of the six identified lupus endotypes. Scatterplots in display the mean±SD for each immunologic/inflammatory and systemic disease indicators in each cluster; statistical differences were found with Dunn's multiple comparisons test. The “least abnormal” lupus cluster is identified by the blue arrow.

FIGS. 3A-J: Lupus patients in the least severe subset are less likely to be characterized by low complement, positive anti-dsDNA status, and leukopenia. Distribution of (FIG. 3A) vasculitis, (FIG. 3B) arthritis, (FIG. 3C) pyuria, (FIG. 3D) rash, (FIG. 3E) alopecia, (FIG. 3F) mucosal ulcers, (FIG. 3G) pleurisy, (FIG. 3H) low complement, (FIG. 3I) anti-dsDNA, and (FIG. 3J) leukopenia among molecular subsets. The likelihood of having low complement, anti-dsDNA, and leukopenia in the IR2 subset is 0.34, 0.34, and 0.00 respectively as compared to the other five subsets combined. Significant differences in expected and observed frequencies between IR2, the “least abnormal subset” and all other subsets (denoted with asterisk above bars) was identified with Chi Square Test. Significant associations between categorical variables and all subsets (denoted with asterisks on the y-axis) were identified using Chi Square Test of Independence

FIGs. 4A-J: Lupus patients in the least severe subset are less likely to be characterized by hematologic involvement. Distribution of (FIG. 4A) CNS, (FIG. 4B) vascular, (FIG. 4C) musculoskeletal, (FIG. 4D) renal, (FIG. 4E) mucocutaneous, (FIG. 4F) cardiovascular/respiratory, (FIG. 4G) immunologic, (FIG. 4H) constitutional, and (FIG. 4I) hematologic domain involvement. (FIG. 4J) Distribution of the number of SLE domains involved. The likelihood of having immunologic and hematologic domain in the IR2 subset is 0.34 and 0.00 respectively as compared to the other five subsets combined. Significant differences in expected and observed frequencies between IR2, the “least abnormal subset” and all other subsets (denoted with asterisk above bars) was identified with Chi Square Test. There were no significant associations identified between categorical variables and all subsets using Chi Square Test of Independence. In FIGS. 4A-4I, solid black areas indicate % of patients without involvement; light areas indicate % having involvement. In FIG. 4J, bars from bottom to top show the % of patients having increasing numbers of SLEDAI domains involved, starting with 2 involved domains in the bottom segment of each bar.

FIG. 5: Identification of lupus endotypes in additional whole blood datasets. K-means clustering of GSVA scores of the 32 features yielded 6 clusters in adult lupus patients from GSE116006 using baseline gene expression. Color labels above the heatmap indicate cluster identity which were randomly generated using the ‘grDevices’ color palette in R. Color bars below the heatmap represent treatment.

FIGS. 6A-B: Subset similarity between two independent datasets. K-means clustering of two independent datasets (top: FIG. 6A) GSE116006 and (bottom: FIG. 6A) GSE88884 reveals four conserved subsets by cosine similarity (FIG. 6B).

FIG. 7: Eight molecular endotypes emerge from clustering of 17 datasets comprising 3,166 lupus patients. The eight endotypes are visualized via k-means clustering.

FIG. 8: Distribution of the Lupus Cell and Immune Score (LuCIS) for 1620 lupus patients across six molecular subsets. LuCIS was calculated for individual lupus patients and was plotted by molecular subset shown as (top) mean±SEM or (bottom) distribution of LuCIS as a violin plot. Significant differences between mean LuCIS of each cluster was analyzed with Dunn's multiple comparisons test.

FIGS. 9A-B: LuCIS correlates with anti-dsDNA and SLEDAI. Linear regression of Anti-dsDNA (FIG. 9A) or SLEDAI (FIG. 9B) with LuCIS in 1612 patients from GSE88884.

FIGS. 10A-C: Molecular subset membership at baseline predicts drug response at 52 weeks. K-means clustering of 32 features in Illuminate-2 lupus samples (FIG. 10A) and their clinical responses by SRI-4 (FIG. 10B) and SRI-5 (FIG. 10C) per gene expression determined endotype. Responses among the treatment groups were ascertained by the Trend Chi Square test. Endotype color labels were randomly generated using the ‘grDevices’ color palette in R. Q2W indicates frequency of drug administration was every 2 weeks. Q4W indicates frequency of drug administration was every 4 weeks A: p<0.05 observed by Trend Chi Square Test for Q2W>Q4W>Placebo, Q2W>Placebo, and Q2W+Q4W>Placebo.

FIGS. 11 A-C: Lupus patients in the least severe subset are less likely to have severe flares during 52 weeks on standard of care. Distribution of severe flares by molecular subset shown as (FIG. 11A) no severe flare or >1 severe flare, or (FIG. 11A) the number of severe flares. The likelihood of having >1 severe flare in the IR2 subset is 0.116 as compared to the other five subsets. Significant differences in expected and observed frequencies between IR2, the “least abnormal subset” and all other subsets (denoted with asterisk above bars) was identified by Chi Square Test, as shown by the contingency tables in (FIG. 11C). Significant associations between categorical variables and all subsets (denoted with asterisks on the y-axis) were identified using Chi Square Test of Independence.

FIGS. 12A-I: Machine learning algorithms can predict lupus endotype membership with high accuracy. Multi-class classification by machine learning analysis categorizes lupus samples into eight patient endotypes (FIGS. 7, 32A). FIGS. 12A-12C: Area under the ROC curve (AUC) (FIG. 12A), confusion matrices (FIG. 12B) and performance metrics (FIG. 12C), of classifier support vector machine. FIGS. 12D-12F: Area under the ROC curve (AUC) (FIG. 12D), confusion matrices (FIG. 12E) and performance metrics (FIG. 12F), of classifier random forest. FIGS. 12G-12I: Area under the ROC curve (AUC) (FIG. 12G), confusion matrices (FIG. 12H) and performance metrics (FIG. 12I), of classifier deep learning (sequential neural network). Each model was trained on 2532 (80%) lupus samples and tested on the remaining 20% (n=634) for a total N=3166 from 16 datasets.

FIG. 13: Schematic Diagram. A schematic of clustering patients from lupus data set(s) according to one non-limiting example of the present disclosure.

FIG. 14: Experimental design of feature selection. Data processing and machine learning workflow to arrive at the 32 features used to stratify patients for the identification of endotypes, according to one non-limiting example of the present disclosure.

FIG. 15: Distribution of Patients in GSE88884 Drug Treatment Cohorts. Active, female SLE patients in GSE88884 Illuminate-1 & 2 were stratified by lupus SoC at baseline into drug treatment cohorts. Each cohort was individually endotyped with k=5 subsets per elbow and silhouette analyses. Resultant endotypes were then compared to the six endotypes identified with all 1620 active, female SLE patients in GSE88884 using cosine similarity. The proportion of patients in each endotype with cosine similarity >0.7 are displayed. The absence of a data point indicates that no endotype in the drug treatment cohort was similar to the reference endotype using this cosine similarity cutoff. Significant deviations in the proportion of patients from the full cohort (gray) are denoted with black asterisks. Significance was determined using the prop_testfunction in R and the bubble plot was generated with the ggplot2 package. HCQ=hydroxychloroquine; MMF=mycophenolate mofetil; MTX=methotrexate; AZA=azathioprine; CTX=Cytoxan (cyclophosphamide).

*p<0.05; **p<0.01; ***p<0.001.

FIGS. 16A-F: Identification of six adult lupus endotypes and their clinical characteristics K-means clustering of GSVA scores of the 32 features in 1620 adult lupus patients from GSE88884 (Illuminate 1 & 2) yielded six clusters using baseline gene expression (FIG. 16A). For FIG. 16A the molecular features (e.g., modules) listed from top to bottom (on the left vertical axis) are IFN, Immunoproteasome, Plasma Cell, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cell, gd T Cell, Anergic/activated T Cell, Oxidative Phosphorylation, Unfolded Protein, TCRAJ, T Cell, TCRA, TCRB, IL23 Complex and Treg. Clinical metadata were summarized for each cluster using baseline values. Metadata was categorized by (FIG. 16B) quantitative immunologic/inflammatory and systemic disease indicators (first row left: SLEDAI, first row right: anti-dsDNA, second row left: antiRNP, second row right: C3, third row: C4), (FIG. 16C) categorical immunologic/inflammatory disease indicators (first row left: lymphopenia, first row right: anti-dsDNA, second row left: antiRNP, second row right: C3 status, third row: C4 status), (FIG. 16D) incidence of subsequent flares, (FIG. 16E) patient ancestry (upper panel: African ancestry, middle panel: European ancestry, lower panel: Native American (Hispanic) ancestry), and (FIG. 16F) medication use (first row left: oral steroids, first row right: antimalarials, second row left: MTX, second row right: AZA, third row: MMF). Labels on x-axes indicate the shorthand name for the endotypes and colors were randomly generated using the ‘grDevices’ color palette in R. IR2=indianred2, DG4=darkgoldenrod4, LG1=lightgoldenrod1, LS3=lightsalmon3, L=lavender, and SB3=slateblue3. Scatterplots in (B) display the mean±SD for each endotype; statistical differences were found with Dunn's multiple comparisons test. Lymphopenia was defined as less than 1 billion lymphocytes per liter. Significant associations between categorical variables and endotypes (denoted with asterisks in titles) were identified using Chi Square Test of Independence. In (C)-(F), odds ratios of IR2 having a positive value for the clinical trait of interest are displayed above the IR2 bar with significance indicated by asterisks. Missing data (n.d.) were excluded from analyses. Heatmap in (A) was generated with the ComplexHeatmap R package and subsequently edited in GraphPad Prism v. 9.4.0 (673). Graphs in (B)-(F) were created in GraphPad Prism v 9.4.0 (673). ESR=erythrocyte sedimentation rate. n.d.=no data. HCQ=hydroxychloroquine. MMF=mycophenolate mofetil. AZA=azathioprine. MTX=methotrexate.

*p<0.05; **p<0.01; ***p<0.001; ****p<0.0001

FIGS. 17A-B: K-means clustering and comparison of lupus and control samples in GSE88884 K-means clustering of GSVA scores of the 32 features in 1,620 lupus patients and 17 healthy controls (CTLs) in GSE88884 (FIG. 17A). 64.7% of the controls were found in IR2′ (IR2′=11, DG4′=6). Cosine similarity of the k-means clusters of the 1,620 lupus patients only versus patients and controls (FIG. 17B). Heatmap in FIG. 17A was generated with GraphPad Prism v. 9.4.0 (673). Cosine similarity plot in FIG. 17B was generated in R with the plot.matrixpackage. For FIG. 17A the molecular features (e.g., modules) listed from top to bottom (on the left vertical axis) are IFN, Immunoproteasome, Plasma Cell, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cell, gd T Cell, Anergic/activated T Cell, Oxidative Phosphorylation, Unfolded Protein, TCRAJ, T Cell, TCRA, TCRB, IL23 Complex and Treg

FIG. 18: SLEDAI manifestations among six adult lupus endotypes in GSE88884 K-means clustering of GSVA scores of the 32 features in 1620 adult lupus patients from GSE88884 (Illuminate 1 & 2) yielded six clusters using baseline gene expression. From top to bottom: vasculitis, pleurisy, pyuria, low complement, rash, anti-dsDNA vasculitis, mucosal ulcers, arthritis. Clinical metadata were summarized for each cluster using baseline values of manifestations defined by SLEDAI. Labels on x-axes indicate the shorthand name for patient clusters and colors were randomly generated using the ‘grDevices’ color palette in R. IR2=indianred2, DG4=darkgoldenrod4, LG1=lightgoldenrod1, LS3=lightsalmon3, L=lavender, and SB3=slateblue3. Significant associations between categorical variables and molecular subset (denoted with asterisks in titles) were identified using Chi Square Test of Independence. Odds ratios of IR2 having a positive value for the clinical trait of interest are displayed above the IR2 bar with significance indicated by asterisks. Graphs were created in GraphPad Prism v 9.4.0 (673). *p<0.05; **p<0.01; ***p<0.001; ****p<0.0001.

FIG. 19: Organ system involvement among six adult lupus endotypes in GSE88884. K-means clustering of GSVA scores of the 32 features in 1620 adult lupus patients from GSE88884 (Illuminate 1 & 2) yielded six clusters using baseline gene expression. From top to bottom: CNS, CV/respiratory, vascular, immunologic, musculoskeletal, constitutional, renal, hematologic, mucocutaneous, number of SLEDAI domains involved. Clinical metadata were summarized for each cluster using baseline values of organ system involvement defined by SLEDAI. Labels on x-axes indicate the shorthand name for patient clusters and colors were randomly generated using the ‘grDevices’ color palette in R. IR2=indianred2, DG4=darkgoldenrod4, LGT=lightgoldenrod1, LS3=lightsalmon3, L=lavender, and SB3=slateblue3. Significant associations between categorical variables and molecular subset (denoted with asterisks in titles) were identified using Chi Square Test of Independence. Odds ratios of IR2 having a positive value for the clinical trait of interest are displayed above the IR2 bar with significance indicated by asterisks. Graphs were created in GraphPad Prism v 9.4.0 (673). #p<0.10, *p<0.05; **p<0.01; ***p<0.001; ****p<0.0001.

FIGS. 20A-F: Comparison of endotypes determined by molecular 32 features and those determined by clinical metadata. K-means clustering of Illuminate-2 active lupus samples and their clinical responses by SRI-4 and SRI-5 per endotype determined by gene expression data and 32 features (FIG. 20A), clinical metadata (FIG. 20B), or GMVAE (FIG. 20C). Responses among the treatment groups were ascertained by the Trend Chi Square test (A, right, top & bottom). (FIGS. 20D-20F) Rand indices comparing the patient clusters derived using clinical data and/or molecular features by the k-means and GMVAE algorithms. The heatmap in (FIG. 20A) is a comparison of the clinical k-means endotypes and molecular endotypes, (E) compares molecular endotypes and clinical GMVAE endotypes, and (F) clinical k-means endotypes and clinical GMVAE endotypes. Endotype color labels were randomly generated using the ‘grDevices’ color palette in R. Q2W indicates frequency of drug administration was every 2 weeks. Q4W indicates frequency of drug administration was every 4 weeks. Heatmaps in (A-C) were generated with the ComplexHeatmap R package. Categorical variables in (B-C) including ancestry, medication, and components of SLEDAI, were binarized. Histograms in (A) were made with ggplot2 in R. Heatmaps in (D-F) were visualized with the Seabom package in Python and Rand indices were calculated with the scikit-learn package. EA=European Ancestry, AA=African Ancestry, NAT=Native American (Hispanic) Ancestry, ANTIMAL=antimalarials, CORTICO=corticosteroids, IMMUNO=immunosuppressants. For FIG. 20A the molecular features (e.g., modules) listed from top to bottom (on the left vertical axis) are IFN, Immunoproteasome, Plasma Cell, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cell, gd T Cell, Anergic/activated T Cell, Oxidative Phosphorylation, Unfolded Protein, TCRAJ, T Cell, TCRA, TCRB, IL23 Complex and Treg.

FIG. 21: Endotypes in GSE88884. Cosine similarity of the identified endotypes in each GSE88884 Illuminate-1&2 combined (n=1620) and GSE88884 Illuminate-2 (n=807). The heatmap was generated with the plot.matrixR package.

FIGS. 22A-D: Machine learning prediction of molecular endotype memberships using clinical metadata as features One-vs.-one ML classifiers predicting the molecular endotype memberships of GSE88884 ILL-2 using baseline clinical metadata as features rather than baseline gene expression. (FIG. 22A) RF, (FIG. 22B) SVM, (FIG. 22C) LR, and (FIG. 22D) GB classifier ROC curves, performance metrics, and classification schema. Clinical metadata used to determine subsets by either k-means clustering or GMVAE were used as input features: patient ancestry, presence of arthritis, proteinuria, low complement, leukopenia, mucosal ulcers, rash, pleurisy, vasculitis, use of antimalarials, use of corticosteroids, use of immunosuppressants, use of non-steroidal anti-inflammatory drugs, and anti-dsDNA, anti-RNP, anti-Sm, anti-SSA, and anti-SSB titers. Figure components were generated in Python v. 3.8.8 using matplotlib.

FIGS. 23A-B: Endotypes of EA adult SLE patients. The k-means clustering pipeline applied to 1118 baseline active lupus samples from Illuminate-1 and 2 of European ancestry identified six endotypes (FIG. 23A). The identified clusters were compared to the six endotypes identified using all 1620 active lupus samples by cosine similarity (FIG. 23B). Endotype color labels were randomly generated using the ‘grDevices’ color palette in R. Heatmap in (FIG. 23A) was generated with the ComplexHeatmapR package. The plot in (FIG. 23B) was generated with the plot.matrixR package. For FIG. 23A the molecular features (e.g., modules) listed from top to bottom (on the left vertical axis) are IFN, Immunoproteasome, Plasma Cell, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cell, gd T Cell, Anergic/activated T Cell, Oxidative Phosphorylation, Unfolded Protein, TCRAJ, T Cell, TCRA, TCRB, IL23 Complex and Treg.

FIGS. 24A-C: Endotypes of AA adult SLE patients. The k-means clustering pipeline applied to 216 baseline active lupus samples from Illuminate-1 and 2 of African ancestry identified six endotypes (FIG. 24A). The identified clusters were compared to the six endotypes identified using all 1620 active lupus samples (FIG. 24B) and to the sixEA patient endotypes (FIG. 24C) by cosine similarity. Endotype color labels were randomly generated using the ‘grDevices’ color palette in R. Heatmap in (FIG. 24A) was generated with the ComplexHeatmapR package. The plots in (FIGS. 24B-24C) were generated with the plot.matrixR package. For FIG. 24A the molecular features (e.g., modules) listed from top to bottom (on the left vertical axis) are IFN, Immunoproteasome, Plasma Cells, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cells, gd T Cells, Anergic/activated T Cell, Oxidative Phosphorylation, Unfolded Protein, TCRAJ, T Cells, TCRA, TCRB, IL23 Complex and Treg.

FIGS. 25A-C: Endotypes of NAA adult SLE patients. The k-means clustering pipeline applied to 232 baseline active lupus samples from Illuminate-1 and 2 of Native American (Hispanic) ancestry identified six endotypes (FIG. 25A). The identified clusters were compared to the six endotypes identified using all 1620 active lupus samples (FIG. 25B) and to the six EA patient endotypes (FIG. 25C) by cosine similarity. Endotype color labels were randomly generated using the ‘grDevices’ color palette in R. Heatmap in (FIG. 25A) was generated with the ComplexHeatmapR package. The plots in (FIGS. 25B-25C) were generated with the plot.matrixR package. For FIG. 25A the molecular features (e.g., modules) listed from top to bottom (on the left vertical axis) are IFN, Immunoproteasome, Plasma Cells, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cells, gd T Cells, Anergic/activated T Cells, Oxidative Phosphorylation, Unfolded Protein, TCRAJ, T Cells, TCRA, TCRB, IL23 Complex and Treg.

FIGS. 26A-D: Identification of lupus endotypes in external whole blood datasets. K-means clustering of GSVA scores of the 32 features yielded 6 clusters in 266 adult lupus patients from GSE45291 (A), 5 clusters in 137 pediatric lupus patients from GSE65391 (B), and 4 clusters from 160 adult lupus patients from GSE116006 (C) using baseline gene expression. Color labels above the heatmap indicate endotype identity which were randomly generated using the ‘grDevices’ color palette in R. If available, ancestry and disease activity (where active indicates SLEDAI >6) are annotated with color bars below each heatmap. Heatmaps were generated with the ComplexHeatmap R package. Color for GSVA enrichment score, Ancestry and Disease activity status for A-C is shown in D. For FIGS. 26A and 26C the molecular features (e.g., modules) listed from top to bottom (on the left vertical axis) are IFN, Immunoproteasome, Plasma Cells, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cells, gd T Cells, Anergic/activated T Cells, Oxidative Phosphorylation, Unfolded Protein, TCRAJ, T Cells, TCRA, TCRB, IL23 Complex and Treg. For FIG. 26B the molecular features (e.g., modules) listed from top to bottom (on the left vertical axis) are IFN, Immunoproteasome, Plasma Cells, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, NK Cell, MHCII, B Cells, gd T Cells, Anergic/activated T Cells, Oxidative Phosphorylation, Unfolded Protein, T Cells, TCRA, IL23 Complex and Treg.

FIG. 27: Determination of k clusters. For each individually endotyped dataset, the elbow method and silhouette analysis were used to determine the optimal number of clusters, or endotypes. Plots were generated in Python using matplotlib and both methods were used in the final determination of k clusters for individual datasets. Datasets are identified by their respective GEO accession number.

FIGS. 28A-D: K-means clustering of lupus and controls samples. K-means clustering of GSVA scores of the 32 features in lupus patients and controls (CTLs) in (A) GSE45291 (266 SLE and 20 CTL), (B) GSE65391 (137 SLE and 57 CTL), (C) GSE138458 (307 SLE and 23 CTL), and (D) GSE185047 (177 SLE and 10 CTL). Endotypes in GSE116006 were featured in a main figure but did not contain control samples, thus datasets in (C) and (D) were clustered with controls to further illustrate the assignation of “abnormally enriched” endotypes. The control samples in GSE138458 were patients with non-autoimmune rheumatic diseases recruited from health fairs, whereas in the other datasets controls were healthy. In (A), 75% of controls were found in M4′(M4′=15 CTLs, Pu′=3 CTLs, C2′=2 CTLs). In (B), 57.9% of controls were found in RB′(RB′=33 CTLs, LB1′=22 CTLs, DOG′=2 CTLs). In (C), 47.8% of controls were found in S2′(S2′=11 CTLs, K2′=4 CTLs, LB1′=3 CTLs, C3′=3 CTLs, S3′=2 CTLs). In (D), all 10 controls were found in O′. Heatmaps were generated with GraphPad Prism v. 9.4.0 (673). Color for GSVA enrichment score, and cohort for A-D is shown in E. For FIGS. 28A and 28D the molecular features (e.g., modules) listed from top to bottom (on the left vertical axis) are IFN, Immunoproteasome, Plasma Cells, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cells, gd T Cells, Anergic/activated T Cells, Oxidative Phosphorylation, Unfolded Protein, TCRAJ, T Cells, TCRA, TCRB, IL23 Complex and Treg. FIG. 28B the molecular features (e.g., modules) listed from top to bottom (on the left vertical axis) are IFN, Immunoproteasome, Plasma Cells, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, NK Cell, MHCII, B Cells, gd T Cells, Anergic/activated T Cells, Oxidative Phosphorylation, Unfolded Protein, T Cells, TCRA, IL23 Complex and Treg. FIG. 28C the molecular features (e.g., modules) listed from top to bottom (on the left vertical axis) are IFN, Immunoproteasome, Plasma Cells, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, NK Cell, MHCII, B Cells, gd T Cells, Anergic/activated T Cells, Oxidative Phosphorylation, Unfolded Protein, T Cells, IL23 Complex and Treg.

FIGS. 29A-29B: Comparison of endotypes in additional lupus datasets with controls. Cosine similarity of k-means clusters identified in SLE patients alone versus SLE+CTLs in GSE45291 (A) and GSE65391 (B). Plots were generated in R using the plot.matrixpackage.

FIGS. 30A-B: Determination of total lupus endotypes Cosine similarity analysis (A) and hierarchical clustering (B) of the endotypes identified in these five datasets led to a final designation of eight transcriptionally distinct endotypes. Endotypes were considered similar after cosine similarity >0.7. Endotypes underlined in red in (A) indicate the unique endotypes. Hierarchical clustering using complete agglomeration and a cut height of 1.8 is displayed in (B). Heatmaps in (A) and (B) were generated using the plot.matrixand ggplot2 R packages, respectively.

FIGS. 31A-E: Clinical characteristics of five pediatric lupus endotypes. Quantitative and categorical clinical metadata were summarized for each cluster identified in GSE65391 using baseline values. Metadata was categorized by (A) quantitative immunologic/inflammatory and systemic disease indicators, (B) qualitative immunologic/inflammatory and systemic disease indicators, (C) medication use, (D) patient ancestry, and (E) kidney disease activity. Labels on x-axes indicate the shorthand name for patient clusters and colors were randomly generated using the ‘grDevices’ color palette in R. RB=rosybrown, LB1=lightblue1, DOG=darkolivegreen, Th1=thistle1, and S3=sienna3. Scatterplots in (A) display the mean±SD for each cluster; statistical differences were found with Dunn's multiple comparisons test. Lymphopenia was defined as less than 1 billion lymphocytes per liter. Low C3 and C4 were defined as less than 0.8 g/L and 0.2 g/L, respectively. Significant associations between categorical variables and endotype cluster (denoted with asterisks) were identified using Chi Square Test of Independence. In (B)-(E), odds ratios of RB having a positive value for the clinical trait of interest are displayed above the RB bar with significance indicated by asterisks. Missing data (n.d.) were excluded from analyses. Graphs were created in GraphPad Prism v 9.1.0 (221). ESR=erythrocyte sedimentation rate. n.d.=no data. HCQ=hydroxychloroquine.

MMF=mycophenolate mofetil. LN=lupus nephritis. *p<0.05; **p<0.01; ***p<0.001; ****p<0.0001

FIGS. 32A-E: Machine learning algorithms can predict lupus endotype membership with high accuracy. Multi-class classification by ML categorized 3,166 lupus patients from 17 datasets into eight patient endotypes. Area under the ROC curve (AUC), performance metrics, and confusion matrices for each of 4 classifiers are summarized: (B) random forest, (C) support vector machine, (D) logistic regression, and (E) gradient boosting. Each model was trained on 1746 lupus samples, validated with 437 lupus samples, and tested on the remaining 938 samples for a total N=3166 from 17 datasets. Eight final endotypes of lupus were determined by k-means clustering of the 3,166 patients' concatenated GSVA enrichment scores of 26/32 features (A). Plots in (B)-(E) were generated in Python using the scikit-learn and matplotlib libraries. Heatmap in (A) was generated with the ComplexHeatmap R package. For FIG. 32A the molecular features (e.g., modules) listed from top to bottom (on the left vertical axis) are IFN, Immunoproteasome, Plasma Cell, Cell Cycle, SNOR Low UP, IL1 Pathway, Inflammasone, Inhibitory Macrophage, Inflammatory Cytokines, Anti-inflammation, TNF, Monocyte, Neutrophil, Granulocyte, LDG, Dendritic Cell, pDC, NK Cell, MHCII, B Cell, gd T Cell, Anergic/activated T Cell, Oxidative Phosphorylation, Unfolded Protein, T Cell, and IL23 Complex. For FIG. 32A the endotypes listed from left to right (on the top horizontal axis) are A, B, C, D, E, F, G and H.

FIGS. 33A-G: LuCIS as a composite metric to summarize module abnormalities in individual lupus patients and estimate disease severity. Logistic regression with ridge penalization was employed to classify the “least abnormal” (A) and “most abnormal” (H) subsets from 17 lupus blood gene expression datasets using 26/32 modules as features. The resulting model produced coefficients that can be used to calculate LuCIS (A). The mean±SEM (top) and distribution (bottom) of LuCIS for the eight endotypes in all 17 datasets (B). Statistical differences between mean LuCIS of the endotypes were evaluated with the Kruskal-Wallis test and Dunn's multiple comparisons. (C) Comparison of mean LuCIS between non-lupus healthy controls and the least transcriptionally perturbed or least “abnormal” lupus endotype from samples in 5 independent datasets for which adequate controls were available (GSE45291, GSE65391, GSE88884 ILL-1, GSE88884 ILL-2, and GSE185047). Missing values for GSVA modules not measured (IG chains, TCRAJ, TCRB, TCRD) in GSE65391 were imputed as described in the Supplement. Pearson correlations between LuCIS and anti-dsDNA titers (D), SLEDAI (E), serum C3 (F), or serum C4 (G). All plots were generated, and statistics were computed, in GraphPad Prism v. 9.4.0 (673).

FIGS. 34A-E: One-vs-rest multi-class classification of lupus endotype memberships. One-vs-rest multi-class classification by machine learning analysis. Area under the ROC curve (AUC), performance metrics, and confusion matrices of each of 4 classifiers are summarized: (A) random forest, (B) support vector machine, (C) logistic regression, (D) gradient boosting, and (E) extreme gradient boosting (XGB). Each model was trained on 1746 lupus samples, validated with 437 samples, and tested on the remaining 938 samples for a total N=3166 from 17 datasets. Plots were created in Python using matplotlib.

FIG. 35: Summary Plot of SHAP values for XGB multi-class ML model. Mean absolute SHAP values of the top 20 features of the XGB multi-class one-vs-rest ML model across patients in eight endotypes identified in 3166 samples from 17 lupus whole blood datasets. A refers to the least perturbed endotype (FIG. 32A) and H refers to the most perturbed endotype (FIG. 32A). The plot was generated in Python using the shapmodule. For each horizontal bar, endotype bars from left to right are sequentially: A, H, G, D, B, E, F, C. The x-axis values are from left to right: 0, 0.5, 1.0, 1.5, 2.0, 2.5, 3.0, 3.5, 4.0 (mean SHAP value average impact on model output magnitude).

FIGS. 36A-E: Distinguishment of endotype B from A. Results of binary classification to distinguish endotype B (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features.

Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIG. 37A-E: Distinguishment of endotype C from A. Results of binary classification to distinguish endotype C (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features.

Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for two exemplary features. The x-axis represents the GSVA score of the primary feature and the color represents the GSVA score of the secondary feature.

FIGS. 38A-E: Distinguishment of endotype D from A. Results of binary classification to distinguish endotype D (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features. Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIGS. 39A-E: Distinguishment of endotype E from A. Results of binary classification to distinguish endotype E (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features. Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIGS. 40A-E: Distinguishment of endotype F from A. Results of binary classification to distinguish endotype F (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features. Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIGS. 41A-E: Distinguishment of endotype G from A. Results of binary classification to distinguish endotype G (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features. Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIGS. 42A-E: Distinguishment of endotype H from A. Results of binary classification to distinguish endotype H (FIG. 32A) from A using the GSVA enrichment scores of 26/32 molecular features present in all 17 datasets (Table 33). (A) ML performance metrics of nine classifiers. LR=logistic regression, RF=random forest, SVM=support vector machine, DTREE=decision trees, ADB=adaptive boosting, NB=naïve Bayes, LDA=linear discriminant analysis, KNN=k nearest neighbors, GB=gradient boosting. (B) RF SHAP summary plot of the top 15 important features ranked top to bottom. Feature value represents the GSVA enrichment score and each dot is a sample. (C) RF SHAP waterfall plot of an exemplary individual sample. Expected SHAP values are displayed on the bottom x-axis (calculated as the average SHAP value across all samples) and the actual SHAP value for that sample is displayed on the top x-axis. GSVA scores are displayed next to y-axis labels for the top 15 features. Values in the boxes in the plot enumerate how much a feature increases or decreases the final model outcome for the exemplary sample. (D) RF SHAP bar plot which enumerates the contribution of each feature across all samples. (E) RF SHAP dependence plots or scatter plots illustrating the impact of each feature toward the final model prediction and its interaction with a second feature for three exemplary features. The x-axis represents the GSVA score of the primary feature and the color represents the GSVA score of the secondary feature.

FIG. 43: SHAP analysis reveals features most distinctive of transcriptional perturbations in seven out of eight lupus endotypes. SHAP analysis of the seven binary ML classifiers distinguishing the seven out of eight most transcriptionally abnormal lupus endotypes (B-H) from the eighth least abnormal endotype (A) reveals the features most contributory to the ML model's classification capacity. The size (area) of the bubbles in the plot enumerate the absolute value of the SHAP contribution of each feature listed on the y-axis. Bubble plot rendered in R using the ggplot2 package. The features shown from top to bottom (left vertical axis) are Anergic/activated T cell, Anti-inflammation, B cell, Cell cycle, Dendritic cell, gd T cell, granulocyte, IFN, IL 1 pathway, IL 23 complex, Immunoproteasome, Inflammasome, Inflammatory Cytokines, Inhibitory Macs, LDG, MHCII, Monocyte, Neutrophill, NK cell, Oxidative Phosphorylation, pDC, Plasma cell, SNOR low up, T cell, TCRA, TNF, Treg, and Unfolded protein.

FIG. 44: Bubble Plot. Bubble plot visualizing number of features required to identify all 8 lupus endotypes.

FIGS. 45A-B: Systemic sclerosis (scleroderma) (SSc) skin endotypes can be predicted from analysis of blood gene expression. (FIG. 45A) K-means clustering of blood from matched SSc patients (GSE179153) into the SSc skin-identified clusters. (FIG. 45B) CART classification of the purple (most severe) cluster using blood gene expression modules. P is the probability of identifying individual patients in the purple cluster. For FIG. 45A the molecular features (e.g., modules) listed from top to bottom (on the left vertical axis) are Monocyte, TNF, Inflammasome, Neutrophil, IL1 Pathway, Inhibitory Macrophage, Granulocyte, SNOR Low UP, Inflammatory Cytokines, pDC, Treg, LDG, IFN, Anti-inflammation, Immunoproteasome, Cell cycle, Dendritic Cell, Oxidative Phosphorylation, Unfolded Protein, Plasma Cells, MHCII, B Cell, IL23 Complex, Anergic/activated T Cell, NK Cell, T cell, and gd T cell.

FIGS. 46A-H show a method of determining effective number of genes for a gene module/Table. FIGS. 46A-D show ARI (Adjusted Rand Index) Line plots for LuGene modules, Monocyte (Table 18, FIG. 46A), IG Chains (Table 9, FIG. 46B), IFN (Table 8, FIG. 46C), LDG (Table 16, FIG. 46D). FIGS. 46E-H show the confusion matrixes showing the cluster memberships of various subsets to the reference population. FIG. 46E shows similarity of the kmeans cluster memberships from random monocyte subset to the reference Monocyte module (all genes). FIG. 46F shows similarity of the kmeans cluster memberships from random IFN subset to the reference IFN module (all genes). FIG. 46G shows similarity of the kmeans cluster memberships from random LDG subset to the reference LDG module (all genes). FIG. 46H shows similarity of the kmeans cluster memberships from random IG chain subset to the reference IG chain module (all genes).

FIGS. 47A-F show SHAP analysis reveals features most distinctive of transcriptional perturbations in the endotypes. FIG. 47A shows SHAP analysis for binary classification of endotype A from endotypes B, C, D, E, F, G and H. FIG. 47B shows SHAP analysis for binary classification of endotype E from endotypes B, C, and D. FIG. 47C shows SHAP analysis for binary classification of endotype F from endotypes B, C, D and E. FIG. 47D shows SHAP analysis for binary classification of endotype D from endotypes B, and C. FIG. 47E shows SHAP analysis for binary classification of endotype G from endotypes B, C, D, E and F. FIG. 47F shows SHAP analysis for binary classification of endotype H from endotypes B, C, D, E, F and G.

FIGS. 48-1 to 48-28 show performance metrics for 28 pairwise binary classifications (FIG. 48-1: group A vs. group B; FIG. 48-2: group A vs. group C; FIG. 48-3:group A vs. group D; FIG. 48-4: group A vs. group E; FIG. 48-5: group A vs. group F; FIG. 48-6: group A vs. group G; FIG. 48-7: group A vs. group H; FIG. 48-8: group B vs. group C; FIG. 48-9: group B vs. group D; FIG. 48-10: group B vs. group E; FIG. 48-11: group B vs. group F; FIG. 48-12: group B vs. group G; FIG. 48-13: group B vs. group H; FIG. 48-14: group C vs. group D; FIG. 48-15: group C vs. group E; FIG. 48-16: group C vs. group F; FIG. 48-17: group C vs. group G; FIG. 48-18: group C vs. group H; 48-19: group D vs. group E; FIG. 48-20: group D vs. group F; FIG. 48-21: group D vs. group G; FIG. 48-22: group D vs. group H; 48-23: group E vs. group F; FIG. 48-24: group E vs. group G; FIG. 48-25: group E vs. group H; FIG. 48-26: group F vs. group G; FIG. 48-27: group F vs. group H; FIG. 48-28: group G vs. group H;) using the genes from top 3 SHAP predictors of each classification. The top 3 SHAP predictors (marked as 1, 2 and 3) of each classification is shown in Table 40.

FIGS. 49-1 to 49-28 show ROC curves for 28 pairwise binary classifications (FIG. 49-1: group A vs. group B; FIG. 49-2: group A vs. group C; FIG. 49-3:group A vs. group D; FIG. 49-4: group A vs. group E; FIG. 49-5: group A vs. group F; FIG. 49-6: group A vs. group G; FIG. 49-7: group A vs. group H; FIG. 49-8: group B vs. group C; FIG. 49-9: group B vs. group D; FIG. 49-10: group B vs. group E; FIG. 49-11: group B vs. group F; FIG. 49-12: group B vs. group G; FIG. 49-13: group B vs. group H; FIG. 49-14: group C vs. group D; FIG. 49-15: group C vs. group E; FIG. 49-16: group C vs. group F; FIG. 49-17: group C vs. group G; FIG. 49-18: group C vs. group H; 49-19: group D vs. group E; FIG. 49-20: group D vs. group F; FIG. 49-21: group D vs. group G; FIG. 49-22: group D vs. group H; 49-23: group E vs. group F; FIG. 49-24: group E vs. group G; FIG. 49-25: group E vs. group H; FIG. 49-26: group F vs. group G; FIG. 49-27: group F vs. group H; FIG. 49-28: group G vs. group H;) using the genes from top 3 SHAP predictors of each classification. The top 3 SHAP predictors (marked as 1, 2 and 3) of each classification is shown in Table 40.

FIGS. 50-1 to 50-28 show performance metrics for 28 pairwise binary classifications (FIG. 50-1: group A vs. group B; FIG. 50-2: group A vs. group C; FIG. 50-3:group A vs. group D; FIG. 50-4: group A vs. group E; FIG. 50-5: group A vs. group F; FIG. 50-6: group A vs. group G; FIG. 50-7: group A vs. group H; FIG. 50-8: group B vs. group C; FIG. 50-9: group B vs. group D; FIG. 50-10: group B vs. group E; FIG. 50-11: group B vs. group F; FIG. 50-12: group B vs. group G; FIG. 50-13: group B vs. group H; FIG. 50-14: group C vs. group D; FIG. 50-15: group C vs. group E; FIG. 50-16: group C vs. group F; FIG. 50-17: group C vs. group G; FIG. 50-18: group C vs. group H; 50-19: group D vs. group E; FIG. 50-20: group D vs. group F; FIG. 50-21: group D vs. group G; FIG. 50-22: group D vs. group H; 50-23: group E vs. group F; FIG. 50-24: group E vs. group G; FIG. 50-25: group E vs. group H; FIG. 50-26: group F vs. group G; FIG. 50-27: group F vs. group H; FIG. 50-28: group G vs. group H;) using the top 10 gene predictors of each binary classification. The top 10 gene predictors of each classification is shown in Table 41.

FIGS. 51-1 to 51-28 show ROC curves for 28 pairwise binary classifications (FIG. 51-1: group A vs. group B; FIG. 51-2: group A vs. group C; FIG. 51-3:group A vs. group D; FIG. 51-4: group A vs. group E; FIG. 51-5: group A vs. group F; FIG. 51-6: group A vs. group G; FIG. 51-7: group A vs. group H; FIG. 51-8: group B vs. group C; FIG. 51-9: group B vs. group D; FIG. 51-10: group B vs. group E; FIG. 51-11: group B vs. group F; FIG. 51-12: group B vs. group G; FIG. 51-13: group B vs. group H; FIG. 51-14: group C vs. group D; FIG. 51-15: group C vs. group E; FIG. 51-16: group C vs. group F; FIG. 51-17: group C vs. group G; FIG. 51-18: group C vs. group H; 51-19: group D vs. group E; FIG. 51-20: group D vs. group F; FIG. 51-21: group D vs. group G; FIG. 51-22: group D vs. group H; 51-23: group E vs. group F; FIG. 51-24: group E vs. group G; FIG. 51-25: group E vs. group H; FIG. 51-26: group F vs. group G; FIG. 51-27: group F vs. group H; FIG. 51-28: group G vs. group H;) using the top 10 gene predictors of each binary classification. The top 10 gene predictors of each classification is shown in Table 41.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “Gini impurity” refers to a measure of how often a randomly chosen element from the set may be incorrectly labeled if it is randomly labeled according to the distribution of labels in the subset.

The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosure.

### Method for Classifying a Lupus Disease State of a Patient

One aspect of the present disclosure is directed to a method for classifying a lupus disease state of a patient. The method can include analyzing a data set comprising or derived from gene expression measurements of at least 2 genes, to classify the lupus disease state of the patient. The dataset can be analyzed to generate an inference indicative of the lupus disease state of the patient. The gene expression measurements can be obtained from a biological sample obtained or derived from the patient. In certain embodiments, the lupus disease state of the patient is group A lupus disease state, group B lupus disease state, group C lupus disease state, group D lupus disease state, group E lupus disease state, group F lupus disease state, group G lupus disease state, or group H lupus disease state. In certain embodiments, classifying the lupus disease state of the patient include classifying whether the patient has the group A lupus disease state, group B lupus disease state, group C lupus disease state, group D lupus disease state, group E lupus disease state, group F lupus disease state, group G lupus disease state, or group H lupus disease state. In certain embodiments, the inference can be whether the data set is indicative of the patient having group A lupus disease state, group B lupus disease state, group C lupus disease state, group D lupus disease state, group E lupus disease state, group F lupus disease state, group G lupus disease state, or group H lupus disease state. In certain embodiments, classifying the lupus disease state of the patient include classifying whether the patient has group A lupus disease state. In certain embodiments, classifying the lupus disease state of the patient include classifying whether the patient has group B lupus disease state. In certain embodiments, classifying the lupus disease state of the patient include classifying whether the patient has group C lupus disease state. In certain embodiments, classifying the lupus disease state of the patient include classifying whether the patient has group D lupus disease state. In certain embodiments, classifying the lupus disease state of the patient include classifying whether the patient has group E lupus disease state. In certain embodiments, classifying the lupus disease state of the patient include classifying whether the patient has group F lupus disease state. In certain embodiments, classifying the lupus disease state of the patient include classifying whether the patient has group G lupus disease state. In certain embodiments, classifying the lupus disease state of the patient include classifying whether the patient has group H lupus disease state. In certain embodiments, classifying the lupus disease state of the patient include classifying whether the patient has group A lupus disease state, or group B lupus disease state. In certain embodiments, classifying the lupus disease state of the patient include classifying whether the patient has group A lupus disease state, or group C lupus disease state. In certain embodiments, classifying the lupus disease state of the patient include classifying whether the patient has group A lupus disease state, or group D lupus disease state. In certain embodiments, classifying the lupus disease state of the patient include classifying whether the patient has group A lupus disease state, or group E lupus disease state. In certain embodiments, classifying the lupus disease state of the patient include classifying whether the patient has group A lupus disease state or group F lupus disease state. In certain embodiments, classifying the lupus disease state of the patient include classifying whether the patient has group A lupus disease state or group G lupus disease state. In certain embodiments, classifying the lupus disease state of the patient include classifying whether the patient has group A lupus disease state or group H lupus disease state. In certain embodiments, classifying the lupus disease state of the patient include classifying whether the patient has group B lupus disease state, or group C lupus disease state. In certain embodiments, classifying the lupus disease state of the patient include classifying whether the patient has group B lupus disease state, or group D lupus disease state. In certain embodiments, classifying the lupus disease state of the patient include classifying whether the patient has group B lupus disease state, or group E lupus disease state. In certain embodiments, classifying the lupus disease state of the patient include classifying whether the patient has group B lupus disease state or group F lupus disease state. In certain embodiments, classifying the lupus disease state of the patient include classifying whether the patient has group B lupus disease state or group G lupus disease state. In certain embodiments, classifying the lupus disease state of the patient include classifying whether the patient has group B lupus disease state or group H lupus disease state. In certain embodiments, classifying the lupus disease state of the patient include classifying whether the patient has group C lupus disease state, or group D lupus disease state. In certain embodiments, classifying the lupus disease state of the patient include classifying whether the patient has group C lupus disease state, or group E lupus disease state. In certain embodiments, classifying the lupus disease state of the patient include classifying whether the patient has group C lupus disease state or group F lupus disease state. In certain embodiments, classifying the lupus disease state of the patient include classifying whether the patient has group C lupus disease state or group G lupus disease state. In certain embodiments, classifying the lupus disease state of the patient include classifying whether the patient has group C lupus disease state or group H lupus disease state. In certain embodiments, classifying the lupus disease state of the patient include classifying whether the patient has group D lupus disease state, or group E lupus disease state. In certain embodiments, classifying the lupus disease state of the patient include classifying whether the patient has group D lupus disease state or group F lupus disease state. In certain embodiments, classifying the lupus disease state of the patient include classifying whether the patient has group D lupus disease state or group G lupus disease state. In certain embodiments, classifying the lupus disease state of the patient include classifying whether the patient has group D lupus disease state or group H lupus disease state. In certain embodiments, classifying the lupus disease state of the patient include classifying whether the patient has group E lupus disease state or group F lupus disease state. In certain embodiments, classifying the lupus disease state of the patient include classifying whether the patient has group E lupus disease state or group G lupus disease state. In certain embodiments, classifying the lupus disease state of the patient include classifying whether the patient has group E lupus disease state or group H lupus disease state. In certain embodiments, classifying the lupus disease state of the patient include classifying whether the patient has group F lupus disease state or group G lupus disease state. In certain embodiments, classifying the lupus disease state of the patient include classifying whether the patient has group F lupus disease state or group H lupus disease state. In certain embodiments, classifying the lupus disease state of the patient include classifying whether the patient has group G lupus disease state or group H lupus disease state. In certain embodiments, the inference is whether the data set is indicative of the patient having group A lupus disease state. In certain embodiments, the inference is whether the data set is indicative of the patient having group B lupus disease state. In certain embodiments, the inference is whether the data set is indicative of the patient having group C lupus disease state. In certain embodiments, the inference is whether the data set is indicative of the patient having group D lupus disease state. In certain embodiments, the inference is whether the data set is indicative of the patient having group E lupus disease state. In certain embodiments, the inference is whether the data set is indicative of the patient having group F lupus disease state. In certain embodiments, the inference is whether the data set is indicative of the patient having group G lupus disease state. In certain embodiments, the inference is whether the data set is indicative of the patient having group H lupus disease state. In certain embodiments, the inference is whether the data set is indicative of the patient having group A lupus disease state, or group B lupus disease state. In certain embodiments, the inference is whether the data set is indicative of the patient having group A lupus disease state, or group C lupus disease state. In certain embodiments, the inference is whether the data set is indicative of the patient having group A lupus disease state, or group D lupus disease state. In certain embodiments, the inference is whether the data set is indicative of the patient having group A lupus disease state, or group E lupus disease state. In certain embodiments, the inference is whether the data set is indicative of the patient having group A lupus disease state, or group F lupus disease state. In certain embodiments, the inference is whether the data set is indicative of the patient having group A lupus disease state, or group G lupus disease state. In certain embodiments, the inference is whether the data set is indicative of the patient having group A lupus disease state, or group H lupus disease state. In certain embodiments, the inference is whether the data set is indicative of the patient having group B lupus disease state, or group C lupus disease state. In certain embodiments, the inference is whether the data set is indicative of the patient having group B lupus disease state, or group D lupus disease state. In certain embodiments, the inference is whether the data set is indicative of the patient having group B lupus disease state, or group E lupus disease state. In certain embodiments, the inference is whether the data set is indicative of the patient having group B lupus disease state, or group F lupus disease state. In certain embodiments, the inference is whether the data set is indicative of the patient having group B lupus disease state, or group G lupus disease state. In certain embodiments, the inference is whether the data set is indicative of the patient having group B lupus disease state, or group H lupus disease state. In certain embodiments, the inference is whether the data set is indicative of the patient having group C lupus disease state, or group D lupus disease state. In certain embodiments, the inference is whether the data set is indicative of the patient having group C lupus disease state, or group E lupus disease state. In certain embodiments, the inference is whether the data set is indicative of the patient having group C lupus disease state, or group F lupus disease state. In certain embodiments, the inference is whether the data set is indicative of the patient having group C lupus disease state, or group G lupus disease state. In certain embodiments, the inference is whether the data set is indicative of the patient having group C lupus disease state, or group H lupus disease state. In certain embodiments, the inference is whether the data set is indicative of the patient having group D lupus disease state, or group E lupus disease state. In certain embodiments, the inference is whether the data set is indicative of the patient having group D lupus disease state, or group F lupus disease state. In certain embodiments, the inference is whether the data set is indicative of the patient having group D lupus disease state, or group G lupus disease state. In certain embodiments, the inference is whether the data set is indicative of the patient having group D lupus disease state, or group H lupus disease state. In certain embodiments, the inference is whether the data set is indicative of the patient having group E lupus disease state, or group F lupus disease state. In certain embodiments, the inference is whether the data set is indicative of the patient having group E lupus disease state, or group G lupus disease state. In certain embodiments, the inference is whether the data set is indicative of the patient having group E lupus disease state, or group H lupus disease state. In certain embodiments, the inference is whether the data set is indicative of the patient having group F lupus disease state, or group G lupus disease state. In certain embodiments, the inference is whether the data set is indicative of the patient having group F lupus disease state, or group H lupus disease state. In certain embodiments, the inference is whether the data set is indicative of the patient having group G lupus disease state, or group H lupus disease state. In certain embodiments, classifying the lupus disease state of the patient can include classifying whether the patient has lupus. In certain embodiments, the inference can be whether the data set is indicative of the patient having lupus.

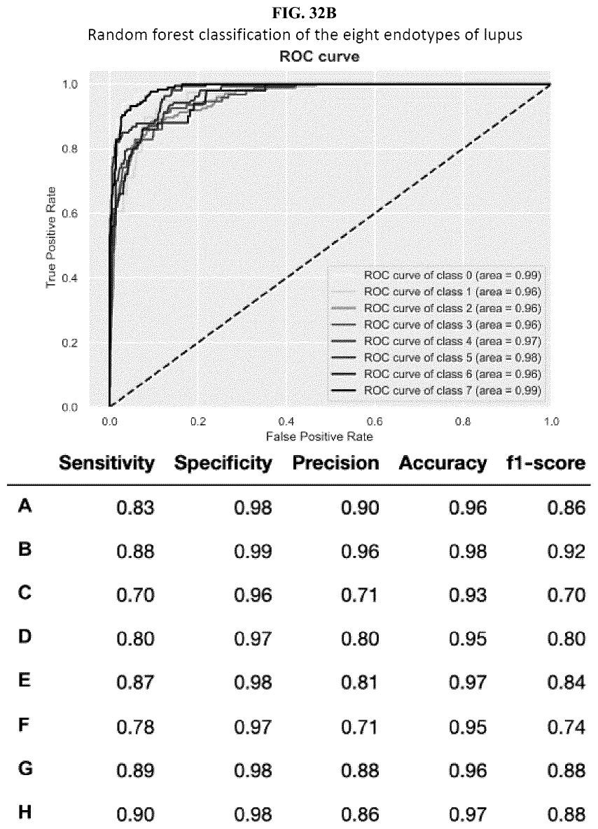

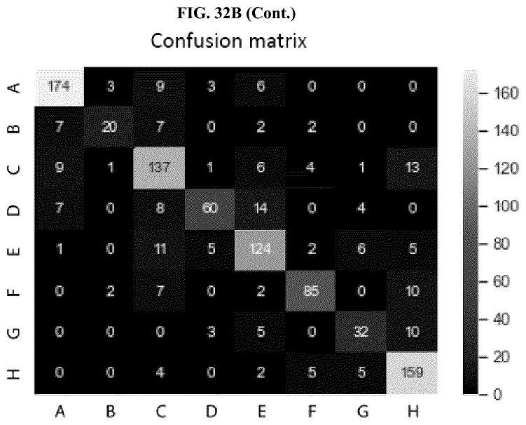

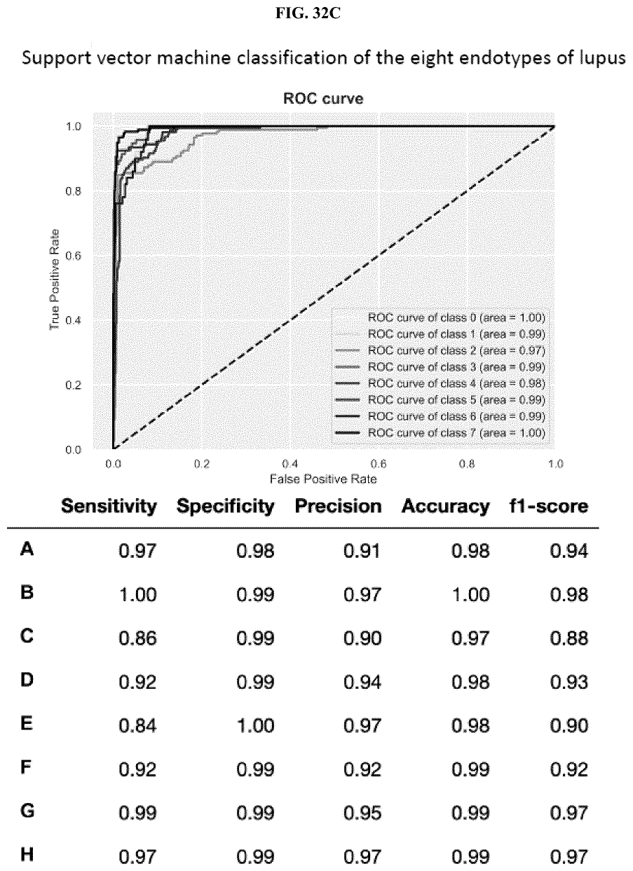

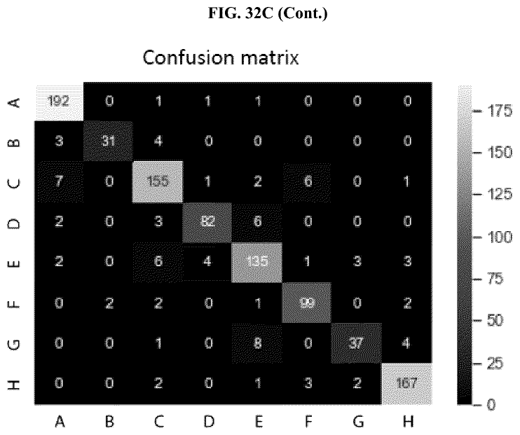

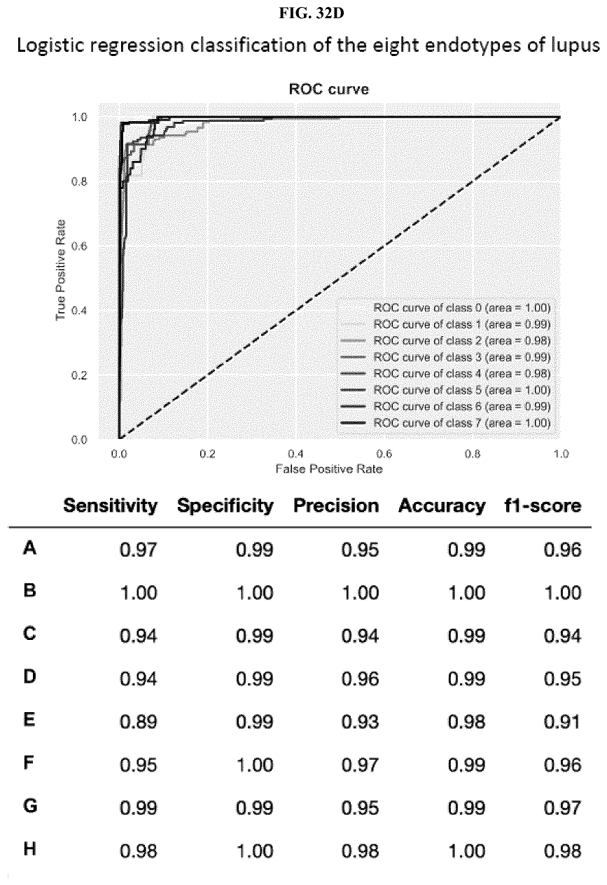

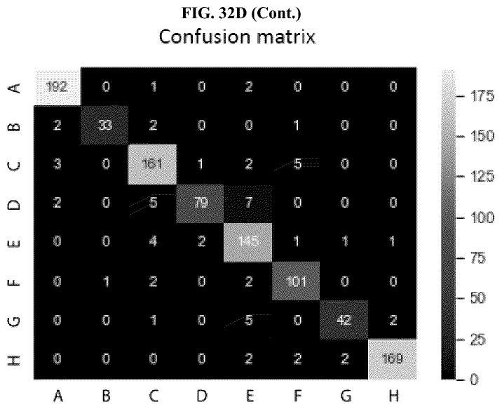

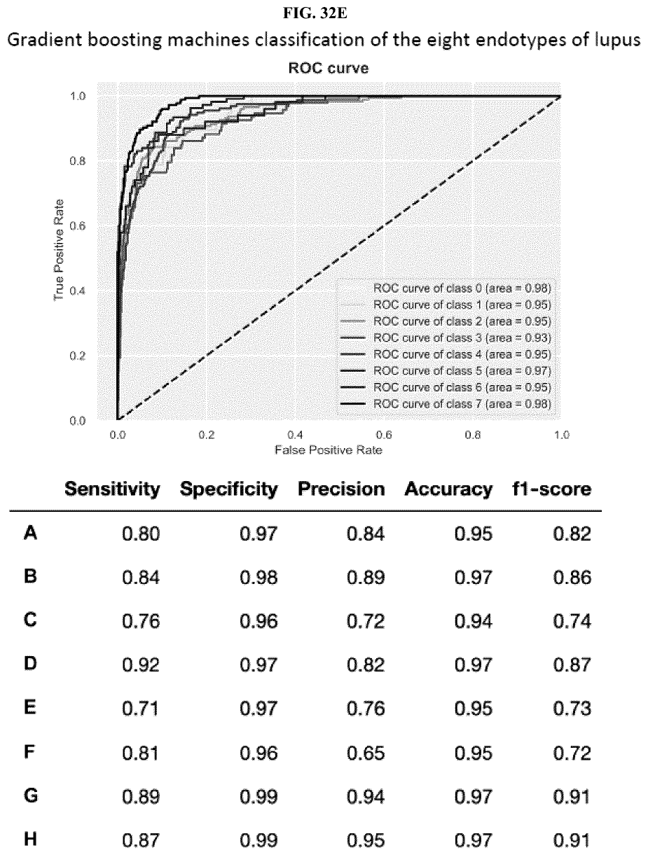

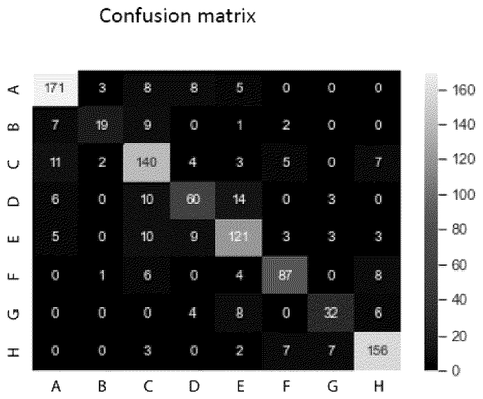

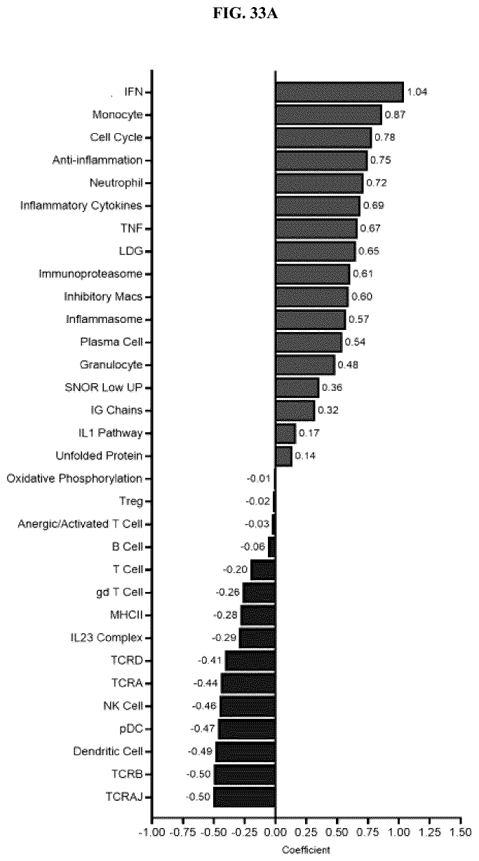

The blood transcriptomic profile of a patient having group A, B, C, D, E, F, G, or H lupus disease state can fall under endotype A, B, C, D, E, F, G, or H, respectively as shown in FIG. 32A. Blood transcriptomic profile of patients of endotype A, e.g., having group A lupus disease state can resemble non-lupus controls. For endotypes B-H, abnormal modules (e.g., modules having abnormal gene expression) compared to endotype A are shown in Table 39. As discussed in example 2, for an endotype for a module, if Z-score falls between −2 and 2 (e.g., −2 to 2), the endotype gene expression for that module is considered within the normal range, whereas if the Z-score is <−2 or >2, the endotype gene expression for that module is considered abnormal. A module can be considered significantly enriched in an endotype, compared to endotype A if the Z score is >2. A module can be considered significantly de-enriched in an endotype, compared to endotype A if the Z score is <−2. Z-score can be calculated using a method as described in Example 2, and can be, Z-score=(endotype module mean GSVA score−endotype A module mean GSVA score)/endotype A module standard deviation GSVA Score. Endotype B can have modules TCRD, gd T Cell, TCRAJ, T Cell, TCRA, TCRB and/or Treg, significantly de-enriched compared to endotype A. The modules TCRD, gd T Cell, TCRAJ, T Cell, TCRA, TCRB and/or Treg, can be de-enriched in a blood sample from a patient of Endotype B, compared to non-lupus control. Endotype C can have module Monocyte significantly enriched compared to endotype A. The module Monocyte, can be enriched in a blood sample from a patient of Endotype C, compared to non-lupus control. Endotype D can have module IFN significantly enriched compared to endotype A. The module IFN, can be enriched in a blood sample from a patient of Endotype D, compared to non-lupus control. Endotype E can have modules IFN, and/or cell cycle significantly enriched compared to endotype A. The modules IFN and/or cell cycle, can be enriched in a blood sample a patient of Endotype E, compared to non-lupus control. Endotype F can have i) modules Anti-inflammation, Monocyte, Neutrophil, and/or Granulocyte significantly enriched compared to endotype A, and/or ii) modules TCRD, gd T Cell, Anergic/activated T Cell, Oxidative Phosphorylation, TCRAJ, T Cell, TCRA, TCRB, and/or Treg, significantly de-enriched compared to endotype A. The modules i) Anti-inflammation, Monocyte, Neutrophil, and/or Granulocyte can be enriched, and/or ii) TCRD, gd T Cell, Anergic/activated T Cell, Oxidative Phosphorylation, TCRAJ, T Cell, TCRA, TCRB, and/or Treg can be de-enriched, in a blood sample from a patient of Endotype F, compared to non-lupus control. Endotype G can have modules IFN, Immunoproteasome, IL1 Pathway, Inflammasome, Inhibitory Macrophages (Inhibitory Macs), Inflammatory Cytokines, Anti-inflammation, TNF, Monocyte, Neutrophil, and/or granulocyte significantly enriched compared to endotype A. The modules IFN, Immunoproteasome, IL1 Pathway, Inflammasome, Inhibitory Macrophages (Inhibitory Macs), Inflammatory Cytokines, Anti-inflammation, TNF, Monocyte, Neutrophil, and/or granulocyte, can be enriched, in a blood sample from a patient of Endotype G, compared to non-lupus control. Endotype H can have i) modules IFN, Inflammasome, Inhibitory Macs, Inflammatory Cytokines, Anti-inflammation, TNF, Monocyte, Neutrophil, and/or granulocyte significantly enriched compared to endotype A, and ii) modules TCRD, gd T Cell, TCRAJ, T Cell, TCRA, TCRB, and/or Treg, significantly de-enriched compared to endotype A. The modules i) IFN, Inflammasome, Inhibitory Macs, Inflammatory Cytokines, Anti-inflammation, TNF, Monocyte, Neutrophil, and/or granulocyte can be enriched, and/or ii) TCRD, gd T Cell, TCRAJ, T Cell, TCRA, TCRB, and/or Treg can be de-enriched in a blood sample from a patient of Endotype H, compared to non-lupus control. The modules and the genes within the modules are listed in Tables: 1 to 32. A patient having group B, C, D, E, F, G or H lupus disease state can have lupus.

In certain embodiments, the at least 2 genes are selected from the genes listed Tables: 1 to 32. Genes listed in Tables: 1 to 32, include all the genes listed in Tables: 1 to 32. In some embodiments, the at least 2 genes are selected from a group of genes listed in 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 Tables of Tables 1 to 32. In certain embodiments, the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all genes, selected from the genes listed in Tables: 1 to 32, e.g., the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all genes, selected from the genes listed in Tables: 1 to 32. In certain embodiments, the at least 2 genes comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, or any value or range there between, genes, selected from genes listed in Tables: 1 to 32. In certain embodiments, the at least 2 genes consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, or any value or range there between, genes, selected from the genes listed in Tables: 1 to 32. In certain embodiments, the at least 2 genes are selected from the genes listed in Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 722, or all genes, selected from the genes listed in Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the at least 2 genes comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 722, or all or any value or range there between, genes, selected from the genes listed in Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the data set comprises or is derived from gene expression measurements of at least 2 genes selected from the genes listed in each of one or more Tables selected from Tables: 1 to 32. In a non-limiting example, 3 Tables, such as Table 1, Table 2 and Table 3 are selected from Tables: 1 to 32, and the data set comprises or is derived from gene expression measurements of at least 2 genes selected from the genes listed in each of the selected Tables, e.g., at least 2 genes selected from the genes listed in Table 1, at least 2 genes selected from the genes listed in Table 2, and at least 2 genes selected from the genes listed in Table 3. The one or more Tables selected from Tables: 1 to 32 can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 Tables. In certain embodiments, the one or more Tables comprise at least 1 Table, e.g., at least 1 Table is selected from Tables: 1 to 32. In certain embodiments, the one or more Tables comprise at least 2 Tables. In certain embodiments, the one or more Tables comprise at least 3 Tables. In certain embodiments, the one or more Tables comprise at least 4 Tables. In certain embodiments, the one or more Tables comprise at least 5 Tables. In certain embodiments, the one or more Tables comprise at least 6 Tables. In certain embodiments, the one or more Tables comprise at least 7 Tables. In certain embodiments, the one or more Tables comprise at least 8 Tables. In certain embodiments, the one or more Tables comprise at least 9 Tables. In certain embodiments, the one or more Tables comprise at least 10 Tables. In certain embodiments, the one or more Tables comprise at least 11 Tables. In certain embodiments, the one or more Tables comprise at least 12 Tables. In certain embodiments, the one or more Tables comprise at least 13 Tables. In certain embodiments, the one or more Tables comprise at least 14 Tables. In certain embodiments, the one or more Tables comprise at least 15 Tables. In certain embodiments, the one or more Tables comprise at least 16 Tables. In certain embodiments, the one or more Tables comprise at least 17 Tables. In certain embodiments, the one or more Tables comprise at least 18 Tables. In certain embodiments, the one or more Tables comprise at least 19 Tables. In certain embodiments, the one or more Tables comprise at least 20 Tables. In certain embodiments, the one or more Tables comprise at least 21 Tables. In certain embodiments, the one or more Tables comprise at least 22 Tables. In certain embodiments, the one or more Tables comprise at least 23 Tables. In certain embodiments, the one or more Tables comprise at least 24 Tables. In certain embodiments, the one or more Tables comprise at least 25 Tables. In certain embodiments, the one or more Tables comprise at least 26 Tables. In certain embodiments, the one or more Tables comprise at least 27 Tables. In certain embodiments, the one or more Tables comprise at least 28 Tables. In certain embodiments, the one or more Tables comprise at least 29 Tables. In certain embodiments, the one or more Tables comprise at least 30 Tables. In certain embodiments, the one or more Tables comprise at least 31 Tables. In certain embodiments, the one or more Tables comprise 32 Tables, i.e., Tables: 1 to 32 are selected. In certain embodiments, the one or more Tables comprise at least 14 Tables, e.g., 14 or more Tables are selected from Tables: 1 to 32, wherein at least Tables: 2; 4; 5; 7; 8; 12; 13; 14; 15; 16; 18; 19; 23; and 31, are selected. In certain embodiments, the one or more Tables comprise at least 16 Tables, wherein at least Tables: 2; 4; 5; 7; 8; 12; 13; 14; 15; 16; 18; 19; 20; 22; 23; and 31, are selected. In certain embodiments, the one or more Tables comprise at least 18 Tables, wherein at least Tables: 2; 4; 5; 7; 8; 11; 12; 13; 14; 15; 16; 18; 19; 20; 22; 23; 24; and 31, are selected. In certain embodiments, the one or more Tables comprise at least 23 Tables, wherein at least Tables: 2; 4; 5; 6; 7; 8; 10; 11; 12; 13; 14; 15; 16; 17; 18; 19; 20; 22; 23; 24; 25; 31; and 32, are selected. In certain embodiments, the one or more Tables comprise at least 21 Tables, wherein at least Tables: 2; 4; 5; 6; 7; 8; 11; 12; 13; 14; 15; 16; 17; 18; 19; 20; 22; 23; 24; 25; and 31, are selected. In certain embodiments, the one or more Tables comprise at least 24 Tables, wherein at least Tables: 2; 3; 4; 5; 6; 7; 8; 10; 11; 12; 13; 14; 15; 16; 17; 18; 19; 20; 22; 23; 24; 25; 31; and 32, are selected. In certain embodiments, the one or more Tables comprise at least 25 Tables, wherein at least Tables: 1; 2; 3; 4; 5; 6; 7; 8; 10; 11; 12; 13; 14; 15; 16; 17; 18; 19; 20; 22; 23; 24; 25; 31; and 32, are selected. In certain embodiments, the one or more Tables comprise at least 26 Tables, wherein at least Tables: 1; 2; 3; 4; 5; 6; 7; 8; 10; 11; 12; 13; 14; 15; 16; 17; 18; 19; 20; 21; 22; 23; 24; 25; 31; and 32, are selected. In certain embodiments, the one or more Tables comprise at least 28 Tables, wherein at least Tables: 2; 4; 5; 6; 7; 8; 9; 10; 11; 12; 13; 14; 15; 16; 17; 18; 19; 20; 22; 23; 24; 25; 27; 28; 29; 30; 31; and 32, are selected. In certain embodiments, the one or more Tables comprise at least 29 Tables, wherein at least Tables: 2; 3; 4; 5; 6; 7; 8; 9; 10; 11; 12; 13; 14; 15; 16; 17; 18; 19; 20; 22; 23; 24; 25; 27; 28; 29; 30; 31; and 32, are selected. In certain embodiments, the one or more Tables are selected from Tables: 1 to 32, based on the feature co-efficient of the Tables. In certain embodiments, if at least X Tables are selected from Tables: 1 to 32, where X is an integer from 1 to 32, at least the Tables having X highest absolute feature co-efficient values are selected. Tables: 1 to 32 lists feature co-efficient and absolute feature co-efficient of the respective Tables. Absolute feature co-efficient of a respective Table (e.g., within Tables: 1 to 32) may denote contribution of the genes listed within the respective Table in classification of the lupus disease state of patients between group A-H lupus disease state. Absolute feature coefficient can be mod of the feature coefficient. In certain embodiments, the one or more Tables selected from Tables: 1 to 32, comprises the Table with the highest absolute feature co-efficient value, i.e., at least Table 8 is selected. In certain embodiments, the one or more Tables selected from Tables: 1 to 32, comprise the Tables with 2 highest absolute feature co-efficient values, i.e., at least Table 8 and Table 18 are selected. In certain embodiments, the one or more Tables selected from Tables: 1 to 32, comprise the Tables with 3 highest absolute feature co-efficient values i.e., at least Table 8, Table 18 and Table 4 are selected. In certain embodiments, the one or more Tables selected from Tables: 1 to 32, comprise the Tables with 4 highest absolute feature co-efficient values. In certain embodiments, the one or more Tables selected from Tables: 1 to 32, comprise the Tables with 5 highest absolute feature co-efficient values. In certain embodiments, the one or more Tables selected from Tables: 1 to 32, comprise the Tables with 6 highest absolute feature co-efficient values. In certain embodiments, the one or more Tables selected from Tables: 1 to 32, comprise the Tables with 7 highest absolute feature co-efficient values. In certain embodiments, the one or more Tables selected from Tables: 1 to 32, comprise the Tables with 8 highest absolute feature co-efficient values. In certain embodiments, the one or more Tables selected from Tables: 1 to 32, comprise the Tables with 9 highest absolute feature co-efficient values. In certain embodiments, the one or more Tables selected from Tables: 1 to 32, comprise the Tables with 10 highest absolute feature co-efficient values. In certain embodiments, the one or more Tables selected from Tables: 1 to 32, comprise the Tables with 11 highest absolute feature co-efficient values.

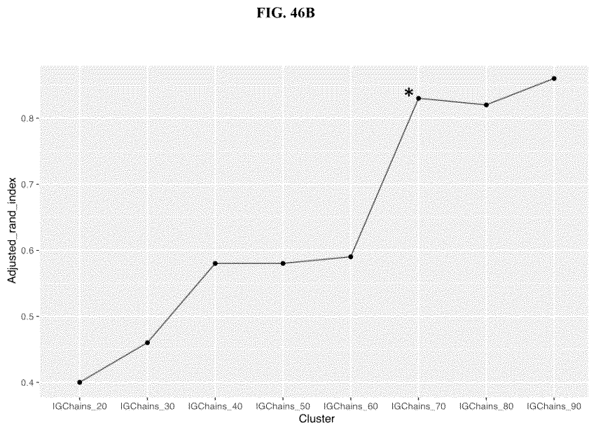

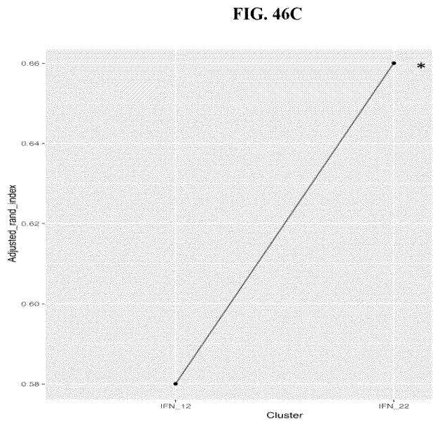

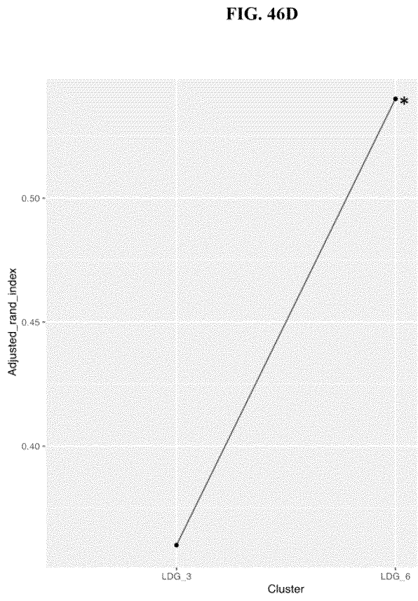

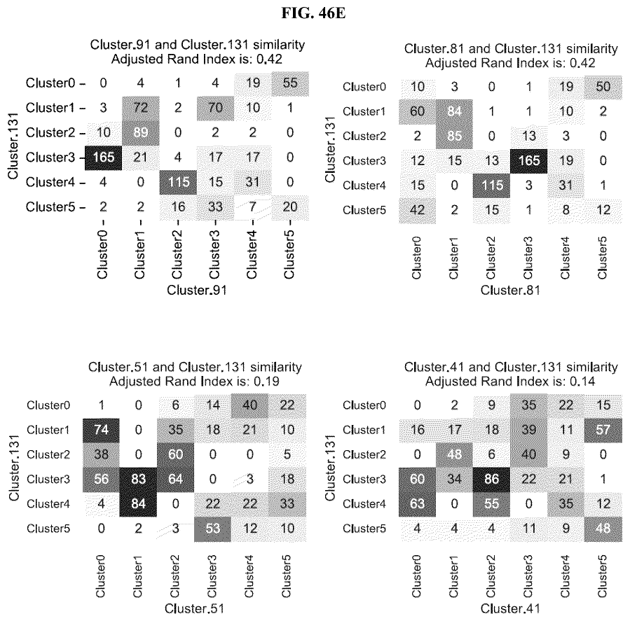

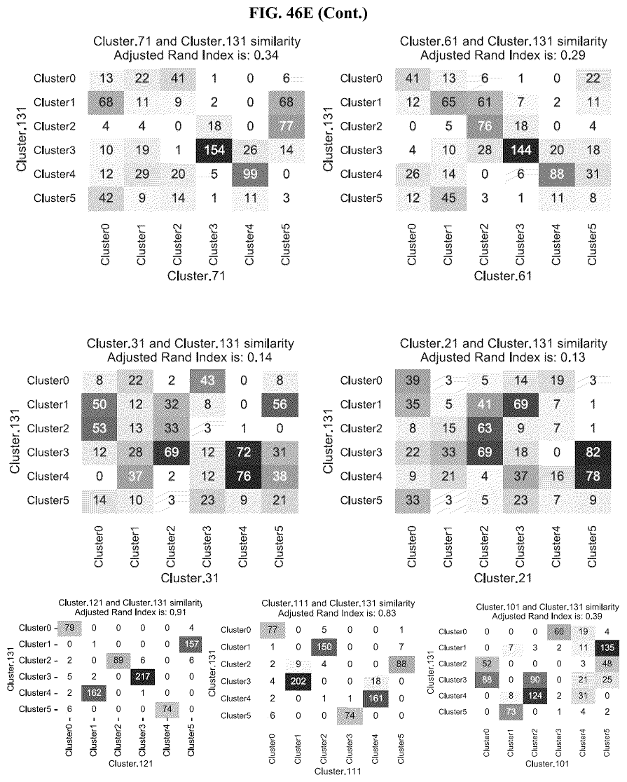

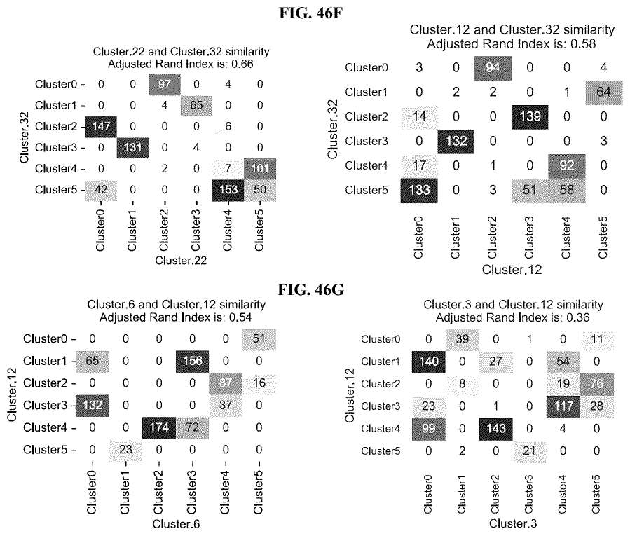

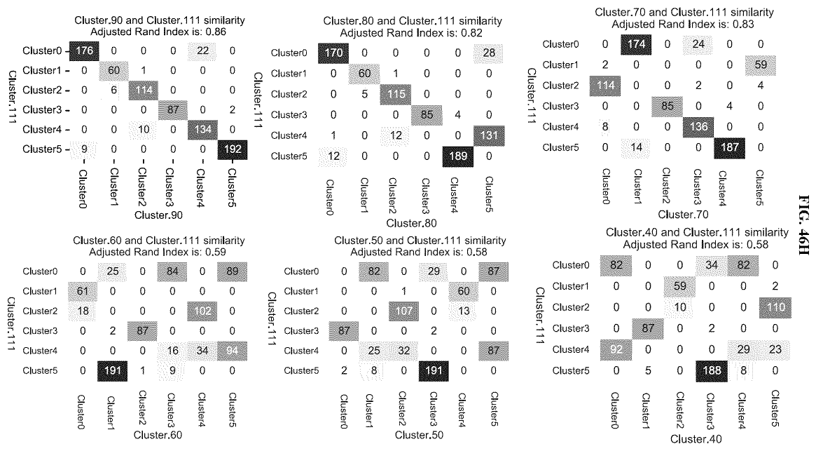

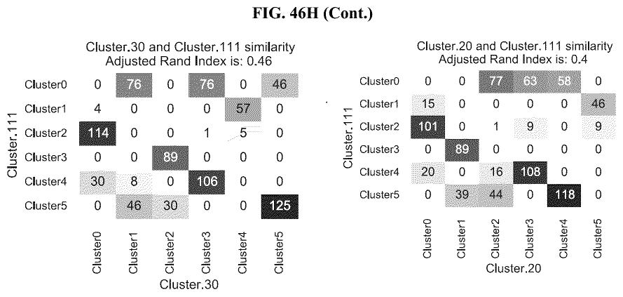

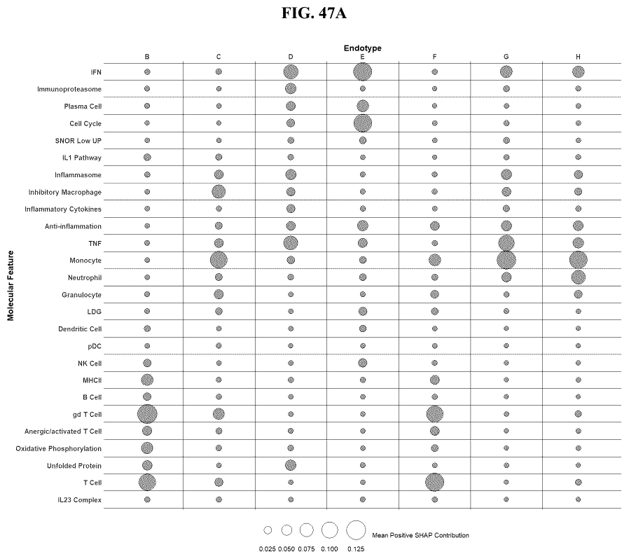

In certain embodiments, the one or more Tables selected from Tables: 1 to 32, comprise the Tables with 12 highest absolute feature co-efficient values. In certain embodiments, the one or more Tables selected from Tables: 1 to 32, comprise the Tables with 13 highest absolute feature co-efficient values. In certain embodiments, the one or more Tables selected from Tables: 1 to 32, comprise the Tables with 14 highest absolute feature co-efficient values. In certain embodiments, the one or more Tables selected from Tables: 1 to 32, comprise the Tables with 15 highest absolute feature co-efficient values. In certain embodiments, the one or more Tables selected from Tables: 1 to 32, comprise the Tables with 16 highest absolute feature co-efficient values. In certain embodiments, the one or more Tables selected from Tables: 1 to 32, comprise the Tables with 17 highest absolute feature co-efficient values. In certain embodiments, the one or more Tables selected from Tables: 1 to 32, comprise the Tables with 18 highest absolute feature co-efficient values. In certain embodiments, the one or more Tables selected from Tables: 1 to 32, comprise the Tables with 19 highest absolute feature co-efficient values. In certain embodiments, the one or more Tables selected from Tables: 1 to 32, comprise the Tables with 20 highest absolute feature co-efficient values. In certain embodiments, the one or more Tables selected from Tables: 1 to 32, comprise the Tables with 21 highest absolute feature co-efficient values. In certain embodiments, the one or more Tables selected from Tables: 1 to 32, comprise the Tables with 22 highest absolute feature co-efficient values. In certain embodiments, the one or more Tables selected from Tables: 1 to 32, comprise the Tables with 23 highest absolute feature co-efficient values. In certain embodiments, the one or more Tables selected from Tables: 1 to 32, comprise the Tables with 24 highest absolute feature co-efficient values. In certain embodiments, the one or more Tables selected from Tables: 1 to 32, comprise the Tables with 25 highest absolute feature co-efficient values. In certain embodiments, the one or more Tables selected from Tables: 1 to 32, comprise the Tables with 26 highest absolute feature co-efficient values. In certain embodiments, the one or more Tables selected from Tables: 1 to 32, comprise the Tables with 27 highest absolute feature co-efficient values. In certain embodiments, the one or more Tables selected from Tables: 1 to 32, comprise the Tables with 28 highest absolute feature co-efficient values. In certain embodiments, the one or more Tables selected from Tables: 1 to 32, comprise the Tables with 29 highest absolute feature co-efficient values. In certain embodiments, the one or more Tables selected from Tables: 1 to 32, comprise the Tables with 30 highest absolute feature co-efficient values. In certain embodiments, the one or more Tables selected from Tables: 1 to 32, comprise the Tables with 31 highest absolute feature co-efficient values. In certain embodiments, for each selected Table the data set comprises or is derived from gene expression measurements of at least 2 genes selected from the genes listed in the selected Table, wherein the number of genes selected from different selected Tables can be the same or different. In certain embodiments, for each selected Table the data set comprises or is derived from gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein the number of genes selected from different selected Tables can be the same or different. In a non-limiting example, 3 Tables, such as Table 1, Table 2 and Table 3 are selected from Tables: 1 to 32, and the data set comprises or is derived from gene expression measurements of 5 genes selected from the genes listed in Table 1, 3 genes selected from the genes listed in Table 2, and 20 genes selected from the genes listed in Table 3. In certain embodiments, for each selected Table the data set comprises or is derived from gene expression measurements of all the genes listed in the selected Table. In a non-limiting example, 3 Tables, such as Table 1, Table 2 and Table 3 are selected from Tables: 1 to 32, and the data set comprises or is derived from gene expression measurements of all the genes listed in each of the selected Tables, e.g., the genes (e.g., 8 genes) listed in Table 1, the genes (e.g., 3 genes) listed in Table 2, and the genes (e.g., 23 genes) listed in Table 3. In certain embodiments, for each selected Table the data set comprises or is derived from gene expression measurements of an effective number of genes selected from the genes listed in the selected Table, wherein the number of genes selected from different selected Tables can be the same or different. In a non-limiting example, 3 Tables, such as Table 1, Table 2 and Table 3 are selected from Tables: 1 to 32, and the data set comprises or is derived from gene expression measurements of effective number of genes selected from the genes listed in each of the selected Tables, e.g., effective number of genes selected from the genes listed in Table 1, effective number of genes selected from the genes listed in Table 2, and effective number of genes selected from the genes listed in Table 3, wherein the number of genes selected from Tables 1, 2, and 3 can be the same or different. The at least 2 genes may or may not include gene(s) that are not listed in Tables 1 to 32. In certain embodiments, the at least 2 genes do not include any gene that is not listed in Tables 1 to 32. A date set disclosed herein, e.g., in this paragraph can be analyzed to classify whether the patient has group A lupus disease state, group B lupus disease state, group C lupus disease state, group D lupus disease state, group E lupus disease state, group F lupus disease state, group G lupus disease state, or group H lupus disease state. The inference based on a dataset (e.g., generated based on the analysis of the data set) disclosed herein, e.g., in this paragraph, can be whether the dataset is indicative of the patient having group A lupus disease state, group B lupus disease state, group C lupus disease state, group D lupus disease state, group E lupus disease state, group F lupus disease state, group G lupus disease state, or group H lupus disease state. Selecting an effective number of genes from a Table can include selecting at least minimum number of genes from the table to obtain desired accuracy, sensitivity, specificity, positive predictive value, and/or negative predictive value in classification of the lupus disease state of the patient. Desired accuracy, sensitivity, specificity, positive predictive value, and/or negative predictive value, can be an accuracy, sensitivity, specificity, positive predictive value, and/or negative predictive value respectively described above or elsewhere herein. In certain embodiments, the desired accuracy, sensitivity, specificity, positive predictive value, and/or negative predictive value, is at least 85%. In certain embodiments, the desired accuracy, sensitivity, specificity, positive predictive value, and/or negative predictive value, is at least 90%. In certain embodiments, the desired accuracy, sensitivity, specificity, positive predictive value, and/or negative predictive value, is at least 95%. As shown in a non-limiting manner, the effective number of genes for a module/Table can be determined using adjusted rand index (ARI) method as described in the Example 2 and FIGS. 46 A-H. In certain embodiments, selecting effective number of genes from a Table (e.g., one of Tables 1 to 32) can include selecting at least about 60%, 65%, 70%, 75%, 80%, 85%, 90%, 95%, 96%, 97%, 98%, 99% or 100% of the genes in the Table. In certain embodiments, selecting an effective number of genes from a Table (e.g., one of Tables 1 to 32) can include selecting at least about 60%, 65%, 70%, 75%, 80%, 85%, 90%, 95%, 96%, 97%, 98%, 99% or 100% of the genes in the Table, where the Table contains 100 or more genes. In certain embodiments, selecting effective number of genes from a Table (e.g., one of Tables 1 to 32) can include selecting at least 70%, genes from the Table, where the Table contains 100 or more genes. In certain embodiments, selecting effective number of genes from a Table (e.g., one of Tables 1 to 32) can include selecting at least about 80%, 90%, 95%, 96%, 97%, 98%, 99%, or 100% of the genes in the Table, where the Table contains less than 100 genes. In certain embodiments, selecting effective number of genes from a Table (e.g., one of Tables 1 to 32) can include selecting all genes from the Table, where the Table contains less than 100 genes. In certain embodiments, collinear genes (such as with r >0.9, >0.8, >0.7, or >0.6) are be removed from the gene set forming the effective number of genes. In some embodiments, an effective number of genes in a Table disclosed herein comprises about 60 percent to about 100 percent of the genes in the Table. In some embodiments, an effective number of genes in a Table disclosed herein comprises about 60 percent to about 65 percent, about 60 percent to about 70 percent, about 60 percent to about 75 percent, about 60 percent to about 80 percent, about 60 percent to about 85 percent, about 60 percent to about 90 percent, about 60 percent to about 95 percent, about 60 percent to about 97 percent, about 60 percent to about 98 percent, about 60 percent to about 99 percent, about 60 percent to about 100 percent, about 65 percent to about 70 percent, about 65 percent to about 75 percent, about 65 percent to about 80 percent, about 65 percent to about 85 percent, about 65 percent to about 90 percent, about 65 percent to about 95 percent, about 65 percent to about 97 percent, about 65 percent to about 98 percent, about 65 percent to about 99 percent, about 65 percent to about 100 percent, about 70 percent to about 75 percent, about 70 percent to about 80 percent, about 70 percent to about 85 percent, about 70 percent to about 90 percent, about 70 percent to about 95 percent, about 70 percent to about 97 percent, about 70 percent to about 98 percent, about 70 percent to about 99 percent, about 70 percent to about 100 percent, about 75 percent to about 80 percent, about 75 percent to about 85 percent, about 75 percent to about 90 percent, about 75 percent to about 95 percent, about 75 percent to about 97 percent, about 75 percent to about 98 percent, about 75 percent to about 99 percent, about 75 percent to about 100 percent, about 80 percent to about 85 percent, about 80 percent to about 90 percent, about 80 percent to about 95 percent, about 80 percent to about 97 percent, about 80 percent to about 98 percent, about 80 percent to about 99 percent, about 80 percent to about 100 percent, about 85 percent to about 90 percent, about 85 percent to about 95 percent, about 85 percent to about 97 percent, about 85 percent to about 98 percent, about 85 percent to about 99 percent, about 85 percent to about 100 percent, about 90 percent to about 95 percent, about 90 percent to about 97 percent, about 90 percent to about 98 percent, about 90 percent to about 99 percent, about 90 percent to about 100 percent, about 95 percent to about 97 percent, about 95 percent to about 98 percent, about 95 percent to about 99 percent, about 95 percent to about 100 percent, about 97 percent to about 98 percent, about 97 percent to about 99 percent, about 97 percent to about 100 percent, about 98 percent to about 99 percent, about 98 percent to about 100 percent, or about 99 percent to about 100 percent of the genes in the Table. In some embodiments, an effective number of genes in a Table disclosed herein comprises about 60 percent, about 65 percent, about 70 percent, about 75 percent, about 80 percent, about 85 percent, about 90 percent, about 95 percent, about 97 percent, about 98 percent, about 99 percent, or about 100 percent of the genes in the Table. In some embodiments, an effective number of genes in a Table disclosed herein comprises at least about 60 percent, about 65 percent, about 70 percent, about 75 percent, about 80 percent, about 85 percent, about 90 percent, about 95 percent, about 97 percent, about 98 percent, or about 99 percent of the genes in the Table.

The data set can be generated from the biological sample obtained or derived from the patient. For example, nucleic acid molecules of the patient in the biological sample can be assessed to obtain the data set. In certain embodiments, the gene expression measurements of the biological sample of the selected genes can be performed using any suitable method known to those of skill in the art including but not limited to DNA sequencing, RNA sequencing, microarray, RNA-Seq, qPCR, northern blotting, fluorescent in situ hybridization, serial analysis of gene expression, tiling arrays or any combination thereof, to obtain the data set. In certain embodiments, the gene expression measurements of the biological sample of the selected genes can be performed using RNA-Seq. RNA-seq can include single cell RNA-seq, and/or bulk RNA-seq. In certain embodiments, the gene expression measurements of the biological sample of the selected genes can be performed using microarray. In certain embodiments, the data set can be derived from the gene expression measurements of the biological sample of the selected genes, wherein the gene expression measurements is analyzed using a suitable data analysis tool including but not limited to a BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), Z-score, gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof, to obtain the dataset. In certain embodiments, the gene expression measurements of the biological sample of the selected genes can be analyzed using GSVA, to obtain the data set. In certain embodiments, the method comprises obtaining and/or deriving the biological sample from the patient. In certain embodiments, the method comprises analyzing the biological sample to obtain the gene expression measurements of the biological sample. In certain embodiments, the method comprises analyzing the gene expression measurements to obtain the dataset. In certain embodiments, the method comprises obtaining and/or deriving the biological sample from the patient, and/or analyzing the biological sample to obtain the gene expression measurement of the biological sample. In certain embodiments, the method comprises obtaining and/or deriving the biological sample from the patient, analyzing the biological sample to obtain the gene expression measurement of the biological sample, and/or analyzing the gene expression measurements to obtain the dataset.

In certain embodiments, analyzing the dataset comprises analyzing gene expression of one or more gene sets formed based on the one or more Tables selected from Tables: 1 to 32, wherein genes selected from each of the selected Table can form a gene set of the one or more gene sets. Genes selected from different selected Tables can form different gene sets of the one or more gene sets. The dataset can comprise gene expression measurement values of the one or more gene sets. The one or more Tables selected (e.g., based on which the one or more gene sets are formed) can comprise the selected Tables as described above or elsewhere herein. In certain embodiments, the one or more Tables selected comprise Tables: 2; 4; 5; 7; 8; 12; 13; 14; 15; 16; 18; 19; 23; and 31. In certain embodiments, the one or more Tables selected comprise Tables: 2; 4; 5; 7; 8; 12; 13; 14; 15; 16; 18; 19; 20; 22; 23; and 31. In certain embodiments, the one or more Tables selected comprise Tables: 2; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the one or more Tables selected comprise Tables: 2; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 3; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 4; 5; 6; 7; 8; 11; 12; 13; 14; 15; 16; 17; 18; 19; 20; 22; 23; 24; 25; and 31. In certain embodiments, the one or more Tables selected comprise Tables: 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 2; 4; 5; 7; 8; 11; 12; 13; 14; 15; 16; 18; 19; 20; 22; 23; 24; and 31. In certain embodiments, the one or more Tables selected comprise Tables: 2; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 3; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 4; 5; 6; 7; 8; 11; 12; 13; 14; 15; 16; 17; 18; 19; 20; 22; 23; 24; 25; and 31. In certain embodiments, the one or more Tables selected comprise Tables: 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 2; 4; 5; 7; 8; 11; 12; 13; 14; 15; 16; 18; 19; 20; 22; 23; 24; and 31. In certain embodiments Tables 1 to 32 are selected. For a selected Table the genes selected from the selected Table can comprise the selected genes as described above or elsewhere herein, such as at least 2 genes, effective number of genes, and/or all genes from the selected Table. In certain embodiments, for each selected Table the genes selected (e.g., that forms the gene set based on the selected Table) comprise at least 2 genes selected from the genes listed in the selected Table, wherein the number of genes selected from different selected Tables can be the same or different. In certain embodiments, for each selected Table the genes selected (e.g., that forms the gene set based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein the number of genes selected from different selected Tables can be the same or different. In certain embodiments, for each selected Table the genes selected (e.g., that forms the gene set based on the selected Table) comprise effective number of genes selected from the genes listed in the selected Table, wherein the number of genes selected from different selected Tables can be the same or different. In certain embodiments, for each selected Table the genes selected (e.g., that forms the gene set based on the selected Table) comprise all the genes listed in the selected Table. Each of the one or more gene sets can be generated based on one of the one or more selected Tables, wherein for each selected Table the genes selected (e.g., at least 2 genes, effective number of genes, and/or all genes) from the selected Table forms a gene set of the one or more gene set. In a non-limiting example, the one or more Tables selected comprise Tables: 1, 2 and 3, and effective number of genes are selected from each of the Table selected, and the one or more gene sets comprise a gene set formed based on Table 1, a gene set formed based on Table 2, and a gene set formed based on Table 3, wherein the gene set formed based on Table 1 comprises effective number of genes selected from the genes listed in Table 1, the gene set formed based on Table 2 comprises effective number of genes selected from the genes listed in Table 2, and the gene set formed based on Table 3 comprises effective number of genes selected from the genes listed in Table 3. Analyzing gene expression of a gene set (e.g., of the one or more gene sets) can include analyzing module eigengenes (MEs) of the gene set/module. In certain embodiments, the gene expression (e.g., in the biological sample) of the one or more gene sets can be analyzed to classify the lupus disease of the patient. In certain embodiments, the gene expression (e.g., in the biological sample) of the one or more gene sets can be analyzed to classify whether the patient has group A lupus disease state, group B lupus disease state, group C lupus disease state, group D lupus disease state, group E lupus disease state, group F lupus disease state, group G lupus disease state, or group H lupus disease state. In certain embodiments, MEs of the one or more gene sets can be analyzed to classify the lupus disease of the patient. In certain embodiments, MEs of the one or more gene sets can be analyzed to classify whether the patient has group A lupus disease state, group B lupus disease state, group C lupus disease state, group D lupus disease state, group E lupus disease state, group F lupus disease state, group G lupus disease state, or group H lupus disease state. The inference indicative of the lupus disease state of the patient can be generated based on gene expression (e.g., in the biological sample) of the one or more gene sets. The inference indicative of the lupus disease state of the patient can be generated based on the MEs of the one or more gene sets.

In certain embodiments, the data set comprises one or more enrichment scores of the patient. The one or more enrichment scores of the patient can be derived from the gene expression measurements of the biological sample, wherein the one or more enrichment scores are generated based on the one or more Tables selected from Tables: 1 to 32, wherein for each selected Table, the genes selected from the selected Table forms an input gene set, based on which at least one enrichment score of the patient, based on the selected Table is generated. The one or more enrichment scores comprise the generated enrichment scores. The at least one enrichment score based on a selected Table can be generated based on enrichment of the input gene set (e.g., containing genes selected from the selected Table) based on the selected Tables in the biological sample. Enrichment can be determined with respect to a reference dataset as described herein. The one or more Tables selected (e.g., based on which the one or more enrichment scores of the patient are generated) can comprise the selected Tables as described above or elsewhere herein. In certain embodiments, the one or more Tables selected comprise Tables: 2; 4; 5; 7; 8; 12; 13; 14; 15; 16; 18; 19; 23; and 31. In certain embodiments, the one or more Tables selected comprise Tables: 2; 4; 5; 7; 8; 12; 13; 14; 15; 16; 18; 19; 20; 22; 23; and 31. In certain embodiments, the one or more Tables selected comprise Tables: 2; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the one or more Tables selected comprise Tables: 2; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 3; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 4; 5; 6; 7; 8; 11; 12; 13; 14; 15; 16; 17; 18; 19; 20; 22; 23; 24; 25; and 31. In certain embodiments, the one or more Tables selected comprise Tables: 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 2; 4; 5; 7; 8; 11; 12; 13; 14; 15; 16; 18; 19; 20; 22; 23; 24; and 31. In certain embodiments Tables 1 to 32 are selected. For a selected Table the genes selected (e.g., that forms the input gene set for generating the at least one enrichment score based on the selected Table) from the selected Table can comprise the selected genes as described above or elsewhere herein, such as at least 2 genes, effective number of genes, and/or all genes from the selected Table. The enrichment score can be determined using the input gene set based on a suitable method including but not limited GSVA, GSEA, or enrichment algorithm. In certain embodiments, the enrichment score is generated using GSVA, and the enrichment score can be GSVA score. In certain embodiments, for each selected Table the genes selected (e.g., that forms the input gene set for generating the at least one enrichment score based on the selected Table) comprise at least 2 genes selected from the genes listed in the selected Table, wherein the number of genes selected from different selected Tables can be the same or different. In certain embodiments, for each selected Table the genes selected (e.g., that forms the input gene set for generating the at least one enrichment score based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein the number of genes selected from different selected Tables can be the same or different. In certain embodiments, for each selected Table the genes selected (e.g., that forms the input gene set for generating the at least one enrichment score based on the selected Table) comprise effective number of genes selected from the genes listed in the selected Table, wherein the number of genes selected from different selected Tables can be the same or different. In certain embodiments, for each selected Table the genes selected (e.g., that forms the input gene set for generating the at least one enrichment score based on the selected Table) comprise all the genes listed in the selected Table. In certain embodiments, for each selected Table one enrichment score is generated based on the selected Table. Each of the one or more enrichment scores of the patient can be generated based on one of the one or more selected Tables, wherein for each selected Table the genes selected (e.g., at least 2 genes, effective number of genes, and/or all genes) from the selected Table forms an input gene set based on which the at least one enrichment score based on the selected Table is generated. Enrichment of the input gene set in the biological sample obtained or derived from the patient can be determined to generate the enrichment score. Enrichment can be determined with respect to a reference data set, as described herein. In a non-limiting example, the one or more Tables selected comprise Tables: 1 and 2, and effective number of genes are selected from genes listed in each of the Table selected, and the dataset comprises the one or more enrichment scores of the patient, wherein the one or more enrichment scores of the patient comprise at least one enrichment score generated based on Table 1, and at least one enrichment score generated based on Table 2, wherein the least one enrichment score generated based on Table 1 is generated based on enrichment of the input gene set (e.g., containing the effective number of genes selected from the genes listed in Table 1) based on Table 1 in the biological sample, and the least one enrichment score generated based on Table 2 is generated based on enrichment of the input gene set (e.g., containing the effective number of genes selected from the genes listed in Table 2) based on Table 2 in the biological sample. In certain embodiments, the data set comprises the one or more enrichment scores of the patient, analyzing the dataset comprises analyzing the one or more enrichment scores to classify the lupus disease state of the patient, and the method can classify the lupus disease state of the patient based on the one or more enrichment scores of the patient. In certain embodiments, the data set comprises the one or more enrichment scores of the patient, analyzing the dataset comprises analyzing the one or more enrichment scores to classify the lupus disease state of the patient, and the method can classify the lupus disease state of the patient based on the one or more enrichment scores of the patient, wherein classifying the lupus disease state of the patient include classifying whether the patient has group A lupus disease state, group B lupus disease state, group C lupus disease state, group D lupus disease state, group E lupus disease state, group F lupus disease state, group G lupus disease state, or group H lupus disease state. The inference indicative of the lupus disease state of the patient can be generated based on the one or more enrichment scores of the patient.

In certain embodiments, the data set is derived from the gene expression measurements of the biological sample using GSVA. In certain embodiments, the data set comprises one or more GSVA scores (e.g., GSVA enrichment scores) of the patient derived from the gene expression measurements of the biological sample using GSVA, wherein the one or more GSVA scores are generated based on the one or more Tables selected from Tables: 1 to 32, wherein for each selected Table, the genes selected from the selected Table forms an input gene set, based on which at least one GSVA score of the patient, based on the selected Table is generated using GSVA The one or more GSVA scores can comprise the generated GSVA scores. The at least one GSVA score based on a selected Table can be generated based on enrichment of the input gene set (e.g., containing genes selected from the selected Table) based on the selected Tables in the biological sample. GSVA can be performed using a suitable method known to those of skill in the art and/or as described in the Examples. The one or more Tables selected (e.g., based on which the one or more GSVA scores of the patient are generated) can comprise the selected Tables as described above or elsewhere herein. In certain embodiments, the one or more Tables selected comprise Tables: 2; 4; 5; 7; 8; 12; 13; 14; 15; 16; 18; 19; 23; and 31. In certain embodiments, the one or more Tables selected comprise Tables: 2; 4; 5; 7; 8; 12; 13; 14; 15; 16; 18; 19; 20; 22; 23; and 31. In certain embodiments, the one or more Tables selected comprise Tables: 2; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the one or more Tables selected comprise Tables: 2; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 3; 4; 5; 6; 7; 8; 9; 10; 11; 12; 13; 14; 15; 16; 17; 18; 19; 20; 22; 23; 24; 25; 27; 28; 29; 30; 31; and 32. In certain embodiments, the one or more Tables selected comprise Tables: 2; 4; 5; 6; 7; 8; 11; 12; 13; 14; 15; 16; 17; 18; 19; 20; 22; 23; 24; 25; and 31. In certain embodiments, the one or more Tables selected comprise Tables: 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 1; 2; 3; 4; 5; 6; 7; 8; 10; 11; 12; 13; 14; 15; 16; 17; 18; 19; 20; 22; 23; 24; 25; 31; and 32. In certain embodiments, the one or more Tables selected comprise Tables: 2; 4; 5; 7; 8; 11; 12; 13; 14; 15; 16; 18; 19; 20; 22; 23; 24; and 31. In certain embodiments Tables 1 to 32 are selected. For a selected Table the genes selected (e.g., that forms the input gene set for generating the at least one GSVA score based on the selected Table) from the selected Table can comprise the selected genes as described above or elsewhere herein, such as at least 2 genes, effective number of genes, and/or all genes from the selected Table. The GSVA scores can be GSVA enrichment scores, and can be generated using GSVA using the respective input gene sets, based on a method as described in the Examples and/or as understood by one of skill in the art. In certain embodiments, for each selected Table the genes selected (e.g., that forms the input gene set for generating the at least one GSVA score based on the selected Table) comprise at least 2 genes selected from the genes listed in the selected Table, wherein the number of genes selected from different selected Tables can be the same or different. In certain embodiments, for each selected Table the genes selected (e.g., that forms the input gene set for generating the at least one GSVA score based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein the number of genes selected from different selected Tables can be the same or different. In certain embodiments, for each selected Table the genes selected (e.g., that forms the input gene set for generating the at least one GSVA score based on the selected Table) comprise effective number of genes selected from the genes listed in the selected Table, wherein the number of genes selected from different selected Tables can be the same or different. In certain embodiments, for each selected Table the genes selected (e.g., that forms the input gene set for generating the at least one GSVA score based on the selected Table) comprises all the genes listed in the selected Table.

In certain embodiments, for each selected Table one GSVA score is generated based on the selected Table. Each of the one or more GSVA scores of the patient can be generated based on one of the one or more selected Tables, wherein for each selected Table the genes selected (e.g., at least 2 genes, effective number of genes, and/or all genes) from the selected Table forms an input gene set based on which the at least one GSVA score based on the selected Table is generated, using GSVA. Enrichment of the input gene set in the biological sample obtained or derived from the patient can be determined to generate the GSVA score. Enrichment can be determined with respect to a reference data set, as described herein. In a non-limiting example, the one or more Tables selected comprise Tables: 1 and 2, and effective number of genes are selected from the genes listed in each of the Table selected, and the dataset comprises the one or more GSVA scores of the patient, wherein the one or more GSVA scores of the patient comprise at least one GSVA score generated based on Table 1, and at least one GSVA score generated based on Table 2, wherein the least one GSVA score generated based on Table 1 is generated based on enrichment of the input gene set (containing the effective number of genes selected from the genes listed in Table 1) based on Table 1 in the biological sample, and the least one GSVA score generated based on Table 2 is generated based on enrichment of the input gene set (containing the effective number of genes selected from the genes listed in Table 2) based on Table 2 in the biological sample. In certain embodiments, the data set comprises the one or more GSVA scores of the patient, analyzing the dataset comprises analyzing the one or more GSVA scores to classify the lupus disease state of the patient, and the method can classify the lupus disease state of the patient based on the one or more GSVA scores of the patient. In certain embodiments, the data set comprises the one or more GSVA scores of the patient, analyzing the dataset comprises analyzing the one or more GSVA scores to classify the lupus disease state of the patient, and the method can classify the lupus disease state of the patient based on the one or more GSVA scores of the patient, wherein classifying the lupus disease state of the patient include classifying whether the patient has group A lupus disease state, group B lupus disease state, group C lupus disease state, group D lupus disease state, group E lupus disease state, group F lupus disease state, group G lupus disease state, or group H lupus disease state. The inference indicative of the lupus disease state of the patient can be generated based on the one or more GSVA scores of the patient.

In certain embodiments, the one or more Tables selected comprise Tables 6, 25, 32, 21, 17, 1, 3, 20, and/or 5, and classifying the lupus disease state of the patient can include classifying whether the patient has the group A lupus disease state, or group B lupus disease state. In certain embodiments, the one or more Tables selected comprise Tables 6, 25, and 21 and classifying the lupus disease state of the patient can include classifying whether the patient has the group A lupus disease state, or group B lupus disease state. In certain embodiments, the one or more Tables selected comprise Tables 18, 15, 6, 13, 7, 31, 25, 19, and/or 16, and classifying the lupus disease state of the patient can include classifying whether the patient has the group A lupus disease state, or group C lupus disease state. In certain embodiments, the one or more Tables selected comprise Tables 18, 15, and 6, and classifying the lupus disease state of the patient can include classifying whether the patient has the group A lupus disease state, or group C lupus disease state. In certain embodiments, the one or more Tables selected comprise Tables 31, 8, 12, 32, 13, 14, 23, 2, and/or 15, and classifying the lupus disease state of the patient can include classifying whether the patient has the group A lupus disease state, or group D lupus disease state. In certain embodiments, the one or more Tables selected comprise Tables 31, 8, and 12, and classifying the lupus disease state of the patient can include classifying whether the patient has the group A lupus disease state, or group D lupus disease state. In certain embodiments, the one or more Tables selected comprise Tables 4, 8, 23, 2, 31, 20, 16, 5, and/or 19, and classifying the lupus disease state of the patient can include classifying whether the patient has the group A lupus disease state, or group E lupus disease state. In certain embodiments, the one or more Tables selected comprise Tables 4, 8, and 23, and classifying the lupus disease state of the patient can include classifying whether the patient has the group A lupus disease state, or group E lupus disease state. In certain embodiments, the one or more Tables selected comprise Tables 25, 6, 18, 1, 17, 7, 2, 19, and/or 21, and classifying the lupus disease state of the patient can include classifying whether the patient has the group A lupus disease state, or group F lupus disease state. In certain embodiments, the one or more Tables selected comprise Tables 25, 6, and 18, and classifying the lupus disease state of the patient can include classifying whether the patient has the group A lupus disease state, or group F lupus disease state. In certain embodiments, the one or more Tables selected comprise Tables 18, 31, 13, 8, 2, 19, 15, 12, and/or 14, and classifying the lupus disease state of the patient can include classifying whether the patient has the group A lupus disease state, or group G lupus disease state. In certain embodiments, the one or more Tables selected comprise Tables 18, 31, and 8, and classifying the lupus disease state of the patient can include classifying whether the patient has the group A lupus disease state, or group G lupus disease state. In certain embodiments, the one or more Tables selected comprise Tables 18, 19, 31, 8, 2, 13, 7, 15, and/or 25, and classifying the lupus disease state of the patient can include classifying whether the patient has the group A lupus disease state, or H lupus disease state. In certain embodiments, the one or more Tables selected comprise Tables 18, 19, and 8, and classifying the lupus disease state of the patient can include classifying whether the patient has the group A lupus disease state, or H lupus disease state.

As an illustrative example, the one or more Tables selected in the described classification method comprise Tables Y1, Y2, and Y3, and classifying the lupus disease state of the patient includes classifying whether the patient has group X lupus disease state or group X1 lupus disease state, wherein X is one of A, B, C, D, E, F, G and H, wherein X1 is one of A, B, C, D, E, F, G and H, wherein X and X1 are different, and wherein Tables Y1, Y2 and Y3 are the Tables set forth herein corresponding to the 3 most important features/modules for distinguishing between group X and group X1. The 3 most important features/modules for distinguishing between each two classification groups are shown in Table 40, wherein the most important feature/module is marked as 1, the 2nd most important module is marked as 2, and the 3rd most important module is marked as 3. The importance of the features/modules in distinguishing 2 groups can be identified using SHAP analysis. In a non-limiting example, X is B, and X1 is C, i.e., classifying the lupus disease state of the patient can include classifying whether the patient has the group B lupus disease state, or C lupus disease state, and from Table 40, Table Y1 is Table 13 (feature/module Inflammasome, most important feature), Table Y2 is Table 32 (feature/module Unfolded protein, 2nd most important feature) and Table Y3 is Table 10 (feature/module IL-1 pathway, 3rd most important feature), i.e., the one or more Tables selected comprises Tables 13, 32, and 10, and classifying the lupus disease state of the patient can include classifying whether the patient has the group B lupus disease state, or C lupus disease state. For a selected Table the genes selected from the selected Table can comprise the selected genes as described above or elsewhere herein, such as at least 2 genes, effective number of genes, and/or all genes from the selected Table. The present invention thus includes distinguishing between each pair of disease state groups possible among A, B, C, D, E, F, G, and H, by selecting at least 2 genes, selecting an effective number of genes, and/or selecting all genes, from the genes disclosed herein in the three tables that correspond to each of the top three most important feature/modules as set forth in Table 40. The present invention also includes distinguishing between each pair of disease state groups possible among A, B, C, D, E, F, G, and H, by selecting at least one gene, and/or selecting all genes, from the genes disclosed in the list of top ten gene predictors set forth in Table 41, that corresponds to the pair of disease state groups being distinguished.

In certain embodiments, the data set comprises gene expression measurement of at least 2 genes selected from the group of genes ATP5A1, CD247, COX15, COX6B2, NDUFA9, NDUFB2-AS1, NDUFC2, NDUFS1, NDUFS7, and SH2D1A, and classifying the lupus disease state of the patient can include classifying whether the patient has the group A lupus disease state, or group B lupus disease state, e.g., classifying the lupus disease state of the patient can include classifying whether the data set is indicative of the patient having the group A lupus disease state, or group B lupus disease state. The lupus disease state of the patient can be classified as group A lupus disease state, or group B lupus disease state based on the dataset. In certain embodiments, the data set comprises gene expression measurement of at least 2, 3, 4, 5, 6, 7, 8, 9 or 10 genes selected from the group of genes ATP5A1, CD247, COX15, COX6B2, NDUFA9, NDUFB2-AS1, NDUFC2, NDUFS1, NDUFS7, and SH2D1A, and classifying the lupus disease state of the patient can include classifying whether the patient has the group A lupus disease state, or group B lupus disease state. In certain embodiments, the data set comprises gene expression measurement of at least 3, genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 4 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 5 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 6 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 7 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 8 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of at least 9 genes selected from the group of genes ATP5A1, CD247, COX15, COX6B2, NDUFA9, NDUFB2-AS1, NDUFC2, NDUFS1, NDUFS7, and SH2D1A. In certain embodiments, the data set comprises gene expression measurement of ATP5A1, CD247, COX15, COX6B2, NDUFA9, NDUFB2-AS1, NDUFC2, NDUFS1, NDUFS7, and SH2D1A.

In certain embodiments, the data set comprises gene expression measurement of at least 2 genes selected from the group of genes ADGRE2, AOAH, BACH1, CLEC4D, CLEC7A, FFAR2, LILRA6, LMNB1, TLR2, and TNFRSF1B, and classifying the lupus disease state of the patient can include classifying whether the patient has the group A lupus disease state, or group C lupus disease state, e.g., classifying the lupus disease state of the patient can include classifying whether the data set is indicative of the patient having group A lupus disease state, or group C lupus disease state. The lupus disease state of the patient can be classified as group A lupus disease state, or group C lupus disease state based on the dataset. In certain embodiments, the data set comprises gene expression measurement of at least 2, 3, 4, 5, 6, 7, 8, 9 or 10 genes selected from the group of genes ADGRE2, AOAH, BACH1, CLEC4D, CLEC7A, FFAR2, LILRA6, LMNB1, TLR2, and TNFRSF1B, and classifying the lupus disease state of the patient can include classifying whether the patient has the group A lupus disease state, or group C lupus disease state. In certain embodiments, the data set comprises gene expression measurement of at least 3, genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 4 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 5 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 6 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 7 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 8 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of at least 9 genes selected from the group of genes ADGRE2, AOAH, BACH1, CLEC4D, CLEC7A, FFAR2, LILRA6, LMNB1, TLR2, and TNFRSF1B. In certain embodiments, the data set comprises gene expression measurement of ADGRE2, AOAH, BACH1, CLEC4D, CLEC7A, FFAR2, LILRA6, LMNB1, TLR2, and TNFRSF1B.

In certain embodiments, the data set comprises gene expression measurement of at least 2 genes selected from the group of genes ACLY, ARSE, CASP1, CASP10, CTNND2, EIF2AK2, GBP1, IF1I30, IL1RN and PSMB8, and classifying the lupus disease state of the patient can include classifying whether the patient has the group A lupus disease state, or group D lupus disease state, e.g., classifying the lupus disease state of the patient can include classifying whether the data set is indicative of the patient having group A lupus disease state, or group D lupus disease state. The lupus disease state of the patient can be classified as group A lupus disease state, or group D lupus disease state based on the dataset. In certain embodiments, the data set comprises gene expression measurement of at least 2, 3, 4, 5, 6, 7, 8, 9 or 10 genes selected from the group of genes ACLY, ARSE, CASP1, CASP10, CTNND2, EIF2AK2, GBP1, IFI30, IL1RN and PSMB8, and classifying the lupus disease state of the patient can include classifying whether the patient has the group A lupus disease state, or group D lupus disease state. In certain embodiments, the data set comprises gene expression measurement of at least 3, genes selected from the group of genes ACLY, ARSE, CASP1, CASP10, CTNND2, EIF2AK2, GBP1, IF1I30, IL1RN and PSMB8. In certain embodiments, the data set comprises gene expression measurement of at least 4 genes selected from the group of genes ACLY, ARSE, CASP1, CASP10, CTNND2, EIF2AK2, GBP1, IF1I30, IL1RN and PSMB8. In certain embodiments, the data set comprises gene expression measurement of at least 5 genes selected from the group of genes ACLY, ARSE, CASP1, CASP10, CTNND2, EIF2AK2, GBP1, 1F130, IL1RN and PSMB8. In certain embodiments, the data set comprises gene expression measurement of at least 6 genes selected from the group of genes ACLY, ARSE, CASP1, CASP10, CTNND2, EIF2AK2, GBP1, 1F130, IL1RN and PSMB8. In certain embodiments, the data set comprises gene expression measurement of at least 7 genes selected from the group of genes ACLY, ARSE, CASP1, CASP10, CTNND2, EIF2AK2, GBP1, 1F130, IL1RN and PSMB8. In certain embodiments, the data set comprises gene expression measurement of at least 8 genes selected from the group of genes ACLY, ARSE, CASP1, CASP10, CTNND2, EIF2AK2, GBP1, 1F130, IL1RN and PSMB8. In certain embodiments, the data set comprises gene expression measurement of at least 9 genes selected from the group of genes ACLY, ARSE, CASP1, CASP10, CTNND2, EIF2AK2, GBP1, IFI30, IL1RN and PSMB8. In certain embodiments, the data set comprises gene expression measurement of the group of genes ACLY, ARSE, CASP1, CASP10, CTNND2, EIF2AK2, GBP1, IF1I30, IL1RN and PSMB8.

In certain embodiments, the data set comprises gene expression measurement of at least 2 genes selected from the group of genes AURKB, CCNE1, EIF2AK2, GBP2, IFITM3, IGHG1, IGLV4-60, IGLV5-45, ISG20, and PTTG1, and classifying the lupus disease state of the patient can include classifying whether the patient has the group A lupus disease state, or group E lupus disease state, e.g., classifying the lupus disease state of the patient can include classifying whether the data set is indicative of the patient having group A lupus disease state, or group E lupus disease state. The lupus disease state of the patient can be classified as group A lupus disease state, or group E lupus disease state based on the dataset. In certain embodiments, the data set comprises gene expression measurement of at least 2, 3, 4, 5, 6, 7, 8, 9 or 10 genes selected from the group of genes AURKB, CCNE1, EIF2AK2, GBP2, IFITM3, IGHG1, IGLV4-60, IGLV5-45, ISG20, and PTTG1, and classifying the lupus disease state of the patient can include classifying whether the patient has the group A lupus disease state, or group E lupus disease state. In certain embodiments, the data set comprises gene expression measurement of at least 3, genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 4 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 5 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 6 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 7 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 8 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of at least 9 genes selected from the group of genes AURKB, CCNE1, EIF2AK2, GBP2, IFITM3, IGHG1, IGLV4-60, IGLV5-45, ISG20, and PTTG1. In certain embodiments, the data set comprises gene expression measurement of AURKB, CCNE1, EIF2AK2, GBP2, IFITM3, IGHG1, IGLV4-60, IGLV5-45, ISG20, and PTTG1.

In certain embodiments, the data set comprises gene expression measurement of at least 2 genes selected from CCL28, CD247, CHIT1, CXCL1, FFAR2, LILRB5, LMNB1, PYHIN1, SECTM1, and SIGLEC5, and classifying the lupus disease state of the patient can include classifying whether the patient has the group A lupus disease state, or group F lupus disease state, e.g., classifying the lupus disease state of the patient can include classifying whether the data set is indicative of the patient having group A lupus disease state, or group F lupus disease state. The lupus disease state of the patient can be classified as group A lupus disease state, or group F lupus disease state based on the dataset. In certain embodiments, the data set comprises gene expression measurement of at least 2, 3, 4, 5, 6, 7, 8, 9 or 10 genes selected from the group of genes CCL28, CD247, CHIT1, CXCL1, FFAR2, LILRB5, LMNB1, PYHIN1, SECTM1, and SIGLEC5, and classifying the lupus disease state of the patient can include classifying whether the patient has the group A lupus disease state, or group F lupus disease state. In certain embodiments, the data set comprises gene expression measurement of at least 3, genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 4 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 5 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 6 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 7 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 8 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of at least 9 genes selected from the group of genes CCL28, CD247, CHIT1, CXCL1, FFAR2, LILRB5, LMNB1, PYHIN1, SECTM1, and SIGLEC5. In certain embodiments, the data set comprises gene expression measurement of CCL28, CD247, CHIT1, CXCL1, FFAR2, LILRB5, LMNB1, PYHIN1, SECTM1, and SIGLEC5.

In certain embodiments, the data set comprises gene expression measurement of at least 2 genes selected from the group of genes APOBR, CASP1, CASP10, FFAR2, MS4A4A, MTF1, SECTM1, SEMA4A, TLR8, and TNFRSF1B, and classifying the lupus disease state of the patient can include classifying whether the patient has the group A lupus disease state, or group G lupus disease state, e.g., classifying the lupus disease state of the patient can include classifying whether the data set is indicative of the patient having group A lupus disease state, or group G lupus disease state. The lupus disease state of the patient can be classified as group A lupus disease state, or group G lupus disease state based on the dataset. In certain embodiments, the data set comprises gene expression measurement of at least 2, 3, 4, 5, 6, 7, 8, 9 or 10 genes selected from the group of genes APOBR, CASP1, CASP10, FFAR2, MS4A4A, MTF1, SECTM1, SEMA4A, TLR8, and TNFRSF1B, and classifying the lupus disease state of the patient can include classifying whether the patient has the group A lupus disease state, or group G lupus disease state. In certain embodiments, the data set comprises gene expression measurement of at least 3, genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 4 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 5 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 6 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 7 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 8 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of at least 9 genes selected from the group of genes APOBR, CASP1, CASP10, FFAR2, MS4A4A, MTF1, SECTM1, SEMA4A, TLR8, and TNFRSF1B. In certain embodiments, the data set comprises gene expression measurement of APOBR, CASP1, CASP10, FFAR2, MS4A4A, MTF1, SECTM1, SEMA4A, TLR8, and TNFRSF1B.

In certain embodiments, the data set comprises gene expression measurement of at least 2 genes selected from the group of genes ADAM8, APOBEC3B, CCL28, CD177, CXCL1, EIF2AK2, FCGR3B, IL10RA, LILRA5, and OSCAR, and classifying the lupus disease state of the patient can include classifying whether the patient has the group A lupus disease state, or group H lupus disease state, e.g., classifying the lupus disease state of the patient can include classifying whether the data set is indicative of the patient having group A lupus disease state, or group H lupus disease state. The lupus disease state of the patient can be classified as group A lupus disease state, or group H lupus disease state based on the dataset. In certain embodiments, the data set comprises gene expression measurement of at least 2, 3, 4, 5, 6, 7, 8, 9 or 10 genes selected from the group of genes ADAM8, APOBEC3B, CCL28, CD177, CXCL1, EIF2AK2, FCGR3B, IL10RA, LILRA5, and OSCAR, and classifying the lupus disease state of the patient can include classifying whether the patient has the group A lupus disease state, or group H lupus disease state. In certain embodiments, the data set comprises gene expression measurement of at least 3, genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 4 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 5 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 6 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 7 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 8 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of at least 9 genes selected from the group of genes ADAM8, APOBEC3B, CCL28, CD177, CXCL1, EIF2AK2, FCGR3B, IL10RA, LILRA5, and OSCAR. In certain embodiments, the data set comprises gene expression measurement of ADAM8, APOBEC3B, CCL28, CD177, CXCL1, EIF2AK2, FCGR3B, IL10RA, LILRA5, and OSCAR.

In certain embodiments, the data set comprises gene expression measurement of at least 2 genes selected from the group of genes CANX, CASP1, CHUK, DERL2, ERGIC2, HERPUD1, IRAK1, IRAK4, RIPK1, and SEC24D, and classifying the lupus disease state of the patient can include classifying whether the patient has the group B lupus disease state, or group C lupus disease state, e.g., classifying the lupus disease state of the patient can include classifying whether the data set is indicative of the patient having group B lupus disease state, or group C lupus disease state. The lupus disease state of the patient can be classified as group B lupus disease state, or group C lupus disease state based on the dataset. In certain embodiments, the data set comprises gene expression measurement of at least 2, 3, 4, 5, 6, 7, 8, 9 or 10 genes selected from the group of genes CANX, CASP1, CHUK, DERL2, ERGIC2, HERPUD1, IRAK1, IRAK4, RIPK1, and SEC24D, and classifying the lupus disease state of the patient can include classifying whether the patient has the group B lupus disease state, or group C lupus disease state. In certain embodiments, the data set comprises gene expression measurement of at least 3, genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of at least 4 genes selected from the group of genes CANX, CASP1, CHUK, DERL2, ERGIC2, HERPUD1, IRAK1, IRAK4, RIPK1, and SEC24D.

In certain embodiments, the data set comprises gene expression measurement of at least 5 genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of at least 6 genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of at least 7 genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of at least 8 genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of at least 9 genes selected from the group of genes CANX, CASP1, CHUK, DERL2, ERGIC2, HERPUD1, IRAK1, IRAK4, RIPK1, and SEC24D. In certain embodiments, the data set comprises gene expression measurement of CANX, CASP1, CHUK, DERL2, ERGIC2, HERPUD1, IRAK1, IRAK4, RIPK1, and SEC24D.

In certain embodiments, the data set comprises gene expression measurement of at least 2 genes selected from the group of genes CALR, EDEM2, EMC9, ERAP1, KDELC1, MANF, NUCB2, PSMB8, SEC24D, and TRDC, and classifying the lupus disease state of the patient can include classifying whether the patient has the group B lupus disease state, or group D lupus disease state, e.g., classifying the lupus disease state of the patient can include classifying whether the data set is indicative of the patient having group B lupus disease state, or group D lupus disease state. The lupus disease state of the patient can be classified as group B lupus disease state, or group D lupus disease state based on the dataset. In certain embodiments, the data set comprises gene expression measurement of at least 2, 3, 4, 5, 6, 7, 8, 9 or 10 genes selected from the group of genes CALR, EDEM2, EMC9, ERAP1, KDELC1, MANF, NUCB2, PSMB8, SEC24D, and TRDC, and classifying the lupus disease state of the patient can include classifying whether the patient has the group B lupus disease state, or group D lupus disease state. In certain embodiments, the data set comprises gene expression measurement of at least 3, genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 4 genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 5 genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 6 genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 7 genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 8 genes selected from the group of genes CALR, EDEM2, EMC9, ERAP1, KDELC1, MANF, NUCB2, PSMB8, SEC24D, and TRDC. In certain embodiments, the data set comprises gene expression measurement of at least 9 genes selected from the group of genes CALR, EDEM2, EMC9, ERAP1, KDELC1, MANF, NUCB2, PSMB8, SEC24D, and TRDC. In certain embodiments, the data set comprises gene expression measurement of CALR, EDEM2, EMC9, ERAP1, KDELC1, MANF, NUCB2, PSMB8, SEC24D, and TRDC.

In certain embodiments, the data set comprises gene expression measurement of at least 2 genes selected from the group of genes ACLY, ARSE, CD38, DERL1, DERL2, EDEM3, EIF2AK2, MANF, NFKB1 and SEC24D, and classifying the lupus disease state of the patient can include classifying whether the patient has the group B lupus disease state, or group E lupus disease state, e.g., classifying the lupus disease state of the patient can include classifying whether the data set is indicative of the patient having group B lupus disease state, or group E lupus disease state. The lupus disease state of the patient can be classified as group B lupus disease state, or group E lupus disease state based on the dataset. In certain embodiments, the data set comprises gene expression measurement of at least 2, 3, 4, 5, 6, 7, 8, 9 or 10 genes selected from the group of genes ACLY, ARSE, CD38, DERL1, DERL2, EDEM3, EIF2AK2, MANF, NFKB1 and SEC24D, and classifying the lupus disease state of the patient can include classifying whether the patient has the group B lupus disease state, or group E lupus disease state. In certain embodiments, the data set comprises gene expression measurement of at least 3, genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 4 genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 5 genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 6 genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 7 genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 8 genes selected from the group of genes ACLY, ARSE, CD38, DERL1, DERL2, EDEM3, EIF2AK2, MANF, NFKB1 and SEC24D. In certain embodiments, the data set comprises gene expression measurement of at least 9 genes selected from the group of genes ACLY, ARSE, CD38, DERL1, DERL2, EDEM3, EIF2AK2, MANF, NFKB1 and SEC24D. In certain embodiments, the data set comprises gene expression measurement of ACLY, ARSE, CD38, DERL1, DERL2, EDEM3, EIF2AK2, MANF, NFKB1 and SEC24D.

In certain embodiments, the data set comprises gene expression measurement of at least 2 genes selected from the group of genes ACSL1, AIM2, ASAP1, CASP1, IL18, IL1B, IL1RN, MTF1, RIPK1, and SPI1, and classifying the lupus disease state of the patient can include classifying whether the patient has the group B lupus disease state, or group F lupus disease state, e.g., classifying the lupus disease state of the patient can include classifying whether the data set is indicative of the patient having group B lupus disease state, or group F lupus disease state. The lupus disease state of the patient can be classified as group B lupus disease state, or group F lupus disease state based on the dataset. In certain embodiments, the data set comprises gene expression measurement of at least 2, 3, 4, 5, 6, 7, 8, 9 or 10 genes selected from the group of genes ACSL1, AIM2, ASAP1, CASP1, IL18, IL1B, IL1RN, MTF1, RIPK1, and SPI1, and classifying the lupus disease state of the patient can include classifying whether the patient has the group B lupus disease state, or group F lupus disease state. In certain embodiments, the data set comprises gene expression measurement of at least 3, genes selected from the group of genes ACSL1, AIM2, ASAP1, CASP1, IL18, IL1B, IL1RN, MTF1, RIPK1, and SPI1. In certain embodiments, the data set comprises gene expression measurement of at least 4 genes selected from the group of genes ACSL1, AIM2, ASAP1, CASP1, IL18, IL1B, IL1RN, MTF1, RIPK1, and SPI1. In certain embodiments, the data set comprises gene expression measurement of at least 5 genes selected from the group of genes ACSL1, AIM2, ASAP1, CASP1, IL18, IL1B, IL1RN, MTF1, RIPK1, and SPI1. In certain embodiments, the data set comprises gene expression measurement of at least 6 genes selected from the group of genes ACSL1, AIM2, ASAP1, CASP1, IL18, IL1B, IL1RN, MTF1, RIPK1, and SPI1. In certain embodiments, the data set comprises gene expression measurement of at least 7 genes selected from the group of genes ACSL1, AIM2, ASAP1, CASP1, IL18, IL1B, IL1RN, MTF1, RIPK1, and SPI1. In certain embodiments, the data set comprises gene expression measurement of at least 8 genes selected from the group of genes ACSL1, AIM2, ASAP1, CASP1, IL18, IL1B, IL1RN, MTF1, RIPK1, and SPI1. In certain embodiments, the data set comprises gene expression measurement of at least 9 genes selected from the group of genes ACSL1, AIM2, ASAP1, CASP1, IL18, IL1B, IL1RN, MTF1, RIPK1, and SPI1. In certain embodiments, the data set comprises gene expression measurement of ACSL1, AIM2, ASAP1, CASP1, IL18, IL1B, IL1RN, MTF1, RIPK1, and SPI1.

In certain embodiments, the data set comprises gene expression measurement of at least 2 genes selected from the group of genes ACLY, ARSE, BHMT, CASP10, CD37, EDEM2, GLS, HOMER2, IL1A, and TNFAIP3, and classifying the lupus disease state of the patient can include classifying whether the patient has the group B lupus disease state, or group G lupus disease state, e.g., classifying the lupus disease state of the patient can include classifying whether the data set is indicative of the patient having group B lupus disease state, or group G lupus disease state. The lupus disease state of the patient can be classified as group B lupus disease state, or group G lupus disease state based on the dataset. In certain embodiments, the data set comprises gene expression measurement of at least 2, 3, 4, 5, 6, 7, 8, 9 or 10 genes selected from the group of genes ACLY, ARSE, BHMT, CASP10, CD37, EDEM2, GLS, HOMER2, IL1A, and TNFAIP3, and classifying the lupus disease state of the patient can include classifying whether the patient has the group B lupus disease state, or group G lupus disease state. In certain embodiments, the data set comprises gene expression measurement of at least 3, genes selected from the group of genes ACLY, ARSE, BHMT, CASP10, CD37, EDEM2, GLS, HOMER2, IL1A, and TNFAIP3. In certain embodiments, the data set comprises gene expression measurement of at least 4 genes selected from the group of genes ACLY, ARSE, BHMT, CASP10, CD37, EDEM2, GLS, HOMER2, IL1A, and TNFAIP3. In certain embodiments, the data set comprises gene expression measurement of at least 5 genes selected from the group of genes ACLY, ARSE, BHMT, CASP10, CD37, EDEM2, GLS, HOMER2, IL1A, and TNFAIP3. In certain embodiments, the data set comprises gene expression measurement of at least 6 genes selected from the group of genes ACLY, ARSE, BHMT, CASP10, CD37, EDEM2, GLS, HOMER2, IL1A, and TNFAIP3. In certain embodiments, the data set comprises gene expression measurement of at least 7 genes selected from the group of genes ACLY, ARSE, BHMT, CASP10, CD37, EDEM2, GLS, HOMER2, IL1A, and TNFAIP3. In certain embodiments, the data set comprises gene expression measurement of at least 8 genes selected from the group of genes ACLY, ARSE, BHMT, CASP10, CD37, EDEM2, GLS, HOMER2, IL1A, and TNFAIP3. In certain embodiments, the data set comprises gene expression measurement of at least 9 genes selected from the group of genes ACLY, ARSE, BHMT, CASP10, CD37, EDEM2, GLS, HOMER2, IL1A, and TNFAIP3. In certain embodiments, the data set comprises gene expression measurement of ACLY, ARSE, BHMT, CASP10, CD37, EDEM2, GLS, HOMER2, IL1A, and TNFAIP3.

In certain embodiments, the data set comprises gene expression measurement of at least 2 genes selected from the group of genes ACSL1, AIM2, AKAP10, CASP10, CD38, CKB, IL18, NAIP, NFKB1, and TYROBP, and classifying the lupus disease state of the patient can include classifying whether the patient has the group B lupus disease state, or group H lupus disease state, e.g., classifying the lupus disease state of the patient can include classifying whether the data set is indicative of the patient having group B lupus disease state, or group H lupus disease state. The lupus disease state of the patient can be classified as group B lupus disease state, or group H lupus disease state based on the dataset. In certain embodiments, the data set comprises gene expression measurement of at least 2, 3, 4, 5, 6, 7, 8, 9 or 10 genes selected from the group of genes ACSL1, AIM2, AKAP10, CASP10, CD38, CKB, IL18, NAIP, NFKB1, and TYROBP, and classifying the lupus disease state of the patient can include classifying whether the patient has the group B lupus disease state, or group H lupus disease state. In certain embodiments, the data set comprises gene expression measurement of at least 3, genes selected from the group of genes ACSL1, AIM2, AKAP10, CASP10, CD38, CKB, IL18, NAIP, NFKB1, and TYROBP. In certain embodiments, the data set comprises gene expression measurement of at least 4 genes selected from the group of genes ACSL1, AIM2, AKAP10, CASP10, CD38, CKB, IL18, NAIP, NFKB1, and TYROBP. In certain embodiments, the data set comprises gene expression measurement of at least 5 genes selected from the group of genes ACSL1, AIM2, AKAP10, CASP10, CD38, CKB, IL18, NAIP, NFKB1, and TYROBP. In certain embodiments, the data set comprises gene expression measurement of at least 6 genes selected from the group of genes ACSL1, AIM2, AKAP10, CASP10, CD38, CKB, IL18, NAIP, NFKB1, and TYROBP. In certain embodiments, the data set comprises gene expression measurement of at least 7 genes selected from the group of genes ACSL1, AIM2, AKAP10, CASP10, CD38, CKB, IL18, NAIP, NFKB1, and TYROBP. In certain embodiments, the data set comprises gene expression measurement of at least 8 genes selected from the group of genes ACSL1, AIM2, AKAP10, CASP10, CD38, CKB, IL18, NAIP, NFKB1, and TYROBP. In certain embodiments, the data set comprises gene expression measurement of at least 9 genes selected from the group of genes ACSL1, AIM2, AKAP10, CASP10, CD38, CKB, IL18, NAIP, NFKB1, and TYROBP. In certain embodiments, the data set comprises gene expression measurement of ACSL1, AIM2, AKAP10, CASP10, CD38, CKB, IL18, NAIP, NFKB1, and TYROBP.

In certain embodiments, the data set comprises gene expression measurement of at least 2 genes selected from the group of genes BLK, CD247, CD3D, CD8A, IGHG1, IGHV3-20, SH2D1A, THEMIS2, TRDC, and TRG-AS1, and classifying the lupus disease state of the patient can include classifying whether the patient has the group C lupus disease state, or group D lupus disease state, e.g., classifying the lupus disease state of the patient can include classifying whether the data set is indicative of the patient having group C lupus disease state, or group D lupus disease state. The lupus disease state of the patient can be classified as group C lupus disease state, or group D lupus disease state based on the dataset. In certain embodiments, the data set comprises gene expression measurement of at least 2, 3, 4, 5, 6, 7, 8, 9 or 10 genes selected from the group of genes BLK, CD247, CD3D, CD8A, IGHG1, IGHV3-20, SH2D1A, THEMIS2, TRDC, and TRG-AS1, and classifying the lupus disease state of the patient can include classifying whether the patient has the group C lupus disease state, or group D lupus disease state. In certain embodiments, the data set comprises gene expression measurement of at least 3, genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 4 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 5 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 6 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 7 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 8 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of at least 9 genes selected from the group of genes BLK, CD247, CD3D, CD8A, IGHG1, IGHV3-20, SH2D1A, THEMIS2, TRDC, and TRG-AS1. In certain embodiments, the data set comprises gene expression measurement of BLK, CD247, CD3D, CD8A, IGHG1, IGHV3-20, SH2D1A, THEMIS2, TRDC, and TRG-AS1.

In certain embodiments, the data set comprises gene expression measurement of at least 2 genes selected from the group of genes AURKB, CCNB1, CCNE1, EIF2AK2, IGHG1, IGHV3-20, IGLL1, IGLV4-3, IGLVI-70, and PTTG1, and classifying the lupus disease state of the patient can include classifying whether the patient has the group C lupus disease state, or group E lupus disease state, e.g., classifying the lupus disease state of the patient can include classifying whether the data set is indicative of the patient having group C lupus disease state, or group E lupus disease state. The lupus disease state of the patient can be classified as group C lupus disease state, or group E lupus disease state based on the dataset. In certain embodiments, the data set comprises gene expression measurement of at least 2, 3, 4, 5, 6, 7, 8, 9 or 10 genes selected from the group of genes AURKB, CCNB1, CCNE1, EIF2AK2, IGHG1, IGHV3-20, IGLL1, IGLV4-3, IGLVI-70, and PTTG1, and classifying the lupus disease state of the patient can include classifying whether the patient has the group C lupus disease state, or group E lupus disease state. In certain embodiments, the data set comprises gene expression measurement of at least 3, genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 4 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 5 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 6 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 7 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 8 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of at least 9 genes selected from the group of genes AURKB, CCNB1, CCNE1, EIF2AK2, IGHG1, IGHV3-20, IGLL1, IGLV4-3, IGLVI-70, and PTTG1. In certain embodiments, the data set comprises gene expression measurement of AURKB, CCNB1, CCNE1, EIF2AK2, IGHG1, IGHV3-20, IGLL1, IGLV4-3, IGLVI-70, and PTTG1.

In certain embodiments, the data set comprises gene expression measurement of at least 2 genes selected from the group of genes CD226, CD247, CD28, CD4, CLEC1OA, HLA-DRB1, HLA-DRB6, IGIP, LY75, and TRDC, and classifying the lupus disease state of the patient can include classifying whether the patient has the group C lupus disease state, or group F lupus disease state, e.g., classifying the lupus disease state of the patient can include classifying whether the data set is indicative of the patient having group C lupus disease state, or group F lupus disease state. The lupus disease state of the patient can be classified as group C lupus disease state, or group F lupus disease state based on the dataset. In certain embodiments, the data set comprises gene expression measurement of at least 2, 3, 4, 5, 6, 7, 8, 9 or 10 genes selected from the group of genes CD226, CD247, CD28, CD4, CLEC1OA, HLA-DRB1, HLA-DRB6, IGIP, LY75, and TRDC, and classifying the lupus disease state of the patient can include classifying whether the patient has the group C lupus disease state, or group F lupus disease state. In certain embodiments, the data set comprises gene expression measurement of at least 3, genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 4 genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 5 genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 6 genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 7 genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 8 genes selected from the group of genes CD226, CD247, CD28, CD4, CLEC1OA, HLA-DRB1, HLA-DRB6, IGIP, LY75, and TRDC. In certain embodiments, the data set comprises gene expression measurement of at least 9 genes selected from from the group of genes CD226, CD247, CD28, CD4, CLEC1OA, HLA-DRB1, HLA-DRB6, IGIP, LY75, and TRDC. In certain embodiments, the data set comprises gene expression measurement of CD226, CD247, CD28, CD4, CLEC1OA, HLA-DRB1, HLA-DRB6, IGIP, LY75, and TRDC.

In certain embodiments, the data set comprises gene expression measurement of at least 2 genes selected from the group of genes ACLY, CASP10, CD37, CD38, DERL1, DERL2, EDEM2, EIF2AK2, IF1I30, and MANF, and classifying the lupus disease state of the patient can include classifying whether the patient has the group C lupus disease state, or group G lupus disease state, e.g., classifying the lupus disease state of the patient can include classifying whether the data set is indicative of the patient having group C lupus disease state, or group G lupus disease state. The lupus disease state of the patient can be classified as group C lupus disease state, or group G lupus disease state based on the dataset. In certain embodiments, the data set comprises gene expression measurement of at least 2, 3, 4, 5, 6, 7, 8, 9 or 10 genes selected from the group of genes ACLY, CASP10, CD37, CD38, DERL1, DERL2, EDEM2, EIF2AK2, IF1I30, and MANF, and classifying the lupus disease state of the patient can include classifying whether the patient has the group C lupus disease state, or group G lupus disease state. In certain embodiments, the data set comprises gene expression measurement of at least 3, genes selected from the group of genes ACLY, CASP10, CD37, CD38, DERL1, DERL2, EDEM2, EIF2AK2, IFI30, and MANF. In certain embodiments, the data set comprises gene expression measurement of at least 4 genes selected from the group of genes ACLY, CASP10, CD37, CD38, DERL1, DERL2, EDEM2, EIF2AK2, 1F130, and MANF. In certain embodiments, the data set comprises gene expression measurement of at least 5 genes selected from the group of genes ACLY, CASP10, CD37, CD38, DERL1, DERL2, EDEM2, EIF2AK2, 1F130, and MANF. In certain embodiments, the data set comprises gene expression measurement of at least 6 genes selected from the group of genes ACLY, CASP10, CD37, CD38, DERL1, DERL2, EDEM2, EIF2AK2, IF130, and MANF. In certain embodiments, the data set comprises gene expression measurement of at least 7 genes selected from the group of genes ACLY, CASP10, CD37, CD38, DERL1, DERL2, EDEM2, EIF2AK2, IF1I30, and MANF. In certain embodiments, the data set comprises gene expression measurement of at least 8 genes selected from the group of genes ACLY, CASP10, CD37, CD38, DERL1, DERL2, EDEM2, EIF2AK2, 1F130, and MANF. In certain embodiments, the data set comprises gene expression measurement of at least 9 genes selected from the group of genes ACLY, CASP10, CD37, CD38, DERL1, DERL2, EDEM2, EIF2AK2, IF130, and MANF. In certain embodiments, the data set comprises gene expression measurement of ACLY, CASP10, CD37, CD38, DERL1, DERL2, EDEM2, EIF2AK2, 1F130, and MANF.

In certain embodiments, the data set comprises gene expression measurement of at least 2 genes selected from the group of genes AURKB, BRCA1, E2F3, EIF2AK2, IFITM3, MCM10, NDC80, PTTG1, SOCS3, and TNFAIP3, and classifying the lupus disease state of the patient can include classifying whether the patient has the group C lupus disease state, or group H lupus disease state, e.g., classifying the lupus disease state of the patient can include classifying whether the data set is indicative of the patient having group C lupus disease state, or group H lupus disease state. The lupus disease state of the patient can be classified as group C lupus disease state, or group H lupus disease state based on the dataset. In certain embodiments, the data set comprises gene expression measurement of at least 2, 3, 4, 5, 6, 7, 8, 9 or 10 genes selected from the group of genes AURKB, BRCA1, E2F3, EIF2AK2, IFITM3, MCM10, NDC80, PTTG1, SOCS3, and TNFAIP3, and classifying the lupus disease state of the patient can include classifying whether the patient has the group C lupus disease state, or group H lupus disease state. In certain embodiments, the data set comprises gene expression measurement of at least 3, genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of at least 4 genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of at least 5 genes selected from AURKB, BRCA1, E2F3, EIF2AK2, IFITM3, MCM10, NDC80, PTTG1, SOCS3, and TNFAIP3. In certain embodiments, the data set comprises gene expression measurement of at least 6 genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of at least 7 genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of at least 8 genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of at least 9 genes selected from the group of genes AURKB, BRCA1, E2F3, EIF2AK2, IFITM3, MCM10, NDC80, PTTG1, SOCS3, and TNFAIP3. In certain embodiments, the data set comprises gene expression measurement of AURKB, BRCA1, E2F3, EIF2AK2, IFITM3, MCM10, NDC80, PTTG1, SOCS3, and TNFAIP3.

In certain embodiments, the data set comprises gene expression measurement of at least 2 genes selected from the group of genes CD3E, HLA-DMA, HLA-DPA1, HLA-DPB2, HLA-DQA2, HLA-DRB1, HLA-DRB6, KLRF1, NCAM1, and TRDC, and classifying the lupus disease state of the patient can include classifying whether the patient has the group D lupus disease state, or group E lupus disease state, e.g., classifying the lupus disease state of the patient can include classifying whether the data set is indicative of the patient having group D lupus disease state, or group E lupus disease state. The lupus disease state of the patient can be classified as group D lupus disease state, or group E lupus disease state based on the dataset. In certain embodiments, the data set comprises gene expression measurement of at least 2, 3, 4, 5, 6, 7, 8, 9 or 10 genes selected from the group of genes CD3E, HLA-DMA, HLA-DPA1, HLA-DPB2, HLA-DQA2, HLA-DRB1, HLA-DRB6, KLRF1, NCAM1, and TRDC, and classifying the lupus disease state of the patient can include classifying whether the patient has the group D lupus disease state, or group E lupus disease state. In certain embodiments, the data set comprises gene expression measurement of at least 3, genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 4 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 5 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 6 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 7 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 8 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of at least 9 genes selected from the group of genes CD3E, HLA-DMA, HLA-DPA1, HLA-DPB2, HLA-DQA2, HLA-DRB1, HLA-DRB6, KLRF1, NCAM1, and TRDC. In certain embodiments, the data set comprises gene expression measurement of the group of genes CD3E, HLA-DMA, HLA-DPA1, HLA-DPB2, HLA-DQA2, HLA-DRB1, HLA-DRB6, KLRF1, NCAM1, and TRDC.

In certain embodiments, the data set comprises gene expression measurement of at least 2 genes selected from the group of genes BLK, CD226, CD247, CD8A, HLA-DQA1, HLA-DQA2, HLA-DRB5, HLA-DRB6, TARP, and TRG-AS1, and classifying the lupus disease state of the patient can include classifying whether the patient has the group D lupus disease state, or group F lupus disease state, e.g., classifying the lupus disease state of the patient can include classifying whether the data set is indicative of the patient having group D lupus disease state, or group F lupus disease state. The lupus disease state of the patient can be classified as group D lupus disease state, or group F lupus disease state based on the dataset. In certain embodiments, the data set comprises gene expression measurement of at least 2, 3, 4, 5, 6, 7, 8, 9 or 10 genes selected from the group of genes BLK, CD226, CD247, CD8A, HLA-DQA1, HLA-DQA2, HLA-DRB5, HLA-DRB6, TARP, and TRG-AS1, and classifying the lupus disease state of the patient can include classifying whether the patient has the group D lupus disease state, or group F lupus disease state. In certain embodiments, the data set comprises gene expression measurement of at least 3, genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 4 genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 5 genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 6 genes selected from BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 7 genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 8 genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of at least 9 genes selected from the group of genes BLK, CD226, CD247, CD8A, HLA-DQA1, HLA-DQA2, HLA-DRB5, HLA-DRB6, TARP, and TRG-AS1. In certain embodiments, the data set comprises gene expression measurement of BLK, CD226, CD247, CD8A, HLA-DQA1, HLA-DQA2, HLA-DRB5, HLA-DRB6, TARP, and TRG-AS1.

In certain embodiments, the data set comprises gene expression measurement of at least 2 genes selected from the group of genes CD14, CLEC5A, LILRA2, LILRA5, LILRA6, LMNB1, NLRC4, OLR1, OSCAR, and SEMA4A, and classifying the lupus disease state of the patient can include classifying whether the patient has the group D lupus disease state, or group G lupus disease state, e.g., classifying the lupus disease state of the patient can include classifying whether the data set is indicative of the patient having group D lupus disease state, or group G lupus disease state. The lupus disease state of the patient can be classified as group D lupus disease state, or group G lupus disease state based on the dataset. In certain embodiments, the data set comprises gene expression measurement of at least 2, 3, 4, 5, 6, 7, 8, 9 or 10 genes selected from the group of genes CD14, CLEC5A, LILRA2, LILRA5, LILRA6, LMNB1, NLRC4, OLR1, OSCAR, and SEMA4A, and classifying the lupus disease state of the patient can include classifying whether the patient has the group D lupus disease state, or group G lupus disease state. In certain embodiments, the data set comprises gene expression measurement of at least 3, genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 4 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 5 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 6 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 7 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 8 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of at least 9 genes selected from the group of genes CD14, CLEC5A, LILRA2, LILRA5, LILRA6, LMNB1, NLRC4, OLR1, OSCAR, and SEMA4A. In certain embodiments, the data set comprises gene expression measurement of the group of genes CD14, CLEC5A, LILRA2, LILRA5, LILRA6, LMNB1, NLRC4, OLR1, OSCAR, and SEMA4A.

In certain embodiments, the data set comprises gene expression measurement of at least 2 genes selected from the group of genes BLK, CD177, CD247, CXCR2, FUT7, LTB4R, OSM, TARP, TRDC, and TRG-AS1, and classifying the lupus disease state of the patient can include classifying whether the patient has the group D lupus disease state, or group H lupus disease state, e.g., classifying the lupus disease state of the patient can include classifying whether the data set is indicative of the patient having group D lupus disease state, or group H lupus disease state. The lupus disease state of the patient can be classified as group D lupus disease state, or group H lupus disease state based on the dataset. In certain embodiments, the data set comprises gene expression measurement of at least 2, 3, 4, 5, 6, 7, 8, 9 or 10 genes selected from the group of genes BLK, CD177, CD247, CXCR2, FUT7, LTB4R, OSM, TARP, TRDC, and TRG-AS1, and classifying the lupus disease state of the patient can include classifying whether the patient has the group D lupus disease state, or group H lupus disease state. In certain embodiments, the data set comprises gene expression measurement of at least 3, genes selected from the group of genes BLK, CD177, CD247, CXCR2, FUT7, LTB4R, OSM, TARP, TRDC, and TRG-AS1. In certain embodiments, the data set comprises gene expression measurement of at least 4 genes selected from the group of genes BLK, CD177, CD247, CXCR2, FUT7, LTB4R, OSM, TARP, TRDC, and TRG-AS1. In certain embodiments, the data set comprises gene expression measurement of at least 5 genes selected from the group of genes BLK, CD177, CD247, CXCR2, FUT7, LTB4R, OSM, TARP, TRDC, and TRG-AS1. In certain embodiments, the data set comprises gene expression measurement of at least 6 genes selected from the group of genes BLK, CD177, CD247, CXCR2, FUT7, LTB4R, OSM, TARP, TRDC, and TRG-AS1. In certain embodiments, the data set comprises gene expression measurement of at least 7 genes selected from the group of genes BLK, CD177, CD247, CXCR2, FUT7, LTB4R, OSM, TARP, TRDC, and TRG-AS1. In certain embodiments, the data set comprises gene expression measurement of at least 8 genes selected from the group of genes BLK, CD177, CD247, CXCR2, FUT7, LTB4R, OSM, TARP, TRDC, and TRG-AS1. In certain embodiments, the data set comprises gene expression measurement of at least 9 genes selected from the group of genes BLK, CD177, CD247, CXCR2, FUT7, LTB4R, OSM, TARP, TRDC, and TRG-AS1. In certain embodiments, the data set comprises gene expression measurement of BLK, CD177, CD247, CXCR2, FUT7, LTB4R, OSM, TARP, TRDC, and TRG-AS1.

In certain embodiments, the data set comprises gene expression measurement of at least 2 genes selected from the group of genes BLK, CCR3, CD247, CD28, CD4, CD8A, CTLA4, HAVCR2, KLRG1, and TRG-AS1, and classifying the lupus disease state of the patient can include classifying whether the patient has the group E lupus disease state, or group F lupus disease state, e.g., classifying the lupus disease state of the patient can include classifying whether the data set is indicative of the patient having group E lupus disease state, or group F lupus disease state. The lupus disease state of the patient can be classified as group E lupus disease state, or group F lupus disease state based on the dataset. In certain embodiments, the data set comprises gene expression measurement of at least 2, 3, 4, 5, 6, 7, 8, 9 or 10 genes selected from the group of genes BLK, CCR3, CD247, CD28, CD4, CD8A, CTLA4, HAVCR2, KLRG1, and TRG-AS1, and classifying the lupus disease state of the patient can include classifying whether the patient has the group E lupus disease state, or group F lupus disease state. In certain embodiments, the data set comprises gene expression measurement of at least 3, genes selected from the group of genes BLK, CCR3, CD247, CD28, CD4, CD8A, CTLA4, HAVCR2, KLRG1, and TRG-AS1. In certain embodiments, the data set comprises gene expression measurement of at least 4 genes selected from the group of genes BLK, CCR3, CD247, CD28, CD4, CD8A, CTLA4, HAVCR2, KLRG1, and TRG-AS1. In certain embodiments, the data set comprises gene expression measurement of at least 5 genes selected from the group of genes BLK, CCR3, CD247, CD28, CD4, CD8A, CTLA4, HAVCR2, KLRG1, and TRG-AS1. In certain embodiments, the data set comprises gene expression measurement of at least 6 genes selected from the group of genes BLK, CCR3, CD247, CD28, CD4, CD8A, CTLA4, HAVCR2, KLRG1, and TRG-AS1. In certain embodiments, the data set comprises gene expression measurement of at least 7 genes selected from the group of genes BLK, CCR3, CD247, CD28, CD4, CD8A, CTLA4, HAVCR2, KLRG1, and TRG-AS1. In certain embodiments, the data set comprises gene expression measurement of at least 8 genes selected from the group of genes BLK, CCR3, CD247, CD28, CD4, CD8A, CTLA4, HAVCR2, KLRG1, and TRG-AS1. In certain embodiments, the data set comprises gene expression measurement of at least 9 genes selected from the group of genes BLK, CCR3, CD247, CD28, CD4, CD8A, CTLA4, HAVCR2, KLRG1, and TRG-AS1. In certain embodiments, the data set comprises gene expression measurement of BLK, CCR3, CD247, CD28, CD4, CD8A, CTLA4, HAVCR2, KLRG1, and TRG-AS1.

In certain embodiments, the data set comprises gene expression measurement of at least 2 genes selected from the group of genes CALR, CD244, CTLA4, DERL2, EMC9, ERAP1, GALNT2, HAVCR2, ICOS, and PSMB8, and classifying the lupus disease state of the patient can include classifying whether the patient has the group E lupus disease state, or group G lupus disease state, e.g., classifying the lupus disease state of the patient can include classifying whether the data set is indicative of the patient having group E lupus disease state, or group G lupus disease state. The lupus disease state of the patient can be classified as group E lupus disease state, or group G lupus disease state based on the dataset. In certain embodiments, the data set comprises gene expression measurement of at least 2, 3, 4, 5, 6, 7, 8, 9 or 10 genes selected from the group of genes CALR, CD244, CTLA4, DERL2, EMC9, ERAP1, GALNT2, HAVCR2, ICOS, and PSMB8, and classifying the lupus disease state of the patient can include classifying whether the patient has the group E lupus disease state, or group G lupus disease state. In certain embodiments, the data set comprises gene expression measurement of at least 3, genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 4 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 5 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 6 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 7 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 8 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 9 genes selected from the group of genes CALR, CD244, CTLA4, DERL2, EMC9, ERAP1, GALNT2, HAVCR2, ICOS, and PSMB8. In certain embodiments, the data set comprises gene expression measurement of CALR, CD244, CTLA4, DERL2, EMC9, ERAP1, GALNT2, HAVCR2, ICOS, and PSMB8.

In certain embodiments, the data set comprises gene expression measurement of at least 2 genes selected from the group of genes CASP1, EIF2AK2, GBP1, IFI30, IFITM3, NEK7, NLRC4, PSMB10, PSMB8, and RIPK1, and classifying the lupus disease state of the patient can include classifying whether the patient has the group E lupus disease state, or group H lupus disease state, e.g., classifying the lupus disease state of the patient can include classifying whether the data set is indicative of the patient having group E lupus disease state, or group H lupus disease state. The lupus disease state of the patient can be classified as group E lupus disease state, or group H lupus disease state based on the dataset. In certain embodiments, the data set comprises gene expression measurement of at least 2, 3, 4, 5, 6, 7, 8, 9 or 10 genes selected from the group of genes CASP1, EIF2AK2, GBP1, IFI30, IFITM3, NEK7, NLRC4, PSMB10, PSMB8, and RIPK1, and classifying the lupus disease state of the patient can include classifying whether the patient has the group E lupus disease state, or group H lupus disease state. In certain embodiments, the data set comprises gene expression measurement of at least 3, genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 4 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 5 genes selected from the group of genes CASP1, EIF2AK2, GBP1, IF130, IFITM3, NEK7, NLRC4, PSMB10, PSMB8, and RIPK1. In certain embodiments, the data set comprises gene expression measurement of at least 6 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 7 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 8 genes selected from the group of genes CASP1, EIF2AK2, GBP1, IF130, IFITM3, NEK7, NLRC4, PSMB10, PSMB8, and RIPK1. In certain embodiments, the data set comprises gene expression measurement of at least 9 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of CASP1, EIF2AK2, GBP1, IF130, IFITM3, NEK7, NLRC4, PSMB10, PSMB8, and RIPK1.

In certain embodiments, the data set comprises gene expression measurement of at least 2 genes selected from the group of genes CALR, CD244, CTLA4, DERL2, EMC9, ERAP1, GALNT2, HAVCR2, ICOS, and PSMB8, and classifying the lupus disease state of the patient can include classifying whether the patient has the group F lupus disease state, or group G lupus disease state, e.g., classifying the lupus disease state of the patient can include classifying whether the data set is indicative of the patient having group F lupus disease state, or group G lupus disease state. The lupus disease state of the patient can be classified as group F lupus disease state, or group G lupus disease state based on the dataset. In certain embodiments, the data set comprises gene expression measurement of at least 2, 3, 4, 5, 6, 7, 8, 9 or 10 genes selected from the group of genes CALR, CD244, CTLA4, DERL2, EMC9, ERAP1, GALNT2, HAVCR2, ICOS, and PSMB8, and classifying the lupus disease state of the patient can include classifying whether the patient has the group F lupus disease state, or group G lupus disease state. In certain embodiments, the data set comprises gene expression measurement of at least 3, genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 4 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 5 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 6 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 7 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 8 genes selected from the group of genes CALR, CD244, CTLA4, DERL2, EMC9, ERAP1, GALNT2, HAVCR2, ICOS, and PSMB8. In certain embodiments, the data set comprises gene expression measurement of at least 9 genes selected from the group of genes CALR, CD244, CTLA4, DERL2, EMC9, ERAP1, GALNT2, HAVCR2, ICOS, and PSMB8. In certain embodiments, the data set comprises gene expression measurement of CALR, CD244, CTLA4, DERL2, EMC9, ERAP1, GALNT2, HAVCR2, ICOS, and PSMB8.

In certain embodiments, the data set comprises gene expression measurement of at least 2 genes selected from the group of genes CASP1, EIF2AK2, GBP1, IFI30, IFITM3, NEK7, NLRC4, PSMB10, PSMB8, and RIPK1, and classifying the lupus disease state of the patient can include classifying whether the patient has the group F lupus disease state, or group H lupus disease state, e.g., classifying the lupus disease state of the patient can include classifying whether the data set is indicative of the patient having group F lupus disease state, or group H lupus disease state. The lupus disease state of the patient can be classified as group F lupus disease state, or group H lupus disease state based on the dataset. In certain embodiments, the data set comprises gene expression measurement of at least 2, 3, 4, 5, 6, 7, 8, 9 or 10 genes selected from the group of genes CASP1, EIF2AK2, GBP1, IFI30, IFITM3, NEK7, NLRC4, PSMB10, PSMB8, and RIPK1, and classifying the lupus disease state of the patient can include classifying whether the patient has the group F lupus disease state, or group H lupus disease state. In certain embodiments, the data set comprises gene expression measurement of at least 3, genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 4 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 5 genes selected from the group of genes CASP1, EIF2AK2, GBP1, IF130, IFITM3, NEK7, NLRC4, PSMB10, PSMB8, and RIPK1. In certain embodiments, the data set comprises gene expression measurement of at least 6 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 7 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of at least 8 genes selected from the group of genes CASP1, EIF2AK2, GBP1, IF130, IFITM3, NEK7, NLRC4, PSMB10, PSMB8, and RIPK1. In certain embodiments, the data set comprises gene expression measurement of at least 9 genes selected from the group of genes CASP1, EIF2AK2, GBP1, IFI30, IFITM3, NEK7, NLRC4, PSMB10, PSMB8, and RIPK1. In certain embodiments, the data set comprises gene expression measurement of CASP1, EIF2AK2, GBP1, IF130, IFITM3, NEK7, NLRC4, PSMB10, PSMB8, and RIPK1.

In certain embodiments, the data set comprises gene expression measurement of at least 2 genes selected from the group of genes ATP5A1, CD160, CD244, CD3E, COX16, COX18, NDUFAF4, NDUFS1, TIMMDC1, and TTC19, and classifying the lupus disease state of the patient can include classifying whether the patient has the group G lupus disease state, or group H lupus disease state, e.g., classifying the lupus disease state of the patient can include classifying whether the data set is indicative of the patient having group G lupus disease state, or group H lupus disease state. The lupus disease state of the patient can be classified as group G lupus disease state, or group H lupus disease state based on the dataset. In certain embodiments, the data set comprises gene expression measurement of at least 2, 3, 4, 5, 6, 7, 8, 9 or 10 genes selected from the group of genes ATP5A1, CD160, CD244, CD3E, COX16, COX18, NDUFAF4, NDUFS1, TIMMDC1, and TTC19, and classifying the lupus disease state of the patient can include classifying whether the patient has the group G lupus disease state, or group H lupus disease state. In certain embodiments, the data set comprises gene expression measurement of at least 3, genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 4 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 5 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 6 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 7 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 8 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of at least 9 genes selected from the group of genes ATP5A1, CD160, CD244, CD3E, COX16, COX18, NDUFAF4, NDUFS1, TIMMDC1, and TTC19. In certain embodiments, the data set comprises gene expression measurement of ATP5A1, CD160, CD244, CD3E, COX16, COX18, NDUFAF4, NDUFS1, TIMMDC1, and TTC19.

In certain embodiments, the analyzing the data set can include providing the data set as an input to a trained machine-learning model. The trained machine-learning model can generate the inference indicative of the lupus disease state of the patient, based on the data set. In certain embodiments, the trained machine-learning model generate the inference whether the data set is indicative of the patient having group A lupus disease state, group B lupus disease state, group C lupus disease state, group D lupus disease state, group E lupus disease state, group F lupus disease state, group G lupus disease state, or group H lupus disease state. In certain embodiments, the trained machine-learning model generate the inference indicative of the lupus disease state of the patient, based on the one or more GSVA scores of the patient. In certain embodiments, the trained machine-learning model generate the inference indicative of the lupus disease state of the patient, based on the one or more enrichment scores of the patient. In certain embodiments, the trained machine-learning model generate the inference indicative of the lupus disease state of the patient, based on the gene expression (e.g., in the biological sample) of the one or more gene sets formed based on the one or more Tables selected from Tables: 1 to 32. In certain embodiments, the trained machine-learning model generate the inference indicative of the lupus disease state of the patient, based on the MEs (e.g., calculated based on the gene expression in the biological sample) of the one or more gene sets formed based on the one or more Tables selected from Tables: 1 to 32. In certain embodiments, the inference is whether the data set is indicative of the patient having group A lupus disease state, group B lupus disease state, group C lupus disease state, group D lupus disease state, group E lupus disease state, group F lupus disease state, group G lupus disease state, or group H lupus disease state. In certain embodiments, the inference is whether the one or more GSVA scores of the patient are indicative of the patient having group A lupus disease state, group B lupus disease state, group C lupus disease state, group D lupus disease state, group E lupus disease state, group F lupus disease state, group G lupus disease state, or group H lupus disease state. In certain embodiments, the inference is whether the one or more enrichment scores of the patient are indicative of the patient having group A lupus disease state, group B lupus disease state, group C lupus disease state, group D lupus disease state, group E lupus disease state, group F lupus disease state, group G lupus disease state, or group H lupus disease state. In certain embodiments, the inference is whether the gene expression (e.g., in the biological sample) of the one or more gene sets formed based on the one or more Tables selected from Tables: 1 to 32, are indicative of the patient having group A lupus disease state, group B lupus disease state, group C lupus disease state, group D lupus disease state, group E lupus disease state, group F lupus disease state, group G lupus disease state, or group H lupus disease state. In certain embodiments, the inference is whether the MEs (e.g., calculated based on the gene expression in the biological sample) of the one or more gene sets formed based on the one or more Tables selected from Tables: 1 to 32, are indicative of the patient having group A lupus disease state, group B lupus disease state, group C lupus disease state, group D lupus disease state, group E lupus disease state, group F lupus disease state, group G lupus disease state, or group H lupus disease state. The trained machine-learning model can be (e.g., has been) trained to generate the inference. The method can classify the lupus disease state of the patient based on the inference.

In certain embodiments, the method classify that the patient has group A lupus disease state, group B lupus disease state, group C lupus disease state, group D lupus disease state, group E lupus disease state, group F lupus disease state, group G lupus disease state, or group H lupus disease state, based on the inference of the trained machine-learning that the data set is indicative of the patient having group A lupus disease state, group B lupus disease state, group C lupus disease state, group D lupus disease state, group E lupus disease state, group F lupus disease state, group G lupus disease state, or group H lupus disease state, respectively. In certain embodiments, the method further comprises receiving, as an output of the trained machine-learning model, the inference; and/or electronically outputting a report classifying the lupus disease state of the patient based on the inference. The trained machine-learning model, can generate the inference, based on comparing the data set to a reference data set. The reference data set can comprise and/or be derived from gene expression measurements from a plurality of reference biological samples. The plurality of reference biological samples can be obtained or derived from a plurality of reference subjects. In certain embodiments, the plurality of reference biological samples comprise i) a first plurality of the reference biological samples obtained or derived from reference subjects having group A lupus disease state, ii) a second plurality of the reference biological samples obtained or derived from reference subjects having group B lupus disease state, iii) a third plurality of the reference biological samples obtained or derived from reference subjects having group C lupus disease state, iv) a fourth plurality of the reference biological samples obtained or derived from reference subjects having group D lupus disease state, v) a fifth plurality of the reference biological samples obtained or derived from reference subjects having group E lupus disease state, vi) a sixth plurality of the reference biological samples obtained or derived from reference subjects having group F lupus disease state, vii) a seventh plurality of the reference biological samples obtained or derived from reference subjects having group G lupus disease state, and/or viii) an eighth plurality of the reference biological samples obtained or derived from reference subjects having group H lupus disease state. In certain embodiments, the plurality reference biological samples comprise a first plurality of reference biological samples obtained or derived from reference subjects having active lupus, a second plurality of reference biological samples obtained or derived from reference subjects having inactive lupus, and/or a third plurality of reference subjects not having lupus. In certain embodiments, the plurality reference biological samples comprise a first plurality of reference biological samples obtained or derived from reference subjects having lupus, a second plurality of reference biological samples obtained or derived from reference subjects not having lupus. In certain embodiments, the reference data set comprise and/or is derived from gene expression measurements from the plurality of reference biological samples of at least 2 genes selected from the genes listed in Tables: 1 to 32. In certain embodiments, the reference data set comprise and/or is derived from gene expression measurements from the plurality of reference biological samples of at least 2 genes selected from genes listed in each of one or more Tables selected from Tables: 1 to 32. The selected genes of the dataset (e.g., gene expression measurements of which the dataset is comprised of or derived from), and the selected genes of the reference data set (e.g., gene expression measurements of which the reference dataset is comprised of or derived from) can at least partially overlap (e.g., one or more of the selected genes can be the same). In certain embodiments, selected genes of the dataset, and selected genes of the reference data are same. In certain embodiments, selected genes of the dataset, and selected genes of the reference data are same, and can be any selected set of genes e.g., of the data set, as described above or elsewhere herein. The Tables selected, and genes selected from a selected Table for the data set and the reference data set can be the same, and can be as described (e.g., for the data set) herein. In certain embodiments, the reference data set comprise a plurality of individual reference data sets. The plurality of individual reference data sets, can be obtained from the plurality of reference subjects. Different individual reference data sets can be obtained from different reference subjects. In certain embodiments, at least one individual reference data set is obtained or derived from each reference subject. A respective individual reference data set can comprise or is derived from gene expression measurements (e.g., of the selected genes of the reference data set), from a respective reference biological sample obtained or derived from a respective reference subject. Each individual reference data set can comprise or is derived from gene expression measurements (e.g., of the selected genes of the reference data set), from a reference biological sample obtained or derived from a reference subject, wherein different individual reference data sets are obtained from different reference subjects. In certain embodiments, the individual reference data sets contain data regarding one or more lupus disease index of the reference subjects. The one or more lupus disease index can include but is not limited to blood anti-double-stranded DNA antibody level, blood anti-ribonucleoprotein (RNP) antibody level, blood complement component 3 (C3) protein level, blood complement component 4 (C4) protein level, SLEDAI score, and LuMOS score. In certain embodiments, the reference data set is derived from the gene expression measurements (e.g., of the selected genes of the reference data set) from the plurality of reference biological samples, wherein the gene expression measurements is analyzed using a suitable data analysis tool including but not limited to a BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), gene set enrichment analysis (GSEA), enrichment algorithm, Z score, multiscale embedded gene co-expression network analysis (MEGENA), weighted gene co-expression network analysis (WGCNA), differential expression analysis, log 2 expression analysis, or any combination thereof, to obtain the reference data set.

In certain embodiments, the gene expression measurements from the plurality of reference biological samples is analyzed using GSVA, to obtain the reference data set. In certain embodiments, the reference data set comprises one or more GSVA scores of the reference biological samples, wherein for a respective reference biological sample the one or more GSVA scores of the respective reference biological sample are generated based on the one or more Tables selected from Tables 1 to 32, wherein for each selected Table, at least 2 genes, effective number of genes, and/or all genes selected from the selected Table forms an input gene set based on which at least one GSVA score of the respective reference biological sample, based on the selected Table is generated. Enrichment of the input gene set in the respective reference biological sample calculated to generate the at least one GSVA score. In certain embodiments, a respective individual reference data set of the plurality of individual reference data sets, comprise one or more GSVA scores of a reference biological sample of the plurality of reference biological samples. In certain embodiments, one or more GSVA scores of each reference biological samples (and/or of the each of the reference subjects) are generated, wherein the one or more GSVA scores of different reference biological samples can be same or different. The one or more GSVA scores of a respective reference biological sample, can be generated based on comparing gene expression measurements of the respective reference biological sample, with the gene expression measurements of the plurality reference biological samples (e.g., of the cohort). The one or more GSVA scores of the patient, can be generated based on comparing gene expression measurements of the biological sample obtained and/or derived from the patient, with the gene expression measurements of the plurality reference biological samples, of the reference dataset. The enrichment of the input gene sets in the biological sample can be determined (e.g., for determining the one or more GSVA scores of the patient) based on comparing the gene expression measurements from the biological sample obtained and/or derived from the patient, with the gene expression measurements from the reference biological samples of the reference dataset. In certain embodiments, the reference data set comprises one or more enrichment scores of the reference biological samples. For a reference biological sample/reference patient the one or more enrichment scores can be generated using a method similar to the method of generating the one or more enrichment scores of the patient, as described above or elsewhere herein. In certain embodiments, for a respective reference biological sample the one or more enrichment scores of the respective reference biological sample are generated based on the one or more Tables selected from Tables 1 to 32, wherein for each selected Table, at least 2 genes, effective number of genes, and/or all genes selected from the selected Table forms an input gene set based on which at least one enrichment score of the respective reference biological sample, based on the selected Table is generated. Enrichment of the input gene set in the respective reference biological sample calculated to generate the at least enrichment score. In certain embodiments, a respective individual reference data set of the plurality of individual reference data sets, comprise one or more enrichment scores of a reference biological sample of the plurality of reference biological samples. In certain embodiments, one or more enrichment scores of each reference biological samples (and/or of the each of the reference subjects) are generated, wherein the one or more enrichment scores of different reference biological samples can be same or different. The one or more enrichment scores of a respective reference biological sample, can be generated based on comparing gene expression measurements of the respective reference biological sample, with the gene expression measurements of the plurality reference biological samples (e.g., of the cohort). The one or more enrichment scores of the patient, can be generated based on comparing gene expression measurements of the biological sample obtained and/or derived from the patient, with the gene expression measurements of the plurality reference biological samples, of the reference dataset. The enrichment of the input gene sets in the biological sample can be determined (e.g., for determining the one or more enrichment scores of the patient) based on comparing the gene expression measurements from the biological sample obtained and/or derived from the patient, with the gene expression measurements from the reference biological samples of the reference dataset. The enrichment score can be determined using the input gene set based on a suitable method including but not limited GSVA, GSEA, or enrichment algorithm. In certain embodiments, the enrichment score is determined using GSVA, and the enrichment score can be GSVA score. In certain embodiments, the reference data set comprises gene expression of one or more gene sets, from the reference biological samples, wherein the one or more gene sets of the reference data set can be same as the one or more gene sets described above or elsewhere herein for the data set. In certain embodiments, the reference data set comprises MEs of the reference biological samples. The MEs of the reference biological samples can be of one or more gene sets (e.g., based on gene expression of which in the reference biological samples the MEs of the reference biological samples are calculated) same as the one or more gene sets described above or elsewhere herein for the data set, (e.g., based on gene expression of which in the biological sample the MEs of the data set are calculated). In certain embodiments, the reference data set can be a reference data set as described in the Examples. In certain embodiments, the reference data set comprises the 17 datasets (e.g., containing 3,166 samples/reference subjects) as described in the Examples and Table 33.

The trained machine learning model can be trained (e.g., can be obtained by training) with the reference data set. In certain embodiments, the reference data set comprises the plurality of individual reference data sets. In certain embodiments, the trained machine learning model is trained to infer the lupus disease state of a respective reference subject based on an respective individual reference data set obtained from a respective reference biological sample from the respective reference subject, wherein the respective individual reference data set comprise or is derived from gene expression measurements (e.g., of the selected genes of the reference data set), from the respective reference biological sample. In certain embodiments, the trained machine learning model is trained based on the one or more GSVA scores of the plurality of reference subjects. In certain embodiments, the trained machine learning model is trained based on the gene expression measurements values (e.g., of the selected genes of the reference data set), from the respective reference biological samples obtained or derived from the plurality of reference subjects. In certain embodiments, the trained machine learning model is trained based on the one or more enrichment scores of the plurality of reference subjects. In certain embodiments, the trained machine learning model is trained based on gene expressions of the one or more gene sets, of the plurality of the reference biological samples from the plurality of reference subjects. In certain embodiments, the trained machine learning model is trained based on the MEs, wherein the MEs are calculated based on the gene expressions of the one or more gene sets, of the reference biological sample from the respective reference subject.

In certain embodiments, the trained machine learning model is trained to infer the lupus disease state of a respective reference subject based on one or more GSVA scores of a respective reference biological sample from the respective reference subject. In certain embodiments, the trained machine learning model is trained to infer the lupus disease state of a respective reference subject based on one or more enrichment scores of a respective reference biological sample from the respective reference subject. In certain embodiments, the trained machine learning model is trained to infer the lupus disease state of a respective reference subject based on gene expression of the one or more gene sets, of the reference biological from the respective reference subject. In certain embodiments, the trained machine learning model is trained to infer the lupus disease state of a respective reference subject based on the MEs, wherein the MEs are calculated based on the gene expression of the one or more gene sets, of the reference biological sample from the respective reference subject. The trained machine learning model can be trained using a suitable method, and suitable reference data set such that the trained machine learning model (e.g., obtained by training) can generate the inference indicative of the lupus disease state of the patient based on the data set, with a desired accuracy, sensitivity, specificity, positive predictive value, and/or negative predictive value. The desired accuracy, sensitivity, specificity, positive predictive value, and/or negative predictive value, can be respectively an accuracy, sensitivity, specificity, positive predictive value, and/or negative predictive value described above or elsewhere herein. In certain embodiments, the reference date set is normalized. In certain embodiments, the reference date set is culled of outliers, were cleaned of background noise and/or was normalized using Robust Multiarray Average (RMA), Guanine Cytosine Robust Multi-Array Analysis (GCRMA), or normexp background correction (NEQC) based on the microarray platform resulting in log 2 transformed expression values. In certain embodiments, the reference date set is culled of outliers; were cleaned of background noise; were removed of data without annotation data; scaled; variance corrected and/or normalized.

Normalizing can be performed using Robust Multiarray Average (RMA), Guanine Cytosine Robust Multi-Array Analysis (GCRMA), Linear Models for Microarray Data, variance stabilizing transformation (VST), or normexp background correction (NEQC) based on the microarray platform resulting in log 2 transformed expression values. The individual reference data set can be an individual reference data set as described above or elsewhere herein. In certain embodiments, the trained machine learning model can be trained using a method, and/or a reference data set as described in the Examples. In certain embodiments, the trained machine learning model can be trained using a reference data set containing the 17 datasets (e.g., containing 3,166 samples/reference subjects) as disclosed in the Examples, using a method described in the Examples. In certain embodiments, a first portion of the reference data set can be used as training data set, and a second portion of the reference data set can be used as validation data set, for training the machine learning model. One-vs.-one and one-vs.-rest multi-class classifications with leave-one-out cross-validation can employed to infer reference subjects lupus disease state one of eight groups, group A-H lupus disease state. In certain embodiments, 0 to 25 fold, such as 0, 1, 2, 3, 4, 5, 6, 7, 8, 9, 10, 11, 12, 13, 14, 15, 16, 17, 18, 19, 20, 21, 22, 23, 24 or 25 fold cross-validation is used. In certain embodiments, 6 fold cross-validation is used. In certain embodiments, 10 fold cross-validation is used. In certain embodiments, oversampling or undersampling correction is made during training of the machine learning model. Synthetic Minority Oversampling Technique (SMOTE) can be applied on the training data to handle class imbalances. In the certain embodiments, the trained machine-learning model generate the inference based on the one or more GSVA scores of the patient, and the trained machine-learning model is trained with a reference dataset comprising the one or more GSVA scores from the plurality of reference biological samples. The one or more GSVA scores of the patient can be generated based on comparing the data set with a reference data set as described above or elsewhere herein. In certain embodiments, the one or more GSVA scores of the patient are generated based on comparing the data set with the reference data set, and the enrichment of expression of input gene sets, (e.g., for calculating the one or more GSVA scores of the patient) in the biological sample obtained or derived from the patient can be measured based comparing gene expression measurements data of the biological sample, with the gene expression measurements data from the plurality of reference samples of the reference data set. The reference data set used for generating the one or more GSVA scores of the patient, and the reference data set used for training the machine learning model can be same or different. In certain embodiments, the reference data set used for generating the one or more GSVA scores of the patient, and the reference data set used for training the machine learning model is the same.

In the certain embodiments, the trained machine-learning model generate the inference based on the one or more enrichment scores of the patient, and the trained machine-learning model is trained with a reference dataset comprising the one or more enrichment scores from the plurality of reference biological samples. The one or more enrichment scores of the patient can be generated based on comparing the data set with a reference data set as described above or elsewhere herein. In certain embodiments, the one or more enrichment scores of the patient are generated based on comparing the data set with the reference data set, and the enrichment of expression of input gene sets, (e.g., for calculating the one or more enrichment scores of the patient) in the biological sample obtained or derived from the patient can be measured based comparing gene expression measurements data of the biological sample, with the gene expression measurements data from the plurality of reference samples of the reference data set.

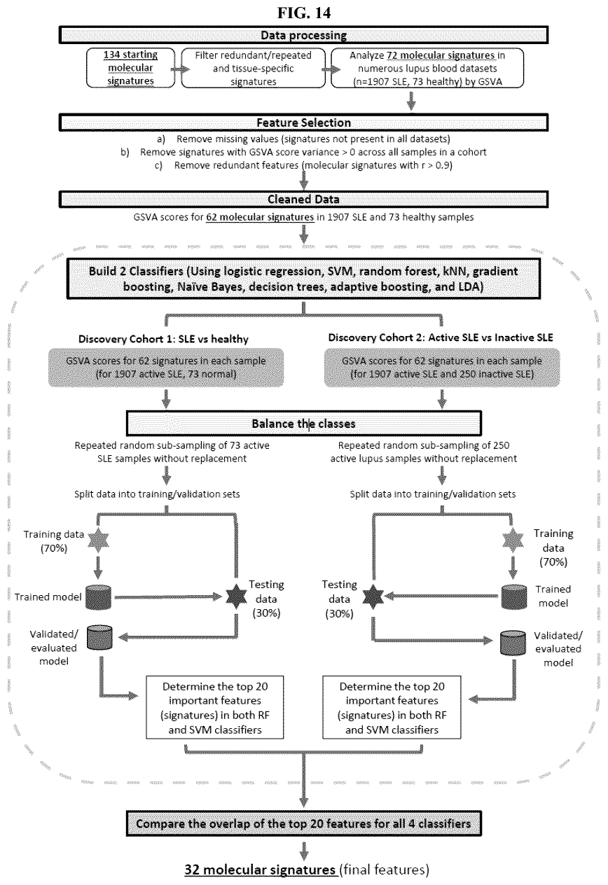

The reference data set used for generating the one or more enrichment scores of the patient, and the reference data set used for training the machine learning model can be same or different. In certain embodiments, the reference data set used for generating the one or more enrichment scores of the patient, and the reference data set used for training the machine learning model is the same. In certain embodiments, the trained machine learning model is trained based on gene expressions of the one or more gene sets, of the plurality of the reference biological samples from the plurality of reference subjects, and the trained machine-learning model generate the inference indicative of the lupus disease state of the patient, based on the gene expression (e.g., in the biological sample) of the one or more gene sets, wherein the one or more gene sets are formed based on the one or more Tables selected from Tables: 1 to 32. In certain embodiments, the trained machine learning model is trained based on the MEs, wherein the MEs are calculated based on the gene expressions of the one or more gene sets, of the reference biological sample from the respective reference subject, and the trained machine-learning model generate the inference indicative of the lupus disease state of the patient, based on the MEs (e.g., calculated based on the gene expression in the biological sample) of the one or more gene sets, wherein the one or more gene sets are formed based on the one or more Tables selected from Tables: 1 to 32. In certain embodiments, the trained machine-learning model that is trained by at least: i) determining GSVA scores for a reference data set comprising lupus samples and healthy samples, the reference data set comprising gene expression measurements of the 62 gene signatures shown in FIG. 14, ii) training a first machine-learning model based on the GSVA scores for the reference data set to generate first inferences of whether the samples of the reference data set are indicative of having lupus or not having lupus, iii) determining a first set of features of the first machine-learning model based on importance of the first set of features to the first inferences, iv) training a second machine-learning model based on the GSVA scores of the lupus samples of the reference data set to generate second inferences of whether the lupus samples of the reference data set are indicative of having active lupus or inactive lupus, v) determining second set of features of the second machine-learning model based on importance of the second set of features to the second inferences, vi) determining a third set of features based on overlap of the first set of features and the second set of features, and vii) determining the trained machine-learning model based on GSVA scores of the third set of features to generate third inferences of whether the samples of the reference data set are indicative having group A lupus disease state, group B lupus disease state, group C lupus disease state, group D lupus disease state, group E lupus disease state, group F lupus disease state, group G lupus disease state, or group H lupus disease state. In certain embodiments, determining GSVA scores for the reference data set comprises determining 62 GSVA scores for each samples of the reference data set, wherein for a respective sample one GSVA score is generated based on each of the 62 gene signatures shown in FIG. 14. In certain embodiments, the trained machine learning model can be obtained using one or more steps as described in FIG. 14.

The trained machine-learning model can be trained (e.g., obtained by training) using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, a Random Forest (RF), deep learning algorithm, linear discriminant analysis (LDA), decision tree learning (DTREE), adaptive boosting (ADB), Classification and Regression Tree (CART), hierarchical clustering, or any combination thereof. The algorithm of the trained machine learning model can be a machine learning classifier, e.g., mentioned in this paragraph. The machine learning classifier (e.g., linear regression, LOG, Ridge regression, Lasso regression, EN regression, SVM, GBM, kNN, GLM, NB classifier, neural network, a RF, deep learning algorithm, LDA, DTREE, ADB, CART, and/or hierarchical clustering) can be trained to obtain the trained machine learning model. In some embodiments, the trained machine learning model, is trained using a supervised machine learning algorithm or an unsupervised machine learning algorithm, e.g., the classifier can be a supervised machine learning algorithm or an unsupervised machine learning algorithm. In certain embodiments, the trained machine learning model is trained using linear regression. In certain embodiments, the trained machine learning model is trained using LOG. In certain embodiments, the trained machine learning model is trained using Ridge regression. In certain embodiments, the trained machine learning model is trained using Lasso regression. In certain embodiments, the trained machine learning model is trained using EN. In certain embodiments, the trained machine learning model is trained using SVM. In certain embodiments, the machine learning model is trained using GBM. In certain embodiments, the trained machine learning model is trained using KNN. In certain embodiments, the trained machine learning model is trained using GLM. In certain embodiments, the trained machine learning model is trained using NB. In certain embodiments, the trained machine learning model is trained using RE. In certain embodiments, the trained machine learning model is trained using deep learning algorithm. In certain embodiments, the trained machine learning model is trained using LDA. In certain embodiments, the trained machine learning model is trained using DTREE. In certain embodiments, the trained machine learning model is trained using ADB. In certain embodiments, the trained machine learning model is trained using CART. In certain embodiments, the trained machine learning model is trained using hierarchical clustering.

In some embodiments, the reference biological samples comprise a blood sample, isolated peripheral blood mononuclear cells (PBMCs), a tissue biopsy sample, nasal fluid, saliva, or any derivative thereof. In some embodiments, the reference biological samples comprise blood samples or any derivative thereof. In some embodiments, the reference biological samples comprise isolated peripheral blood mononuclear cells (PBMCs) or any derivative thereof. In some embodiments, the reference biological samples comprise tissue biopsy samples or any derivative thereof. Tissue can be skin tissue, or kidney tissue. The reference subjects can be human.

The inference can have a confidence value between 0 and 1. In certain embodiments, the confidence value of the inference is between 0 and 1, such as, 0, 0.1, 0.2, 0.3, 0.4, 0.5, 0.6, 0.7, 0.8, 0.9 or 1, or any value or ranges there between that the patient has the group A lupus disease state, the group B lupus disease state, the group C lupus disease state, the group D lupus disease state, the group E lupus disease state, the group F lupus disease state, the group G lupus disease state, or the group H lupus disease state.

The trained machine-learning model can have a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The ROC-AUC curve can be for lupus disease state classification of the reference subjects.

In certain embodiments, the lupus disease state of the patient is classified based on a lupus disease risk score. The lupus disease risk score can be generated from the data set. In certain embodiments, the lupus disease risk score is generated based on the one or more GSVA scores of the patient. In certain embodiments, the lupus disease state of the patient is classified based on comparing the lupus disease risk score of the patient to one or more reference values.

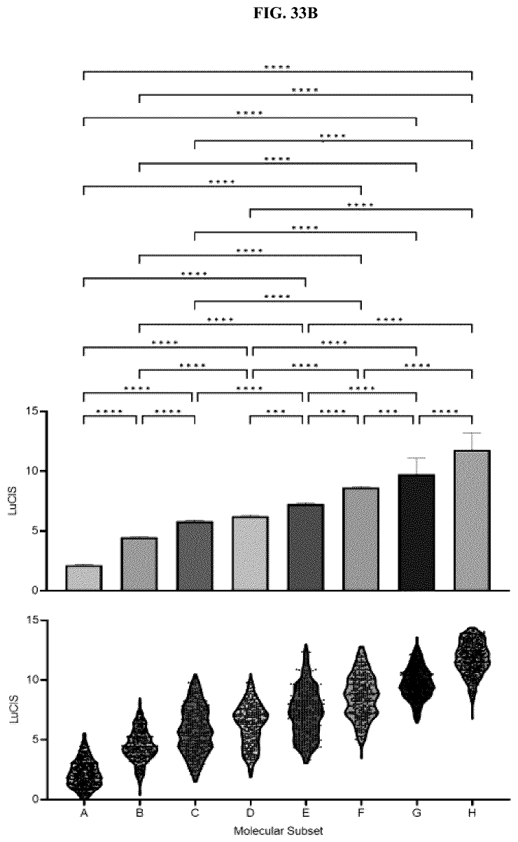

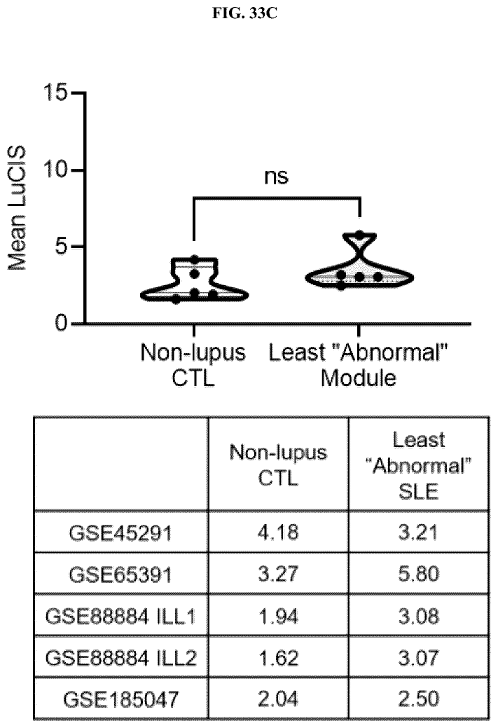

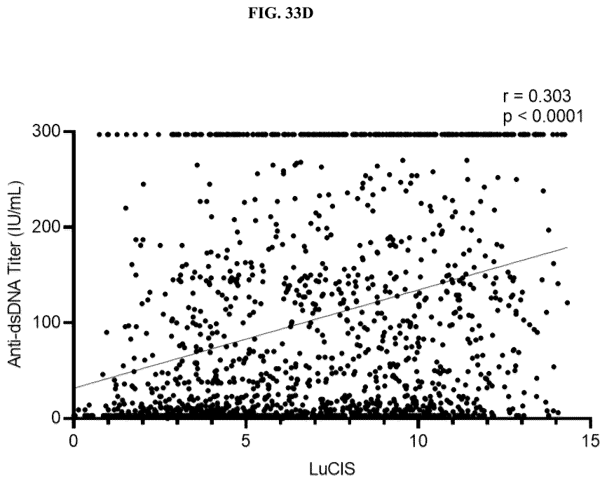

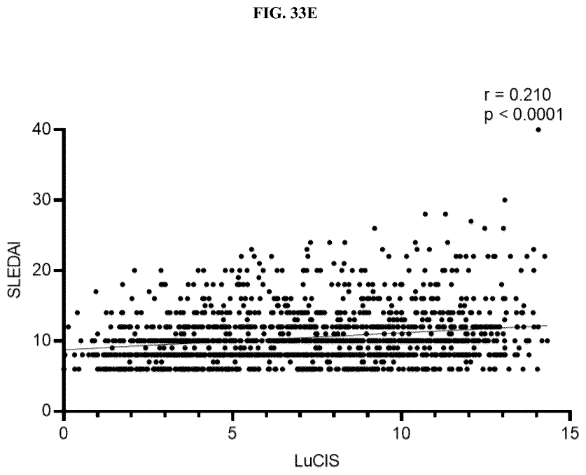

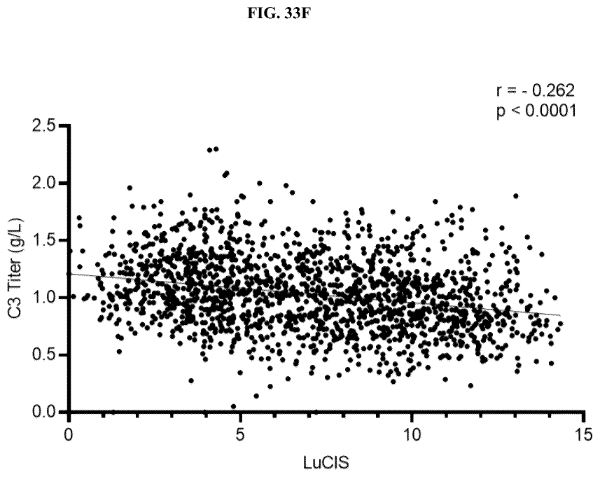

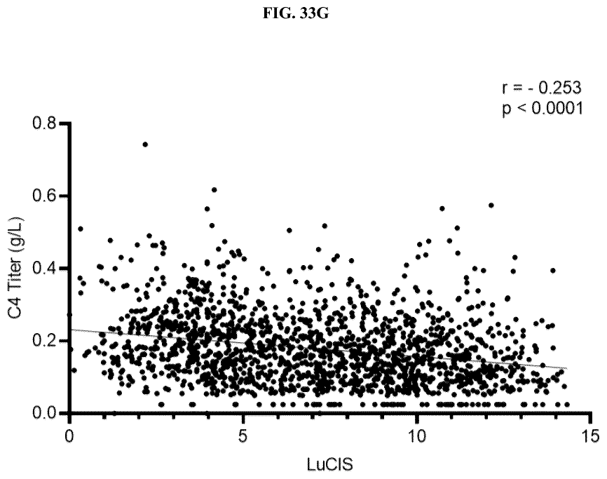

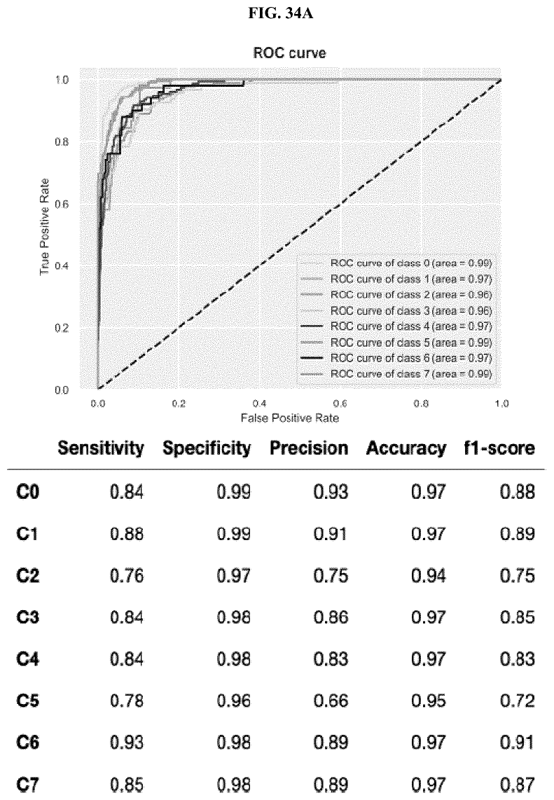

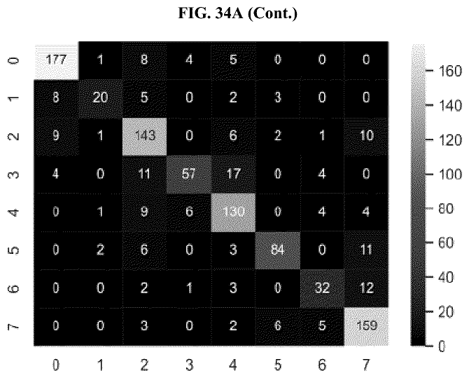

In certain embodiments, the lupus disease state of the patient is classified as the group A lupus disease state, the group B lupus disease state, the group C lupus disease state, the group D lupus disease state, the group E lupus disease state, the group F lupus disease state, the group G lupus disease state, or the group H lupus disease state, based on comparing the lupus disease risk score of the patient to one or more reference values. In certain embodiments, generating the lupus disease risk score of the patient comprises developing one or more weighted GSVA scores of the patient from the one or more GSVA scores of the patient, and summing the one or more weighted GSVA scores to obtain the lupus disease risk score of the patient. For a respective GSVA score of the one or more GSVA scores of the patient, a weighted GSVA score is obtained by multiplying the respective GSVA score with its respective weight factor, wherein the respective weight factor is determined based on contribution of the input gene set based on which the respective GSVA score is generated, on the classification of the lupus disease state of the patient. In certain particular embodiments, the one or more GSVA scores of the patient are binarized, and the binarized GSVA scores are multiplied with the respective weight factors to obtain the weighted GSVA scores. In certain embodiments, binarizing the one or more GSVA scores includes replacing all GSVA scores (e.g., of the one or more GSVA scores) above a threshold value with a first value, and replacing all GSVA scores (e.g., of the one or more GSVA scores) equal to or below the threshold value with a second value. In certain particular embodiments, the threshold value is 0, the first value is 1, and the second value is 0. The one or more GSVA scores of the patient can be generated using a method as described above or elsewhere herein. In certain embodiments, the weight factors can be the (feature) coefficient values of FIG. 33A. As shown in FIG. 33A, (feature) co-efficient for modules are, IFN (Table 8)=1.04; Monocyte (Table 18)=0.87; Cell Cycle (Table 4)=0.78; Anti-inflammation (Table 2)=0.75; Neutrophil (Table 19)=0.72; Inflammatory Cytokines (Table 14)=0.69; TNF (Table 31)=0.67; LDG (Table 16)=0.65; Immunoproteasome (Table 12)=0.61; Inhibitory Macs (Table 15)=0.60; Inflammasome (Table 13)=0.57; Plasma cell (Table 23)=0.54; Granulocyte (Table 7)=0.48; SNOR low Up (Table 24)=0.36; IG Chains (Table 9)=0.32; IL 1 pathway (Table 10)=0.17; Unfolded Protein (Table 32)=0.14; Oxidative Phosphorylation (Table 21)=−0.01; Treg (Table 26)=−0.02; Anergic/activated T cell (Table 1)=−0.03; B Cell (Table 3)=-0.06; T Cell (Table 25)=−0.20; gd T Cell (Table 6)=−0.26; MHCII (Table 17)=−0.28; IL 23 complex (Table 11)=−0.29 TCRD (Table 30)=−0.41, TCRA (Table 27)=−0.44; NK cell (Table 20)=−0.46, pDC (Table 22)=−0.47; Dendritic cell (Table 5)=−0.49; TCRB (Table 29)=−0.50; and TCRAJ (Table 28)=−0.50. In certain embodiments, binarized GSVA score generated from a respective module/Table can be multiplied with the (feature) co-efficient (e.g., weight factor) of the respective module/Table to obtain the weighted GSVA scores. The weight factors can be determined using a method as described in the Examples. In certain embodiments, the weight factors are determined at least by, determining GSVA scores of the 32 molecular features/modules (Table 1 to 32) of the lupus patients in the least abnormal endotype and lupus patients in the most abnormal endotype, and the GSVA scores were input into a ridge regression algorithm with penalty, wherein the resulting model provided the weight factors. In certain embodiments, the GSVA enrichment scores of the 32 molecular features/modules (Table 1 to 32) calculated for lupus patients in the bookend clusters of GSE88884 ILL1 & ILL2 (i.e., the least abnormal endotype (indianred2) and the most abnormal endotype (slateblue3)) were input into a ridge regression algorithm with penalty, to obtain the weight factors. In certain embodiments, the GSVA enrichment scores of the 32 molecular features/modules (Table 1 to 32) calculated for lupus patients in the endotype A (e.g., having group A lupus disease state) and lupus patients in the endotype H, of a reference data set, were input into a ridge regression algorithm with penalty, to obtain the weight factors. In certain embodiments, the ridge regression model can be generated using glmnet from the ‘caret’ R package v. 6.0-92.[45]. In certain embodiments, the weight factors are calculated based on training a machine learning model, wherein the trained machine learning model can generate an inference indicative of the lupus disease state of the patient based on the one or more GSVA scores of the patient. In certain embodiments, the one or more GSVA scores lupus patients in the endotype A and lupus patients in the endotype H can be used, e.g., for training. In certain embodiments, the one or more GSVA scores lupus patients in the least abnormal endotype (indianred2) and lupus patients in the most abnormal endotype (slateblue3) of GSE88884 ILL1 & ILL2 can be used, e.g., for training. The trained machine learning model can be a trained machine learning model as described above or elsewhere herein, and/or can be trained according a method as described above or elsewhere herein. The input gene sets based on which the one or more GSVA scores of the patient are generated can be features of the machine learning model. The feature co-efficients of the features can be the weight factors. The weight factor for a respective GSVA score can be the feature co-efficient of the input gene set (e.g., a feature) based on which the GSVA score is generated. The feature co-efficient, can be the average feature co-efficients of the iterations run during training the model. In certain embodiments, the lupus disease risk score is generated based on the one or more enrichment scores of the patient. The lupus disease risk score is based on the one or more enrichment scores can be generated using a similar method as described in above, in this paragraph, wherein enrichment scores in place of the GSVA scores can be used.

In certain embodiments, the method can include determining one or more gene features/gene sets significantly enriched in the biological sample obtained or derived from the patient. Classifying the lupus disease state of the patient, such determining whether the patient has group A, B, C, D, E, F, G or H lupus disease state can determine the one or more gene features significantly enriched in the biological sample obtained or derived from the patient. In certain embodiments, the dataset can be analyzed to determine the one or more gene features/gene sets significantly enriched in the patient. In certain embodiments, the one or more gene features/gene sets significantly enriched in the patient can be determined based on a Z-score method. In certain embodiments, a gene feature/gene set can be considered significantly enriched in the biological sample obtained or derived from the patient, when Z-score of the patient for the gene feature/gene set, is greater than 0.5, 1, 1.5, 2, 2.5, or 3. In certain embodiments, a gene feature/gene set can be considered significantly enriched in the biological sample obtained or derived from the patient, when the Z-score of the patient for the gene feature/gene set, is greater than 2. The Z-score of the patient for a gene feature/gene set can be calculated as, =(GSVA score of the gene feature/gene set of the patient −mean GSVA score of the gene feature/gene set for endotype A)/standard deviation of the gene feature/gene set GSVA for endotype A. GSVA score of the gene feature/gene set of the patient, can be a GSVA score generated using the gene feature/gene set as input gene set for GSVA, e.g., a GSVA score generated based on enrichment of the gene feature/gene set (e.g., set of genes within the gene feature/gene set) in the biological sample obtained or derived from the patient. Mean GSVA score and the standard deviation for endotype A can be calculated based on a reference dataset described above or elsewhere herein. The GSVA score of the gene feature/gene set of the patient, and GSVA scores of the gene feature/gene set for endotype A, can be calculated based on a method as described above or elsewhere herein. The genes (e.g., set of genes, at least 2 genes, effective number of genes, and/or all genes) selected from the one or more selected Tables can form the one or more gene features, wherein genes selected (e.g., at least 2 genes, effective number of genes, and/or all genes) from each selected Table can form a gene feature, and genes selected from different selected Tables form different gene features. The Tables selected and genes selected from the selected Tables as can as described above or elsewhere herein.

In certain embodiments, the method comprises performing Shapley Additive Explanations (SHAP) analysis on the trained machine learning model and the data set to determine contribution of one or more gene features/gene sets on the classification of the lupus disease state of the patient. The genes (e.g., set of genes, at least 2 genes, effective number of genes, and/or all genes) selected from the one or more selected Tables (e.g., the one or more Tables selected from Tables 1 to 32) can form the one or more gene features/gene sets, wherein genes selected from each selected Table can form a gene feature/gene set, and genes selected from different selected Tables form different gene features/gene sets. The Tables selected and genes selected from the selected Tables as can as described above or elsewhere herein. The one or more gene features/gene sets comprises the gene features/gene sets formed based on the Tables selected (e.g., the one or more Tables selected from Tables 1 to 32). The contribution of the one or more gene features/gene sets to the lupus disease state classification of the patient can be determined based on the SHAP values obtained from the SHAP analysis. Gene features/gene sets having higher contribution to the lupus disease state classification of the patient can have higher absolute SHAP values, among the absolute SHAP values of the one or more gene features/gene sets. For the SHAP analysis, the one or more gene features/gene sets can be the features of the trained machine learning model, and for each gene feature/gene set, the GSVA score generated based on enrichment of the gene feature/gene set in the biological sample (e.g., from the patient); the enrichment score generated based on enrichment of the gene feature/gene set in the biological sample; gene expression value of the gene feature/gene set in the biological sample; or ME calculated based on the gene expression of the gene feature in the biological sample, can be the feature value of the gene feature/gene set for the data set.

The method can classify the lupus disease state of the patient with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lupus disease state of the patient with a sensitivity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lupus disease state of the patient with a specificity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lupus disease state of the patient with a positive predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lupus disease state of the patient with a negative predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method classify the lupus disease state of the patient with an accuracy of about 85% to about 100%. In some embodiments, the method classify the lupus disease state of the patient with an accuracy of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classify the lupus disease state of the patient with an accuracy of about 85%, about 90%, about 92%, about 94%, about 95%, about 96%, about 98%, about 99%, about 99.3%, about 99.5%, about 99.8%, or about 100%. In some embodiments, the method classify the lupus disease state of the patient with an accuracy of at least about 85%, about 90%, about 92%, about 94%, about 95%, about 96%, about 98%, about 99%, about 99.3%, about 99.5%, or about 99.8%. In some embodiments, the method classify the lupus disease state of the patient with an accuracy of about 70% to about 100%. In some embodiments, the method classify the lupus disease state of the patient with an accuracy of about 70% to about 75%, about 70% to about 80%, about 70% to about 85%, about 70% to about 90%, about 70% to about 95%, about 70% to about 97.5%, about 70% to about 100%, about 75% to about 80%, about 75% to about 85%, about 75% to about 90%, about 75% to about 95%, about 75% to about 97.5%, about 75% to about 100%, about 80% to about 85%, about 80% to about 90%, about 80% to about 95%, about 80% to about 97.5%, about 80% to about 100%, about 85% to about 90%, about 85% to about 95%, about 85% to about 97.5%, about 85% to about 100%, about 90% to about 95%, about 90% to about 97.5%, about 90% to about 100%, about 95% to about 97.5%, about 95% to about 100%, or about 97.5% to about 100%. In some embodiments, the method classify the lupus disease state of the patient with an accuracy of about 70%, about 75%, about 80%, about 85%, about 90%, about 95%, about 97.5%, or about 100%. In some embodiments, the method classify the lupus disease state of the patient with an accuracy of at least about 70%, about 75%, about 80%, about 85% about 90%, about 95%, or about 97.5%.

In some embodiments, the method classify the lupus disease state of the patient with a sensitivity of about 85% to about 100%. In some embodiments, the method classify the lupus disease state of the patient with a sensitivity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classify the lupus disease state of the patient with a sensitivity of about 85%, about 90%, about 92%, about 94%, about 95%, about 96%, about 98%, about 99%, about 99.3%, about 99.5%, about 99.8%, or about 100%. In some embodiments, the method classify the lupus disease state of the patient with a sensitivity of at least about 85%, about 90%, about 92%, about 94%, about 95%, about 96%, about 98%, about 99%, about 99.3%, about 99.5%, or about 99.8%. In some embodiments, the method classify the lupus disease state of the patient with a sensitivity of about 70% to about 100%. In some embodiments, the method classify the lupus disease state of the patient with a sensitivity of about 70% to about 75%, about 70% to about 80%, about 70% to about 85%, about 70% to about 90%, about 70% to about 95%, about 70% to about 97.5%, about 70% to about 100%, about 75% to about 80%, about 75% to about 85%, about 75% to about 90%, about 75% to about 95%, about 75% to about 97.5%, about 75% to about 100%, about 80% to about 85%, about 80% to about 90%, about 80% to about 95%, about 80% to about 97.5%, about 80% to about 100%, about 85% to about 90%, about 85% to about 95%, about 85% to about 97.5%, about 85% to about 100%, about 90% to about 95%, about 90% to about 97.5%, about 90% to about 100%, about 95% to about 97.5%, about 95% to about 100%, or about 97.5% to about 100%. In some embodiments, the method classify the lupus disease state of the patient with a sensitivity of about 70%, about 75%, about 80%, about 85%, about 90%, about 95%, about 97.5%, or about 100%. In some embodiments, the method classify the lupus disease state of the patient with a sensitivity of at least about 70%, about 75%, about 80%, about 85%, about 90%, about 95%, or about 97.5%.

In some embodiments, the method classify the lupus disease state of the patient with a specificity of about 85% to about 100%. In some embodiments, the method classify the lupus disease state of the patient with a specificity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classify the lupus disease state of the patient with a specificity of about 85%, about 90%, about 92%, about 94%, about 95%, about 96%, about 98%, about 99%, about 99.3%, about 99.5%, about 99.8%, or about 100%. In some embodiments, the method classify the lupus disease state of the patient with a specificity of at least about 85%, about 90%, about 92%, about 94%, about 95%, about 96%, about 98%, about 99%, about 99.3%, about 99.5%, or about 99.8%. In some embodiments, the method classify the lupus disease state of the patient with a specificity of about 70% to about 100%. In some embodiments, the method classify the lupus disease state of the patient with a specificity of about 70% to about 75%, about 70% to about 80%, about 70% to about 85%, about 70% to about 90%, about 70% to about 95%, about 70% to about 97.5%, about 70% to about 100%, about 75% to about 80%, about 75% to about 85%, about 75% to about 90%, about 75% to about 95%, about 75% to about 97.5%, about 75% to about 100%, about 80% to about 85%, about 80% to about 90%, about 80% to about 95%, about 80% to about 97.5%, about 80% to about 100%, about 85% to about 90%, about 85% to about 95%, about 85% to about 97.5%, about 85% to about 100%, about 90% to about 95%, about 90% to about 97.5%, about 90% to about 100%, about 95% to about 97.5%, about 95% to about 100%, or about 97.5% to about 100%. In some embodiments, the method classify the lupus disease state of the patient with a specificity of about 70%, about 75%, about 80%, about 85%, about 90%, about 95%, about 97.5%, or about 100%. In some embodiments, the method classify the lupus disease state of the patient with a specificity of at least about 70%, about 75%, about 80%, about 85%, about 90%, about 95%, or about 97.5%.

In some embodiments, the method classify the lupus disease state of the patient with a positive predictive value of about 85% to about 100%. In some embodiments, the method classify the lupus disease state of the patient with a positive predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classify the lupus disease state of the patient with a positive predictive value of about 85%, about 90%, about 92%, about 94%, about 95%, about 96%, about 98%, about 99%, about 99.3%, about 99.5%, about 99.8%, or about 100%. In some embodiments, the method classify the lupus disease state of the patient with a positive predictive value of at least about 85%, about 90%, about 92%, about 94%, about 95%, about 96%, about 98%, about 99%, about 99.3%, about 99.5%, or about 99.8%. In some embodiments, the method classify the lupus disease state of the patient with a positive predictive value of about 70% to about 100%. In some embodiments, the method classify the lupus disease state of the patient with a positive predictive value of about 70% to about 75%, about 70% to about 80%, about 70% to about 85%, about 70% to about 90%, about 70% to about 95%, about 70% to about 97.5%, about 70% to about 100%, about 75% to about 80%, about 75% to about 85%, about 75% to about 90%, about 75% to about 95%, about 75% to about 97.5%, about 75% to about 100%, about 80% to about 85%, about 80% to about 90%, about 80% to about 95%, about 80% to about 97.5%, about 80% to about 100%, about 85% to about 90%, about 85% to about 95%, about 85% to about 97.5% about 85% to about 100%, about 90% to about 95%, about 90% to about 97.5%, about 90% to about 100%, about 95% to about 97.5%, about 95% to about 100%, or about 97.5% to about 100%. In some embodiments, the method classify the lupus disease state of the patient with a positive predictive value of about 70%, about 75%, about 80%, about 85%, about 90%, about 95%, about 97.5%, or about 100%. In some embodiments, the method classify the lupus disease state of the patient with a positive predictive value of at least about 70%, about 75%, about 80%, about 85%, about 90%, about 95%, or about 97.5%.

In some embodiments, the method classify the lupus disease state of the patient with a negative predictive value of about 85% to about 100%. In some embodiments, the method classify the lupus disease state of the patient with a negative predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classify the lupus disease state of the patient with a negative predictive value of about 85%, about 90%, about 92%, about 94%, about 95%, about 96%, about 98%, about 99%, about 99.3%, about 99.5%, about 99.8%, or about 100%. In some embodiments, the method classify the lupus disease state of the patient with a negative predictive value of at least about 85%, about 9000, about 92%, about 94%, about 95%, about 96%, about 98%, about 99%, about 99.3%, about 99.5%, or about 99.8%. In some embodiments, the method classify the lupus disease state of the patient with a negative predictive value of about 70% to about 100%. In some embodiments, the method classify the lupus disease state of the patient with a negative predictive value of about 70% to about 75%, about 70% to about 80%, about 70% to about 85%, about 70% to about 90%, about 70% to about 95%, about 70% to about 97.5%, about 70% to about 100%, about 75% to about 80%, about 75% to about 85%, about 75% to about 90%, about 75% to about 95%, about 75% to about 97.5%, about 75% to about 100%, about 80% to about 85%, about 80% to about 90%, about 80% to about 95%, about 80% to about 97.5%, about 80% to about 100%, about 85% to about 90%, about 85% to about 95%, about 85% to about 97.5%, about 85% to about 100%, about 90% to about 95%, about 90% to about 97.5%, about 90% to about 100%, about 95% to about 97.5%, about 95% to about 100%, or about 97.5% to about 100%. In some embodiments, the method classify the lupus disease state of the patient with a negative predictive value of about 70%, about 75%, about 80%, about 85%, about 90%, about 95%, about 97.5%, or about 100%. In some embodiments, the method classify the lupus disease state of the patient with a negative predictive value of at least about 70%, about 75%, about 80%, about 85%, about 90%, about 95%, or about 97.5%.

The trained machine-learning model can have the accuracy, sensitivity, specificity, positive predictive value, negative predictive value, and ROC-AUC value, described above, and the accuracy, sensitivity, specificity, positive predictive value, and negative predictive value, of the method can be based on the classification parameters of the trained machine-learning model, as described above or elsewhere herein and/or as understood by one of skill in the art. The accuracy, sensitivity, specificity, positive predictive value, and negative predictive value, of the method can be calculated based on the ROC-AUC curve of the trained machine model for classification of lupus disease state of reference subjects/patients. The accuracy, sensitivity, specificity, positive predictive value, negative predictive value, and/or ROC-AUC value, can have a value as shown in the Examples. The accuracy, sensitivity, specificity, positive predictive value, and negative predictive value can be based on classification of the patient between the lupus disease state groups. The accuracy, sensitivity, specificity, positive predictive value, and negative predictive value can be based on classification of the patient between groups A-H lupus disease state.

In certain embodiments, the machine learning model has a Receiver operating characteristic (ROC) curve having an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The ROC-AUC curve can be for lupus disease state classification of the reference subjects.

In some embodiments, the machine learning model has a ROC curve with an AUC of about 0.85 to about 1. In some embodiments, the machine learning model has a ROC curve with an AUC of about 0.85 to about 0.9, about 0.85 to about 0.92, about 0.85 to about 0.94, about 0.85 to about 0.95, about 0.85 to about 0.96, about 0.85 to about 0.98, about 0.85 to about 0.99, about 0.85 to about 0.993, about 0.85 to about 0.995, about 0.85 to about 0.998, about 0.85 to about 1, about 0.9 to about 0.92, about 0.9 to about 0.94, about 0.9 to about 0.95, about 0.9 to about 0.96, about 0.9 to about 0.98, about 0.9 to about 0.99, about 0.9 to about 0.993, about 0.9 to about 0.995, about 0.9 to about 0.998, about 0.9 to about 1, about 0.92 to about 0.94, about 0.92 to about 0.95, about 0.92 to about 0.96, about 0.92 to about 0.98, about 0.92 to about 0.99, about 0.92 to about 0.993, about 0.92 to about 0.995, about 0.92 to about 0.998, about 0.92 to about 1, about 0.94 to about 0.95, about 0.94 to about 0.96, about 0.94 to about 0.98, about 0.94 to about 0.99, about 0.94 to about 0.993, about 0.94 to about 0.995, about 0.94 to about 0.998, about 0.94 to about 1, about 0.95 to about 0.96, about 0.95 to about 0.98, about 0.95 to about 0.99, about 0.95 to about 0.993, about 0.95 to about 0.995, about 0.95 to about 0.998, about 0.95 to about 1, about 0.96 to about 0.98, about 0.96 to about 0.99, about 0.96 to about 0.993, about 0.96 to about 0.995, about 0.96 to about 0.998, about 0.96 to about 1, about 0.98 to about 0.99, about 0.98 to about 0.993, about 0.98 to about 0.995, about 0.98 to about 0.998, about 0.98 to about 1, about 0.99 to about 0.993, about 0.99 to about 0.995, about 0.99 to about 0.998, about 0.99 to about 1, about 0.993 to about 0.995, about 0.993 to about 0.998, about 0.993 to about 1, about 0.995 to about 0.998, about 0.995 to about 1, or about 0.998 to about 1. In some embodiments, the machine learning model has a ROC curve with an AUC of about 0.85, about 0.9, about 0.92, about 0.94, about 0.95, about 0.96, about 0.98, about 0.99, about 0.993, about 0.995, about 0.998, or about 1. In some embodiments, the machine learning model has a ROC curve with an AUC of at least about 0.85, about 0.9, about 0.92, about 0.94, about 0.95, about 0.96, about 0.98, about 0.99, about 0.993, about 0.995, or about 0.998. In some embodiments, the machine learning model has a ROC curve with an AUC of about 0.7 to about 1. In some embodiments, the machine learning model has a ROC curve with an AUC of about 0.7 to about 0.75, about 0.7 to about 0.8, about 0.7 to about 0.85, about 0.7 to about 0.9, about 0.7 to about 0.95, about 0.7 to about 0.975, about 0.7 to about 1, about 0.75 to about 0.8, about 0.75 to about 0.85, about 0.75 to about 0.9, about 0.75 to about 0.95, about 0.75 to about 0.975, about 0.75 to about 1, about 0.8 to about 0.85, about 0.8 to about 0.9, about 0.8 to about 0.95, about 0.8 to about 0.975, about 0.8 to about 1, about 0.85 to about 0.9, about 0.85 to about 0.95, about 0.85 to about 0.975, about 0.85 to about 1, about 0.9 to about 0.95, about 0.9 to about 0.975, about 0.9 to about 1, about 0.95 to about 0.975, about 0.95 to about 1, or about 0.975 to about 1. In some embodiments, the machine learning model has a ROC curve with an AUC of about 0.7, about 0.75, about 0.8, about 0.85, about 0.9, about 0.95, about 0.975, or about 1. In some embodiments, the machine learning model has a ROC curve with an AUC of at least about 0.7, about 0.75, about 0.8, about 0.85, about 0.9, about 0.95, or about 0.975. The ROC-AUC curve can be for lupus disease state classification of the reference subjects.

The biological sample (e.g., obtained and/or derived from the patient) can comprise a blood sample, isolated peripheral blood mononuclear cells (PBMCs), tissue biopsy sample, nasal fluid, saliva, urine, stool, or any derivative thereof. In certain embodiments, the biological sample comprises a blood sample or any derivative thereof. In certain embodiments, the biological sample comprises isolated PBMCs, or any derivative thereof. In certain embodiments, the biological sample comprises tissue biopsy sample, or any derivative thereof. The tissue can be skin tissue, or kidney tissue. In certain embodiments, the biological sample comprises nasal fluid sample, or any derivative thereof. In certain embodiments, the biological sample comprises saliva sample, or any derivative thereof. In certain embodiments, the biological sample comprises urine sample, or any derivative thereof. In certain embodiments, the biological sample comprises stool sample, or any derivative thereof. The patient can be a human patient.

In certain embodiments, the method comprises recommending, selecting and/or administering a treatment to the patient based on the lupus disease state classification of the patient. In certain embodiments, the method comprises administering a treatment to the patient based on the lupus disease state classification of the patient. In certain embodiments, the method comprises administering the treatment to the patient based on the lupus disease state classification of the patient, wherein the method can be directed to treating lupus disease state of a patient. The treatment can be configured to treat, reduce severity, and/or reduce risk of developing lupus. In certain embodiments, the treatment is configured to treat lupus. In certain embodiments, the treatment is configured to reduce severity of lupus. In certain embodiments, the treatment is configured to reduce risk of developing lupus.

In certain embodiments, the treatment is based on the contribution of the one or more gene features/gene sets on the classification of the lupus disease state of the patient. The contribution of the one or more gene features/gene sets can be determined using the SHAP analysis as described above or elsewhere herein. In certain embodiments, the treatment targets the gene feature/gene set having highest contribution, second highest contribution, third highest contribution, fourth highest contribution, fifth highest contribution or any combination thereof, as determined by SHAP analysis. In certain embodiments, the treatment targets at least one gene feature/gene set out of the gene features/gene sets having top 10, top 9, top 8, top 7, top 6, top 5, top 4, top 3 or top 2 absolute SHAP values among the absolute SHAP values of the one or more gene features/gene sets, wherein the SHAP values are obtained from the SHAP analysis on the trained machine learning model and the data set to determine contribution of one or more gene features/gene sets on the classification of the lupus disease state of the patient. In certain embodiments, the treatment targets at least one gene feature/gene set out of the gene features/gene sets having top 10 absolute SHAP values among the absolute SHAP values of the one or more gene features/gene sets. In certain embodiments, the treatment targets at least one gene feature/gene set out of the gene features/gene sets having top 8 absolute SHAP values among the absolute SHAP values of the one or more gene features/gene sets. In certain embodiments, the treatment targets at least one gene feature/gene set out of the gene features/gene sets having top 5 absolute SHAP values among the absolute SHAP values of the one or more gene features/gene sets. In certain embodiments, the treatment targets at least one gene feature/gene set out of the gene features/gene sets having top 3 absolute SHAP values among the absolute SHAP values of the one or more gene features/gene sets. In certain embodiments, the treatment targets the gene feature/gene set having the top absolute SHAP value among the absolute SHAP values of the one or more gene features/gene sets. Treatment targeting a gene feature/gene set formed based on Table 8, (e.g., containing at least 2 genes, effective number of genes, or all genes selected from the genes listed in Table 8) can comprise a IFN inhibitor such as Anifrolumab. Treatment targeting a gene feature/gene set formed based on Table 23, (e.g., containing at least 2 genes, effective number of genes, or all genes selected from the genes listed in Table 23), can comprise a Plasma cell inhibitor such as belimumab, mycophenolate, Bortezomib, Carfilzomib, Ixazomib, isatuximab, daratumumab, elotuzumab, or any combination thereof. Treatment targeting a gene feature/gene set formed based on Table 10, (e.g., containing at least 2 genes, effective number of genes, or all genes selected from the genes listed in Table 10) can comprise a IL1 inhibitor such as Anakinra, and/or Canakinumab.

Treatment targeting a gene feature/gene set formed based on Table 31, (e.g., containing at least 2 genes, effective number of genes, or all genes selected from the genes listed in Table 31) can comprise a TNF inhibitor such as Adalimumab, Certolizumab pegol, Etanercept, Golimumab, Infliximab, or any combination thereof. Treatment targeting a gene feature/gene set formed based on Table 19, (e.g., containing at least 2 genes, effective number of genes, or all genes selected from the genes listed in Table 19) can comprise a Neutrophil function inhibitor such as Dasatinib, Apremilast, Roflumilast, or any combination thereof. Treatment targeting a gene feature/gene set formed based on Table 20, (e.g., containing at least 2 genes, effective number of genes, or all genes selected from the genes listed in Table 20) can comprise a NK cell inhibitor such as Azathioprine (AZA). Treatment targeting a gene feature/gene set formed based on Table 3, (e.g., containing at least 2 genes, effective number of genes, or all genes selected from the genes listed in Table 3) can comprise a B cell inhibitor such as Belimumab, Rituximab, Obinutuzumab, Ocrelizumab, Ofatumumab, Inebilizumab, or any combination thereof. In certain embodiments, the treatment targets a gene feature/gene set significantly enriched in the biological sample obtained or derived from the patient. In certain embodiments, the gene feature/gene set significantly enriched in the biological sample obtained or derived from the patient can be determined based on a Z-score method, as described herein. In certain embodiments, the IFN module is significantly enriched in the biological sample obtained or derived from the patient, such as a gene feature/gene set containing effective number of genes, and/or all genes selected from the genes listed in Table 8 has a Z-score greater than 2, and the treatment can comprise a IFN inhibitor such as Anifrolumab. In certain embodiments, the plasma cells module is significantly enriched in the biological sample obtained or derived from the patient, such as a gene feature/gene set containing effective number of genes, and/or all genes selected from the genes listed in Table 23 has a Z-score greater than 2, and the treatment can comprise a Plasma cell inhibitor such as belimumab, mycophenolate, Carfilzomib, Bortezomib, Ixazomib, isatuximab, daratumumab, elotuzumab, or any combination thereof. In certain embodiments, the IL1 pathway module is significantly enriched in the biological sample obtained or derived from the patient, such as a gene feature/gene set containing effective number of genes, and/or all genes selected from the genes listed in Table 10 has a Z-score greater than 2, and the treatment can comprise a IL1 inhibitor such as Anakinra, and/or Canakinumab. In certain embodiments, the TNF Waddel Up module is significantly enriched in the biological sample obtained or derived from the patient, such as a gene feature/gene set containing effective number of genes, and/or all genes selected from the genes listed in Table 31 has a Z-score greater than 2, and the treatment can comprise a TNF inhibitor such as Adalimumab, Certolizumab pegol, Etanercept, Golimumab, Infliximab, or any combination thereof. In certain embodiments, the Neutrophil module is significantly enriched in the biological sample obtained or derived from the patient, such as a gene feature/gene set containing effective number of genes, and/or all genes selected from the genes listed in Table 19 has a Z-score greater than 2, and the treatment can comprise a Neutrophil function inhibitor such as Dasatinib, Apremilast, Roflumilast, or any combination thereof. Treatment when the NK cell module is significantly enriched in the biological sample obtained or derived from the patient, such as a gene feature/gene set containing effective number of genes, and/or all genes selected from the genes listed in Table 20 has a Z-score greater than 2, can comprise a NK cell inhibitor such as Azathioprine. In certain embodiments, the B cells module is significantly enriched in the biological sample obtained or derived from the patient, such as a gene feature/gene set containing effective number of genes, and/or all genes selected from the genes listed in Table 3 has a Z-score greater than 2, and the treatment can comprise a B cell inhibitor such as Belimumab, Rituximab, Obinutuzumab, Ocrelizumab, Ofatumumab, Inebilizumab, or any combination thereof. The treatment may or may not target every gene feature/gene set that is enriched in the biological sample. The genes selected from the one or more selected Tables (e.g., from Tables 1 to 32) can form one or more gene features/gene sets, wherein genes selected from each selected Table can form a gene feature/gene set, and genes selected from different selected Tables form different gene features/gene sets. The Tables selected and genes selected from the selected Tables as can as described above or elsewhere herein.

The treatment can comprise a pharmaceutical composition. The patient can be a human patient. In certain embodiments, the treatment comprises a neutrophil function inhibitor, a TNF inhibitor, an IL1 inhibitor, a Plasma cell inhibitor, a NK cell inhibitor, IFN inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, Ocrelizumab, Ofatumumab, and Inebilizumab. In certain embodiments, the treatment comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof. In certain embodiments, the treatment for, group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, NK cell inhibitor, a neutrophil function inhibitor, a TNF inhibitor, an IL1 inhibitor, a Plasma cell inhibitor or any combination thereof; group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof; and/or group H lupus disease state comprises an IFN inhibitor, a neutrophil function inhibitor, a TNF inhibitor, an IL1 inhibitor, a Plasma cell inhibitor or any combination thereof. In certain embodiments, the treatment for group B lupus disease state comprises a neutrophil function inhibitor. In certain embodiments, the treatment for group C lupus disease state comprises a neutrophil function inhibitor, a TNF inhibitor, an IL1 inhibitor, an IFN inhibitor or any combination thereof. In certain embodiments, the treatment for group D lupus disease state comprises a B cell inhibitor, an IFN inhibitor, NK cell inhibitor, a neutrophil function inhibitor, a TNF inhibitor, an IL1 inhibitor, a Plasma cell inhibitor or any combination thereof. In certain embodiments, the treatment for group E lupus disease state comprises an IFN inhibitor, a neutrophil function inhibitor, a TNF inhibitor, a Plasma cell inhibitor or any combination thereof. In certain embodiments, the treatment for group F lupus disease state comprises an IFN inhibitor, a neutrophil function inhibitor, a TNF inhibitor, an IL1 inhibitor, or any combination thereof. In certain embodiments, the treatment for group G lupus disease state comprises a B cell inhibitor, an IFN inhibitor, a neutrophil function inhibitor, a TNF inhibitor, an IL1 inhibitor, a Plasma cell inhibitor or any combination thereof. In certain embodiments, the treatment for group H lupus disease state comprises an IFN inhibitor, a neutrophil function inhibitor, a TNF inhibitor, an IL1 inhibitor, a Plasma cell inhibitor or any combination thereof. The treatment for an endotype (e.g., treatment recommended, selected and/or administered to a patient having endotype B, C, D, E, F, G or H lupus disease state) can be based on the modules enriched and/or modules de-enriched in the endotype, compared to the non-lupus control (e.g., endotype A). In certain embodiments, the treatment for an endotype (e.g., treatment recommended, selected and/or administered to a patient classified as having the endotype) can be based on the modules enriched in the endotype, compared to the non-lupus control (e.g., endotype A). As a non-limiting example, for an endotype (e.g., B-H) a module can be considered enriched, if the “mean GSVA score of the module for the endotype” minus “mean GSVA score of the module for the endotype A” is >0. Drugs inhibiting the pathways associated with the module can be targets for that endotype. Treatment based on an enriched module can be target the module, e.g., can comprise drugs inhibiting biological/molecular pathways associated with the module. For example, Belimumab (Benlysta) can be used as treatment for modules with enriched B Cell module, and/or plasma cell module. Treatment for group B lupus disease state can be recommended to, selected for and/or administered to a patient classified as having group B lupus disease state. Treatment for group C lupus disease state can be recommended to, selected for and/or administered to a patient classified as having group C lupus disease state. Treatment for group D lupus disease state can be recommended to, selected for and/or administered to a patient classified as having group D lupus disease state. Treatment for group E lupus disease state can be recommended to, selected for and/or administered to a patient classified as having group E lupus disease state. Treatment for group F lupus disease state can be recommended to, selected for and/or administered to a patient classified as having group F lupus disease state. Treatment for group G lupus disease state can be recommended to, selected for and/or administered to a patient classified as having group G lupus disease state. Treatment for group H lupus disease state can be recommended to, selected for and/or administered to a patient classified as having group H lupus disease state. In certain embodiments, the treatment for, group B lupus disease state comprises Belimumab, Dasatinib, Roflumilast and/or Apremilast; group C lupus disease state comprises Anifrolumab, Anakinra, Canakinumab, Adalimumab, Certolizumab pegol, Etanercept, Golimumab, Inflximab, Dasatinib, Roflumilast, Apremilast, or any combination thereof; group D lupus disease state comprises Belimumab, Rituximab, Obinutuzumab, Ocrelizumab, Ofatumumab, Inebilizumab, Anifrolumab, Mycophenolate, AZA, Bortezomib, Isatuximab, Elotuzumab, Carfilzomib, Ixazomib, Daratumumab, Anakinra, Canakinumab, Adalimumab, Certolizumab pegol, Etanercept, Golimumab, Infiximab, Dasatinib, Roflumilast, Apremilast or any combination thereof; group E lupus disease state comprises Anifrolumab, Mycophenolate, Bortezomib, Isatuximab, Elotuzumab, Carfilzomib, Ixazomib, Daratumumab, Adalimumab, Certolizumab pegol, Etanercept, Golimumab, Infiximab, Dasatinib, Roflumilast, Apremilast or any combination thereof; group F lupus disease state comprises Anifrolumab, Anakinra, Canakinumab, Adalimumab, Certolizumab pegol, Etanercept, Golimumab, Infiximab, Dasatinib, Roflumilast, Apremilast, Belimumab or any combination thereof; group G lupus disease state comprises Belimumab, Rituximab, Obinutuzumab, Ocrelizumab, Ofatumumab, Inebilizumab, Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast or any combination thereof; and/or group H lupus disease state comprises Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Infiximab, Dasatinib, Roflumilast, Apremilast, Belimumab or any combination thereof. In certain embodiments, the treatment for group B lupus disease state comprises Belimumab, Dasatinib, Roflumilast and/or Apremilast. In certain embodiments, the treatment for group C lupus disease state comprises Anifrolumab, Anakinra, Canakinumab, Adalimumab, Certolizumab pegol, Etanercept, Golimumab, Infiximab, Dasatinib, Roflumilast, Apremilast, or any combination thereof. In certain embodiments, the treatment for group D lupus disease state comprises Belimumab, Rituximab, Obinutuzumab, Ocrelizumab, Ofatumumab, Inebilizumab, Anifrolumab, Mycophenolate, AZA, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast or any combination thereof. In certain embodiments, the treatment for group E lupus disease state comprises Anifrolumab, Mycophenolate, Bortezomib, Isatuximab, Elotuzumab, Carfilzomib, Ixazomib, Daratumumab, Adalimumab, Certolizumab pegol, Etanercept, Golimumab, Inflximab, Dasatinib, Roflumilast, Apremilast or any combination thereof. In certain embodiments, the treatment for group F lupus disease state comprises Anifrolumab, Anakinra, Canakinumab, Adalimumab, Certolizumab pegol, Etanercept, Golimumab, Inflximab, Dasatinib, Roflumilast, Apremilast, Belimumab or any combination thereof. In certain embodiments, the treatment for group G lupus disease state comprises Belimumab, Rituximab, Obinutuzumab, Ocrelizumab, Ofatumumab, Inebilizumab, Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast or any combination thereof. In certain embodiments, the treatment for group H lupus disease state comprises Anifrolumab, Mycophenolate, Bortezomib, Isatuximab, Elotuzumab, Carfilzomib, Ixazomib, Daratumumab, Anakinra, Canakinumab, Adalimumab, Certolizumab pegol, Etanercept, Golimumab, Inflximab, Dasatinib, Roflumilast, Apremilast, Belimumab or any combination thereof.

In certain embodiments, the method further comprises monitoring the lupus disease state of the patient, wherein the monitoring comprises assessing (e.g., classifying) the lupus disease state of the patient at a plurality of different time points. A difference in the assessment of the lupus disease state of the patient among the plurality of time points can be indicative of one or more clinical indications selected from the group consisting of. (i) a diagnosis of the lupus disease state of the patient, (ii) a prognosis of the lupus disease state of the patient, and (iii) an efficacy or non-efficacy of a course of treatment for treating the lupus disease state of the patient. In certain embodiments, the patient has been administered a treatment, and the method can assess an efficacy or non-efficacy of the treatment, for treating the lupus disease state of the patient.

The method can determine whether a patient is a candidate for treatment with the lupus drug based on the lupus disease state classification of the patient.

Lupus can be any type of lupus including but not limited to systemic lupus erythematosus (SLE), cutaneous lupus erythematosus, drug-induced lupus, and neonatal lupus.

In certain embodiments lupus can be SLE.

One aspect of the present disclosure is directed to the use of a data set described above or elsewhere herein. One aspect of the present disclosure is directed to the use of the one or more gene sets described above or elsewhere herein. The one or more gene sets can be formed based on the one or more Tables selected from Tables: 1 to 32, wherein genes selected from each of the selected Table can form a gene set of the one or more gene sets, and genes selected from different selected Tables can form different gene sets of the one or more gene sets. Each gene set of the one or more gene sets can be generated based on one of the one or more selected Tables, wherein for each selected Table the genes selected (e.g., at least 2 genes, effective number of genes, and/or all genes) from the selected Table forms a gene set, and genes selected from different selected Tables can form different gene sets of the one or more gene sets. The one or more Tables selected, genes selected from Tables selected can be as described above or elsewhere herein.

Method for Identifying a Patient as a Candidate for Treatment with a Lupus Drug

In an aspect of the present disclosure a method for identifying a patient as a candidate for treatment with a lupus drug. The method comprises analyzing a data set comprising or derived from gene expression measurements of at least 2 genes to generate an inference on whether the patient is a candidate for treatment with the lupus drug. The gene expression measurements are obtained from a biological sample of the patient.

In certain embodiments, the at least 2 genes are selected from genes listed Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. Genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32, include all the genes listed in Tables 1-32. In some embodiments, the at least 2 genes are selected from a group of genes listed in 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of Tables 1-32. In certain embodiments, the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, or any value or range there between, genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989 or any value or range there between, genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes comprise at least 1 gene from each of Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes comprise independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes from Table: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; or 32, or any combination thereof. In certain embodiments, the at least 2 genes comprise independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes listed in each Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes are selected from genes listed Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 722, or all genes, selected from genes listed in Tables: 1; 2; 3; 4; 5; 6; 7; 8; 10; 11; 12; 13; 14; 15; 16; 17; 18; 19; 20; 21; 22; 23; 24; 25; 31; and 32. In certain embodiments, the data set comprises or is derived from gene expression measurements of at least 2 genes selected from genes listed in each of one or more Tables selected from Tables: 1 to 32. The one or more Tables selected from Tables: 1 to 32 can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 Tables. In certain embodiments, the one or more Tables comprise at least 1 Table, e.g., at least 1 Table is selected from Tables: 1 to 32. In certain embodiments, the one or more Tables comprise at least 2 Tables. In certain embodiments, the one or more Tables comprise at least 3 Tables. In certain embodiments, the one or more Tables comprise at least 4 Tables. In certain embodiments, the one or more Tables comprise at least 5 Tables. In certain embodiments, the one or more Tables comprise at least 6 Tables. In certain embodiments, the one or more Tables comprise at least 7 Tables. In certain embodiments, the one or more Tables comprise at least 8 Tables. In certain embodiments, the one or more Tables comprise at least 9 Tables. In certain embodiments, the one or more Tables comprise at least 10 Tables. In certain embodiments, the one or more Tables comprise at least 11 Tables. In certain embodiments, the one or more Tables comprise at least 12 Tables. In certain embodiments, the one or more Tables comprise at least 13 Tables. In certain embodiments, the one or more Tables comprise at least 14 Tables. In certain embodiments, the one or more Tables comprise at least 15 Tables. In certain embodiments, the one or more Tables comprise at least 16 Tables. In certain embodiments, the one or more Tables comprise at least 17 Tables. In certain embodiments, the one or more Tables comprise at least 18 Tables. In certain embodiments, the one or more Tables comprise at least 19 Tables. In certain embodiments, the one or more Tables comprise at least 20 Tables. In certain embodiments, the one or more Tables comprise at least 21 Tables. In certain embodiments, the one or more Tables comprise at least 22 Tables. In certain embodiments, the one or more Tables comprise at least 23 Tables. In certain embodiments, the one or more Tables comprise at least 24 Tables. In certain embodiments, the one or more Tables comprise at least 25 Tables. In certain embodiments, the one or more Tables comprise at least 26 Tables. In certain embodiments, the one or more Tables comprise at least 27 Tables. In certain embodiments, the one or more Tables comprise at least 28 Tables. In certain embodiments, the one or more Tables comprise at least 29 Tables. In certain embodiments, the one or more Tables comprise at least 30 Tables. In certain embodiments, the one or more Tables comprise at least 31 Tables. In certain embodiments, the one or more Tables comprise 32 Tables, e.g., Tables: 1 to 32 are selected. In certain embodiments, the one or more Tables comprise at least 14 Tables, e.g., 14 or more Tables are selected from Tables: 1 to 32, wherein at least Tables: 2; 4; 5; 7; 8; 12; 13; 14; 15; 16; 18; 19; 23; and 31, are selected. In certain embodiments, the one or more Tables comprise at least 16 Tables, wherein at least Tables: 2; 4; 5; 7; 8; 12; 13; 14; 15; 16; 18; 19; 20; 22; 23; and 31, are selected. In certain embodiments, the one or more Tables comprise at least 23 Tables, wherein at least Tables: 2; 4; 5; 6; 7; 8; 10; 11; 12; 13; 14; 15; 16; 17; 18; 19; 20; 22; 23; 24; 25; 31; and 32, are selected. In certain embodiments, the one or more Tables comprise at least 26 Tables, wherein at least Tables: 1; 2; 3; 4; 5; 6; 7; 8; 10; 11; 12; 13; 14; 15; 16; 17; 18; 19; 20; 21; 22; 23; 24; 25; 31; and 32, are selected. In certain embodiments, the one or more Tables are selected from Tables: 1 to 32, based on the feature co-efficient of the Tables. In certain embodiments, if at least X Tables are selected from Tables: 1 to 32, where X is an integer from 1 to 32, at least the Tables having X highest absolute feature co-efficient values are selected. In certain embodiments, the one or more Tables selected from Tables: 1 to 32, comprises the Table with the highest absolute feature co-efficient value, i.e., at least Table 8 is selected. In certain embodiments, the one or more Tables selected from Tables: 1 to 32, comprises the Tables with 2 highest absolute feature co-efficient values, i.e., at least Table 8 and Table 18 are selected. In certain embodiments, the one or more Tables selected from Tables: 1 to 32, comprises the Tables with 3 highest absolute feature co-efficient values. In certain embodiments, the one or more Tables selected from Tables: 1 to 32, comprises the Tables with 4 highest absolute feature co-efficient values. In certain embodiments, the one or more Tables selected from Tables: 1 to 32, comprises the Tables with 5 highest absolute feature co-efficient values. In certain embodiments, the one or more Tables selected from Tables: 1 to 32, comprises the Tables with 6 highest absolute feature co-efficient values. In certain embodiments, the one or more Tables selected from Tables: 1 to 32, comprises the Tables with 7 highest absolute feature co-efficient values. In certain embodiments, the one or more Tables selected from Tables: 1 to 32, comprises the Tables with 8 highest absolute feature co-efficient values. In certain embodiments, the one or more Tables selected from Tables: 1 to 32, comprises the Tables with 9 highest absolute feature co-efficient values. In certain embodiments, the one or more Tables selected from Tables: 1 to 32, comprises the Tables with 10 highest absolute feature co-efficient values. In certain embodiments, the one or more Tables selected from Tables: 1 to 32, comprises the Tables with 11 highest absolute feature co-efficient values. In certain embodiments, the one or more Tables selected from Tables: 1 to 32, comprises the Tables with 12 highest absolute feature co-efficient values. In certain embodiments, the one or more Tables selected from Tables: 1 to 32, comprises the Tables with 13 highest absolute feature co-efficient values. In certain embodiments, the one or more Tables selected from Tables: 1 to 32, comprises the Tables with 14 highest absolute feature co-efficient values. In certain embodiments, the one or more Tables selected from Tables: 1 to 32, comprises the Tables with 15 highest absolute feature co-efficient values. In certain embodiments, the one or more Tables selected from Tables: 1 to 32, comprises the Tables with 16 highest absolute feature co-efficient values. In certain embodiments, the one or more Tables selected from Tables: 1 to 32, comprises the Tables with 17 highest absolute feature co-efficient values. In certain embodiments, the one or more Tables selected from Tables: 1 to 32, comprises the Tables with 18 highest absolute feature co-efficient values. In certain embodiments, the one or more Tables selected from Tables: 1 to 32, comprises the Tables with 19 highest absolute feature co-efficient values. In certain embodiments, the one or more Tables selected from Tables: 1 to 32, comprises the Tables with 20 highest absolute feature co-efficient values. In certain embodiments, the one or more Tables selected from Tables: 1 to 32, comprises the Tables with 21 highest absolute feature co-efficient values. In certain embodiments, the one or more Tables selected from Tables: 1 to 32, comprises the Tables with 22 highest absolute feature co-efficient values. In certain embodiments, the one or more Tables selected from Tables: 1 to 32, comprises the Tables with 23 highest absolute feature co-efficient values. In certain embodiments, the one or more Tables selected from Tables: 1 to 32, comprises the Tables with 24 highest absolute feature co-efficient values. In certain embodiments, the one or more Tables selected from Tables: 1 to 32, comprises the Tables with 25 highest absolute feature co-efficient values. In certain embodiments, the one or more Tables selected from Tables: 1 to 32, comprises the Tables with 26 highest absolute feature co-efficient values. In certain embodiments, the one or more Tables selected from Tables: 1 to 32, comprises the Tables with 27 highest absolute feature co-efficient values. In certain embodiments, the one or more Tables selected from Tables: 1 to 32, comprises the Tables with 28 highest absolute feature co-efficient values. In certain embodiments, the one or more Tables selected from Tables: 1 to 32, comprises the Tables with 29 highest absolute feature co-efficient values. In certain embodiments, the one or more Tables selected from Tables: 1 to 32, comprises the Tables with 30 highest absolute feature co-efficient values. In certain embodiments, the one or more Tables selected from Tables: 1 to 32, comprises the Tables with 31 highest absolute feature co-efficient values. The at least 2 genes may or may not include gene(s) that are not listed in Tables 1 to 32. In certain embodiments, the at least 2 genes do not include any gene that are not listed in Tables 1 to 32. In certain embodiments, for each selected Table the data set comprises or is derived from gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein gene selected from different selected Tables can be same or different. In certain embodiments, for each selected Table the data set comprises or is derived from gene expression measurements of effective number of genes selected from the genes listed in the selected Table, wherein gene selected from different selected Table can be same or different. The at least 2 genes may or may not include gene(s) that are not listed in Tables 1 to 32.

In certain embodiments, the data set comprises an enrichment score derived from gene expression measurements. The enrichment score can be derived by enrichment assessment of the at least 2 genes of the date set. Analyzing a data set can include analyzing the enrichment score, wherein the enrichment score can be analyzed to generate the inference indicating whether the patient is a candidate for treatment with the lupus drug. In certain embodiments, the enrichment assessment is performed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment assessment is performed using GSVA. In certain embodiments, the data set comprises one or more GSVA scores (e.g., GSVA enrichment scores) of the patient derived from the gene expression measurements of the biological sample using GSVA, wherein the one or more GSVA scores are generated based on the one or more Tables selected from Tables: 1 to 32, wherein for each selected Table, the genes selected from the selected Table forms an input gene set based on which at least one GSVA score of the patient, based on the selected Table is generated using GSVA, and wherein the one or more GSVA scores comprise the generated GSVA scores. The at least one GSVA score based on a selected Table can be generated based on enrichment of the genes selected from the selected Table in the biological sample. GSVA can be performed using a method as described in the Examples. The one or more Tables selected (e.g., based on which the one or more GSVA of the patient scores are generated) can comprise the Tables as described above or elsewhere herein. For a selected Table the genes selected (e.g., that forms the input gene set for generating the at least one GSVA score based on the selected Table) from the selected Table can comprise the selected genes as described above or elsewhere herein. The GSVA scores can be GSVA enrichment scores, and can be generated using GSVA using the respective input gene sets, based on a method as described in the Examples and/or as understood by one of skill in the art. In certain embodiments, for each selected Table the genes selected (e.g., that forms the input gene set for generating the at least one GSVA score based on the selected Table) comprise at least 2 genes selected from the genes listed in the selected Table, wherein gene selected from different selected Tables can be same or different. In certain embodiments, for each selected Table the genes selected (e.g., that forms the input gene set for generating the at least one GSVA score based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein gene selected from different selected Tables can be same or different. In certain embodiments, for each selected Table the genes selected (e.g., that forms the input gene set for generating the at least one GSVA score based on the selected Table) comprise effective number of genes selected from the genes listed in the selected Table, wherein gene selected from different selected Tables can be same or different. In certain embodiments, for each selected Table one GSVA score is generated based on the selected Table

The analyzing can include providing the data set as an input to a trained machine-learning model trained to generate the inference indicating whether the patient is a candidate for treatment with the lupus drug. In certain embodiments, the input comprises the gene expression measurements of the at least 2 genes of the dataset. In certain embodiments, the input comprises the enrichment score obtained from the dataset. In certain embodiments, the method further includes receiving, as an output of the trained machine-learning model, the inference indicating whether the patient is a candidate for treatment with the lupus drug; and/or electronically outputting a report indicating whether the patient is a candidate for treatment with the lupus drug.

The trained machine-learning model, can generate the inference indicating whether the patient is a candidate for treatment with the lupus drug, by comparing the data set to a reference data set. The machine-learning model can be trained using the reference data set. In some embodiments, the reference data set contains gene expression measurements of the at least 2 genes of a plurality of reference biological samples from a plurality of reference subjects. The plurality of the reference subjects can have lupus. A first portion of the plurality of reference subjects have been administered with the lupus drug, and a second portion of the plurality of reference subjects were not administered with the lupus drug. A respective individual reference data set of the plurality of individual reference data sets can contain i) gene expression measurements of the least 2 genes of a reference biological sample from a reference subject, and ii) data regarding administration of the lupus drug to the reference subject. Data regarding administration of the lupus drug to the reference subject can include data on whether or not the reference subject received the lupus drug, and/or dosage at which the lupus drug was administered. The plurality of individual reference data sets can be obtained from the plurality of reference subjects. In some embodiments, different individual reference data sets are obtained from different reference subjects. In some embodiments, each of the individual reference data set contains i) gene expression measurements of the least 2 genes of a reference biological sample from one reference subject, and ii) data regarding administration of the lupus drug to the reference subject, and, wherein different individual reference data sets are obtained from different reference subjects. In certain embodiments, oversampling or undersampling correction is made during training of the machine learning model. The genes of the data set and genes of the reference data set can at least partially overlap. In some embodiments, the reference biological sample is a blood sample, isolated peripheral blood mononuclear cells (PBMCs), a tissue biopsy sample, nasal fluid, saliva, or any derivative thereof. In some embodiments, the reference biological sample is a blood sample or any derivative thereof. In some embodiments, the reference biological sample is isolated peripheral blood mononuclear cells (PBMCs) or any derivative thereof. The reference subjects can be human. In certain embodiments, the trained machine-learning model is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof. In certain embodiments, the trained machine-learning model is developed using a linear regression method. In certain embodiments, the trained machine-learning model is developed using a LOG method. In certain embodiments, the trained machine-learning model is developed using a Ridge regression method. In certain embodiments, the trained machine-learning model is developed using a Lasso regression method. In certain embodiments, the trained machine-learning model is developed using an EN regression method. In certain embodiments, the trained machine-learning model is developed using a SVM method. In certain embodiments, the trained machine-learning model is developed using a GBM method. In certain embodiments, the trained machine-learning model is developed using a kNN method. In certain embodiments, the trained machine-learning model is developed using a GLM method. In certain embodiments, the trained machine-learning model is developed using a NB classifier method. In certain embodiments, the trained machine-learning model is developed using a RF method. In certain embodiments, the trained machine-learning model is developed using a deep learning algorithm method. In certain aspects, the deep learning algorithm method can be sequential neural network. In certain embodiments, the trained machine-learning model is developed using a LDA method. In certain embodiments, the trained machine-learning model is developed using a DTREE method. In certain embodiments, the trained machine-learning model is developed using an adaptive boosting (ADB) method.

The inference can include classification that the patient is a candidate for treatment with the lupus drug. In certain embodiments, the inference comprises a confidence value between 0 and 1 that the patient is a candidate for treatment with the lupus drug.

The classification that the patient is a candidate for treatment with the lupus drug can have, an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The classification that the patient is a candidate for treatment with the lupus drug can have, a sensitivity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The classification that the patient is a candidate for treatment with the lupus drug can have, a specificity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The classification that the patient is a candidate for treatment with the lupus drug can have, a positive predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The classification that the patient is a candidate for treatment with the lupus drug, a negative predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model classify that the patient is a candidate for treatment with the lupus drug with a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In certain embodiments, the analyzing comprises calculating a risk score for the patient based at least on the gene expression measurements of the at least 2 genes, and generating the inference on whether the patient is a candidate for treatment with the lupus drug based at least on the risk score of the patient.

The biological sample can be a blood sample, isolated peripheral blood mononuclear cells (PBMCs), tissue biopsy sample, nasal fluid, saliva, urine, stool, or any derivative thereof. In certain embodiments, the biological sample is a blood sample or any derivative thereof. In certain embodiments, the biological sample is isolated peripheral blood mononuclear cells (PBMCs), or any derivative thereof.

In certain embodiments, the method comprises administering to the patient the lupus drug based on the inference that the patient is a candidate for treatment with the lupus drug. The lupus drug can be Belimumab, Prednisone, Mycophenolate such as Mycophenolate mofetil, Azathioprine, Voclosporin, Cyclophosphamide, Methylprednisolone, Anifrolumab, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Rituximab, Obinutuzumab, Ocrelizumab, Ofatumumab, Inebilizumab, or any combination thereof. In certain embodiment the lupus drug comprises belimumab. The patient can be a human patient.

### Method for Developing a Biomarker Assay for Identifying a Treatment Candidate for a Lupus Drug

In an aspect of the present disclosure, a method for developing a biomarker assay for identifying a treatment candidate for a lupus drug. The method can include any one of, any combination of, or all of steps (a) to (e). Step (a) can include obtaining a reference data set comprising a plurality of individual reference data sets, wherein a respective individual reference data set of the plurality of individual reference data sets comprises i) gene expression measurements of at least 2 genes selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32; of a reference patient, and ii) data regarding the reference patient's one or more lupus disease index, at a time point before administering, and at least one time point after administering the lupus drug to the reference patient. Step (b) can include training a machine learning model using the reference data set, wherein the machine learning model is trained to infer a training patient's response to the lupus drug based on gene expression measurements of the at least 2 genes of step (a) of the training patient, at a time point before administering, and at least one time point after administering the lupus drug to the training patient. Step (c) can include determining feature importance values of one or more predictors of the machine learning model, wherein the one or more predictors comprises the at least 2 genes of step (a). Step (d) includes selecting 2 to 30 gene predictors of the machine learning model based at least on the feature importance values determined in step (c). Step (e) includes developing an assay capable of measuring expression and/or encoding of the 2 to 30 genes selected in step (d) in a biological sample, to obtain the biomarker assay.

In certain embodiments, the assay is capable of measuring encoding of the 2 to 30 genes selected in step (d), in the biological sample. In certain embodiments, the assay is capable of measuring expression of the 2 to 30 genes selected in step (d), in the biological sample.

In certain embodiments, in step (d) 2, 3, 4, 5, 6, 7, 8, 9, 10, 11, 12, 13, 14, 15, 16, 17, 18, 19, 20, 21, 22, 23, 24, 25, 26, 27, 28, 29, or 30, or any range there between gene predictors of the machine learning model is selected. The 2 to 30 gene predictors of the machine learning model selected in step (d) can have top 2 to 30 feature importance values determined in step (c).

The one or more lupus disease index can include blood anti-double-stranded DNA antibody level, blood anti-ribonucleoprotein (RNP) antibody level, blood complement component 3 (C3) protein level, blood complement component 4 (C4) protein level, SLEDAI score, and LuMOS score. The training patient's response to the lupus drug can include a measurement of change of the training patient's blood anti-double-stranded DNA antibody level, blood anti-ribonucleoprotein (RNP) antibody level, blood complement component 3 (C3) protein level, blood complement component 4 (C4) protein level, SLEDAI score, LuMOS score, or any combination thereof, between the time point before administration, and the at least one time point after administration of the lupus drug to the training patient.

Genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32, include all the genes listed in Tables 1-32. In some embodiments, the at least 2 genes are selected from a group of genes listed in 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of Tables 1-32. In certain embodiments, the at least 2 genes comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32.

In certain embodiments, the at least 2 genes comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, or any value or range there between, genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 550, 600, 650, 700, 750, 800, 850, 900, 950, 970, 980, 989, or all, or any value or range there between, genes, selected from genes listed in Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes comprise at least 1 gene from each of Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32. In certain embodiments, the at least 2 genes comprise independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes from Table: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; or 32, or any combination thereof. In certain embodiments, the at least 2 genes comprise independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes listed in each Tables: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; and 32.

The lupus drug can be any lupus drug, including any lupus drug candidate known in the art, described above or elsewhere herein, or described in the literature. In some embodiments, the lupus drug comprises a drug that is approved for use in treating lupus in human patients by at least one drug-approval authority. In some embodiments, the lupus drug is a lupus drug candidate in a testing phase. In some embodiments, the methods of the invention are used to assess patients suitable for participation in a clinical trial for the lupus drug. In some embodiments, the lupus drug comprises Belimumab, Prednisone, Mycophenolate such as Mycophenolate mofetil, Azathioprine, Voclosporin, Cyclophosphamide, Methylprednisolone, Anifrolumab, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Rituximab, Obinutuzumab, Ocrelizumab, Ofatumumab, Inebilizumab, or any combination thereof. In certain embodiment the lupus drug comprises belimumab. The patient can be a human patient.

In certain embodiments, the trained machine-learning model of step (b) is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof. In certain embodiments, the trained machine-learning model is developed using a linear regression method. In certain embodiments, the trained machine-learning model is developed using a LOG method. In certain embodiments, the trained machine-learning model is developed using a Ridge regression method. In certain embodiments, the trained machine-learning model is developed using a Lasso regression method. In certain embodiments, the trained machine-learning model is developed using an EN regression method.

In certain embodiments, the trained machine-learning model is developed using a SVM method.

In certain embodiments, the trained machine-learning model is developed using a GBM method.

In certain embodiments, the trained machine-learning model is developed using a kNN method.

In certain embodiments, the trained machine-learning model is developed using a GLM method.

In certain embodiments, the trained machine-learning model is developed using a NB classifier method. In certain embodiments, the trained machine-learning model is developed using a RF method. In certain embodiments, the trained machine-learning model is developed using a deep learning algorithm method. In certain aspects, the deep learning algorithm method can be sequential neural network. In certain embodiments, the trained machine-learning model is developed using a LDA method. In certain embodiments, the trained machine-learning model is developed using a DTREE method. In certain embodiments, the trained machine-learning model is developed using an adaptive boosting (ADB) method.

The biological sample can be a blood sample, isolated peripheral blood mononuclear cells (PBMCs), tissue biopsy sample, nasal fluid, saliva, urine, stool, or any derivative thereof.

In certain embodiments, the biological sample is a blood sample or any derivative thereof. In certain embodiments, the biological sample is isolated peripheral blood mononuclear cells (PBMCs), or any derivative thereof. Certain aspects, are directed to a biomarker assay developed according to a method described above or elsewhere herein. Certain aspects, are directed to a kit comprising the biomarker assay developed according to a method described above or elsewhere herein, and/or a biomarker assay of described above or elsewhere herein.

### Non-Transitory Computer Readable Medium

Another aspect of the present disclosure provides a non-transitory computer readable medium comprising machine executable code that, upon execution by one or more computer processors, implements any of the methods above or elsewhere herein.

### System Comprising One or More Computer Processors and Computer Memory Coupled Thereto

Another aspect of the present disclosure provides a system comprising one or more computer processors and computer memory coupled thereto. The computer memory comprises machine executable code that, upon execution by the one or more computer processors, implements any of the methods above or elsewhere herein.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles.

Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash© Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Biological Data Analysis

Certain embodiments, of the present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample may be optionally pre-treated or processed prior to use.

A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness.

For example, a method as described herein may be performed on a subject prior to, and after, treatment with a first, second, and/or third disease condition therapy to measure the disease's progression or regression in response to the first, second, and/or third disease condition therapy. The first, second, and/or third disease can be as described above.

After obtaining a sample from the subject, the sample may be processed to generate datasets indicative of a disease or disorder of the subject. For example, a presence, absence, or quantitative assessment of nucleic acid molecules of the sample from a panel of condition-associated genomic loci or nucleotide polymorphism may be indicative of first, second, and/or third disease condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

The BIG-C(Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of. Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet.

Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that may also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, S1P1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that may be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety).

The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in first, second and/or third disease patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

### BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-C® categories are cross-examined with the GO and KEGG terms to obtain additional information and insights.

A sample BIG-C® workflow may comprise the following steps. First, SLE genomic datasets are derived from whole blood, peripheral blood mononuclear cells, affected tissues, and purified immune cells. Second, datasets are analyzed using DE analysis (as shown by a differential expression heatmap) or Weighted Gene Coexpression Network Analysis (WGCNA) (as shown by a gene coexpression plot). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories (e.g., as described by Labonte et al. 2018, “Identification of alterations in macrophage activation associated with disease activity in systemic lupus erythematosus,” PloS one, 13(12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ may be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. When combined with BIG-C® categories, the cellular activity may be correlated to specific functions within a given cell type.

A sample I-Scope™ workflow may comprise the following steps. First, candidate genes are identified from SLE (systemic lupus erythematosus) datasets potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ may be used downstream of the BIG-C® (Biologically Informed Gene-Clustering) tool to understand which tissue cell types are present. In conjunction with I-Scope™ (which provides information related to immune cells), T-Scope™ may be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories, ultimately allowing for cellular activity analysis across multiple samples and disease states. When combined with BIG-C® categories, the cellular activity may be correlated to specific functions within a given tissue cell type.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

### CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+autoimmune patients. The cloud-based genomic platform may leverage results from RNAseq and microarray experiments in conjunction with clinical information, such as medication and lab tests, to provide undiscovered insights.

CellScan™ may go beyond typical 'omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. An example list of reference databases for the content in CellScan™, with both human and mouse species-specific identifiers supported.

### MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 1 may be performed of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25(10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between −1 and +1 indicating levels of down-regulation and up-regulation respectively.

A sample MS-Scoring™ 2 workflow may comprise the following steps. First, a signaling pathway of interest is selected from the MS-Scoring™ 2 menu Second, a raw gene expression data is inputted into the MS-Scoring™ 2 tool. Third, enrichment of signaling pathway(s) is assessed on a patient by patient basis. Fourth, the data may then be used to drive insight for the target signaling pathways in individual patient samples.

Results from GSVA Analysis on SLE (systemic lupus erythematosus) signaling pathways may be, e.g., as described by Hanzelmann et al., “GSVA: Gene Set Variation Analysis for Microarray and RNA-Seq Data,” BMC Bioinformatics, vol. 14, no. 1, 2013, p. 7, which is incorporated herein by reference in its entirety.

### CoLTs®(Combined Lupus Treatment Scoring) Analysis Tool

A scoring method called CoLTs®, or Combined Lupus Treatment Scoring, may be configured to assessing and prioritizing the repositioning potential of drug therapies. CoLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available.

CoLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs® prediction of Stelara/Ustekinumab to be atop priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155, 2018, pp. 1330-1339, which is incorporated herein by reference in its entirety). CoLTs® may be calibrated on SoC (Standard of Care) therapies for the individual autoimmune disease being assessed.

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25(10), 1150-1170, which is incorporated herein by reference in its entirety). For example, CoLT Scoring® of SOC Therapies in Lupus (Belimumab, HCQ, and Rituximab) may be performed.

### Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

Target-Scoring™ may be configured to assessing and prioritizing the potential of molecular targets for further development of drug therapies. The Target-Scoring™ tool is very similar to CoLTs® except it approaches the need for new SLE therapies from a different angle.

Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers may better prioritize the development of novel drug therapies addressing the assessed targets of interest.

Target-Scoring™ may utilize 19 different scoring categories to derive a composite score that ranges from −13 to +27 for the suitability of a gene target for SLE therapy development.

Target-Scoring™ may be validated through repeated scoring of potential therapies as well as by clinicians (e.g., clinicians specializing in the field of immunology).

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module may comprise computer systems that connect laboratory hardware or instrumentation with computer systems that process laboratory data. In one embodiment, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as first, second, and/or third disease condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as first, second, and/or third disease condition) and individuals not having the condition (e.g., healthy individuals, or individuals who do not have first, second, and/or third disease condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as first, second, and/or third disease condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%.

This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as first, second, and/or third disease condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as first, second, and/or third disease condition), a prognosis of one or more conditions (e.g., a disease or disorder, such as first, second, and/or third disease condition), a risk of having one or more conditions (e.g., a disease or disorder, such as first, second, and/or third disease condition), a treatment history of one or more conditions (e.g., a disease or disorder, such as first, second, and/or third disease condition), a history of previous treatment for one or more conditions (e.g., a disease or disorder, such as first, second, and/or third disease condition), a history of prescribed medications, a history of prescribed medical devices, age, height, weight, sex, smoking status, and one or more symptoms of the subject.

For example, the disease or disorder may comprise one or more of lupus, coronary artery disease (CAD), myocardial infraction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis. As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as first, second, and/or third disease condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as first, second, and/or third disease condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as first, second, and/or third disease condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as first, second, and/or third disease condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more.

The classification of samples may assign an output value of “positive” or 1 if the sample indicates that the subject has a probability of having one or more conditions (e.g., a disease or disorder, such as first, second, and/or third disease condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as first, second, and/or third disease condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as first, second, and/or third disease condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%, {2%, 98%}, {5%, 95%}{10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as first, second, and/or third disease condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as first, second, and/or third disease condition). In some embodiments, the sample is independent of samples used to train the trained algorithm.

The trained algorithm may be trained with a first number of independent training samples associated with a presence of the condition (e.g., a disease or disorder, such as first, second, and/or third disease condition) and a second number of independent training samples associated with an absence of the condition (e.g., a disease or disorder, such as first, second, and/or third disease condition). The first number of independent training samples associated with presence of the condition (e.g., a disease or disorder, such as first, second, and/or third disease condition) may be no more than the second number of independent training samples associated with absence of the condition (e.g., a disease or disorder, such as first, second, and/or third disease condition). The first number of independent training samples associated with a presence of the condition (e.g., a disease or disorder) may be equal to the second number of independent training samples associated with an absence of the condition (e.g., a disease or disorder, such as first, second, and/or third disease condition). The first number of independent training samples associated with a presence of the condition (e.g., a disease or disorder, such as first, second, and/or third disease condition) may be greater than the second number of independent training samples associated with an absence of the condition (e.g., a disease or disorder, such as first, second, and/or third disease condition).

The trained algorithm may comprise a classifier configured to identify the presence (e.g., positive test result) or absence (e.g., negative test result) of one or more conditions (e.g., a disease or disorder, such as first, second, and/or third disease condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as first, second, and/or third disease condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as first, second, and/or third disease condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as first, second, and/or third disease condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as first, second, and/or third disease condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as first, second, and/or third disease condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample.

As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as first, second, and/or third disease condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples.

### Kits

The present disclosure provides kits for identifying or monitoring a disease or disorder (e.g., first, second, and/or third disease condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., first, second, and/or third disease condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., first, second, and/or third disease condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%.

In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

### EXAMPLES

The following illustrative examples are representative of embodiments of the software applications, systems, and methods described herein and are not meant to be limiting in any way.

### Example 1: Identification of Lupus Endotypes

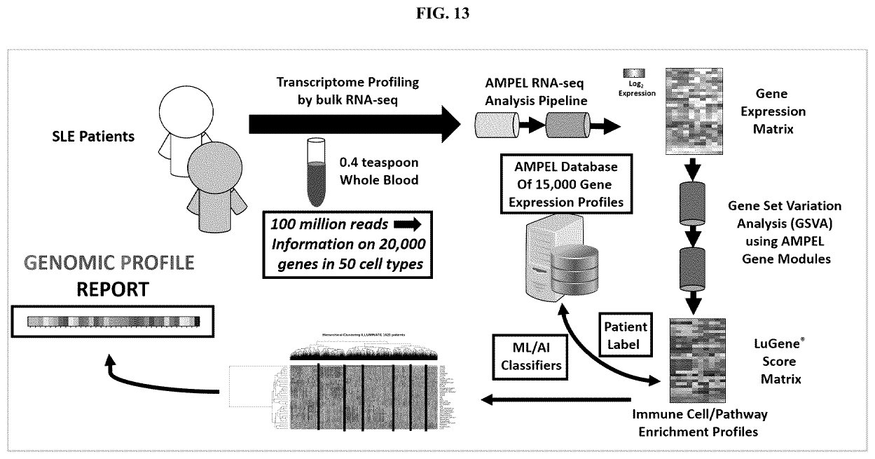

Lupus patients and healthy controls from various datasets were clustered using the schematic shown in FIG. 13.

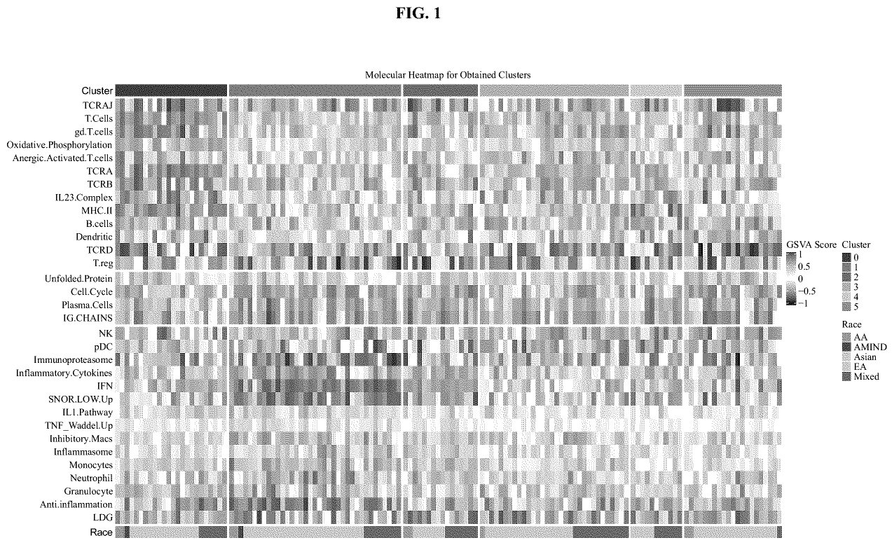

As shown in FIG. 1, six adult lupus endotypes were identified from data set GSE88884 ILLUMINATE-1 and 2. 1620 adult lupus patients from GSE88884, were clustered using expression of genes of the 32 Modules (Tables: 1 to 32). Gene expression matrix obtained from whole blood samples, were analyzed by gene set variation analysis (GSVA) using the 32 modules. k-means clustering of GSVA scores of the 32 features yielded six clusters using baseline gene expression. Color labels above the heatmap indicate patient clusters and colors were randomly generated in R. Color labels below the heatmap indicate patient ancestry.

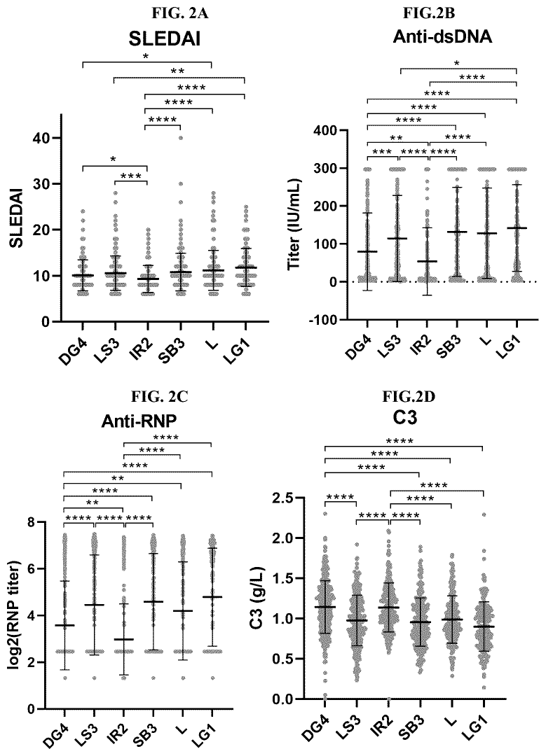

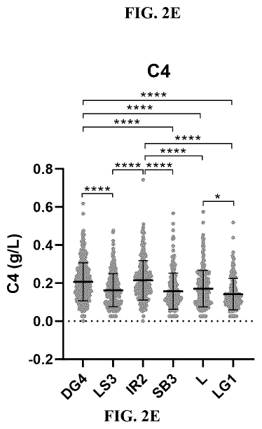

Clinical characteristics of the six identified lupus endotypes (FIG. 1), are shown in FIGS. 2A-E. Scatterplots in display the mean±SD for each immunologic/inflammatory and systemic disease indicators in each cluster; statistical differences were found with Dunn's multiple comparisons test. IR2 was the least “least abnormal” lupus cluster. FIG. 2A shows SLEDAI score. FIG. 2B: shows blood anti-double-stranded DNA antibody level. FIG. 2C shows blood anti-ribonucleoprotein (RNP) antibody level. FIG. 2D shows blood complement component 3 (C3) protein level. FIG. 2E shows blood complement component 4 (C4) protein level.

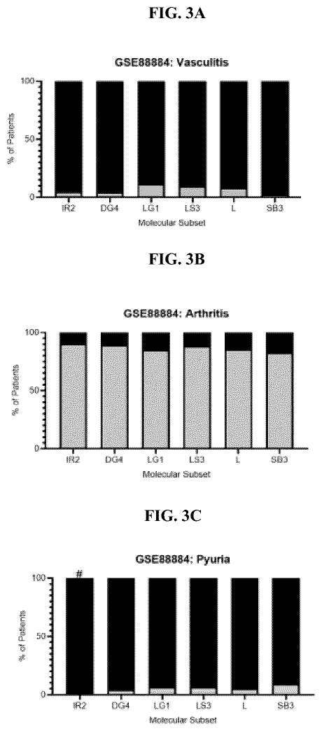

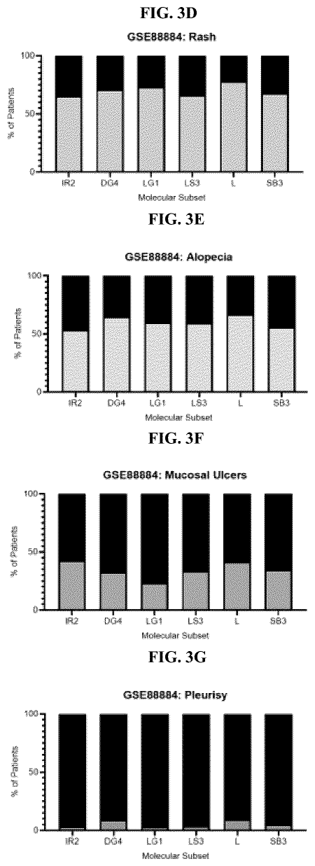

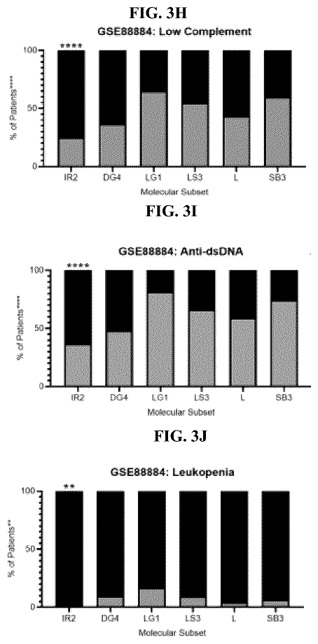

FIGS. 3A-J show Lupus patients in the least severe subset are less likely to be characterized by low complement, positive anti-dsDNA status, and leukopenia. Distribution of (FIG. 3A) vasculitis, (FIG. 3B) arthritis, (FIG. 3C) pyuria, (FIG. 3D) rash, (FIG. 3E) alopecia, (FIG. 3F) mucosal ulcers, (FIG. 3G) pleurisy, (FIG. 3H) low complement, (FIG. 3I) anti-dsDNA, and (FIG. 3J) leukopenia among molecular subsets. The likelihood of having low complement, anti-dsDNA, and leukopenia in the IR2 subset is 0.34, 0.34, and 0.00 respectively as compared to the other five subsets combined. Significant differences in expected and observed frequencies between IR2, the “least abnormal subset” and all other subsets (denoted with asterisk above bars) was identified with Chi Square Test. Significant associations between categorical variables and all subsets (denoted with asterisks on the y-axis) were identified using Chi Square Test of Independence

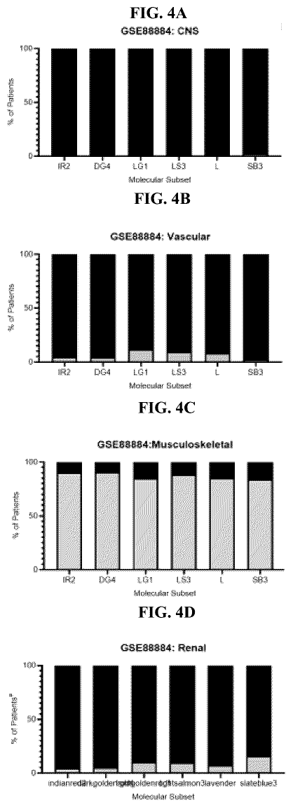

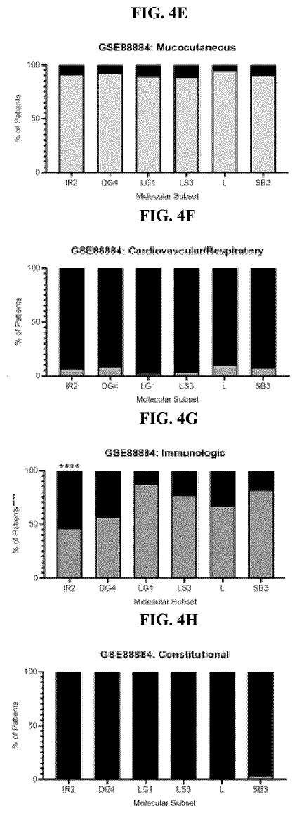

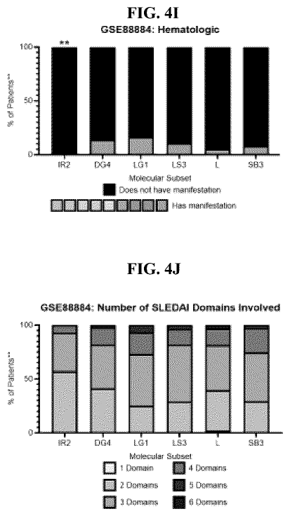

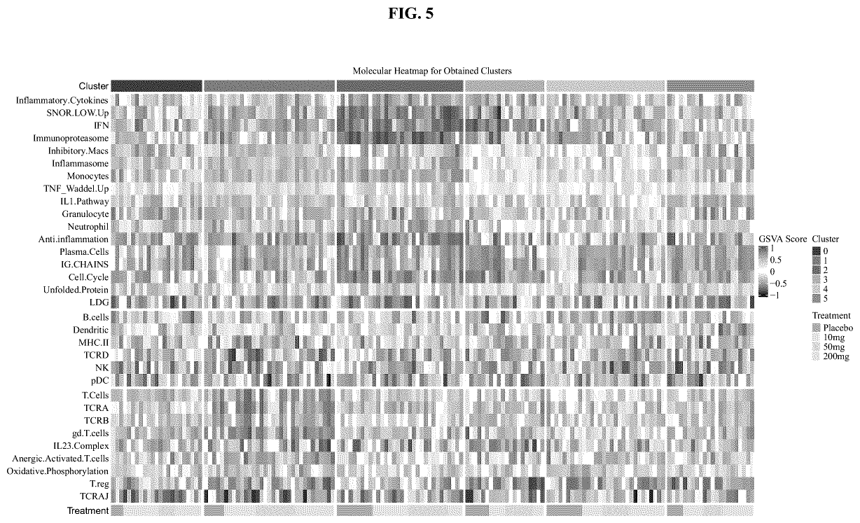

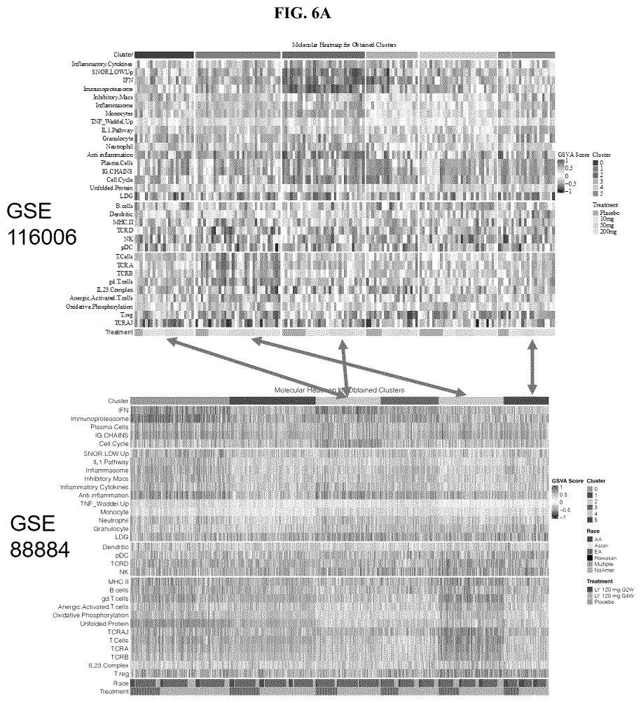

FIGS. 4A-J show Lupus patients in the least severe subset are less likely to be characterized by hematologic involvement. Distribution of (FIG. 4A) CNS, (FIG. 4B) vascular, (FIG. 4C) musculoskeletal, (FIG. 4D) renal, (FIG. 4E) mucocutaneous, (FIG. 4F) cardiovascular/respiratory, (FIG. 4G) immunologic, (FIG. 4H) constitutional, and (FIG. 4I) hematologic domain involvement. (FIG. 4J) Distribution of the number of SLE domains involved. The likelihood of having immunologic and hematologic domain in the IR2 subset is 0.34 and 0.00 respectively as compared to the other five subsets combined. Significant differences in expected and observed frequencies between IR2, the “least abnormal subset” and all other subsets (denoted with asterisk above bars) was identified with Chi Square Test. There were no significant associations identified between categorical variables and all subsets using Chi Square Test of Independence.

FIG. 5 shows identification of lupus endotypes in additional whole blood datasets. K-means clustering of GSVA scores of the 32 modules (Tables: 1 to 32) yielded 6 clusters in adult lupus patients from GSE116006 using baseline gene expression. Color labels above the heatmap indicate cluster identity which were randomly generated using the ‘grDevices’ color palette in R. Color bars below the heatmap represent treatment.

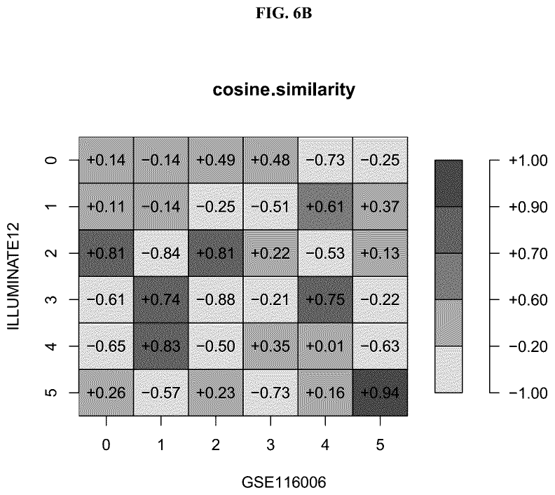

Subsets obtained from two independent datasets (GSE88884 and GSE116006) were checked for similarity using cosine similarity. FIGS. 6A-B shows subset similarity between two independent datasets. K-means clustering of two independent datasets GSE116006 (top, FIG. 6A) and GSE88884 (bottom: FIG. 6A) reveals four conserved subsets by cosine similarity (FIG. 6B).

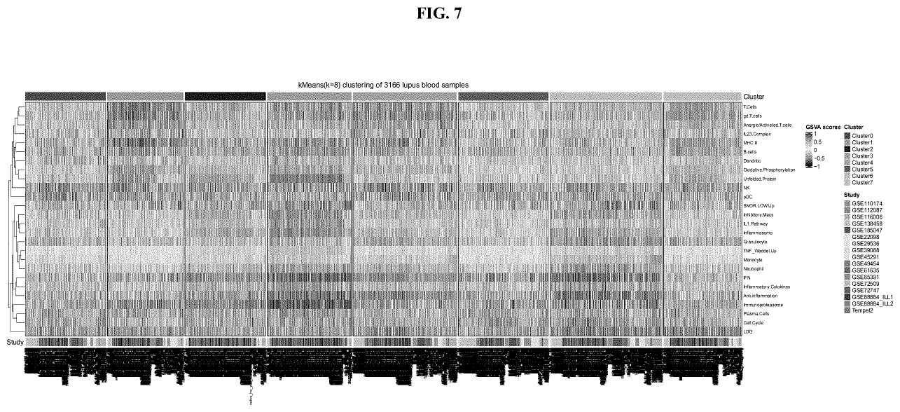

Eight molecular endotypes emerge from clustering of 17 datasets comprising 3,166 lupus patients (FIG. 7, FIG. 32A). The eight endotypes are visualized via k-means clustering.

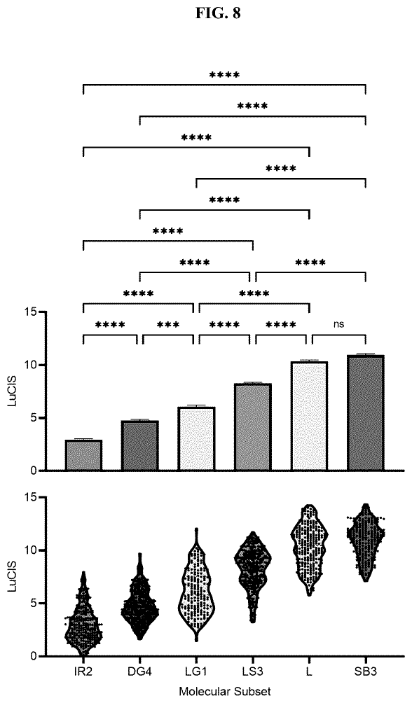

FIG. 8 show distribution of the Lupus Cell and Immune Score (LuCIS) for 1620 lupus patients across six molecular subsets. LuCIS was calculated for individual lupus patients and was plotted by molecular subset shown as (top) mean±SEM or (bottom) distribution of LuCIS as a violin plot. Significant differences between mean LuCIS of each cluster was analyzed with Dunn's multiple comparisons test.

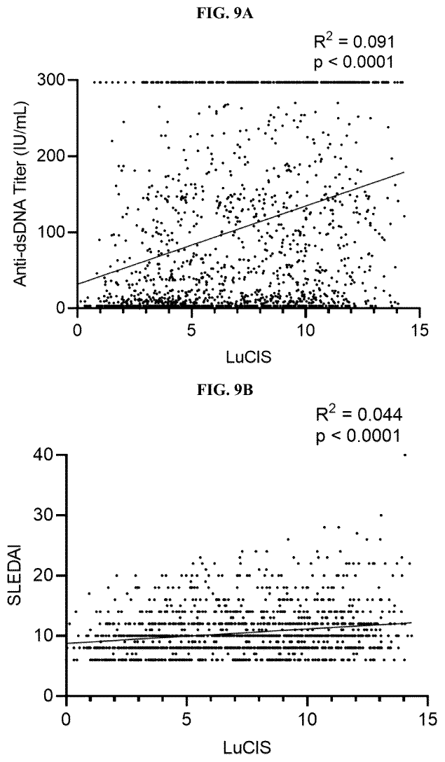

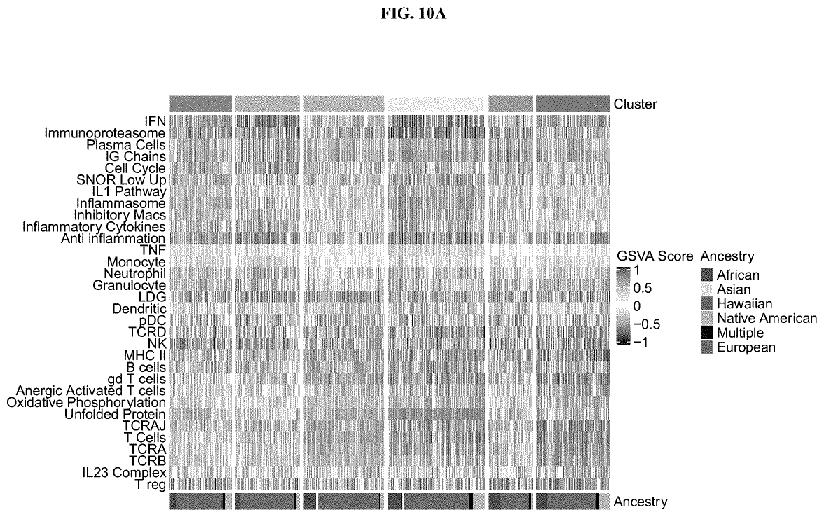

FIGS. 9A-B show LuCIS correlates with anti-dsDNA and SLEDAI. Linear regression of Anti-dsDNA (FIG. 9A) or SLEDAI (FIG. 9B) with LuCIS in 1612 patients from GSE88884.

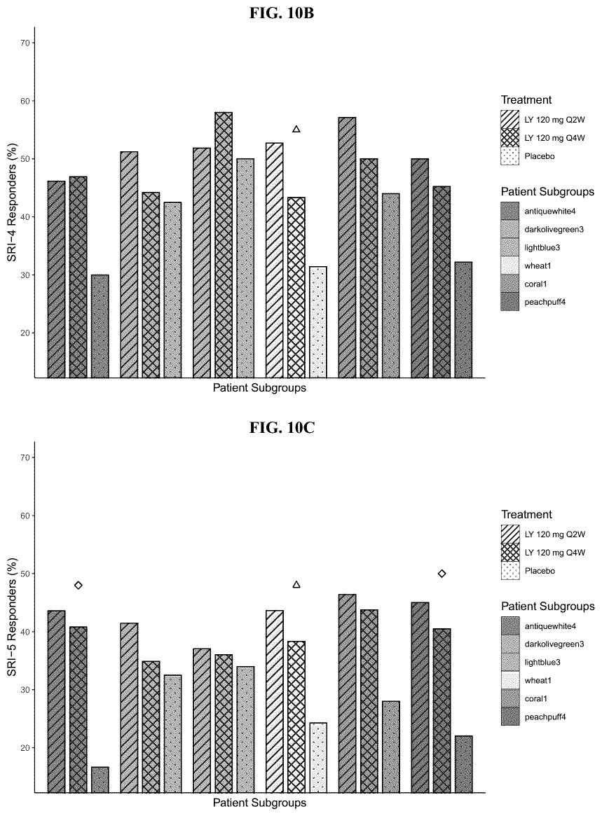

FIGS. 10A-C show molecular subset membership at baseline predicts drug response at 52 weeks. K-means clustering of 32 features in Illuminate-2 lupus samples (FIG. 10A) and their clinical responses by SRI-4 (FIG. 10B) and SRI-5 (FIG. 10C) per gene expression determined endotype. Responses among the treatment groups (shown in groups of 3 bars, from left to right) were ascertained by the Trend Chi Square test. Endotype color labels were randomly generated using the ‘grDevices’ color palette in R. Q2W indicates frequency of drug administration was every 2 weeks. Q4W indicates frequency of drug administration was every 4 weeks A: p<0.05 observed by Trend Chi Square Test for Q2W>Q4W>Placebo, Q2W>Placebo, and Q2W+Q4W>Placebo. For Q2W, and Q4W the dose amount was 120 mg. For FIGS. 10B and 10C, for each group of 3 bars, from left to right, left most bar is for Q2W treatment, middle bar is Q4W treatment, and right most bar is for placebo.

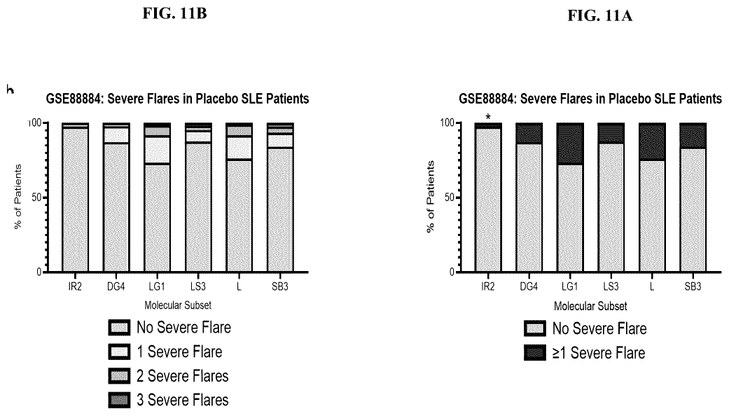

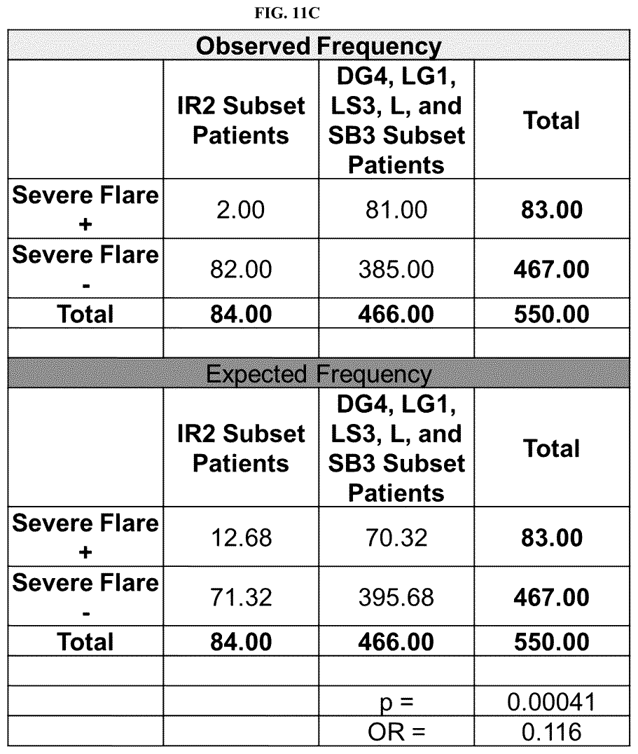

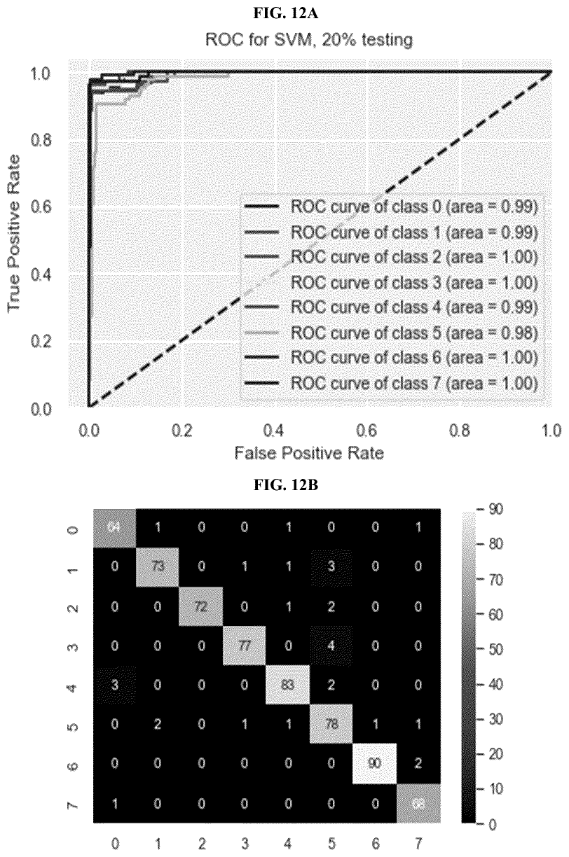

FIGS. 11 A-C show lupus patients in the least severe subset are less likely to have severe flares during 52 weeks on standard of care. Distribution of severe flares by molecular subset shown as (FIG. 11A) no severe flare or >1 severe flare, or (FIG. 11A) the number of severe flares. The likelihood of having >1 severe flare in the IR2 subset is 0.116 as compared to the other five subsets. Significant differences in expected and observed frequencies between IR2, the “least abnormal subset” and all other subsets (denoted with asterisk above bars) was identified by Chi Square Test, as shown by the contingency tables in (FIG. 11C). Significant associations between categorical variables and all subsets (denoted with asterisks on the y-axis) were identified using Chi Square Test of Independence.

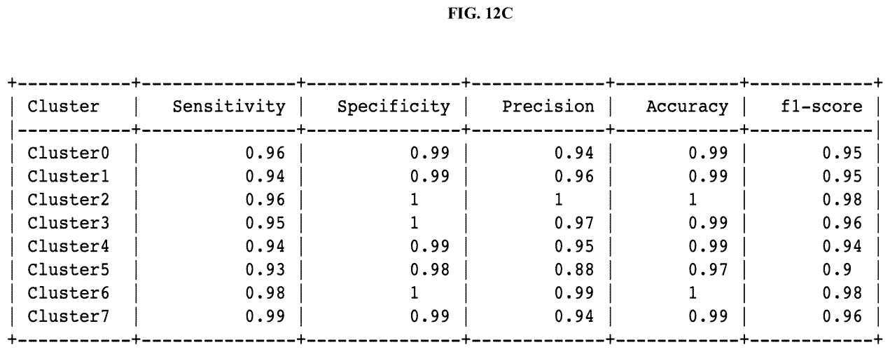

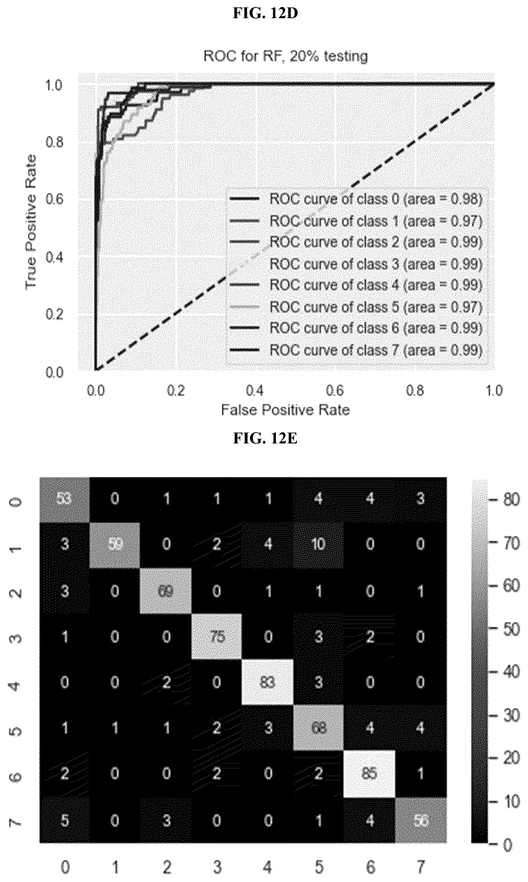

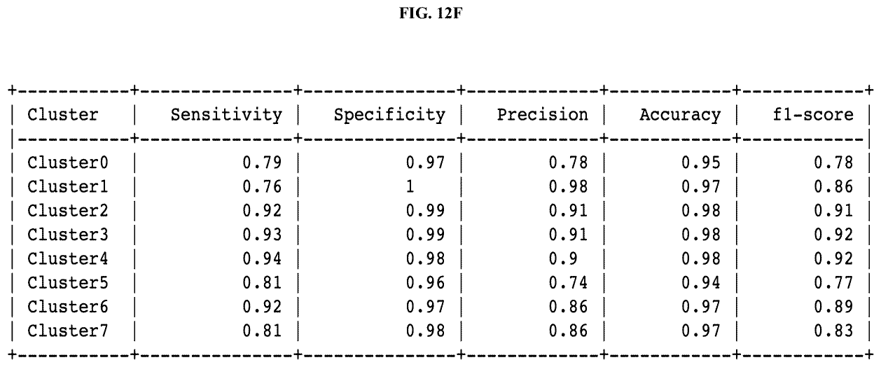

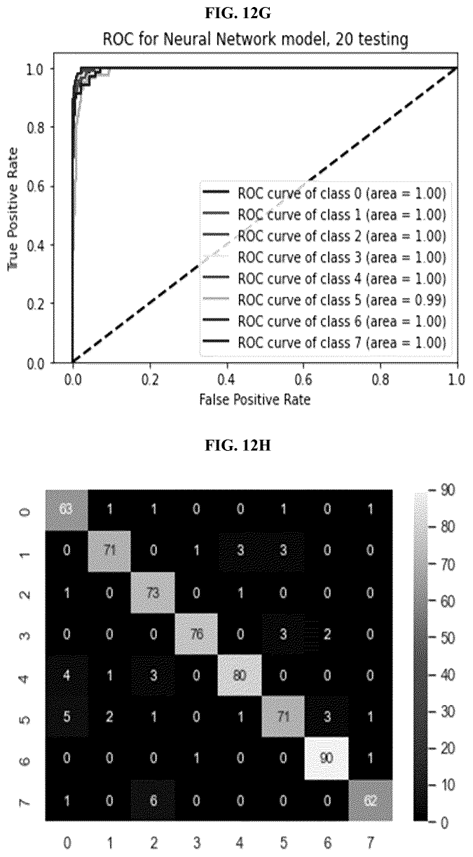

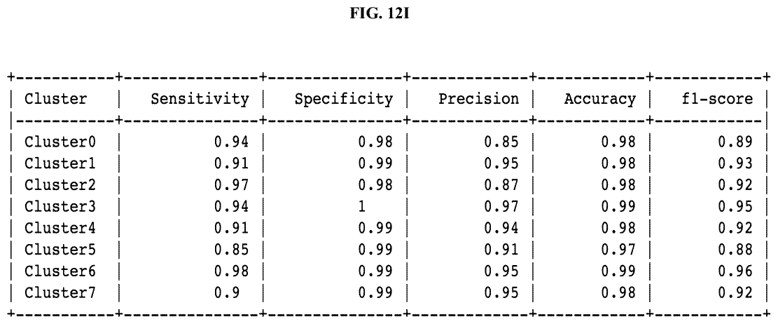

FIGS. 12A-I show machine learning algorithms can predict lupus endotype membership with high accuracy. Multi-class classification by machine learning analysis categorizes lupus samples into eight patient endotypes (FIG. 7, FIG. 32A). FIGS. 12A-C: Area under the ROC curve (AUC) (FIG. 12A), confusion matrices (FIG. 12B) and performance metrics (FIG. 12C), of classifier support vector machine. FIGS. 12D-F: Area under the ROC curve (AUC) (FIG. 12D), confusion matrices (FIG. 12E) and performance metrics (FIG. 12F), of classifier random forest. FIGS. 12G-I: Area under the ROC curve (AUC) (FIG. 12G), confusion matrices (FIG. 12H) and performance metrics (FIG. 12I), of classifier deep learning (sequential neural network). Each model was trained on 2532 (80%) lupus samples and tested on the remaining 20% (n=634) for a total N=3166 from 16 datasets.

Tables 1 to 32: 32 Cellular and Process Gene Modules. 32 Gene Modules Listing Genes. Genes are Listed by: Gene Symbol | Gene Entrez ID ∥)

TABLE 1
Module: Anergic/Activated T cells
Feature co-efficient = −0.03; Absolute feature co-efficient = 0.03

CD160 | 11126 || CD244 | 51744 || CTLA4 | 1493 || HAVCR2 | 84868 ||
ICOS | 29851 || KLRG1 | 10219 || LAG3 | 3902 || PDCD1 | 5133 ||

TABLE 2
Module: Anti inflammation
Feature co-efficient = 0.75; Absolute feature co-efficient = 0.75

IL1RN | 3557 || SOCS3 | 9021 || TNFAIP3 | 7128 ||

TABLE 3
Module: B cells
Feature co-efficient = −0.06; Absolute feature co-efficient = 0.06

BANK1 | 55024 || BLK | 640 || BLNK | 29760 || CD19 | 930 || CD22 |
933 || CD79A | 973 || CD79B | 974 || DAPP1 | 27071 || FCRL1 |
115350 || FCRL2 | 79368 || FCRL3 | 115352 || FCRLA | 84824 ||
GON4L | 54856 || GPR183 | 1880 || IGHD | 3495 || IGHM | 3507 ||
KLHL6 | 89857 || MS4A1 | 931 || PAX5 | 5079 || PLCL2 | 23228 ||
SH3BP5 | 9467 || VPREB1 | 7441 || ZNF318 | 24149 ||

TABLE 4
Module: Cell Cycle
Feature co-efficient = 0.78; Absolute feature co-efficient = 0.78

ASPM | 259266 || AURKA | 6790 || AURKB | 9212 || BRCA1 | 672 ||
CCNB1 | 891 || CCNB2 | 9133 || CCNE1 | 898 || CDC20 | 991 ||
CENPM | 79019 || CEP55 | 55165 || E2F3 | 1871 || GINS2 | 51659 ||
MCM10 | 55388 || MCM2 | 4171 || MKI67 | 4288 | NCAPG | 64151 ||
NDC80 | 10403 || PTTG1 | 9232 || TYMS | 7298 ||

TABLE 5
Module: Dendritic
Feature co-efficient = −0.49; Absolute feature co-efficient = 0.49

CLEC10A | 10462 || CLEC12A | 160364 || CLEC9A | 283420 || CSF1R |
1436 || IGIP | 492311 | LILRA4 | 23547 || LY75 | 4065 || XCR1 | 2829 ||

TABLE 6
Module: gd T cells
Feature co-efficient = −0.26; Absolute feature co-efficient = 0.26

TRG-AS1 | 100506776 ∥ BLK | 640 ∥ TARP | 445347 ∥ TRDC | 28526 ∥ CD3E | 916 ∥
CD3G | 917 ∥

TABLE 7
Module: Granulocyte
Feature co-efficient = 0.48; Absolute feature co-efficient = 0.48

CD177 | 57126 ∥ CLC | 1178 ∥ CTSS | 1520 ∥ CXCR2 | 3579 ∥ DEFA1 | 1667 ∥ FUT7 |
2529 ∥ LTB4R | 1241 ∥ MMP25 | 64386 ∥ OSM | 5008 ∥ RETN | 56729 ∥

TABLE 8
Module: IFN
Feature co-efficient = 1.04; Absolute feature co-efficient = 1.04

EIF2AK2 | 5610 ∥ GBP1 | 2633 ∥ GBP2 | 2634 ∥ GBP4 | 115361 ∥ HERC5 | 51191 ∥
HERC6 | 55008 ∥ IFI27 | 3429 ∥ IFI30 | 10437 ∥ IFI35 | 3430 ∥ IFI44 | 10561 ∥ IFI44L |
10964 ∥ IFI6 | 2537 ∥ IFIT1 | 3434 ∥ IFIT2 | 3433 ∥ IFIT3 | 3437 ∥ IFIT5 | 24138 ∥ IFITM1
| 8519 ∥ IFITM2 | 10581 ∥ IFITM3 | 10410 ∥ ISG15 | 9636 ∥ ISG20 | 3669 ∥ MX1 | 4599 ∥
MX2 | 4600 ∥ OAS1 | 4938 ∥ OAS2 | 4939 ∥ OAS3 | 4940 ∥ OASL | 8638 ∥ RSAD2 |
91543 ∥ SAMD9 | 54809 ∥ SAMD9L | 219285 ∥ SP100 | 6672 ∥ SP110 | 3431 ∥

TABLE 9
Module: IG CHAINS
Feature co-efficient = 0.32; Absolute feature co-efficient = 0.32

IGHA1 | 3493 ∥ IGHA2 | 3494 ∥ IGHD2-15 | 28503 ∥ IGHD2-2 | 28505 ∥ IGHD2-21 |
28502 ∥ IGHD3-10 | 28499 ∥ IGHD3-16 | 28498 ∥ IGHD3-3 | 28501 ∥ IGHD3-9 | 28500 ∥
IGHG1 | 3500 ∥ IGHG2 | 3501 ∥ IGHG3 | 3502 ∥ IGHG4 | 3503 ∥ IGHJ1 | 28483 ∥ IGHJ2 |
28481 ∥ IGHJ3 | 28479 ∥ IGHJ4 | 28477 ∥ IGHJ5 | 28476 ∥ IGHJ6 | 28475 ∥ IGHV1-18 |
28468 ∥ IGHV1-2 | 28474 ∥ IGHV1-24 | 28467 ∥ IGHV1-3 | 28473 ∥ IGHV1-45 | 28466 ∥
IGHV1-46 | 28465 ∥ IGHV1-58 | 28464 ∥ IGHV1-69-2 | 28458 ∥ IGHV2-26 | 28455 ∥
IGHV2-5 | 28457 ∥ IGHV2-70 | 28454 ∥ IGHV3-13 | 28449 ∥ IGHV3-15 | 28448 ∥
IGHV3-20 | 28445 ∥ IGHV3-21 | 28444 ∥ IGHV3-23 | 28442 ∥ IGHV3-33 | 28434 ∥
IGHV3-43 | 28426 ∥ IGHV3-48 | 28424 ∥ IGHV3-49 | 28423 ∥ IGHV3-53 | 28420 ∥
IGHV3-62 | 28416 ∥ IGHV3-64 | 28414 ∥ IGHV3-7 | 28452 ∥ IGHV3-72 | 28410 ∥
IGHV3-73 | 28409 ∥ IGHV3-74 | 28408 ∥ IGHV4-28 | 28400 ∥ IGHV4-30-2 | 28398 ∥
IGHV4-34 | 28395 ∥ IGHV4-39 | 28394 ∥ IGHV4-59 | 28392 ∥ IGHV4-61 | 28391 ∥
IGHV5-51 | 28388 ∥ IGHV6-1 | 28385 ∥ IGHV7-81 | 28378 ∥ IGKC | 3514 ∥ IGKJ1 |
28950 ∥ IGKJ2 | 28949 ∥ IGKJ3 | 28948 ∥ IGKJ4 | 28947 ∥ IGKJ5 | 28946 ∥ IGKV1-16 |
28938 ∥ IGKV1-17 | 28937 ∥ IGKV1-27 | 28935 ∥ IGKV1-5 | 28299 ∥ IGKV1-6 | 28943 ∥
IGKV1-9 | 28941 ∥ IGKV1D-16 | 28901 ∥ IGKV1D-17 | 28900 ∥ IGKV1D-43 | 28891 ∥
IGKV1D-8 | 28904 ∥ IGKV2-24 | 28923 ∥ IGKV2D-26 | 28884 ∥ IGKV2D-29 | 28882 ∥
IGKV2D-30 | 28881 ∥ IGKV3-20 | 28912 ∥ IGKV3D-20 | 28874 ∥ IGKV3D-7 | 28877 ∥
IGKV4-1 | 28908 ∥ IGKV5-2 | 28907 ∥ IGLC2 | 3538 ∥ IGLC7 | 28834 ∥ IGLJ6 | 28828 ∥
IGLV10-54 | 28772 ∥ IGLV1-36 | 28826 ∥ IGLV1-40 | 28825 ∥ IGLV1-47 | 28822 ∥
IGLV2-11 | 28816 ∥ IGLV2-18 | 28814 ∥ IGLV2-23 | 28813 ∥ IGLV2-33 | 28811 ∥ IGLV2-
8 | 28817 ∥ IGLV3-1 | 28809 ∥ IGLV3-10 | 28803 ∥ IGLV3-12 | 28802 ∥ IGLV3-16 | 28799
∥ IGLV3-19 | 28797 ∥ IGLV3-21 | 28796 ∥ IGLV3-25 | 28793 ∥ IGLV3-27 | 28791 ∥
IGLV3-32 | 28787 ∥ IGLV4-3 | 28786 ∥ IGLV4-60 | 28785 ∥ IGLV4-69 | 28784 ∥ IGLV5-
37 | 28783 ∥ IGLV5-45 | 28781 ∥ IGLV6-57 | 28778 ∥ IGLV7-43 | 28776 ∥ IGLV8-61 |
28774 ∥ IGLV9-49 | 28773 ∥ IGLVI-70 | 28763 ∥

TABLE 10
Module: IL1 Pathway
Feature co-efficient = 0.17; Absolute feature co-efficient = 0.17

CHUK | 1147 ∥ IKBKB | 3551 ∥ NFKB1 | 4790 ∥ MAP2K1 | 5604 ∥ MAP2K4 | 6416 ∥
IKBKG | 8517 ∥ IL1A | 3552 ∥ IL1R1 | 3554 ∥ IRAK1 | 3654 ∥ MAP3K3 | 4215 ∥ MYD88
| 4615 ∥ MAP2K6 | 5608 ∥ MAP3K7 | 6885 ∥ TRAF6 | 7189 ∥ TAB1 | 10454 ∥ TAB2 |
23118 ∥ IRAK4 | 51135 ∥ TOLLIP | 54472 ∥ MAP3K8 | 1326 ∥ IL1RAP | 3556 ∥ IRAK2 |
3656 ∥ MAPK8 | 5599 ∥ RELA | 5970 ∥ TAB3 | 257397 ∥ IL1B | 3553 ∥ UBE2N | 7334 ∥
SQSTM1 | 8878 ∥ IRAK3 | 11213 ∥

TABLE 11
Module: IL23 Complex
Feature co-efficient = −0.29; Absolute feature co-efficient = 0.29

IL12B | 3593 ∥ IL12RB1 | 3594 ∥ IL23A | 51561 ∥ IL23R | 149233 ∥

TABLE 12
Module: Immunoproteosome
Feature co-efficient = 0.61; Absolute feature co-efficient = 0.61

PSMB10 | 5699 ∥ PSMB8 | 5696 ∥ PSMB9 | 5698 ∥

TABLE 13
Module: Inflammasome
Feature co-efficient = 0.57; Absolute feature co-efficient = 0.57

AIM2 | 9447 ∥ CASP1 | 834 ∥ CASP5 | 838 ∥ CTSB | 1508 ∥ GSDMB | 55876 ∥ GSDMD |
79792 ∥ NAIP | 4671 ∥ NEK7 | 140609 ∥ NLRC4 | 58484 ∥ NLRP1 | 22861 ∥ NLRP3 |
114548 ∥ NOD2 | 64127 ∥ P2RX7 | 5027 ∥ PANX1 | 24145 ∥ PYCARD | 29108 ∥ RIPK1 |
8737

TABLE 14
Module: Inflammatory Cytokines
Feature co-efficient = 0.69; Absolute feature co-efficient = 0.69

IL1A | 3552 ∥ CXCL10 | 3627 ∥ CXCL11 | 6373 ∥ CXCL9 | 4283 ∥ TNFSF13B | 10673 ∥
CCL8 | 6355 ∥ TNF | 7124 ∥ IL1B | 3553 ∥ IL18 | 3606 ∥

TABLE 15
Module: Inhibitory Macs
Feature co-efficient = 0.60; Absolute feature co-efficient = 0.60

AOAH | 313 ∥ BACH1 | 571 ∥ CD200R1 | 131450 ∥ CD300A | 11314 ∥ CD163 | 9332 ∥
CLEC7A | 64581 ∥ SCARB1 | 949 ∥ MS4A4A | 51338 ∥ IL10 | 3586 ∥

TABLE 16
Module: LDG
Feature co-efficient = 0.65; Absolute feature co-efficient = 0.65

AZU1 | 566 ∥ CAMP | 820 ∥ CEACAM6 | 4680 ∥ CEACAM8 | 1088 ∥ CTSG | 1511 ∥
DEFA4 | 1669 ∥ ELANE | 1991 ∥ LCN2 | 3934 ∥ LTF | 4057 ∥ MPO | 4353 ∥ OLFM4 |
10562 ∥ RNASE3 | 6037 ∥

TABLE 17
Module: MHC II
Feature co-efficient = −0.28; Absolute feature co-efficient = 0.28

HLA-DMA | 3108 ∥ HLA-DMB | 3109 ∥ HLA-DPA1 | 3113 ∥ HLA-DPB1 | 3115 ∥ HLA-
DPB2 | 3116 ∥ HLA-DQA1 | 3117 ∥ HLA-DQA2 | 3118 ∥ HLA-DQB1 | 3119 ∥ HLA-
DQB2 | 3120 ∥ HLA-DRA | 3122 ∥ HLA-DRB1 | 3123 ∥ HLA-DRB3 | 3125 ∥ HLA-DRB4
| 3126 ∥ HLA-DRB5 | 3127 ∥ HLA-DRB6 | 3128 ∥

TABLE 18
Module: Monocyte
Feature co-efficient = 0.87; Absolute feature co-efficient = 0.87

ACE | 1636 ∥ ADAM8 | 101 ∥ ADAMDEC1 | 27299 ∥ ADGRE1 | 2015 ∥ ADGRE2 |
30817 ∥ APOBEC3B | 9582 ∥ APOBEC3G | 60489 ∥ APOBR | 55911 ∥ ART4 | 420 ∥
C1QA | 712 ∥ C1QC | 714 ∥ C2 | 717 ∥ C4A | 720 ∥ C4B | 721 ∥ C4BPA | 722 ∥ C4BPB |
725 ∥ C5 | 727 ∥ C6 | 729 ∥ C8A | 731 ∥ C9 | 735 ∥ CCL17 | 6361 ∥ CCL18 | 6362 ∥ CCL22
| 6367 ∥ CCL28 | 56477 ∥ CCL7 | 6354 ∥ CCL8 | 6355 ∥ CD14 | 929 ∥ CD163 | 9332 ∥
CD209 | 30835 ∥ CD300C | 10871 ∥ CD300E | 342510 ∥ CD5L | 922 ∥ CD80 | 941 ∥ CFB |
629 ∥ CFD | 1675 ∥ CFP | 5199 ∥ CHI3L1 | 1116 ∥ CHIT1 | 1118 ∥ CLEC12A | 160364 ∥
CLEC12B | 387837 ∥ CLEC4D | 338339 ∥ CLEC4E | 26253 ∥ CLEC5A | 23601 ∥
CLEC7A | 64581 ∥ CLEC9A | 283420 ∥ CSF1R | 1436 ∥ CXCL1 | 2919 ∥ CXCL10 | 3627
∥ CXCL11 | 6373 ∥ CXCL13 | 10563 ∥ CXCL8 | 3576 ∥ CXCL9 | 4283 ∥ CYBB | 1536 ∥
F12 | 2161 ∥ FCGR3B | 2215 ∥ FFAR2 | 2867 ∥ HMMR | 3161 ∥ HVCN1 | 84329 ∥ IGSF6 |
10261 ∥ IL10RA | 3587 ∥ IL12A | 3592 ∥ IL12B | 3593 ∥ IL1RAP | 3556 ∥ IL20 | 50604 ∥
IL23A | 51561 ∥ IL27 | 246778 ∥ IL31RA | 133396 ∥ LAMP3 | 27074 ∥ LGALS12 | 85329
∥ LGALS4 | 3960 ∥ LGALS9 | 3965 ∥ LGALS9B | 284194 ∥ LGALS9C | 654346 ∥
LILRA2 | 11027 ∥ LILRA5 | 353514 ∥ LILRA6 | 79168 ∥ LILRB5 | 10990 ∥ LMNB1 |
4001 ∥ LY86 | 9450 ∥ LYZ | 4069 ∥ MARCO | 8685 ∥ MEGF10 | 84466 ∥ MERTK | 10461
∥ MFGE8 | 4240 ∥ MPEG1 | 219972 ∥ MRC1 | 4360 ∥ MS4A4A | 51338 ∥ MSR1 | 4481 ∥
NTSR1 | 4923 ∥ OCM | 654231 ∥ OLR1 | 4973 ∥ OSCAR | 126014 ∥ OTOF | 9381 ∥
PDCD1LG2 | 80380 ∥ PILRA | 29992 ∥ PLA2G2D | 26279 ∥ PLA2G5 | 5322 ∥ PYHIN1 |
149628 ∥ S100A8 | 6279 ∥ S100A9 | 6280 ∥ S1PR5 | 53637 ∥ SCARB1 | 949 ∥ SCARF2 |
91179 ∥ SECTM1 | 6398 ∥ SEMA4A | 64218 ∥ SERPINB9 | 5272 ∥ SERPING1 | 710 ∥
SIGLEC1 | 6614 ∥ SIGLEC14 | 100049587 ∥ SIGLEC5 | 8778 ∥ SIGLEC7 | 27036 ∥
SLC11A1 | 6556 ∥ SLITRK4 | 139065 ∥ SMPDL3B | 27293 ∥ SPIC | 121599 ∥ STAB2 |
55576 ∥ STAP2 | 55620 ∥ TEK | 7010 ∥ TGM2 | 7052 ∥ TIMD4 | 91937 ∥ TLR2 | 7097 ∥
TLR8 | 51311 ∥ TNF | 7124 ∥ TNFAIP8L2 | 79626 ∥ TNFRSF1B | 7133 ∥ TNIP3 | 79931 ∥
TULP1 | 7287 ∥ UBD | 10537 ∥ VENTX | 27287 ∥ VSTM1 | 284415 ∥

TABLE 19
Module: Neutrophil
Feature co-efficient = 0.72; Absolute feature co-efficient = 0.72

ABHD3 | 171586 ∥ ADAM8 | 101 ∥ CD177 | 57126 ∥ CD83 | 9308 ∥ CLEC6A | 93978 ∥
LY6E | 4061 ∥ S100A6 | 6277 ∥

TABLE 20
Module: NK
Feature co-efficient = −0.46; Absolute feature co-efficient = 0.46

KLRF1 | 51348 ∥ NCAM1 | 4684 ∥ NCR1 | 9437 ∥ NCR3 | 259197 ∥ SH2D1B | 117157 ∥

TABLE 21
Module: Oxidative Phosphorylation
Feature co-efficient = −0.01; Absolute feature co-efficient = 0.01

ATP5A1 | 498 || ATP5B | 506 || ATP5D | 513 || ATP5E | 514 || ATP5F1 | 515 || ATP5G1 |
516 || ATP5G2 | 517 || ATP5G3 | 518 || ATP5H | 10476 || ATP5I | 521 || ATP5J | 522 ||
ATP5J2 | 9551 || ATP5L | 10632 || ATP5O | 539 || ATP5S | 27109 || BCS1L | 617 || CEP89
| 84902 || COA1 | 55744 || COA3 | 28958 || COA4 | 51287 || COA5 | 493753 || COA6 |
388753 || COA7 | 65260 || COX10 | 1352 || COX10-AS1 | 100874058 || COX11 | 1353 ||
COX14 | 84987 || COX15 | 1355 || COX16 | 51241 || COX17 | 10063 || COX18 | 285521 ||
COX19 | 90639 || COX20 | 116228 || COX4I1 | 1327 || COX4I2 | 84701 || COX5A | 9377 ||
COX5B | 1329 || COX6A1 | 1337 || COX6A2 | 1339 || COX6B1 | 1340 || COX6B2
125965 || COX6C | 1345 || COX7A1 | 1346 || COX7A2 | 1347 || COX7A2L | 9167 ||
COX7B | 1349 || COX7B2 | 170712 || COX7C | 1350 || COX8A | 1351 || COX8C | 341947
|| CYC1 | 1537 || CYCS | 54205 || DNAJC15 | 29103 || MT-ATP6 | 4508 || MT-ATP8 |
4509 || MT-CO1 | 4512 || MT-CO2 | 4513 || MT-CO3 | 4514 || MT-CYB | 4519 || MT-ND1
| 4535 || MT-ND2 | 4536 || MT-ND3 | 4537 || MT-ND4 | 4538 || MT-ND4L | 4539 || MT-
ND5 | 4540 || MT-ND6 | 4541 || NDUFA1 | 4694 || NDUFA10 | 4705 || NDUFA11 |
126328 || NDUFA12 | 55967 || NDUFA13 | 51079 || NDUFA2 | 4695 || NDUFA3 | 4696 ||
NDUFA4 | 4697 || NDUFA4L2 | 56901 || NDUFA5 | 4698 || NDUFA6 | 4700 || NDUFA7 |
4701 || NDUFA8 | 4702 || NDUFA9 | 4704 || NDUFAB1 | 4706 || NDUFAF1 | 51103 ||
NDUFAF2 | 91942 || NDUFAF3 | 25915 || NDUFAF4 | 29078 || NDUFAF5 | 79133 ||
NDUFAF6 | 137682 || NDUFAF7 | 55471 || NDUFAF8 | 284184 || NDUFB1 | 4707 ||
NDUFB10 | 4716 || NDUFB11 | 54539 || NDUFB2 | 4708 || NDUFB2-AS1 | 100134713 ||
NDUFB3 | 4709 || NDUFB4 | 4710 || NDUFB5 | 4711 || NDUFB6 | 4712 || NDUFB7 | 4713
|| NDUFB8 | 4714 || NDUFB9 | 4715 || NDUFC1 | 4717 || NDUFC2 | 4718 || NDUFS1 |
4719 || NDUFS2 | 4720 || NDUFS3 | 4722 || NDUFS4 | 4724 || NDUFS5 | 4725 || NDUFS6
| 4726 || NDUFS7 | 374291 || NDUFS8 | 4728 || NDUFV1 | 4723 || NDUFV2 | 4729 ||
NDUFV3 | 4731 || NUBPL | 80224 || OXA1L | 5018 || RFESD | 317671 || SCO1 | 6341 ||
SCO2 | 9997 || SLC25A4 | 291 || SURF1 | 6834 || TACO1 | 51204 || TIMMDC1 | 51300 ||
TMEM126B | 55863 || TRAP1 | 10131 || TTC19 | 54902 || UQCC1 | 55245 || UQCC2 |
84300 || UQCC3 | 790955 || UQCR10 | 29796 || UQCR11 | 10975 || UQCRB | 7381 ||
UQCRC1 | 7384 || UQCRC2 | 7385 || UQCRFS1 | 7386 || UQCRH | 7388 || UQCRHL |
440567 || UQCRQ | 27089 ||

TABLE 22
Module: pDC
Feature co-efficient = −0.47; Absolute feature co-efficient = 0.47

CLEC4C | 170482 || NRP1 | 8829 || IL3RA | 3563 ||

TABLE 23
Module: Plasma Cells
Feature co-efficient = 0.54; Absolute feature co-efficient = 0.54

C19orf10 | 56005 || IGH | 3492 || IGHD | 3495 || IGHG1 | 3500 || IGHMBP2 | 3508 ||
IGHV2-5 | 28457 || IGHV3-20 | 28445 || IGHV3-23 | 28442 || IGHV4-28 | 28400 ||
IGHV4-31 | 28396 || IGHV4-34 | 28395 || IGK | 50802 || IGKC | 3514 || IGL | 3535 ||
IGLJ3 | 28831 || IGLL1 | 3543 || IGLV@ | 3546 || IGLV1-40 | 28825 || IGLV1-44 | 28823 ||
IGLV2-14 | 28815 || IGLV2-5 | 28818 || IGLV3-1 | 28809 || IGLV3-19 | 28797 || IGLV3-25
| 28793 || IGLV4-3 | 28786 || IGLV4-60 | 28785 || IGLV5-45 | 28781 || IGLV6-57 | 28778 ||
IGLVI-70 | 28763 || MZB1 | 51237 || PRDM1 | 639 || SDC1 | 6382 || THEMIS2 | 9473 ||
TNFRSF17 | 608 ||

TABLE 24
Module: SNOR LOW Up
Feature co-efficient = 0.36; Absolute feature co-efficient = 0.36

CEACAM1 | 634 || FCGR1A | 2209 || LGALS1 | 3956 || SNORD24 |
26820 || SNORD44 | 26806 || SNORD47 | 26802 || SNORD80 | 26774 ||

TABLE 25
Module: T Cells
Feature co-efficient = −0.20; Absolute feature co-efficient = 0.20

CCR3 | 1232 || CD226 | 10666 || CD247 | 919 || CD28 | 940 || CD3D | 915 || CD3E | 916 ||
CD3G | 917 || CD4 | 920 || CD5 | 921 || CD8A | 925 || CD8B | 926 || ETS1 | 2113 || GATA3
| 2625 || GRAP2 | 9402 || LEF1 | 51176 || SH2D1A | 4068 || TRAC | 28755 || TRBC1 |
28639 || TRDC | 28526 ||

TABLE 26
Module: T reg
Feature co-efficient = −0.02; Absolute feature co-efficient = 0.02

FOXP3 | 50943 || IKZF2 | 22807 ||

TABLE 27
Module: TCRA
Feature co-efficient = −0.44; Absolute feature co-efficient = 0.44

TRAV10 | 28676 || TRAV1-1 | 28693 || TRAV1-2 | 28692 || TRAV12-1 | 28674 ||
TRAV12-2 | 28673 || TRAV12-3 | 28672 || TRAV13-1 | 28671 || TRAV13-2 | 28670 ||
TRAV14DV4 | 28669 || TRAV16 | 28667 || TRAV17 | 28666 || TRAV18 | 28665 ||
TRAV19 | 28664 || TRAV2 | 28691 || TRAV20 | 28663 || TRAV21 | 28662 || TRAV22 |
28661 || TRAV23DV6 | 28660 || TRAV24 | 28659 || TRAV25 | 28658 || TRAV26-1 |
28657 || TRAV26-2 | 28656 || TRAV27 | 28655 || TRAV29DV5 | 28653 || TRAV3 | 28690
|| TRAV30 | 28652 || TRAV34 | 28648 || TRAV35 | 28647 || TRAV36DV7 | 28646 ||
TRAV38-1 | 28644 || TRAV38-2DV8 | 28643 || TRAV39 | 28642 || TRAV4 | 28689 ||
TRAV40 | 28641 || TRAV41 | 28640 || TRAV5 | 28688 || TRAV7 | 28686 || TRAV8-1 |
28685 || TRAV8-2 | 28684 || TRAV8-3 | 28683 || TRAV8-4 | 28682 || TRAV8-6 | 28680 ||
TRAV8-7 | 28679 || TRAV9-1 | 28678 || TRAV9-2 | 28677 ||

TABLE 28
Module: TCRAJ
Feature co-efficient = −0.50; Absolute feature co-efficient = 0.50

TRAJ10 | 28745 || TRAJ11 | 28744 || TRAJ12 | 28743 || TRAJ13 | 28742 || TRAJ14 | 28741
|| TRAJ15 | 28740 || TRAJ16 | 28739 || TRAJ17 | 28738 || TRAJ18 | 28737 || TRAJ19 |
28736 || TRAJ20 | 28735 || TRAJ21 | 28734 || TRAJ22 | 28733 || TRAJ23 | 28732 ||
TRAJ24 | 28731 || TRAJ25 | 28730 || TRAJ26 | 28729 || TRAJ27 | 28728 || TRAJ28 | 28727
|| TRAJ29 | 28726 || TRAJ3 | 28752 || TRAJ30 | 28725 || TRAJ31 | 28724 || TRAJ32 |
28723 || TRAJ33 | 28722 || TRAJ34 | 28721 || TRAJ35 | 28720 || TRAJ36 | 28719 ||
TRAJ37 | 28718 || TRAJ38 | 28717 || TRAJ39 | 28716 || TRAJ4 | 28751 || TRAJ40 | 28715
|| TRAJ41 | 28714 || TRAJ42 | 28713 || TRAJ43 | 28712 || TRAJ44 | 28711 || TRAJ45 |
28710 || TRAJ46 | 28709 || TRAJ47 | 28708 || TRAJ48 | 28707 || TRAJ49 | 28706 || TRAJ5
| 28750 || TRAJ50 | 28705 || TRAJ52 | 28703 || TRAJ53 | 28702 || TRAJ54 | 28701 ||
TRAJ56 | 28699 || TRAJ57 | 28698 || TRAJ58 | 28697 || TRAJ59 | 28696 || TRAJ6 | 28749
|| TRAJ61 | 28694 || TRAJ7 | 28748 || TRAJ8 | 28747 || TRAJ9 | 28746 ||

TABLE 29
Module: TCRB
Feature co-efficient = −0.50; Absolute feature co-efficient = 0.50

TRBC2 | 28638 || TRBJ2-1 | 28629 || TRBJ2-2 | 28628 || TRBJ2-2P | 28627 || TRBJ2-3 |
28626 || TRBJ2-4 | 28625 || TRBJ2-5 | 28624 || TRBJ2-6 | 28623 || TRBJ2-7 | 28622 ||
TRBV1 | 28621 || TRBV10-1 | 28585 || TRBV10-2 | 28584 || TRBV11-1 | 28582 ||
TRBV11-2 | 28581 || TRBV19 | 28568 || TRBV2 | 28620 || TRBV20-1 | 28567 || TRBV21-
1 | 28566 || TRBV23-1 | 28564 || TRBV24-1 | 28563 || TRBV25-1 | 28562 || TRBV27 |
28560 || TRBV28 | 28559 || TRBV3-1 | 28619 || TRBV4-1 | 28617 || TRBV4-2 | 28616 ||
TRBV5-1 | 28614 || TRBV5-3 | 28612 || TRBV5-4 | 28611 || TRBV5-5 | 28610 || TRBV5-6
| 28609 || TRBV5-7 | 28608 || TRBV6-1 | 28606 || TRBV6-4 | 28603 || TRBV6-5 | 28602 ||
TRBV6-6 | 28601 || TRBV6-7 | 28600 || TRBV6-8 | 28599 || TRBV7-1 | 28597 || TRBV7-3
| 28595 || TRBV7-4 | 28594 || TRBV7-5 | 28593 || TRBV7-6 | 28592 || TRBV7-7 | 28591 ||
TRBV9 | 28586 ||

TABLE 30
Module: TCRD
Feature co-efficient = −0.41; Absolute feature co-efficient = 0.41

TRDC | 28526 || TRDJ1 | 28522 || TRDJ2 | 28521 || TRDJ3 | 28520 ||
TRDJ4 | 28519 || TRDV1 | 28518 || TRDV2 | 28517 || TRDV3 | 28516 ||

TABLE 31
Module: TNF_Waddel Up
Feature co-efficient = 0.67; Absolute feature co-efficient = 0.67

ACLY | 47 || ACSL1 | 2180 || ADGRE2 | 30817 || AK3 | 50808 || AKAP10 | 11216 ||
AMPD3 | 272 || APOL3 | 80833 || ARID3A | 1820 || ARSE | 415 || ASAP1 | 50807 ||
B4GALT5 | 9334 || BCL2A1 | 597 || BHLHE41 | 79365 || BHMT | 635 || BIRC3 | 330 ||
BRCA1 | 672 || CALD1 | 800 || CASP1 | 834 || CASP10 | 843 || CCL15 | 6359 || CCL20 |
6364 || CCL23 | 6368 || CCL3L1 | 6349 || CD37 | 951 || CD38 | 952 || CD83 | 9308 ||
CDKN3 | 1033 || CKB | 1152 || CR2 | 1380 || CTNND2 | 1501 || CXCL1 | 2919 || CXCL2 |
2920 || CXCL3 | 2921 || CXCL8 | 3576 || CYP27B1 | 1594 || DAB2 | 1601 || EBI3 | 10148 ||
EGR1 | 1958 || EGR2 | 1959 || EPB41 | 2035 || EREG | 2069 || ETAA1 | 54465 || F3 | 2152 ||
FABP1 | 2168 || FBXL2 | 25827 || FCER2 | 2208 || FCGR2A | 2212 || FLJ11129 | 54674 ||
FLNA | 2316 || G0S2 | 50486 || GBP1 | 2633 || GCH1 | 2643 || GJB2 | 2706 || GLS | 2744 ||
GMIP | 51291 || GP1BA | 2811 || GRK3 | 157 || HCAR3 | 8843 || HHEX | 3087 || HOMER2
| 9455 || HP | 3240 || ICAM1 | 3383 || IDO1 | 3620 || IFI44 | 10561 || IKBKG | 8517 || IL16 |
3603 || IL18 | 3606 || IL1A | 3552 || IL1B | 3553 || IL1RN | 3557 || IL6 | 3569 || INHBA |
3624 || INSIG1 | 3638 || ITGA6 | 3655 || KITLG | 4254 || KLF1 | 10661 || KMO | 8564 ||
LGALS3BP | 3959 || MAP3K4 | 4216 || MARCKS | 4082 || MGLL | 11343 || MMP19 |
4327 || MN1 | 4330 || MRPS15 | 64960 || MSC | 9242 || MTF1 | 4520 || MX1 | 4599 ||
NAMPT | 10135 || NELL2 | 4753 || NFKB1 | 4790 || NFKB2 | 4791 || NFKBIA | 4792 ||
NFKBIZ | 64332 || NKX3-2 | 579 || NR3C1 | 2908 || OAS3 | 4940 || PATJ | 10207 ||
PDE4DIP | 9659 || PDPN | 10630 || PIAS4 | 51588 || PLAUR | 5329 || PTGES | 9536 ||
PTGS2 | 5743 || RELB | 5971 || RPGR | 6103 || RPS9 | 6203 || SDC4 | 6385 || SERPIND1 |
3053 || SFRP1 | 6422 || SH3BP5 | 9467 || SLAMF1 | 6504 || SLC30A4 | 7782 || SOD2 |
6648 || SPI1 | 6688 || SSPN | 8082 || STAT4 | 6775 || TAF15 | 8148 || TAP2 | 6891 || TBX3 |
6926 || TFF1 | 7031 || TNF | 7124 || TNFAIP2 | 7127 || TNFAIP3 | 7128 || TNFRSF11A |
8792 || TRAF1 | 7185 || TSC22D1 | 8848 || TYROBP | 7305 || UBE2C | 11065 || VEGFA |
7422 || WT1 | 7490 ||

TABLE 32
Module: Unfolded Protein
Feature co-efficient = 0.14; Absolute feature co-efficient = 0.14

B4GALT3 | 8703 || CALR | 811 || CALU | 813 || CANX | 821 || CDS2 | 8760 || CHST12 |
55501 || CHST2 | 9435 || DERL1 | 79139 || DERL2 | 51009 || DNAJC3 | 5611 || EDEM2 |
55741 || EDEM3 | 80267 || EMC9 | 51016 || ERAP1 | 51752 || ERGIC2 | 51290 || ERO1L |
30001 || EXT1 | 2131 || GALNT2 | 2590 || GOLT1B | 51026 || HERPUD1 | 9709 || HYOU1
| 10525 || IER3IP1 | 51124 || IMPAD1 | 54928 || KDELC1 | 79070 || KDELR2 | 11014 ||
LMAN2 | 10960 || LPGAT1 | 9926 || MAN1A1 | 4121 || MANEA | 79694 || MANF | 7873 ||
NUCB2 | 4925 || PDIA4 | 9601 || PDIA6 | 10130 || PIGK | 10026 || PPIB | 5479 || SEC24D |
9871 || SEC61G | 23480 || SPCS3 | 60559 || SSR1 | 6745 || SSR3 | 6747 || TRAM1 | 23471 ||
TRAM2 | 9697 || UGGT1 | 56886 || XBP1 | 7494 ||

### Example 2: Analysis of Transcriptomic Features Reveals Molecular Endotypes of SLE with Clinical Implications

The absence of a typical disease pattern is a major limiting feature in understanding the pathogenesis of and developing more effective therapies for systemic lupus erythematosus (SLE, or lupus) [1]. Clinical heterogeneity of SLE has complicated diagnosis and undermined clinical trials and thereby impeded precision medicine strategies [2]. Therefore, efforts have been undertaken to identify molecular endotypes of SLE, that is, subsets of patients defined by distinct pathobiological functions, biomarkers, or other disease mechanisms.

Determination of endotypes has already been employed in the management of allergic disease and is just beginning to be conceptualized in other autoimmune conditions and cancer [3-8]. One goal of endotyping of lupus is to identify groups of patients expected to be more likely to respond to specific treatments, thereby increasing the likelihood of success of clinical care and clinical trials [9-10]. Heterogeneity in lupus can manifest at the level of gene expression in peripheral blood [9, 11-14], suggesting that molecular profiling might serve as the basis of identifying specific lupus endotypes with clinical implications.

To date, several groups have reported subclassifications of SLE based on transcriptomics [9-10, 12, 15-21], but, in general, these have been single-center studies of limited numbers of patients and a broad consensus of recognized subsets has not emerged. Moreover, these studies have not considered confounding variables on gene expression, such as ancestry or medication nor have the findings been translated into clinical care or clinical trial design [13, 22]. Here, we present an approach to identify molecular endotypes of lupus patients based on transcriptomic profiling employing 32 immune and inflammatory-related features. We leverage transcriptomic data, machine learning (ML), and contemporary bioinformatics to subclassify 3,166 lupus patients into eight endotypes and develop an accompanying clinical metric, the Lupus Cell and Immune Score (LuCIS), to estimate a patient's lupus-related immunologic activity.

Part of the current challenge of realizing precision medicine in lupus is related to the application of imprecise clinical tools to evaluate a biologically complex disease. Although patient subsets based on age and comorbidities have been suggested to associate with differences in prognosis and treatment [1], to our knowledge, this is the first transcriptomic-based molecular endotyping approach that has staging and prognostic implications. Translation of these findings into the clinic may serve to facilitate personalized medicine.

### Methods

Patient Involvement. Patients were not directly recruited or involved in this study.

Datasets and Derivation of 32 Molecular Features A total of 17 transcriptomic lupus datasets were utilized in this study (Table 33). Determination of lupus endotypes was based on enrichment of 32 molecular features (FIG. 14). The module name and genes within the 32 molecular features/modules are listed in Tables 1-32.

Gene Set Variation Analysis (GSVA) and K-means Clustering. GSVA is a non-parametric, unsupervised statistical method for determining enrichment of a pre-defined set of genes in an individual sample. Calculated GSVA enrichment scores for the 32 gene sets (Tables 1-32) in each patient were used as input to k-means clustering for determination of subsets.

Clinical Response Measurement and Evaluation. Two clinical response metrics, SLEDAI response index (SRI)-4 and SRI-5, from the ILLUMINATE-2 trial (GSE88884) were used to assess clinical responders to tabalumab in the transcriptomic-determined endotypes. In each patient subset, samples were categorized by treatment received: placebo, or administration of tabalumab every 2 (Q2W) or 4 weeks (Q4W) [23]. Responses among treatment groups were then determined by the Trend Chi Square test using the ‘coin’ package in R [24].

Clinical metadata from both GSE88884 and GSE65391 was used to characterize the transcriptomic-determined endotypes. Additional metadata for GSE88884 included severe flare, defined by the SELENA-SLEDAI Flare Index [25]. Significant associations of clinical traits and endotypes were determined by the Kruskal-Wallis and Dunn's multiple comparisons test in GraphPad Prism v. 9.1.0(221) or the Chi Square test of independence.

ML Classification of Final Endotypes. 3,166 lupus patients from all 17 datasets were endotyped for the development of an ML classification tool. GSVA enrichment scores of 26 out of the 32 molecular features that were present in all 17 datasets (i.e., unaffected by inter-platform differences) were input into the k-means clustering pipeline to arrive at eight endotypes.

Precise contributions by important features for each molecular endotype were identified using SHapley Additive ExPlanations (SHAP) [26]. Feature contribution and SHAP value plots, dependence plots, and waterfall plots were carried out and visualized in Python v. 3.8.8 using the shap module v. 0.39.0.

Lupus Cell and Immune Score (LuCIS). To summarize the data generated by k-means clustering of the 32 GSVA enrichment scores, a composite score, the Lupus Cell and Immune Score (LuCIS), was developed using ridge-penalized logistic regression (RPLR).

The relationships between LuCIS, SLEDAI and anti-dsDNA titers were determined by linear regression carried out in GraphPad Prism v. 9.4.0 (673) with LuCIS as the dependent variable.

Normalization of Raw Data Files. Microarray data (Affymetrix and Illumina): Raw data of each transcriptomic dataset was downloaded from Gene Expression Omnibus (GEO). All statistical analysis was conducted using R v. 4.0.4 and relevant Bioconductor packages. To inspect raw data files for outliers, PCA plots were generated for each dataset. Datasets culled of outliers were cleaned of background noise and normalized using either Robust Multiarray Average (RMA), GCRMA, or normexp background correction (NEQC) based on the microarray platform resulting in log 2 transformed expression values into R expression set objects (e-sets). Analysis was conducted using normalized datasets prepared using both standard Affymetrix chip definition files (CDF), as well as custom made BrainArray CDFs. Illumina CDFs were used for the Illumina datasets.

RNA-sequencing (RNA-seq) data: Raw data files were downloaded from NCBI Sequence Read Archive (SRA) website using SRA toolkit (version 2.10) and converted to FASTQ files using fastq dump. Quality of the FASTQ files was checked using FASTQC software (version 0.11.9). Adapters and bad quality reads were trimmed using Trimmomatic software (Unix-based tool version 0.38). Good quality reads were aligned to the human reference genome (hg38) using STAR aligner (version 2.7). STAR-aligned reads were saved as .sam files and were converted to .bam files using sambamba (version 0.8). Read counts were summarized using the featureCounts function of the Subread R package (version 1.61). Count normalization and log transformation were carried out using the DESeq2 (version 1.32) R package.

Gene Set Variation Analysis (GSVA). GSVA (version 1.25.0) for R/Bioconductor is a non-parametric, unsupervised method for estimating the variation of pre-defined gene sets in samples of transcriptomic expression datasets [33]. GSVA works by transforming a gene matrix (gene-by-sample) into a gene set-by-sample matrix resulting in an enrichment score for each sample and pre-defined gene set. The inputs for the GSVA algorithm were a matrix of log 2 expression values and a collection of pre-defined input gene sets. Enrichment scores (GSVA scores) were calculated non-parametrically using a Kolmogorov-Smirnoff (KS)-like random walk statistic. The enrichment scores are the difference between the largest positive and negative random walk deviation from zero, respectively, for a given sample and gene set.

The enrichment scores take on values between −1 and 1, where 1 represents enrichment of every gene in a particular gene set among the samples analyzed compared to every other gene not included in the specified gene set, whereas −1 represents a relative lack of enrichment. Each gene in a gene set is given a rank based on expression values and the KS-like random walk statistic is calculated.

Out of 134 previously published gene sets and unpublished gene sets developed to represent immune cell signatures subsequently validated with flow cytometry [13, 34-37], 72 were used for GSVA after removing tissue-specific and redundant gene sets.

Derivation of 32 Molecular Features. Feature selection began with GSVA of 72 pre-defined gene sets, reduced from 134 starting gene sets, of samples from Datasets 3, 7-8, and 17 (Table 33). GSVA was run on each dataset separately [33]. Low intensity genes were filtered and only those with IQR>0 across all the samples were considered for analysis. GSVA was also carried out separately for two cohorts within each dataset: 1) lupus samples and healthy controls, and 2) active (SLEDAI >6) lupus samples and inactive (SLEDAI <6) lupus samples. For each of these two stratifications, GSVA enrichment scores from each dataset were concatenated, providing a sufficiently large cohort for feature extraction and stratification.

Various feature selection techniques were employed to remove the noise and select features that contributed the most to the prediction variable. The concatenated GSVA score matrices were used as inputs. The analysis was carried out as follows:
1) Two GSVA concatenated matrices of enrichment scores were referred to as Discovery Cohort 1 and Discovery Cohort 2. Discovery Cohort 1 was comprised of 1907 SLE samples and 73 non-SLE healthy controls from the four aforementioned datasets. Discovery Cohort 2 was comprised of 1657 active SLE samples and 250 inactive SLE samples from the four aforementioned datasets.
2) Feature refinement was carried out independently in each discovery cohort in Python v. 3.8.2 using scikit-leam (version 0.24.1) [38]. First, the data were checked for missing values; none were detected and subsequently removed. Second, the features were checked for low variance; none were identified and subsequently removed. Next, features were correlated with one another to check for and remove redundant features. Feature redundancy was assessed by computing Pearson correlations and plotting the results. Pairs of features with correlation coefficient r >0.9 were identified as highly correlated. Out of a pair of collinear features, the feature with the lowest correlation coefficient was removed. Nine features, or gene sets, were identified with r >0.9 and excluded: CD8T-NK-NKT, CD8 T Cells, Antigen Presentation, Mitochondrial Small Ribosome, Mitochondrial Transcription, Mitochondrial Large Ribosome, TCA Cycle, IG Chains, and Cell Cycle.
3) Feature importance, a property of ML models where a score is computed for each feature, was next assessed. The higher the score, the more important or relevant that feature is towards the output variable. Feature importance was used to identify the top features that can predict lupus samples from healthy controls, and the top features distinguishing active lupus from inactive lupus. Of the nine ML algorithms (logistic regression (LR), support vector machine (SVM), random forest (RF), k-nearest-neighbors (kNN), gradient boosting (GB), naïve Bayes (NB), decision trees (DTREE), adaptive boosting (ADB), and linear discriminant analysis (LDA)) tested in two independent binary classifications aiming to separate these groups, RF and SVM performed the best by evaluation of sensitivity, specificity, Cohen kappa score, f1-score, and accuracy. Gini importance, or mean decrease in impurity, represents feature importance in RF; meanwhile, for SVM, the permutation importance function was used to calculate the feature importance scores. With these metrics, the top 20 features of RF and SVM in each discovery cohort were combined and selected. The “IG Chains” gene set was re-added because of its biologic importance in lupus and other redundant features were removed for a final 32 features [13, 39]. Feature selection and ML were carried out in Python v. 3.8.8 using scikit-leam v. 0.24.1 and GSVA v 1.38.2 was carried out in R version 4.0.4 [33, 38, 40].

Binary Classification to Derive Features. Two independent binary classifications on the discovery cohorts were carried out in Python v. 3.8.2 using scikit-learn (version 0.24.1) [38]. Several linear, nonlinear, and ensemble ML algorithms were implemented to distinguish lupus from healthy, non-lupus controls and active lupus from inactive lupus. Since there were data imbalances in both discovery cohorts, subsampling without replacement was carried out by creating 20 different folds/subsets by randomly selecting 73 lupus samples to match with the minority class in Discovery Cohort 1 and by creating 7 different folds/subsets of 250 random active lupus samples to match with the minority class in Discovery Cohort 2. The data from each fold were split into 70% training and 30% validation, and ML classifiers were built on the training data and evaluated on the validation data. Feature importance scores were computed for RF and SVM classifiers. Average performance measures were calculated from all 20 folds of Discovery Cohort 1 and 7 folds of Discovery Cohort 2. Average gene importance scores of the respective folds were computed. ROC curves and PR curves were plotted using the matplotlib (version 3.3.4) Python library [41].

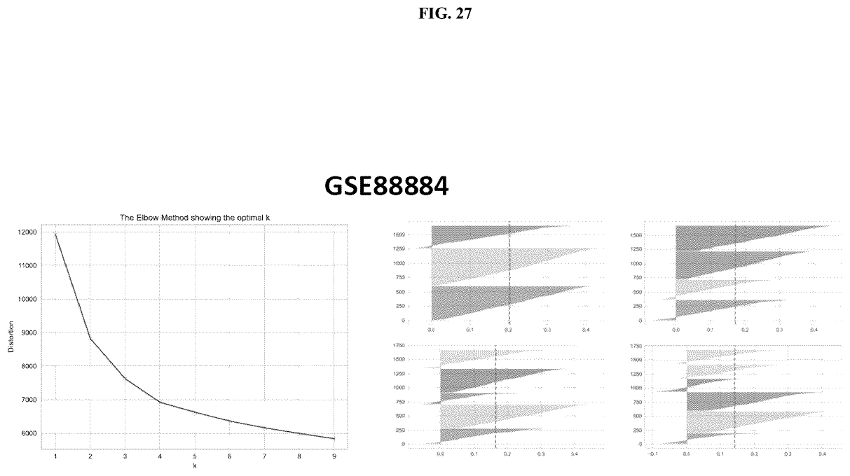

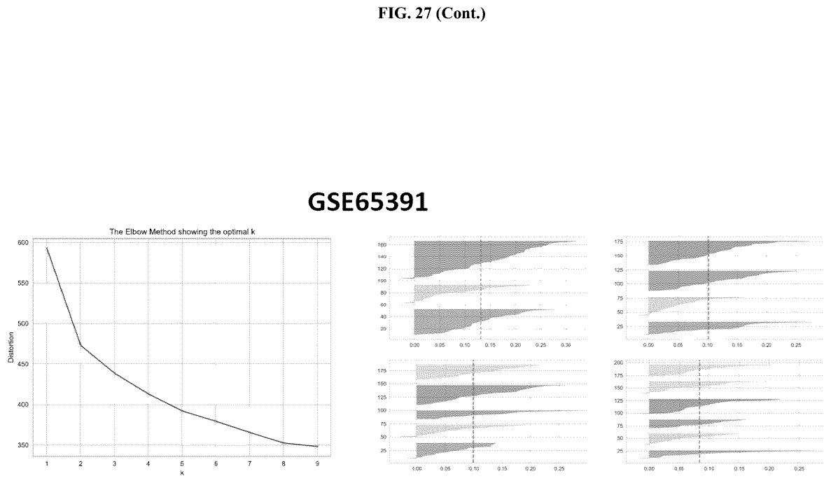

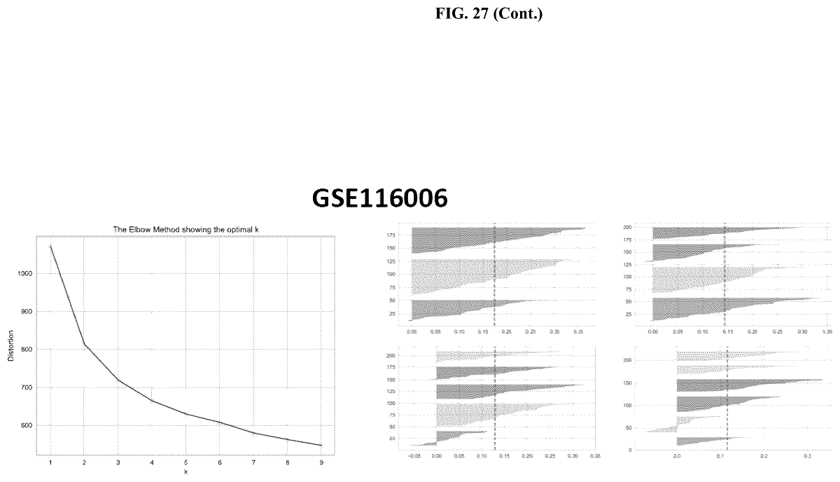

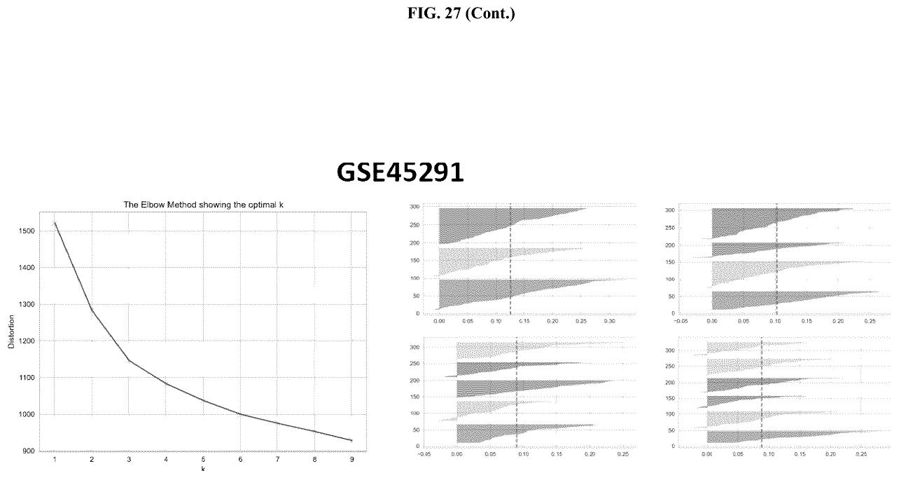

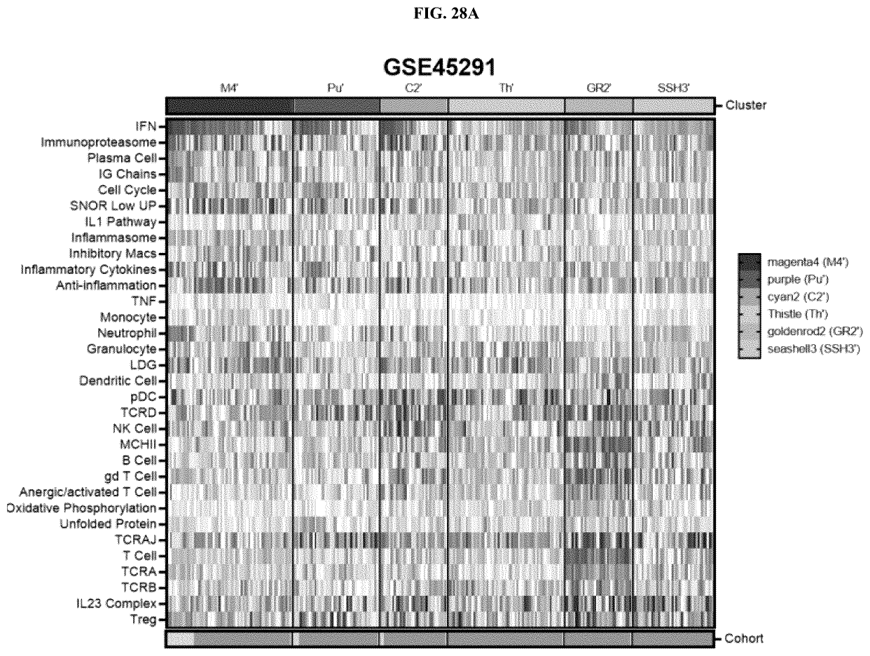

K-Means Clustering. Unsupervised algorithms like clustering were carried out to classify the lupus samples into subgroups/clusters of varying immunologic/inflammatory activity. Baseline GSVA enrichment scores of the 32 derived molecular signatures in two combined datasets, GSE88884 (ILL-1 and ILL-2), were used as input into five clustering algorithms: k-means, hierarchical, self-organizing maps, spectral, and Gaussian mixture modeling. These datasets were chosen for preliminary proof-of-concept because of the large number of samples and extensive associated clinical metadata. The resulting clusters from these methods were compared to one another by adjusted rand index and silhouette scores were computed for each method (Table 34). K-means clustering with the most stable clusters identified after 5000 iterations and minimal loss function was selected as the algorithm of choice based on having the highest silhouette score. The number of k clusters for each dataset was determined by elbow and silhouette plots (FIG. 27). Clusters were designated with a randomly generated color using the ‘grDevices’ color palette in R. Comparison of clusters from different datasets were compared by cosine similarity using the ‘lsa’ R package [42]. Cosine similarity >0.7 was considered highly similar.

K-means clustering was conducted using the scikit-learn (version 0.24.1) Python library [38]. The chosen parameters for the k-means algorithm were as follows: number of k clusters determined by elbow and silhouette methods for each dataset/cohort, method of initialization was set as k-means++, maximum number of iterations=300, and 5000 runs of the algorithm with a different centroid seed were set. The final clustering results were determined by the best output of the 5000 consecutive runs by the sum of squared distances of samples to their closest cluster center.

Gaussian Mixture Variational Autoencoder (GMVAE). GMVAEs are powerful generative models which feature a pair of connected networks: an encoder, which converts high-dimensional input data into a smaller and denser representation by introducing latent variables, and a decoder, which outputs the probability distribution of the data [43]. 20 clinical variables including ancestry, SLEDAI components, five autoantibody titers, and medication use from the lupus patients enrolled in ILLUMINATE-2 (GSE88884) were used as input for deep, unsupervised clustering by the GMVAE algorithm. The categorical clinical variables were binarized. GMVAE with back-propagation optimization identified six clusters.

Machine Learning of Final Endotypes. With the labels from GSVA and k-means clustering of 3,166 lupus samples into eight endotypes, the samples were then split into training and validation (Table 33, Datasets 7-8, 14, 16-17, n=2183) and test (Table 33, remaining datasets, n=983) sets. One-vs.-one and one-vs.-rest multi-class classifications with leave-one-out cross-validation were employed to predict sample membership into one of eight lupus endotypes. Training data (n=2183) were further split into 80% training and 20% validation data. Synthetic Minority Oversampling Technique (SMOTE) was applied on the training data to handle class imbalances [44]. RF, SVM, LR, and GB were employed in one-vs.-one and one-vs.-rest multi-class classification and extreme GB (XGB) was additionally employed in one-vs.-rest multi-class classification. The ML models were built on training data, optimized, if necessary, using validation data by fine-tuning parameters, and their performances evaluated on the test sets based on sensitivity, specificity, Cohen's kappa, f1-score, and accuracy. Non-lupus healthy controls were excluded from these analyses. ML was carried out in Python v. 3.8.8 using the scikit-learn (version 0.24.1) library. Receiver operation characteristic (ROC) curves and precision-recall (PR) curves were plotted using the matplotlib (version 3.3.4) Python library.

Lupus Cell and Immune Score (LuCIS). The GSVA enrichment scores of the 32 molecular features calculated for each lupus patient in the bookend clusters of GSE88884 ILL1 & ILL2 (i.e., the least abnormal endotype (indianred2) and the most abnormal endotype (slateblue3)) were input into a ridge regression algorithm with penalty. The resulting model provided the coefficients for LuCIS. To calculate a LuCIS value for each lupus patient, the GSVA enrichment scores for each module were binarized into zero or one based upon whether the GSVA score was less than zero or greater than zero, respectively. The binarized GSVA scores for each module were multiplied by the LuCIS coefficient in each patient and summed to create a raw LuCIS, which was normalized to a positive value. The ridge regression model was generated using glmnet from the ‘caret’ R package v. 6.0-92 [45].

GSVA Enrichment Score Imputation. Because of inter-platform differences (i.e., microarray chips are restricted by their specific libraries), some GSVA gene sets were not represented across all datasets. To overcome this limitation, GSVA enrichment scores were imputed for each patient based upon their known relationship to other represented gene sets.

Some GSVA scores were imputed on a dataset-by-dataset basis. In these cases, the gene set with the highest Pearson correlation was used to first estimate a coefficient describing the relationship between the gene set of interest (i.e., the one that requires score imputation) and the correlated gene set. For each of these gene sets of interest (i.e., IG chains, TCRA, TCRAJ, TCRB, TCRD, and Treg), the most correlated gene set was found by computing Pearson correlations between GSVA enrichment scores of all 32 features in GSE88884 ILL-1 and ILL-2.

These datasets were used as a reference (their GSVA scores were computed separately, then concatenated) due to the large number of samples and because all 32 gene sets were represented on the corresponding microarray chip. Next, a value of one was added to the GSVA scores of the “highest correlated modules” and the “modules of interest” so that there would not be any negative GSVA scores. Then, for each sample, the transformed score of the “module of interest” was divided by the transformed score of the “highest correlated module.” The mean of these calculations across the patients is the coefficient describing the relationship between the two modules.

In the dataset requiring imputation, a value of one was added to the GSVA scores of the “highest correlated module,” multiplied by the appropriate coefficient, and then a value of one was subtracted to arrive at the GSVA enrichment score of the gene set of interest.

Binary Classification to Characterize Individual Endotypes from Normal and SHAP. Seven individual binary ML classifications were performed comparing the seven more transcriptionally abnormal molecular endotypes with the least abnormal endotype using Python v. 3.8.2 using scikit-leam (version 0.24.1) [38]. Similar to the multi-class classification methodology, training data (Table 33, Datasets 7-8, 14, 16-17) was divided into training (80%) and validation (20%) sets for which classifiers were optimized if necessary and performance metrics evaluated on unseen test data (Table 33, remaining 12 datasets). For each classification, the top 15 contributors of each abnormal endotype were determined using SHAP. SHAP dependence plots were generated to illustrate the impact of each feature on the final model outcome and its interaction with another feature. SHAP waterfall plots were also generated for individual samples to visualize and deconvolute the mathematical contribution of each feature to the overall SHAP value. SHAP bar plots were additionally generated to summarize the information from the waterfall plots across all samples.

### Results

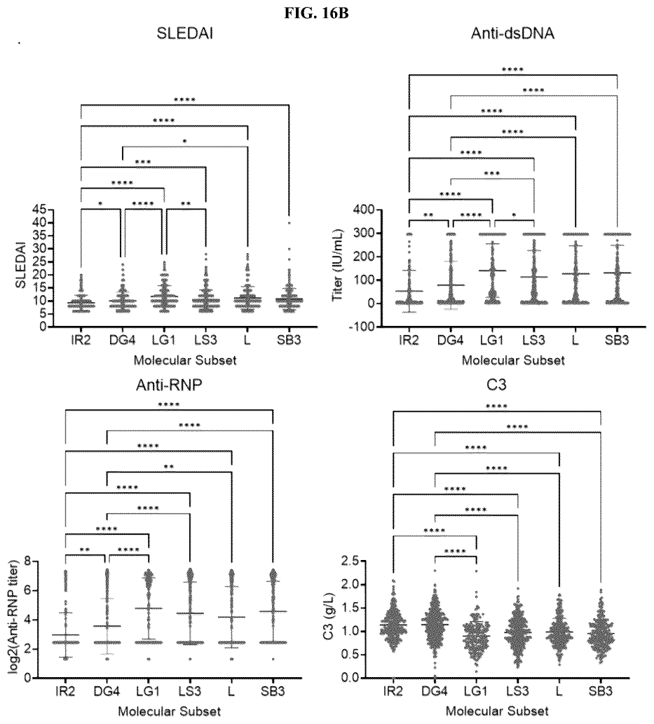

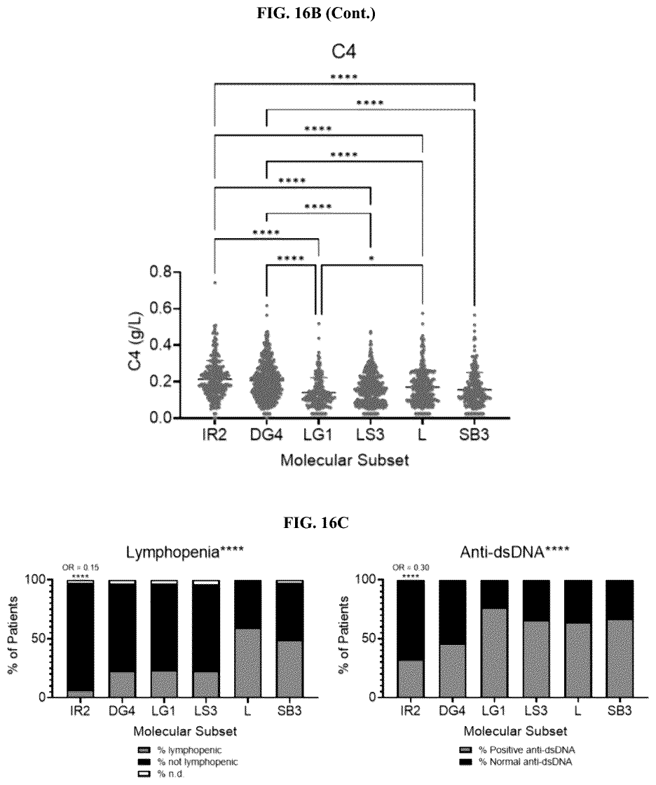

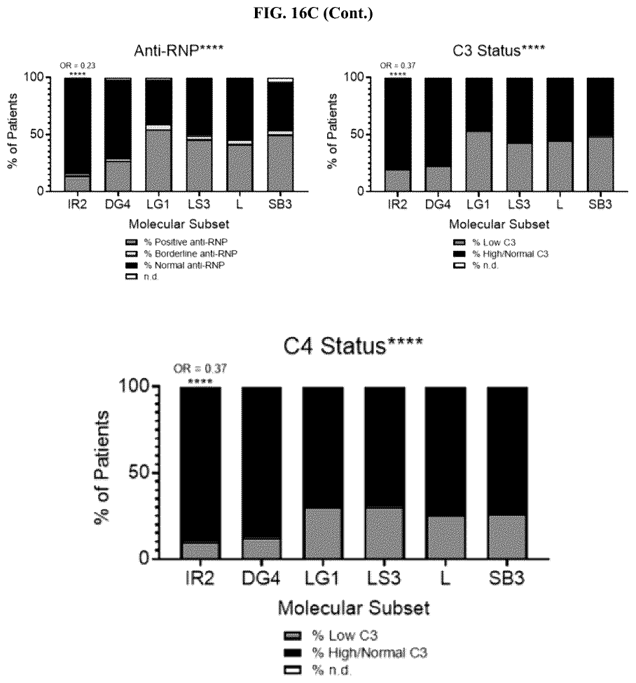

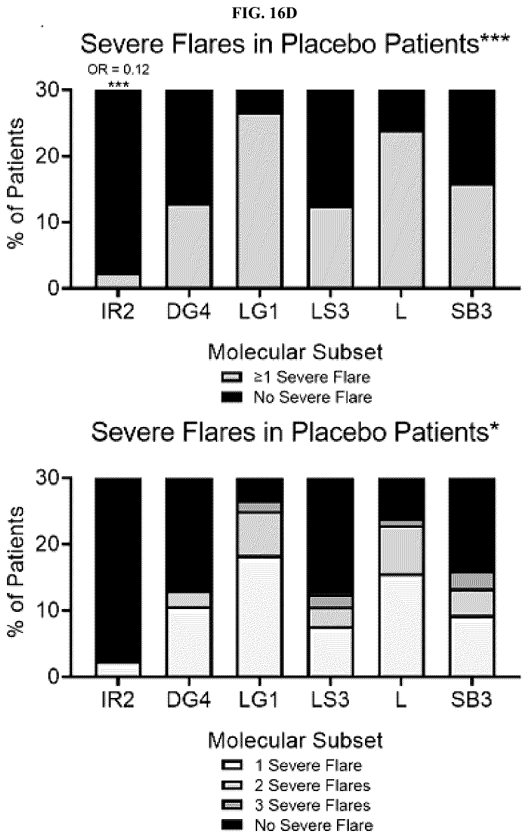

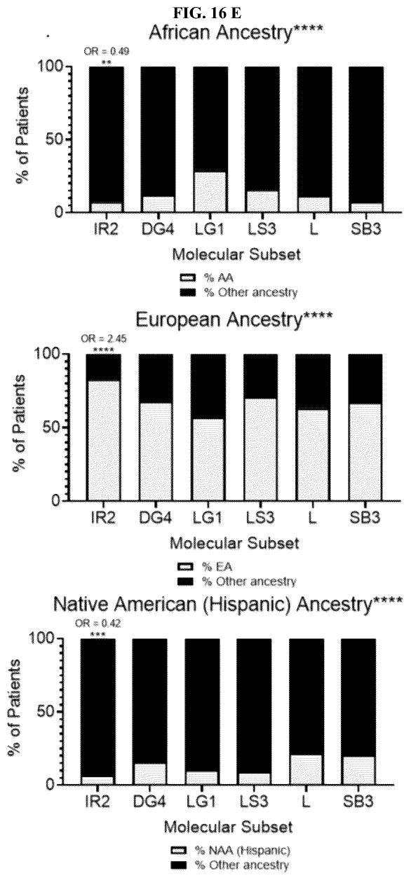

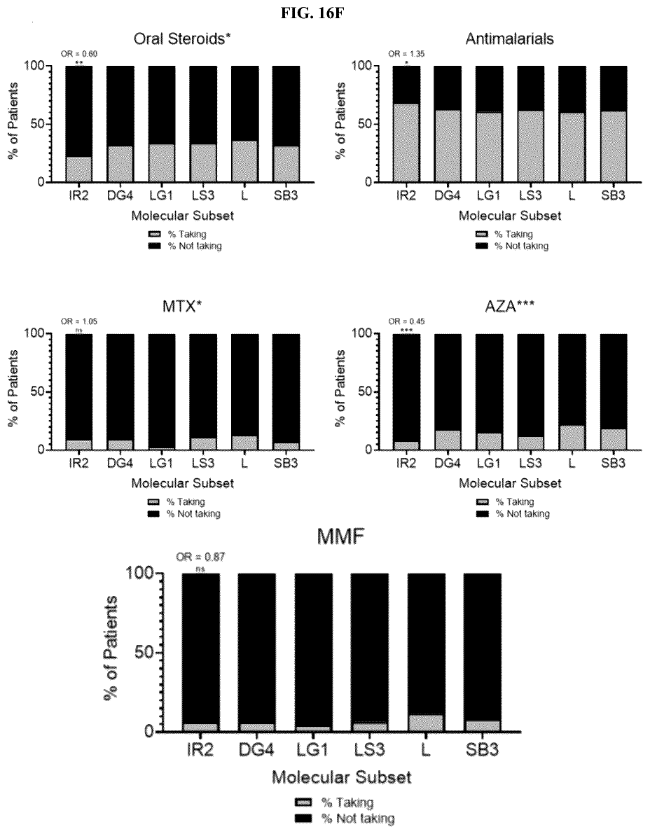

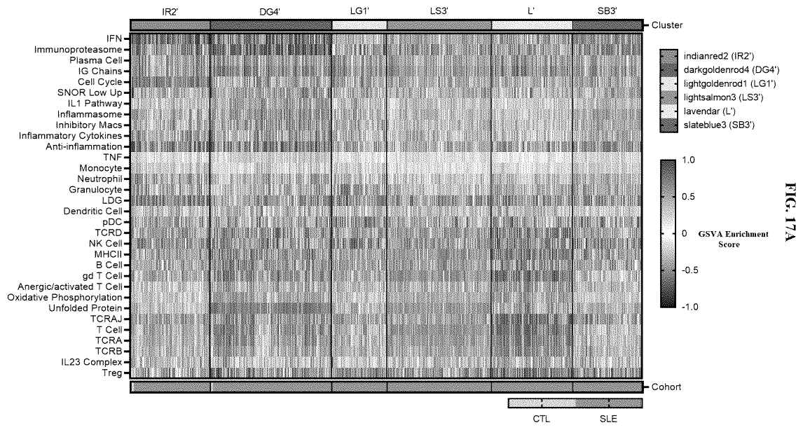

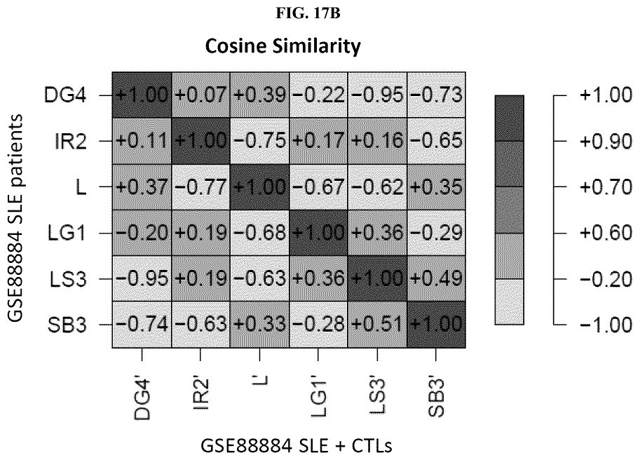

Molecular Endotypes in a Prototypic Dataset (GSE88884). Six molecular subsets (endotypes) within 1,620 active, female lupus patients were identified in the combined datasets from the Illuminate clinical trials (GSE88884) by k-means clustering of GSVA enrichment scores of 32 immune cell/inflammatory pathway gene sets (FIG. 16A). Notably, the endotype designated by the color indianred2 (IR2) manifested the least number of abnormally enriched gene modules (hereafter referred to as features), whereas the endotype designated slateblue3 (SB3) had the greatest number of abnormally enriched features, with other clusters arrayed between. Abnormally enriched transcriptional profiles were based upon reference to non-lupus controls; that is, when lupus patients and controls were re-clustered together, the endotype containing the greatest number of control samples was designated as the “least abnormally enriched” and transcriptional profiles deviating from this reference were characterized as increasingly more perturbed (FIG. 17).

Despite all patients having SLEDAI >6, analysis of the associated metadata revealed significant differences in SLEDAI, autoantibody titers, lymphopenia and serum complement levels, with IR2 having the lowest SLEDAI, lowest antibody titers and highest complement levels, whereas endotypes lightsalmon3 (LS3), lightgoldenrod1 (LGT), lavender (L), and SB3 manifested more abnormal clinical characteristics (FIG. 16B-C). By these systemic measures, molecular subset IR2 exhibited the lowest disease activity. On the other hand, LGT exhibited the most disease activity, followed closely by SB3. Endotypes associated with more disease activity were characterized by various combinations of enrichment of features for PCs, MCs, neutrophils, inflammatory cytokines, and lymphopenia (FIG. 16A). Patients of each ancestry were noted in each subset, though AA lupus patients were most enriched in LGT and least enriched in IR2.

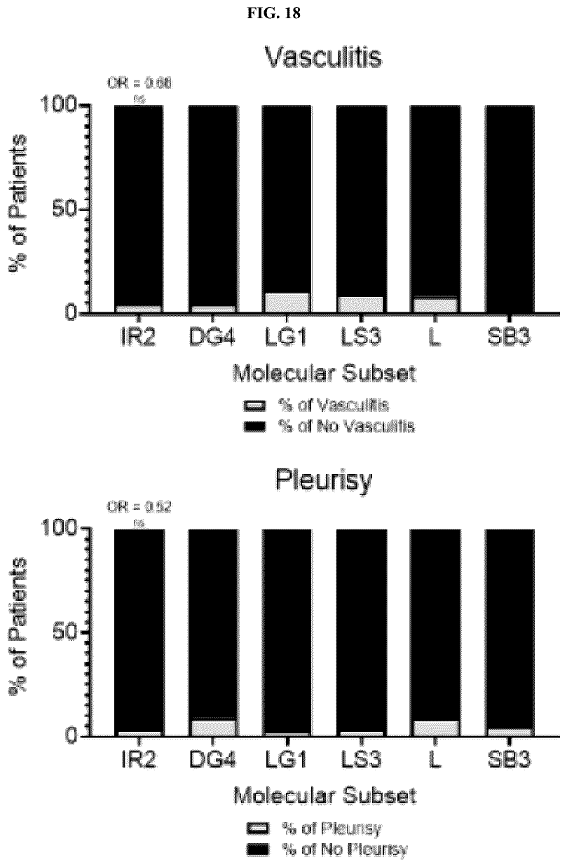

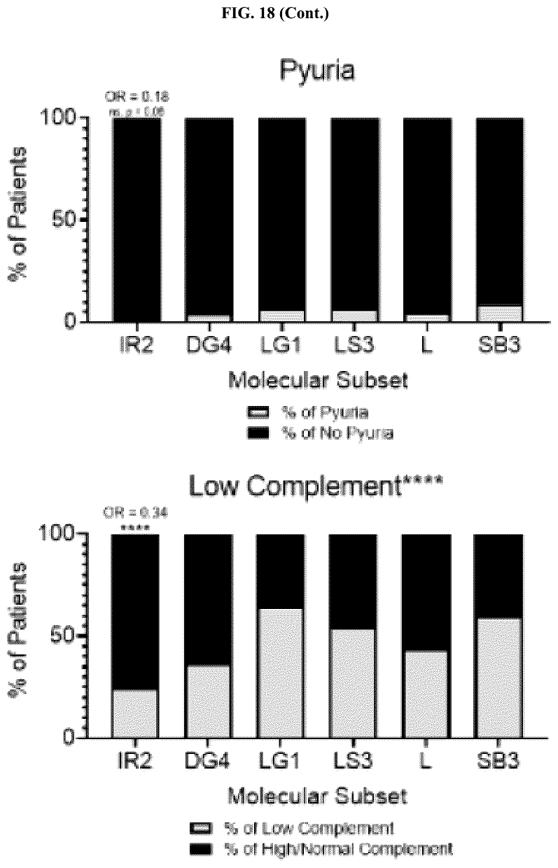

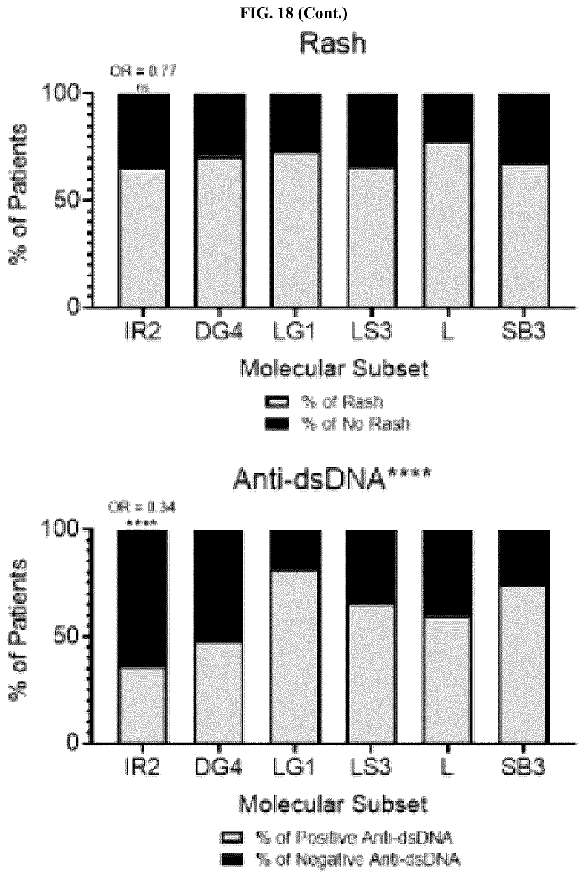

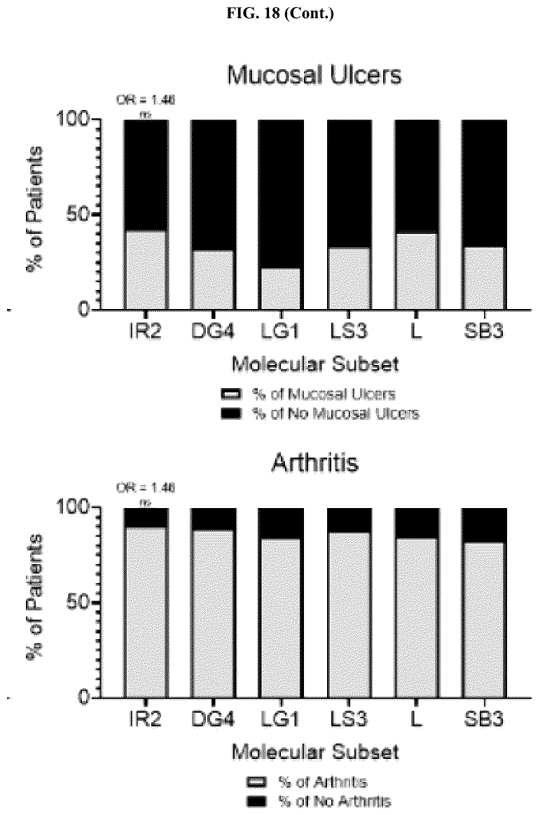

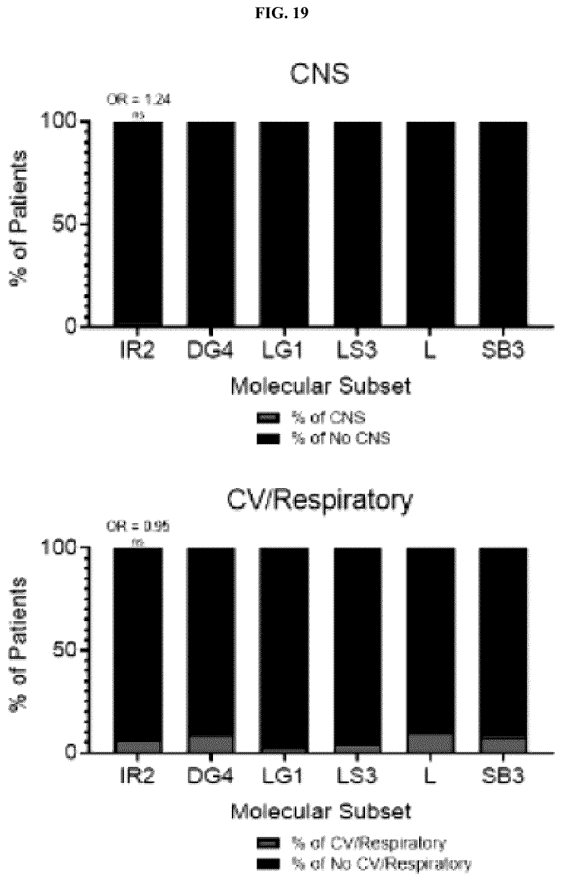

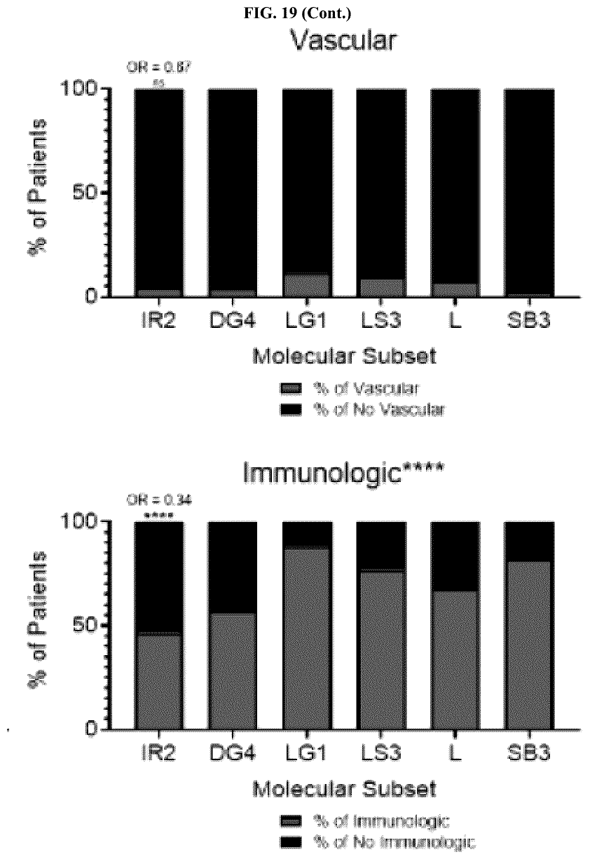

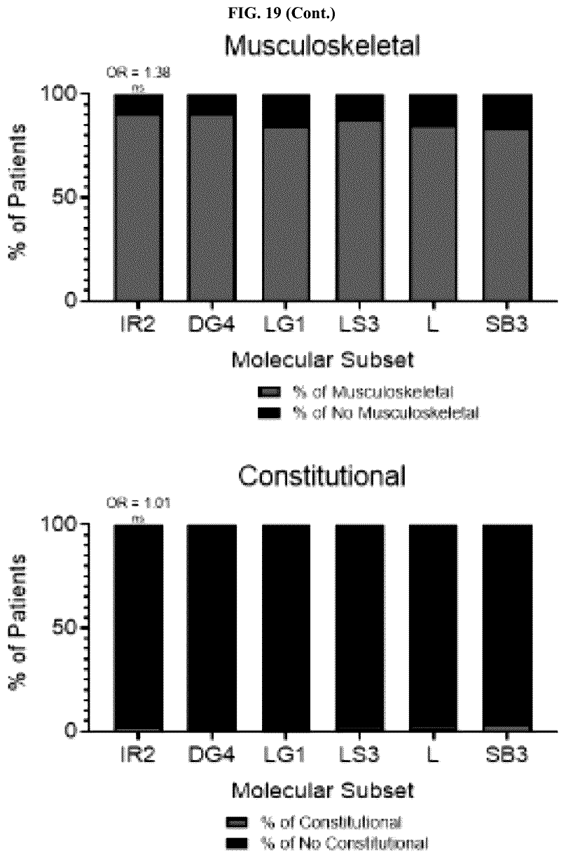

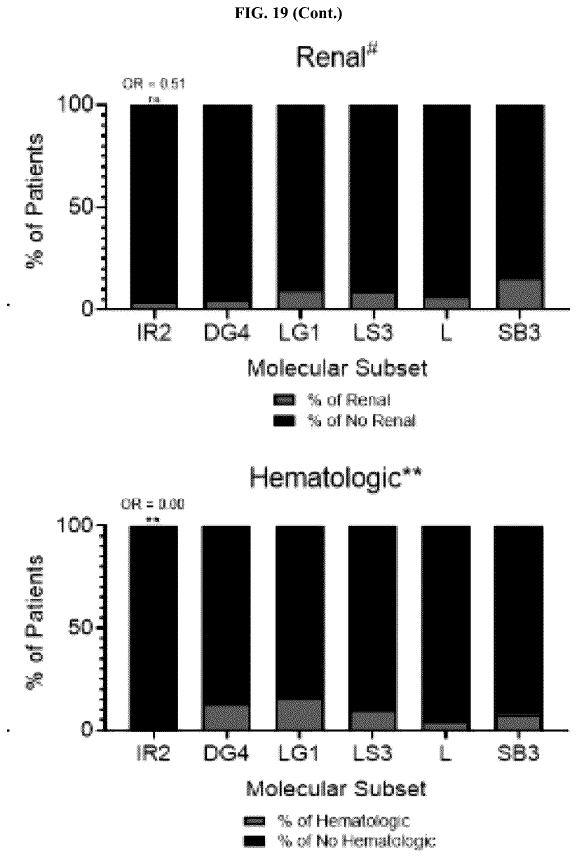

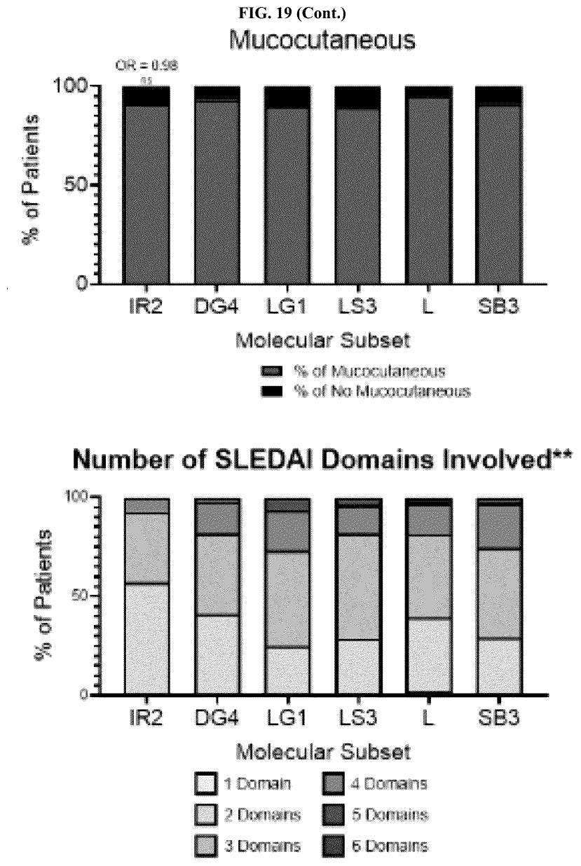

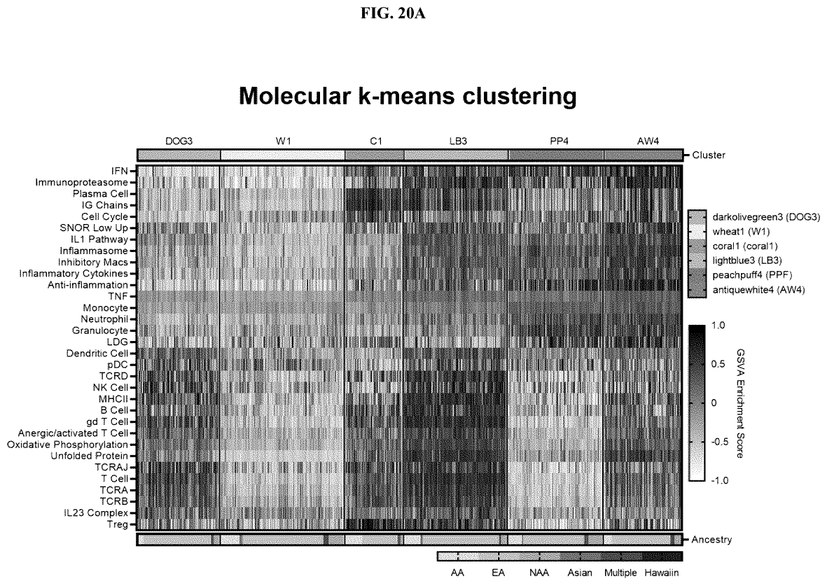

Of note, the patients in IR2 receiving standard of care (SoC) had a lower frequency of severe flares over the subsequent 52 weeks as compared to the patients in other subsets (OR=0.116, p=0.00041, FIG. 16D, Table 35). Significant relationships between endotype membership and ancestry (African, European, or Hispanic) (FIG. 16E) and medication use (oral steroids, azathioprine, or methotrexate) were identified (FIG. 16F). Few other clinical characteristics significantly differed among endotypes except for differences in the involvement of renal, immunologic, and hematologic organ systems, and the overall number of SLEDAI domains involved (FIG. 18-19).

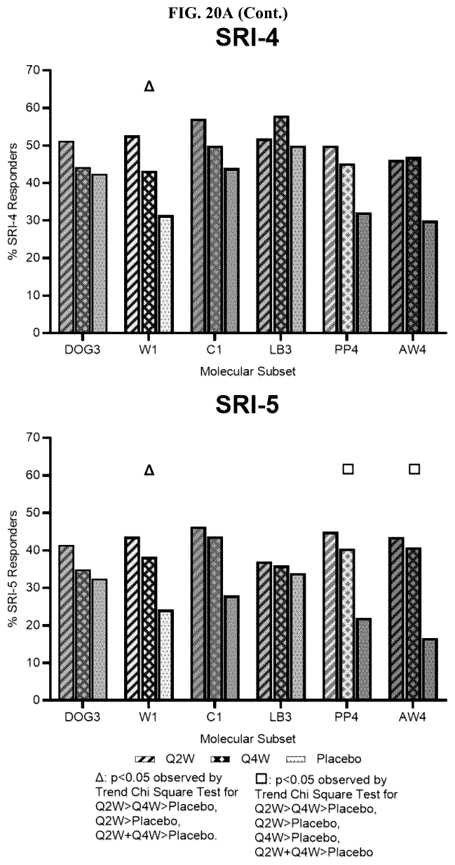

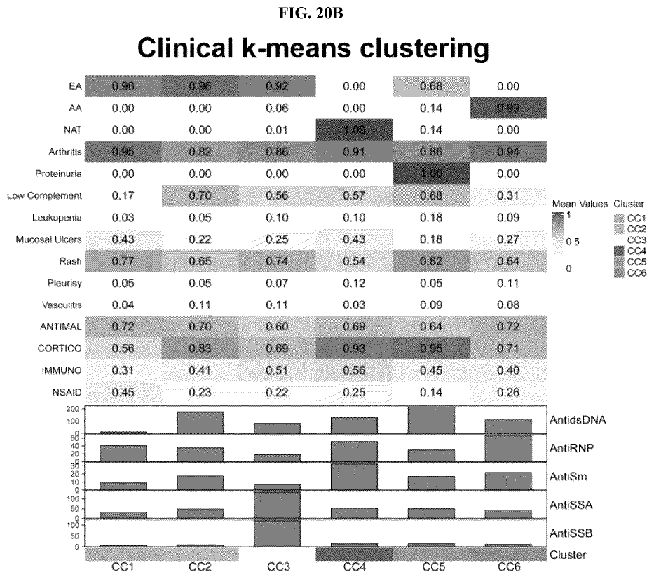

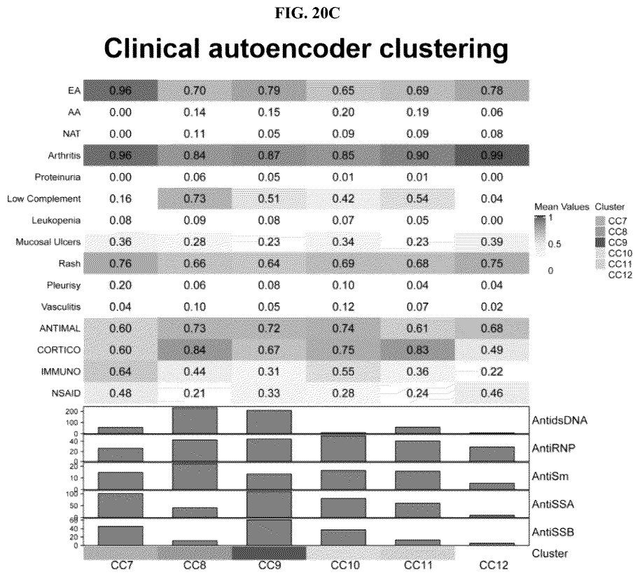

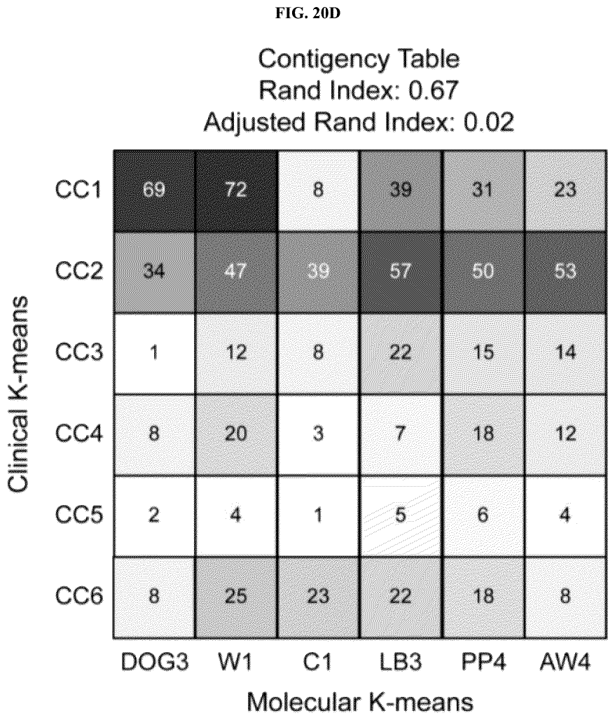

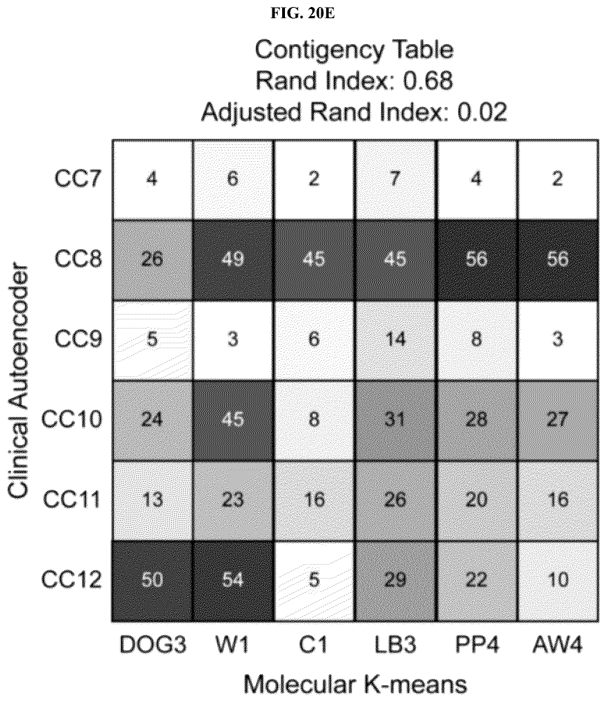

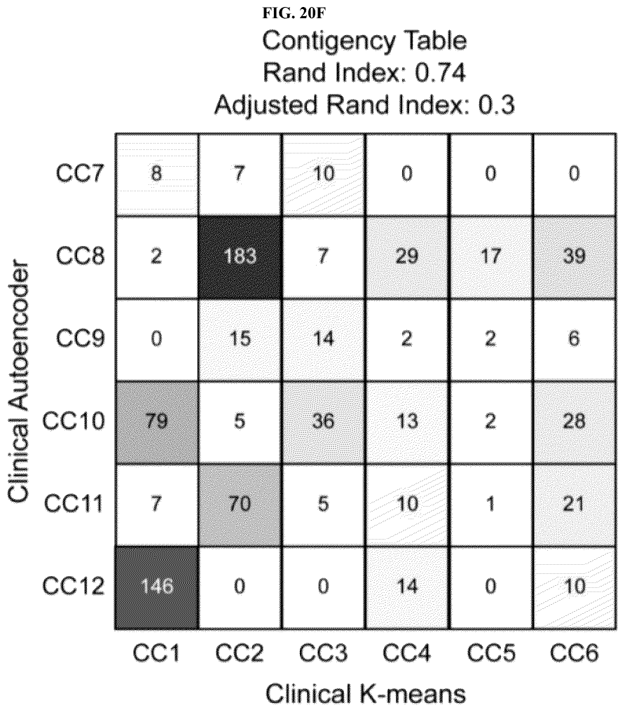

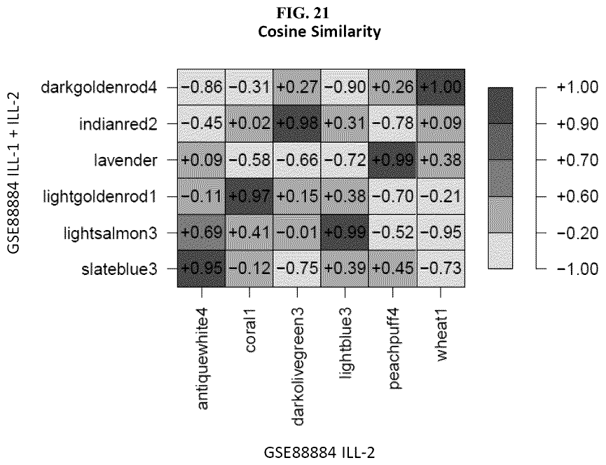

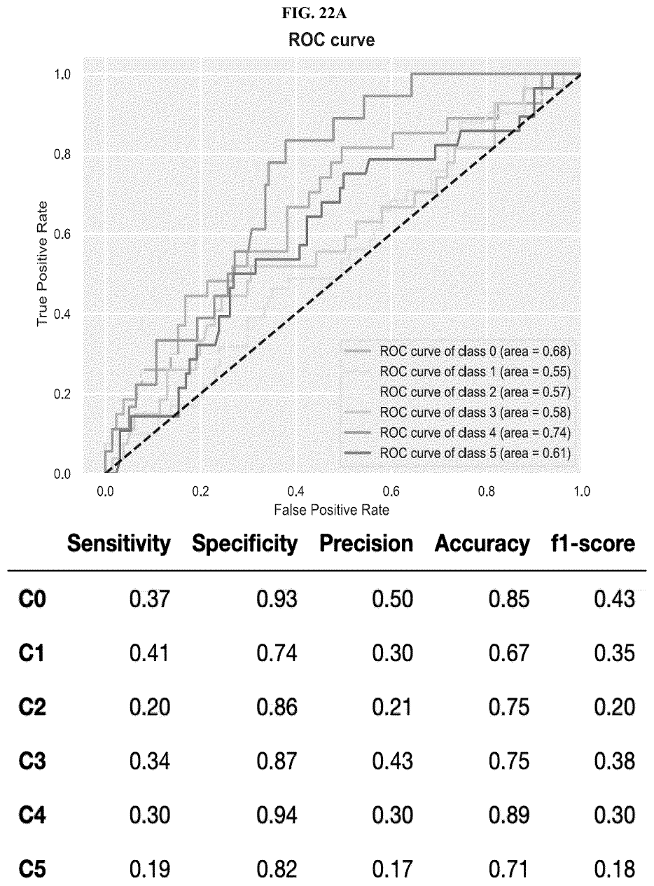

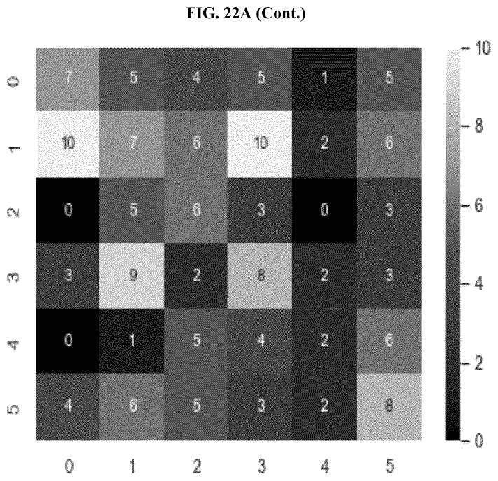

To further explore the clinical utility of our molecular endotyping, we applied the k-means clustering pipeline to the patients belonging to the successful Illuminate trial, GSE88884 ILL-2 [27], and identified six endotypes similar to those found in the combined trial datasets (FIG. 20A, FIG. 21). We examined clinical response to the investigational product, tabalumab, by two metrics: SRI-5, used in the trial, and the more standard SRI-4 [28], among these endotypes. We identified three responsive groups by SRI-5 (AW4, W1, and PP4) and one responsive group by SRI-4 (WI). Subsets Wheat1 (W1), Peachpuff4 (PP4), and Antiquewhite4 (AW4) were shown to have an increased response to the drug as compared to placebo. Of note, the endotype with the least immunologic activity (DOG3) was not responsive by either metric. By cosine similarity, Wheat1 (W1) is similar to subset B, Peachpuff4 (PP4) is similar to subsets F and H, and Antiquewhite4 (AW4) is similar to subset G. Thereby, in patients similar to those found in Subset B, F, G and H, patients were shown to be more likely to respond to tabalumab, and drugs that target BAFF, such as belimumab could be considered.

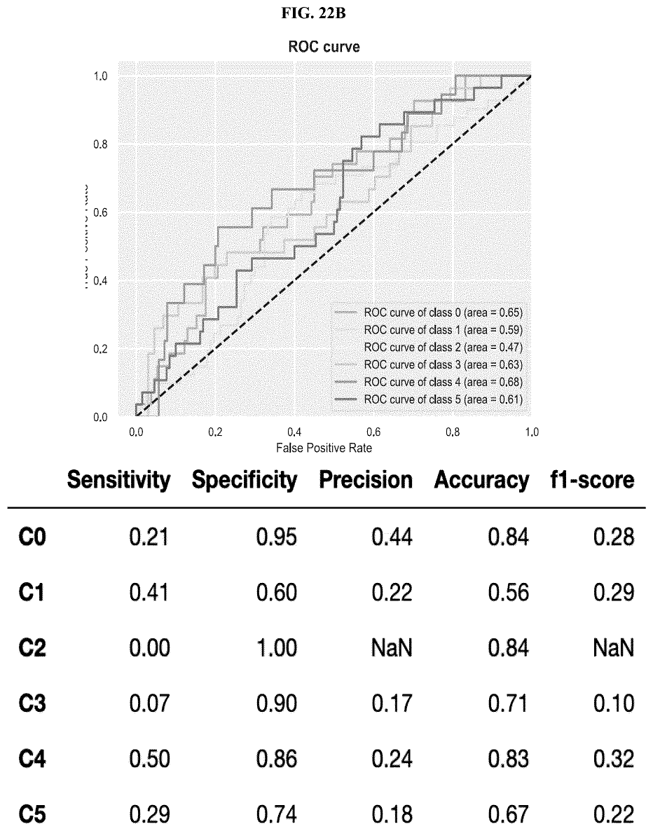

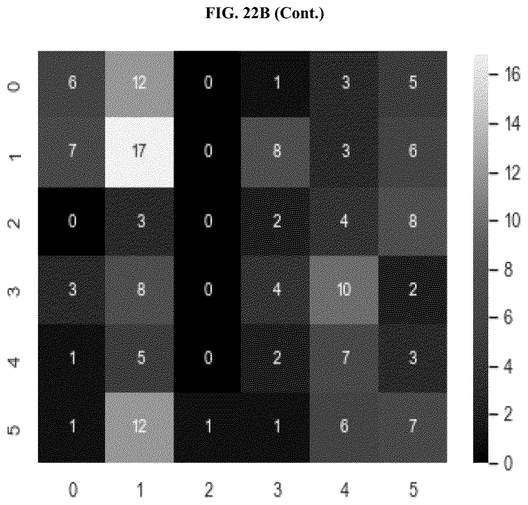

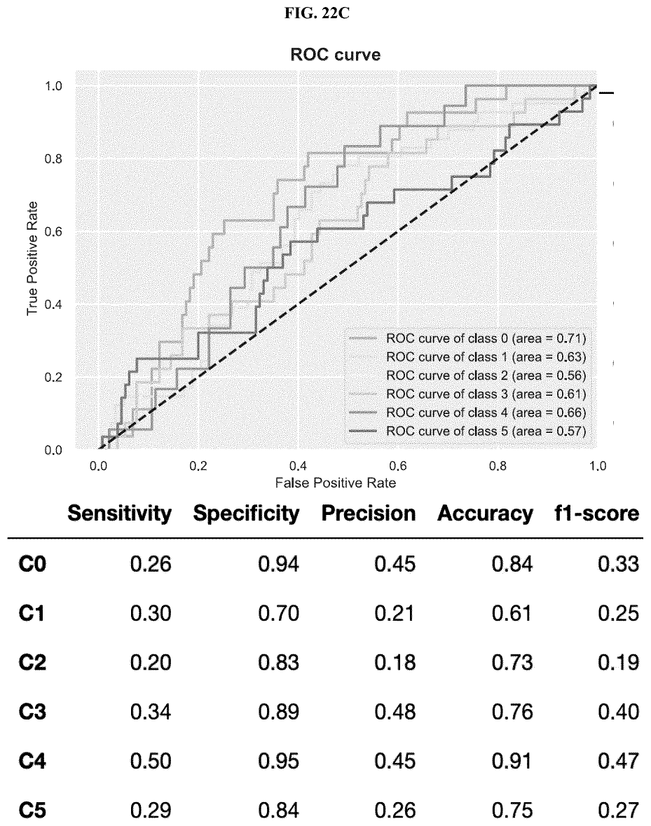

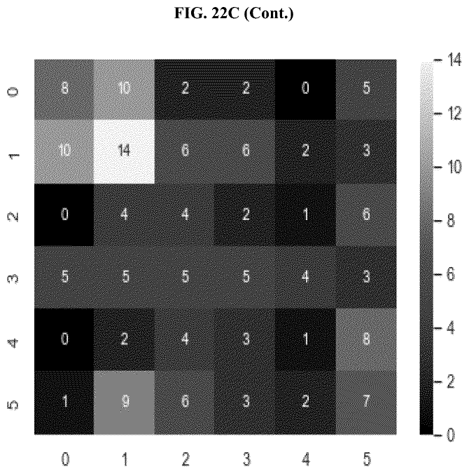

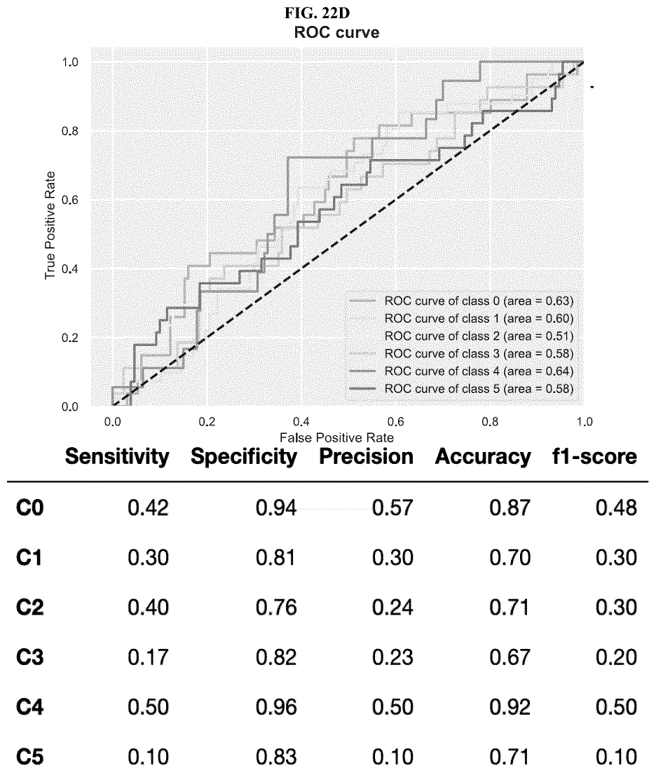

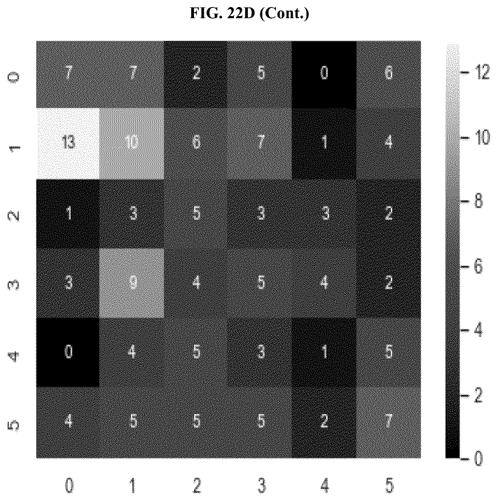

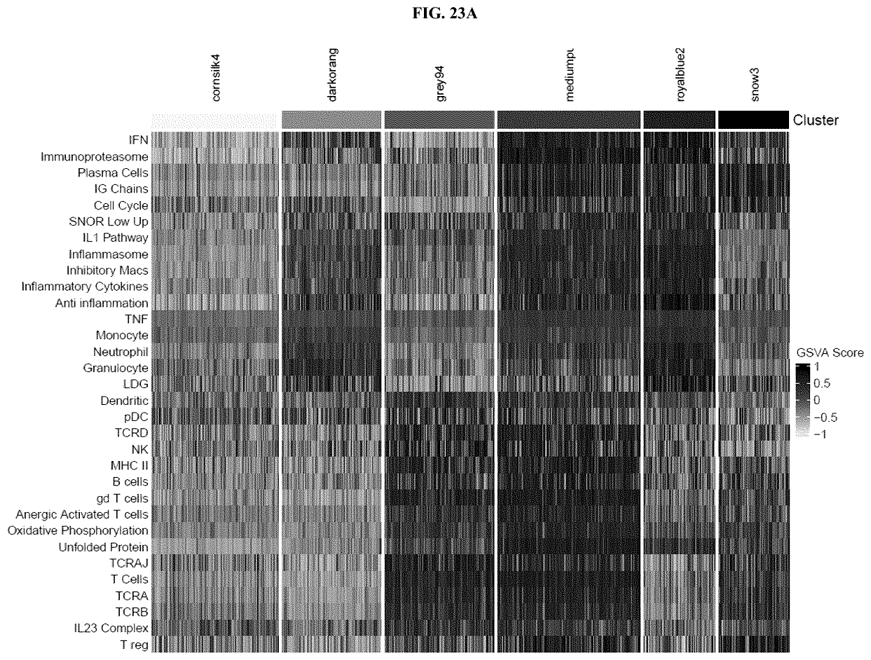

To determine whether clinical characteristics alone could identify the endotypes, we employed the clustering pipeline on ILL-2 lupus patient clinical metadata. With k-means, six subsets based on clinical features alone were identified (FIG. 20B). Another six subsets were identified by separate employment of a variational autoencoder to determine whether a deep learning algorithm would alternatively be able to identify the molecular subsets (FIG. 20C). By adjusted rand index, the clinically determined subsets were significantly different from the molecular subsets (FIG. 20D-E) and from each other (FIG. 20F). The clinical k-means subsets were largely dictated by ancestry (FIG. 20B), whereas the clinical autoencoder subsets were ancestrally heterogeneous (FIG. 20C). Finally, we employed four one-vs.-one ML classifiers using the same clinical data as features to predict molecular endotype memberships (FIGS. 22A-D). Performance was suboptimal, with a mean RF classifier precision of 32%, further indicating that clinical characteristics are insufficient features to identify the molecular endotypes.

K-means clustering is a partition algorithm with the advantage of placing each sample in a distinct cluster. We were interested to understand how removal of specific populations of patients with characteristics demonstrated to change gene expression, and smaller datasets with inactive lupus patients, would affect the k-means clustering. Because we previously found that ancestry and SoC therapies, such as mycophenolate, can significantly influence gene expression [13], we sought to determine whether similar endotypes could be found in lupus patients of self-identified European, African, or NAA/Hispanic ancestries and in patients with or without corticosteroid and/or mycophenolate treatment.

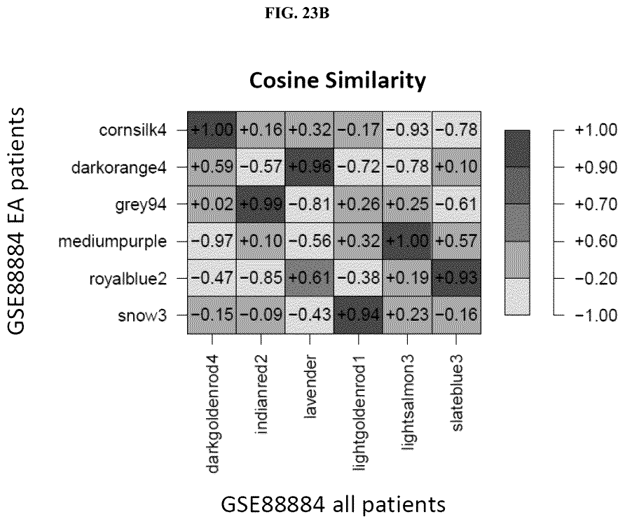

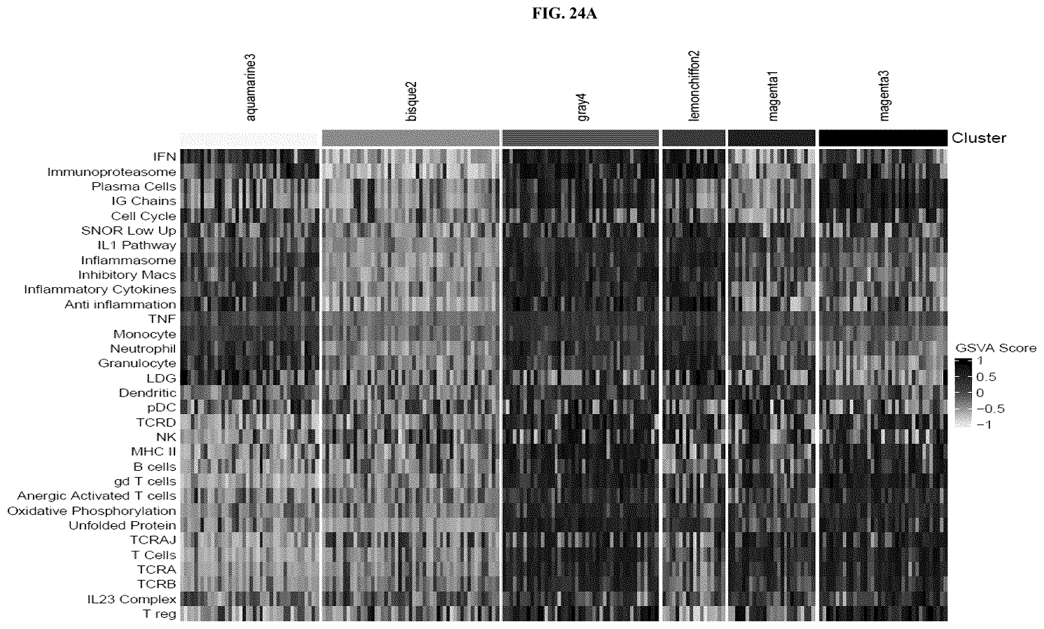

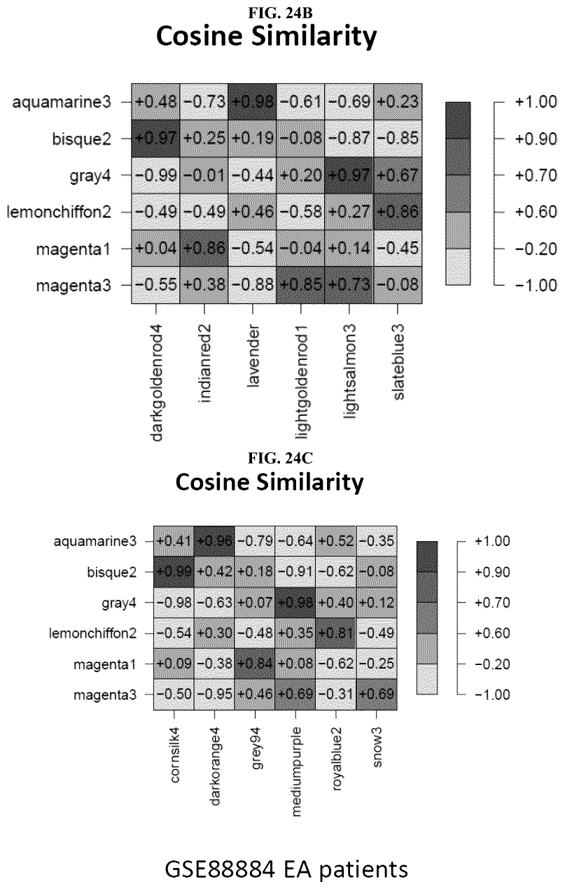

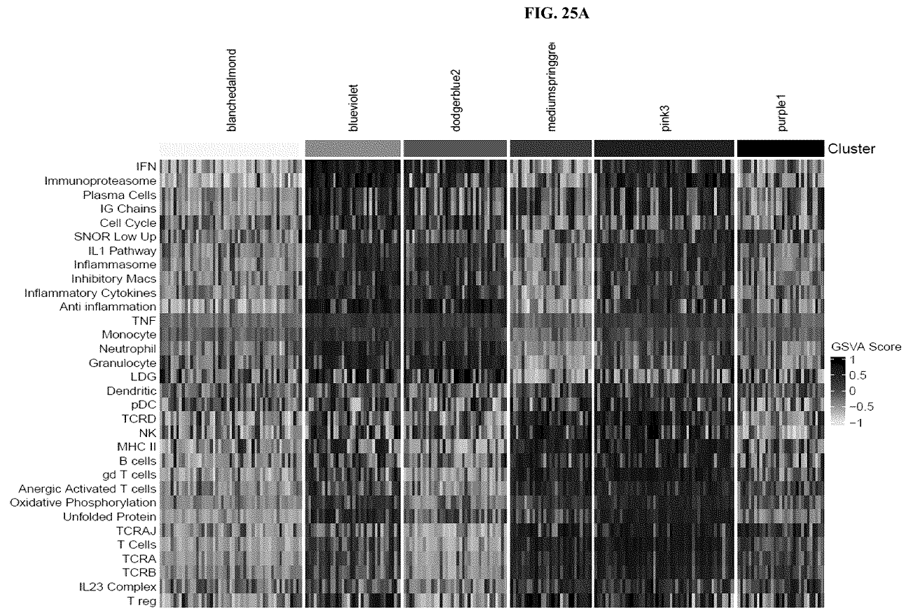

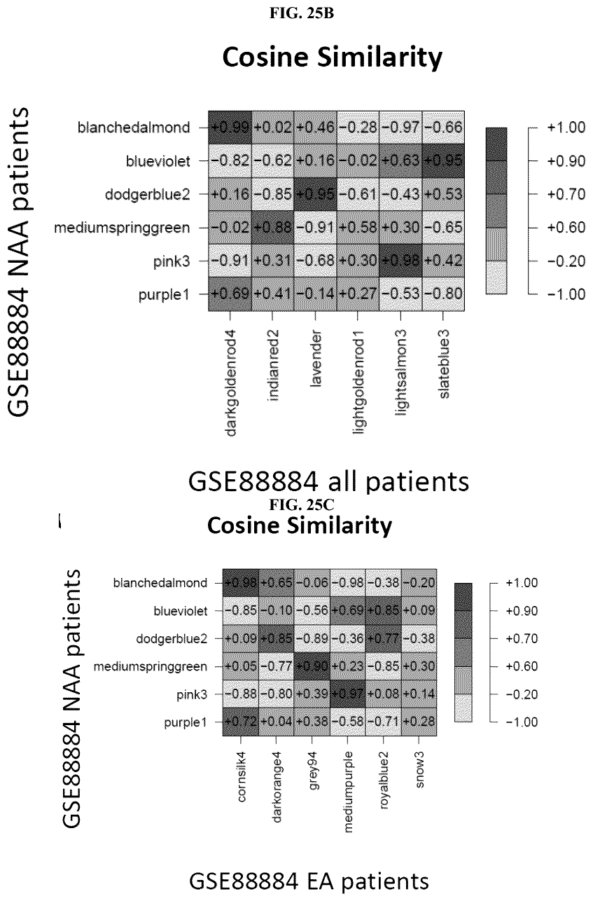

When only EA patients were clustered (n=1118), the same six endotypes as the full cohort were represented (FIG. 23). Similar results were seen in the AA population (n=216, FIG. 24), though subtle differences existed in endotype formation in the PC-enriched AA patients compared to EA patients. Endotype magenta3, highly enriched in PCs but depleted in LDGs and neutrophils, was formed from the AA patients comprising LG1 and LS3 from the full cohort (FIG. 16A); magenta3 was similar to snow3 and medium purple in EA patients, both enriched in PCs, but differentiating in inflammatory pathways/cell types, NK cells, and other features (FIG. 23). When only NAA/Hispanic patients were clustered (n=232), five out of six endotypes from the full cohort were identified and a new endotype, purple1, emerged (FIG. 25). Thus, different feature enrichment among ancestral cohorts contributed to slight differences in endotype identification.

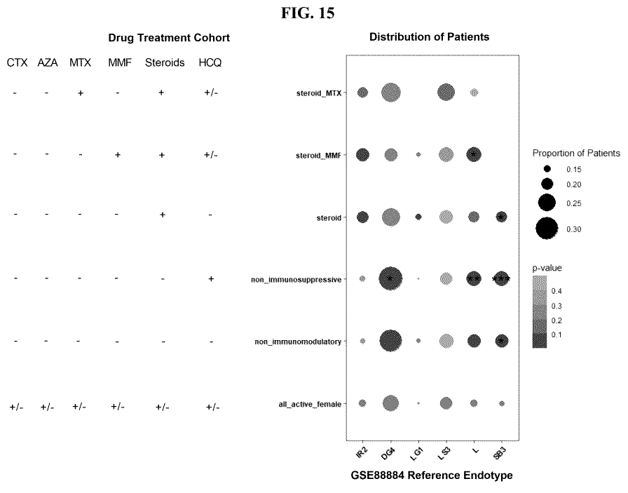

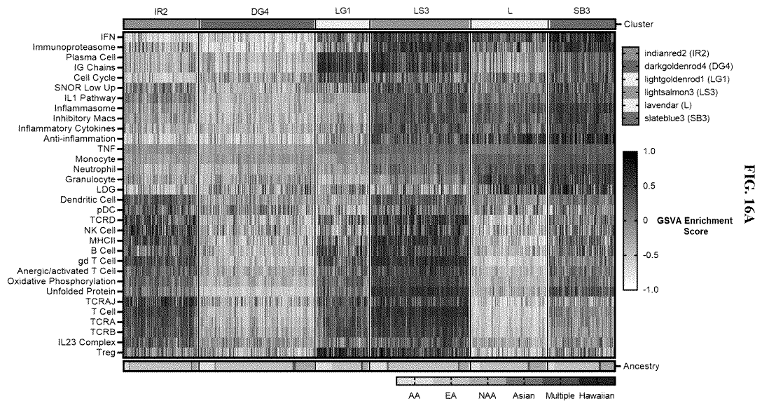

We repeated these analyses among patients stratified by immunomodulatory or immunosuppressive agents at baseline and compared distributions of patients to the endotypes in the full prototypic cohort to identify which endotypes were maintained (cosine similarity >0.7, FIG. 15). Treatment with mycophenolate or methotrexate in combination with steroids appeared to deplete the most perturbed endotype, SB3, whereas this endotype was expanded in groups treated without SoC SLE therapies, without immunosuppressive agents, and without steroids alone. Methotrexate also appeared to deplete endotype LG1, which exhibited the highest disease activity. The distribution of patients in the least perturbed endotype, IR2, also increased with treatment by steroids and immunosuppressive agents.

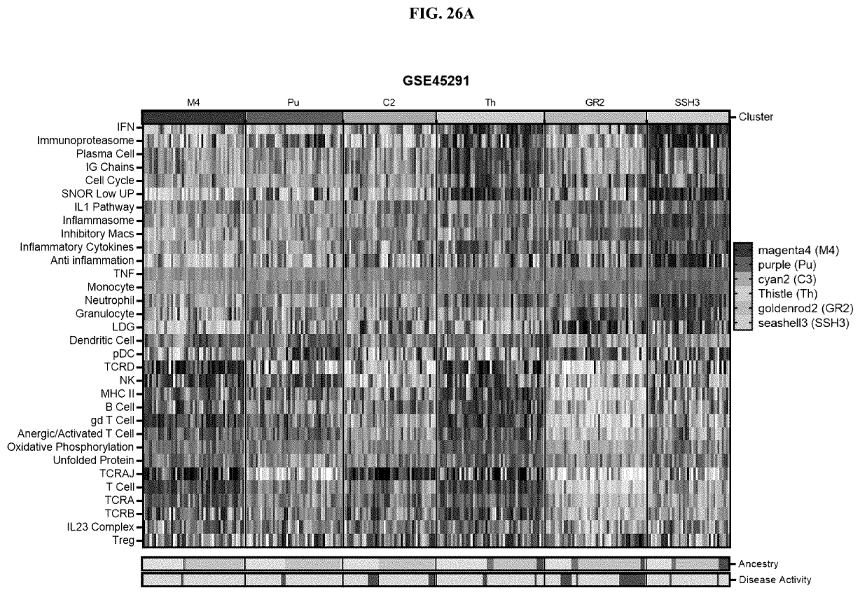

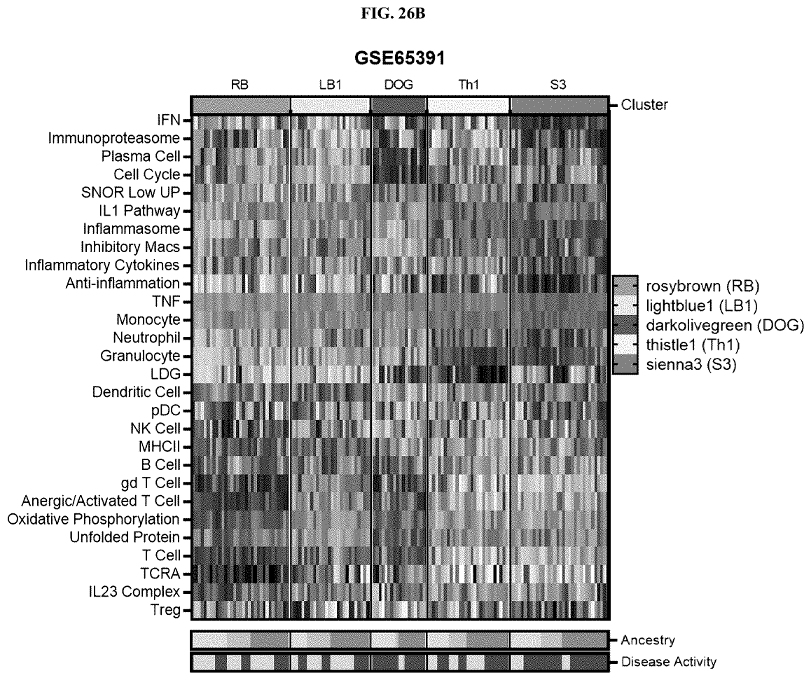

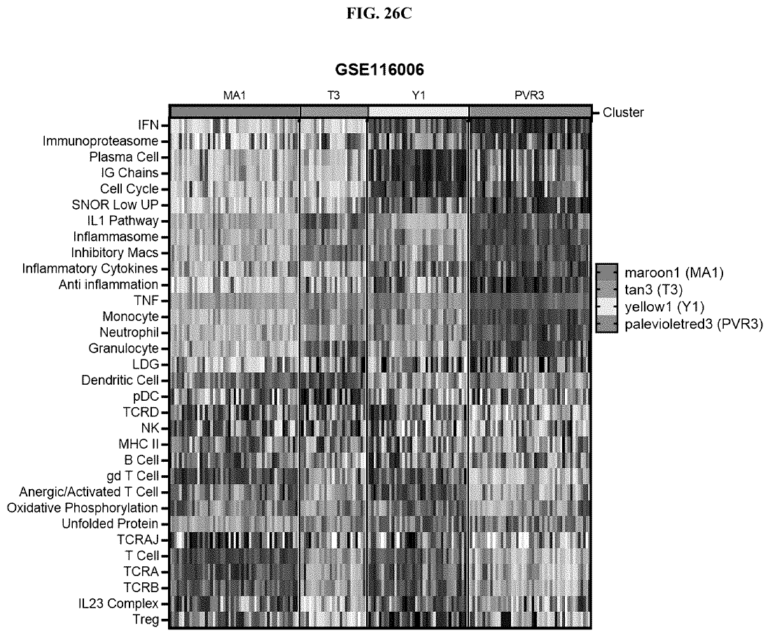

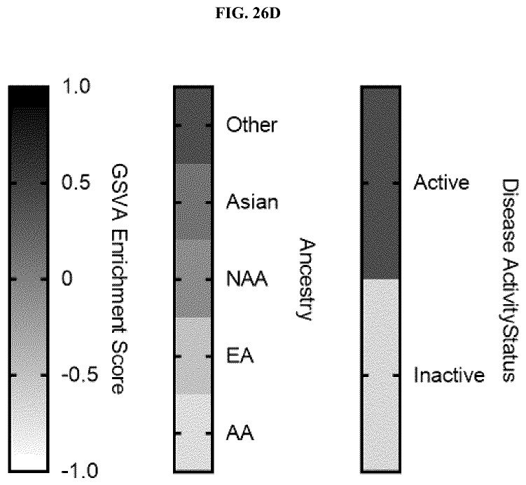

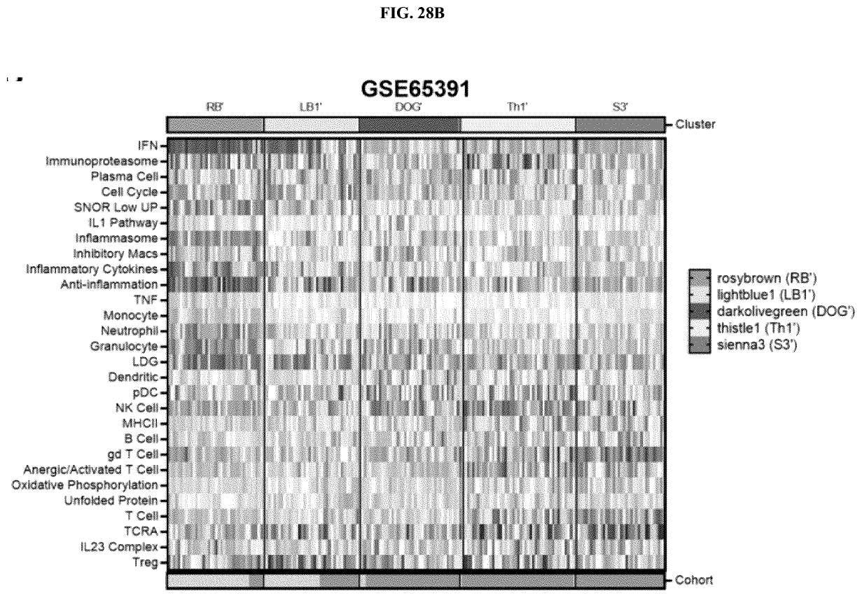

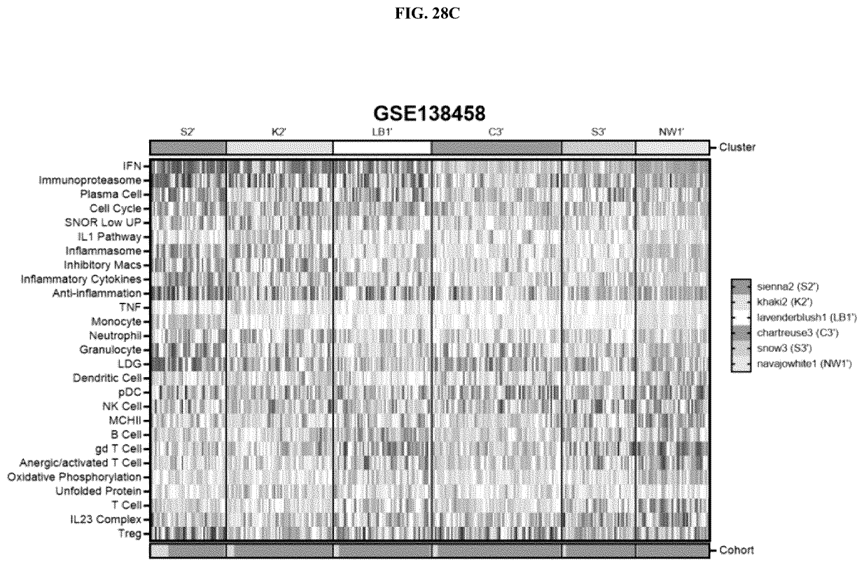

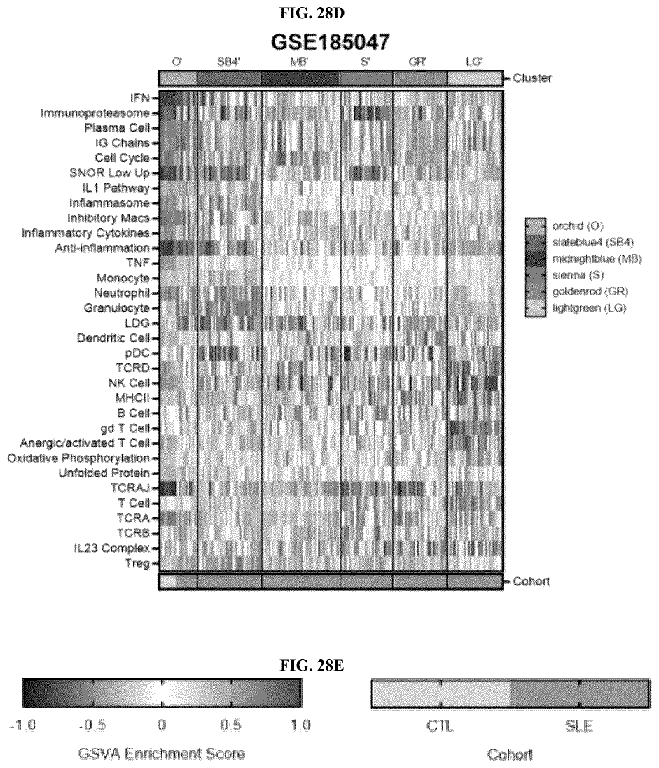

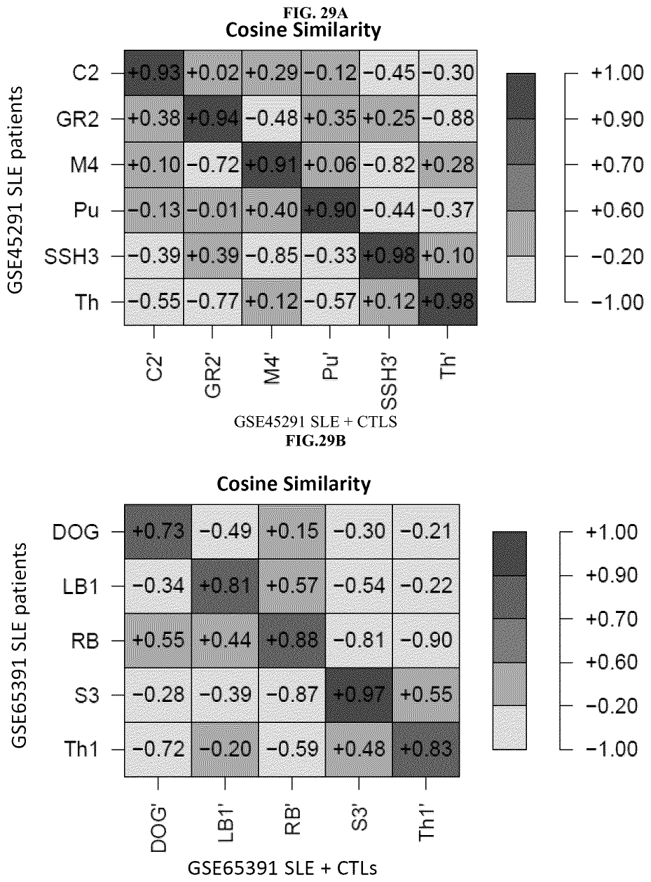

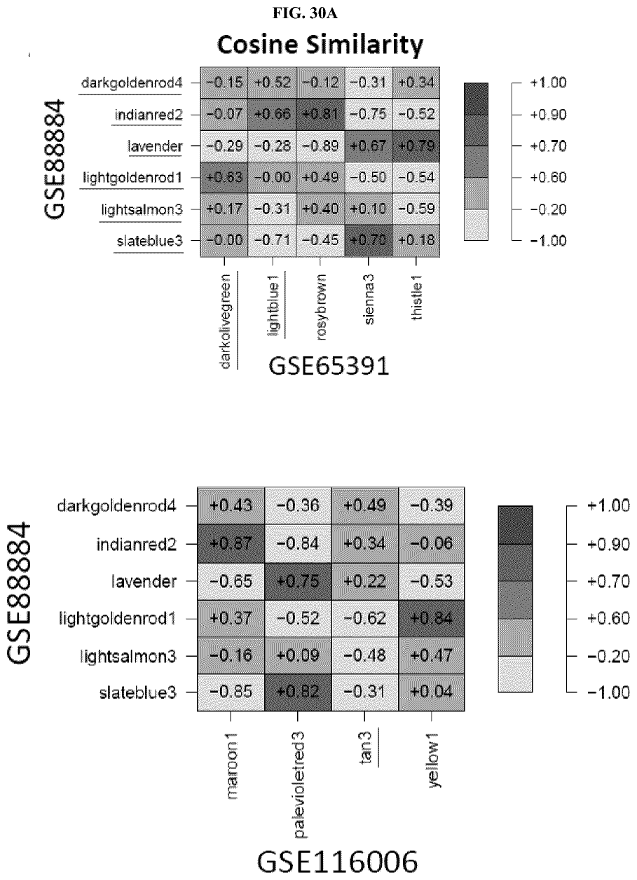

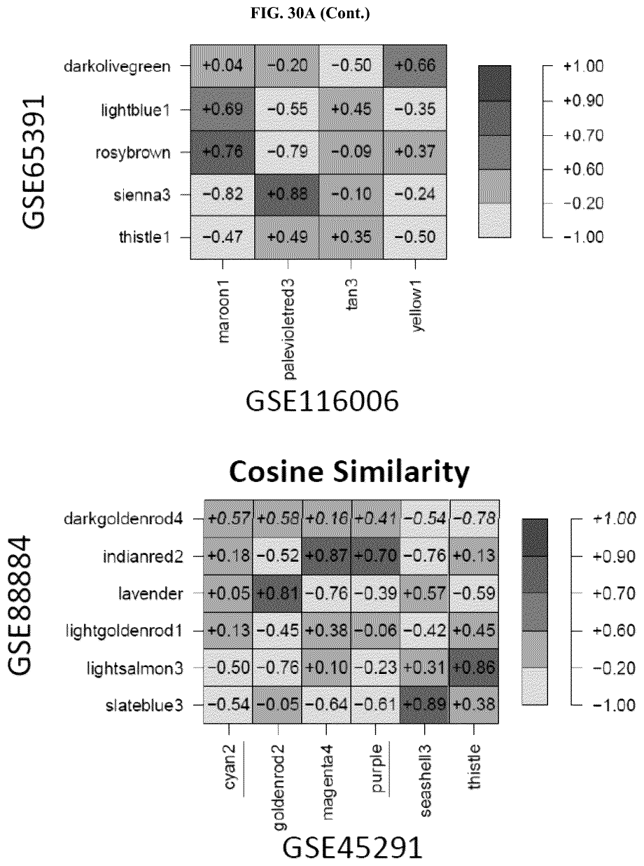

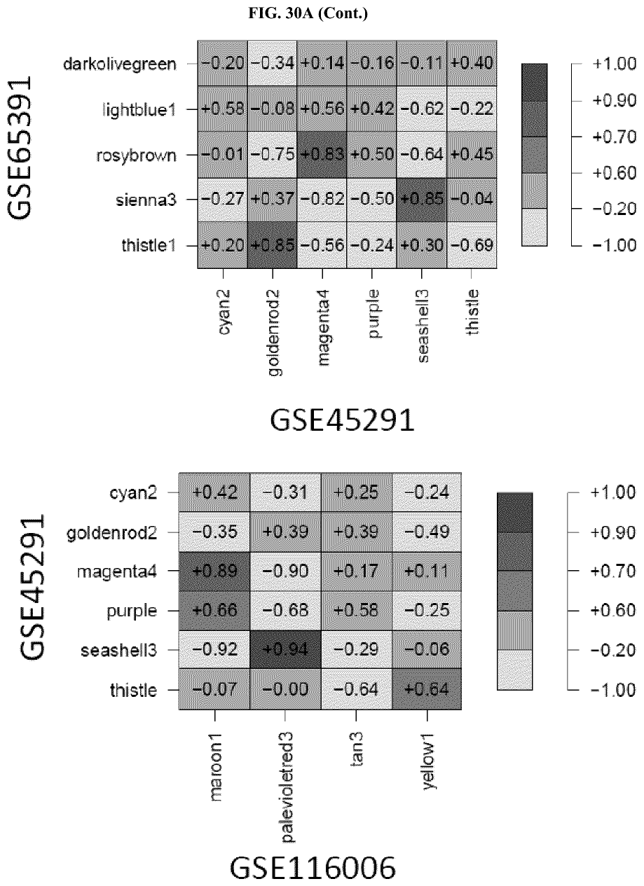

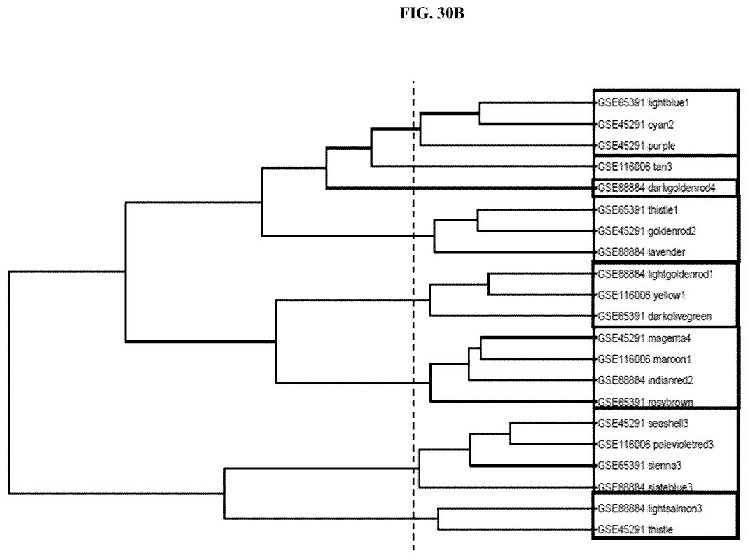

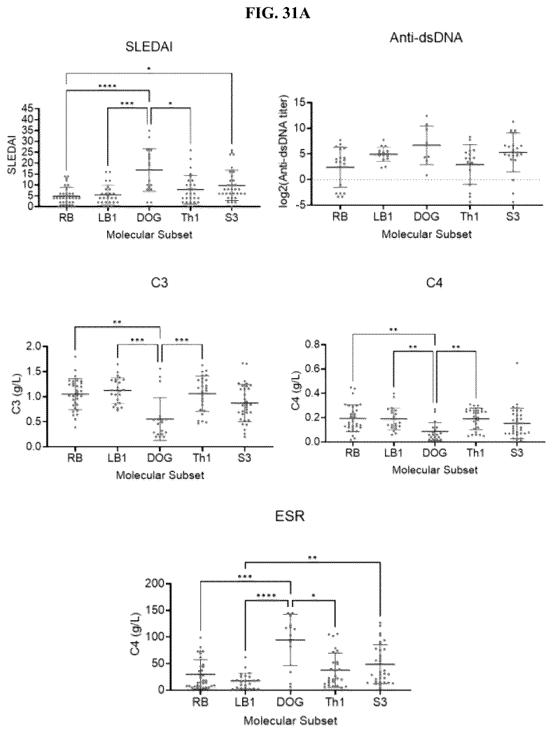

Endotype Determination in Additional, Unrelated Datasets. Next, we extended our endotyping method to include small datasets, two with both active and inactive patients, and a large RNA-seq dataset, to determine if additional endotypes could be detected (Table 33, Datasets 14, 16-17). Six endotypes were identified in a cohort of 266 adult lupus patients, five endotypes were identified in a cohort of 137 pediatric lupus patients, and four endotypes were identified in 160 adult lupus patients participating in a clinical trial of an anti-IL-6 monoclonal antibody (FIG. 26, FIG. 27). Similar to the proof-of-concept Illuminate dataset (GSE88884), non-lupus controls tended to cluster in the “least perturbed” endotype with similar enrichment patterns as IR2 (FIG. 28, FIG. 29). Cosine similarity analysis indicated that several of these endotypes were reproducible among all datasets. Combining all unique endotypes across datasets indicated that among these 2,183 patients, 11 endotypes were identifiable (FIG. 30A). However, even after considering cosine similarity >0.7, hierarchical clustering of the mean GSVA scores of the individual endotypes suggested that the expansion from eight to 11 endotypes occurs within a very small statistical space (FIG. 30B) and suggested further likeness between subsets not captured by cosine similarity; thus, we reduced the total number of likely identifiable endotypes in any given lupus blood dataset to eight. This made sense as three of the unique endotypes were similar to other subsets but narrowly missed the cutoff (FIG. 30A).

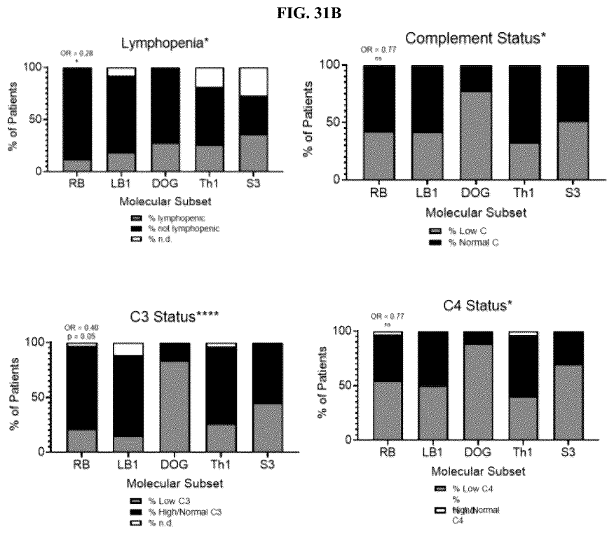

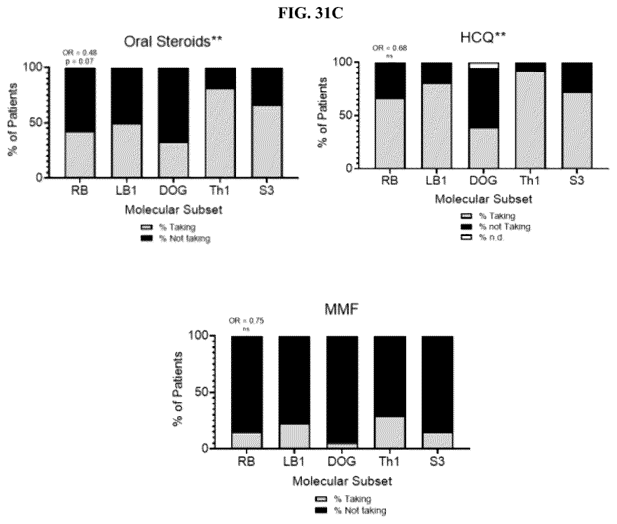

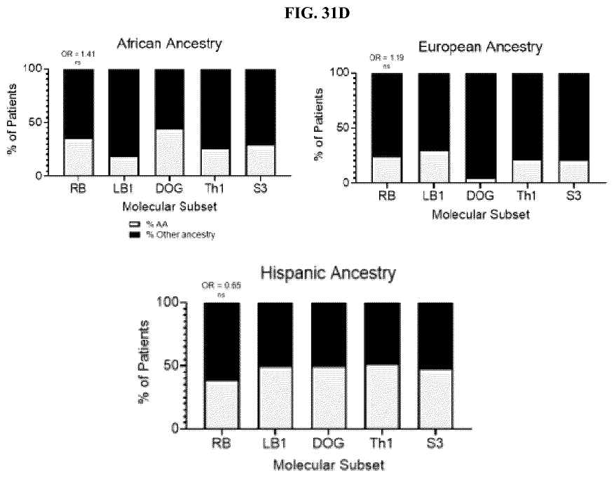

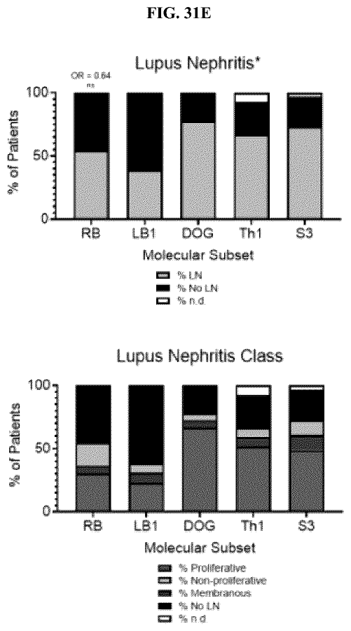

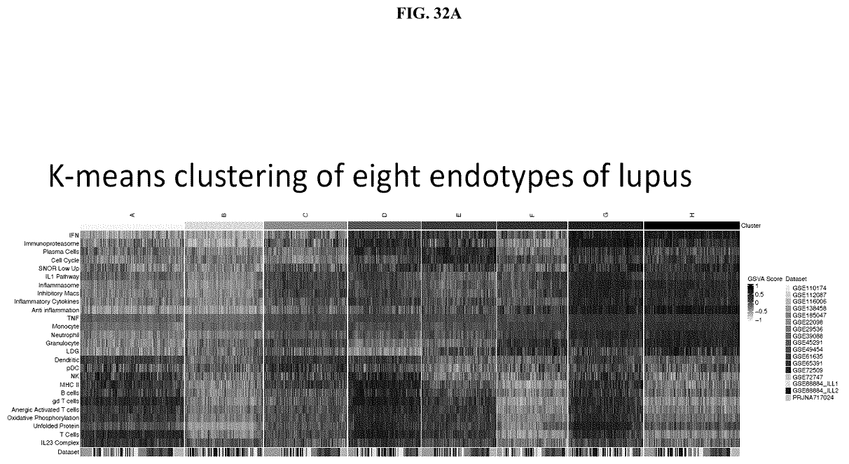

Because extensive metadata accompanied the pediatric lupus patients (GSE65391), we analyzed it similarly as that of the Illuminate trials (GSE88884) and found significant differences among endotypes with regard to SLEDAI, complement levels, and ESR, with DOG exhibiting the most disease activity (FIG. 31A). Although mean anti-dsDNA levels varied among endotypes and were highest in DOG, intra-group variation was too high to detect significant intergroup differences. Pediatric endotypes also differed by occurrence of lymphopenia (p<0.05), corticosteroid use (p<0.01) and HCQ use (p<0.01), but, interestingly, not ancestry (p>0.05, FIG. 31B-D). However, the most active endotype, DOG, contained the highest proportions of AA and NAA/Hispanic patients and patients with proliferative nephritis (FIG. 31E).

Machine Learning (ML) to Classify SLE Samples into Endotypes. Next, we concatenated the GSVA enrichment scores of 26/32 measurable features across 17 datasets of lupus blood gene expression to leverage the statistical power of 3,166 samples (Table 33). We input these scores into our bioinformatics pipeline with k=8 and observed eight endotypes labeled A-H (FIG. 32A) with no fewer than 300 patients each. Then, using these GSVA scores and endotype membership determinations, the data were partitioned into training (Table 33, Datasets 6-7, 14, 16-17) and testing (Table 33, remaining 12 datasets) sets. The training data was further partitioned into training (80%) and validation (20%) sets and one-vs.-one multi-class classification by multiple algorithms was carried out to predict endotype memberships. ROC curves for random forest (RF), support vector machine (SVM), logistic regression (LR), and gradient boosting (GB) models were generated on the 12 test datasets and demonstrated high predictive capabilities (FIG. 32B-E). LR had the highest precision overall with sensitivity ranging from 89%-100% and specificity ranging from 99%-100% for the eight endotypes (FIG. 32D).

With the identification of eight endotypes representing the apparent universe of lupus patients and high predictive capability of ML algorithms, we sought to reduce the information from gene expression profiles into a novel clinical metric, designated LuCIS, to display the range of molecular abnormalities numerically. An RPLR model was employed to calculate a LuCIS value for each individual patient based on her binarized GSVA enrichment score (FIG. 33A). These scores were then compared to the placement of each patient in our eight lupus endotypes and showed increasing LuCIS values for each endotype A-H (FIG. 33B).

To contextualize LuCIS values, we calculated the scores of healthy, non-lupus controls in five datasets for which adequate control samples were available and reported their mean scores for each of the datasets (FIG. 33C). The mean LuCIS values of the least abnormal lupus endotypes were not significantly different from those of the non-lupus controls, indicating that LuCIS is reflective of the least abnormal endotypes' resemblance to a normal transcriptional profile.

Finally, to explore LuCIS as a clinical metric, we investigated the relationship between LuCIS and anti-dsDNA titer (FIG. 33D), SLEDAI (FIG. 33E), serum C3 (FIG. 33F), and serum C4 (FIG. 33G) in GSE88884 for which full clinical data were available. Positive correlations were identified (p<0.0001) between LuCIS and anti-dsDNA titer or SLEDAI, whereas negative correlations were identified (p<0.0001) between LuCIS and C3 or C4; however, coefficients were weak, suggesting that LuCIS is related to these metrics but may provide additional information not captured by SLEDAI or serology that is reflective of immunologic activity.

Use of SHAP to Determine the Most Important Features of Endotypes. At this point, we had developed two novel tools: one bioinformatic (high-performing ML algorithms) to predict endotype membership and one clinical (LuCIS) to parameterize molecular perturbations, both predicated on the identification of eight overall lupus endotypes and both carrying prognostic relevance. Therefore, as a final exercise, we interrogated the specific features contributing to the endotype groupings using additional ML classification and Shapley Additive Explanations (SHAP).

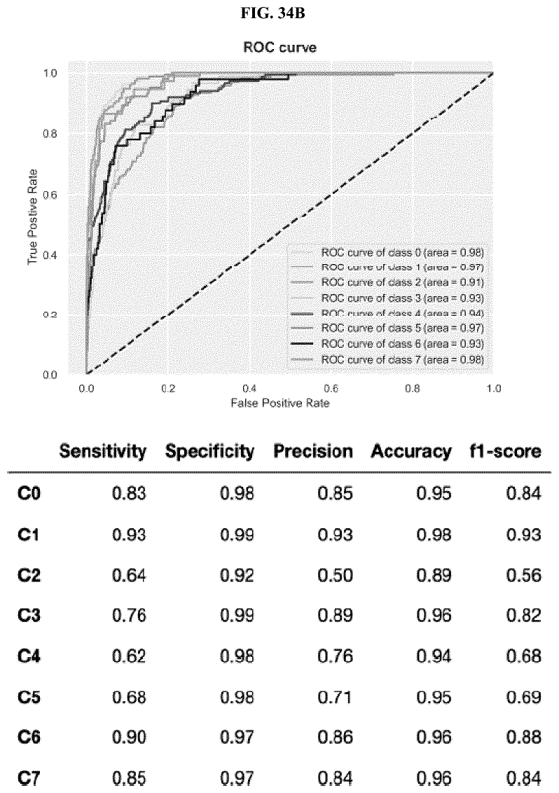

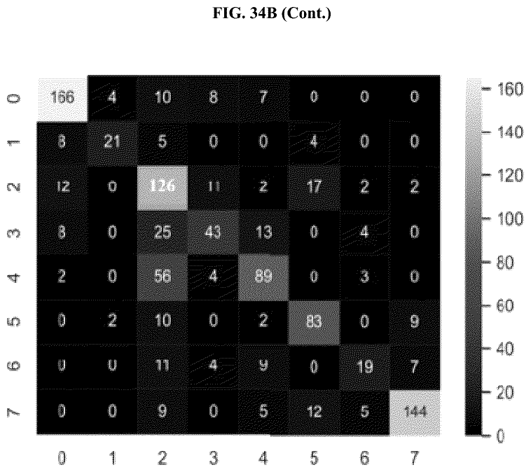

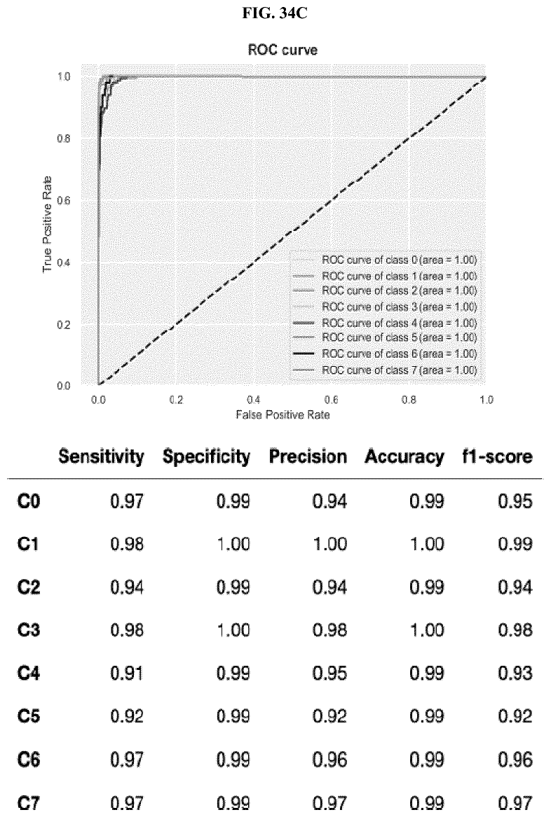

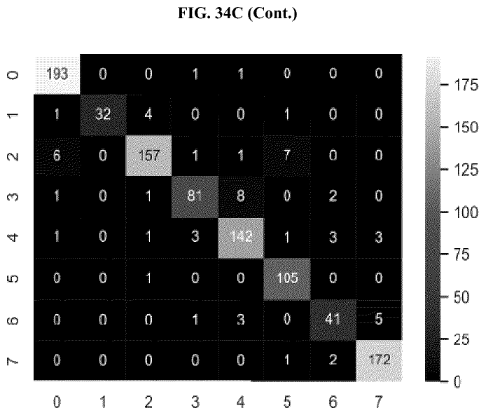

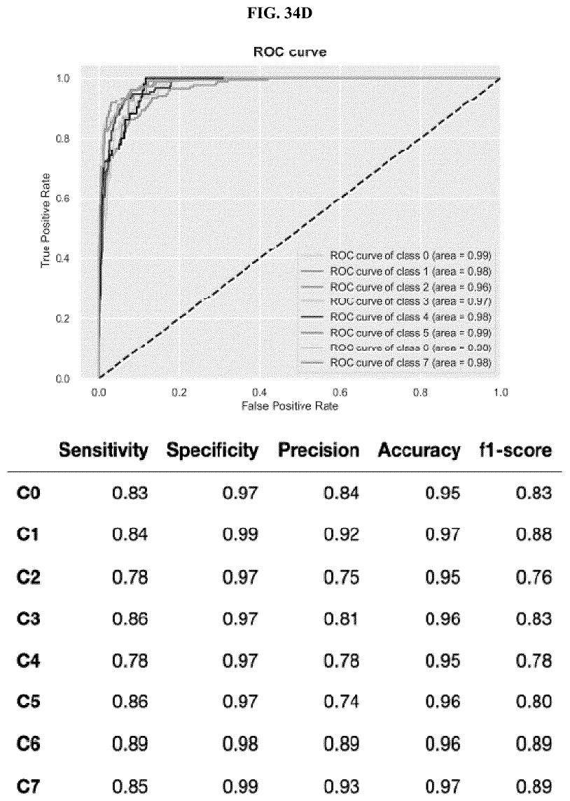

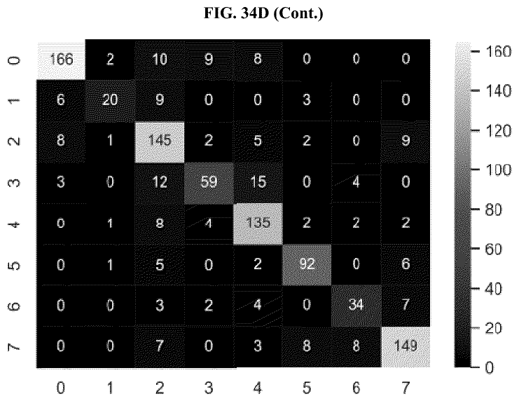

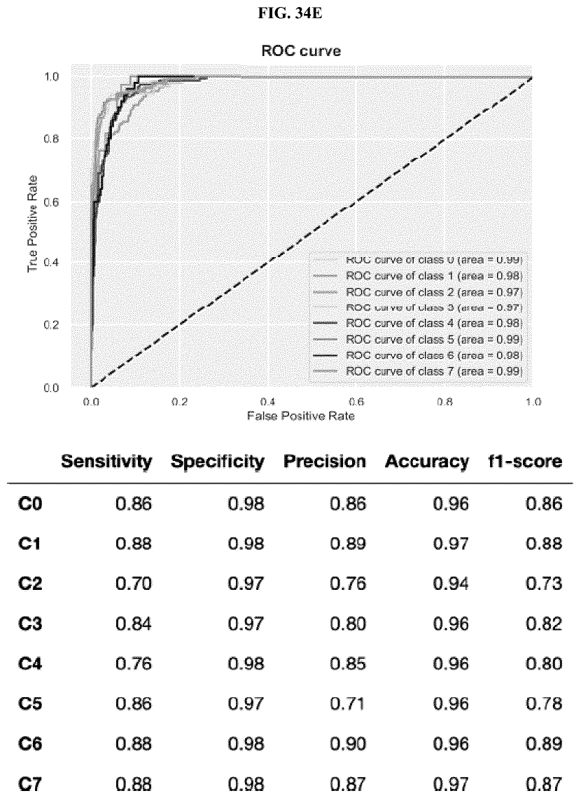

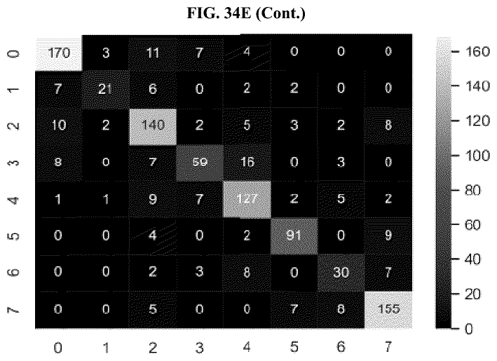

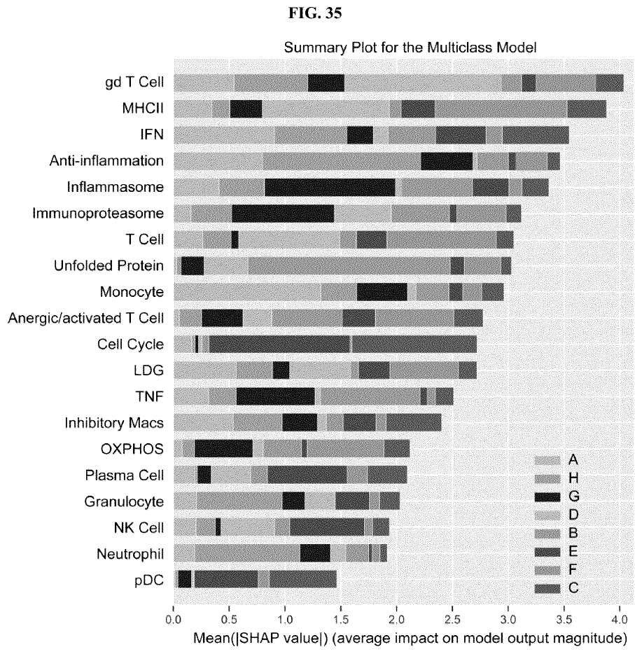

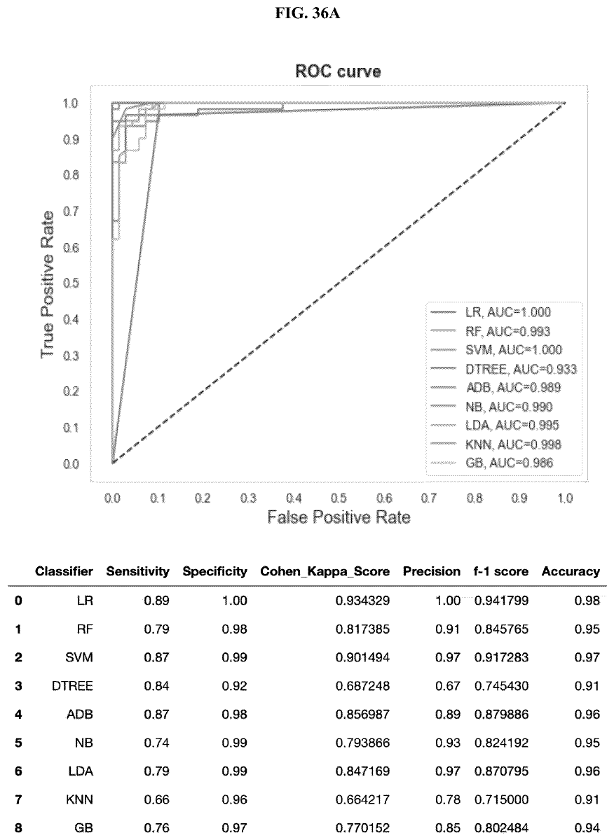

We generated another set of ML classifiers using one-vs.-rest multi-class classification (FIG. 34) and used SHAP to compute the contribution of each feature. A summary of mean absolute SHAP values across patients in the eight endotypes from the extreme GB classifier revealed the top 20 features contributing to the model, with gamma delta T cells, MHCII, and IFN being the overall most impactful (FIG. 35). Anti-inflammation, granulocyte, and neutrophil features most distinguished the most perturbed endotype H (FIG. 32A), whereas monocytes, anti-inflammation, and IFN features most impacted the least perturbed endotype A.

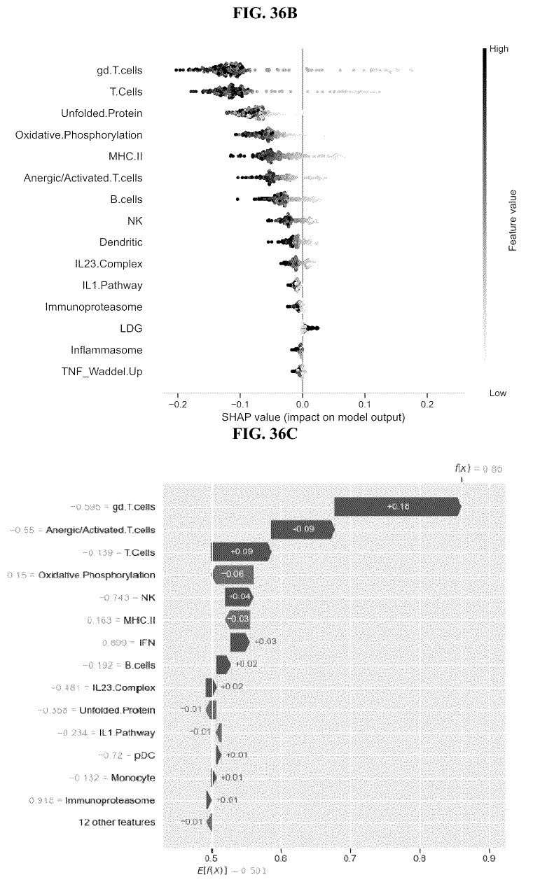

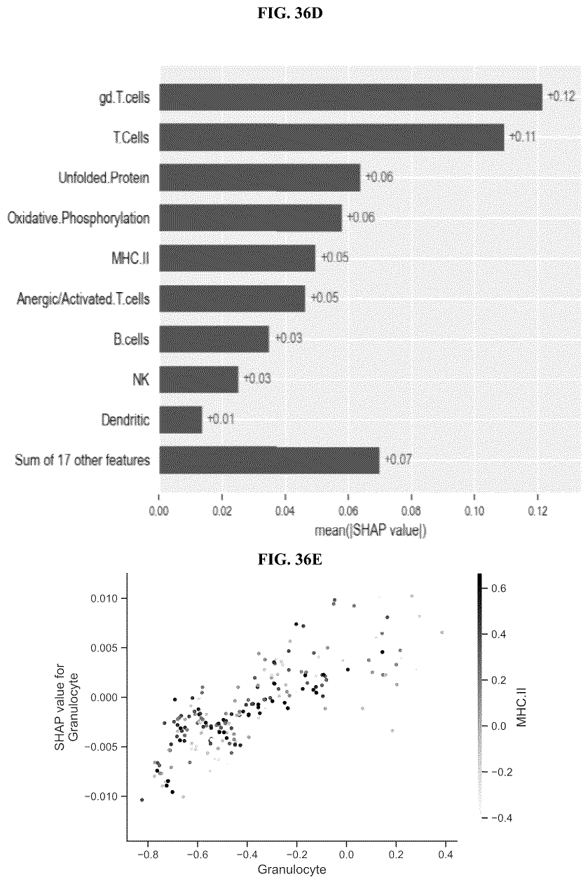

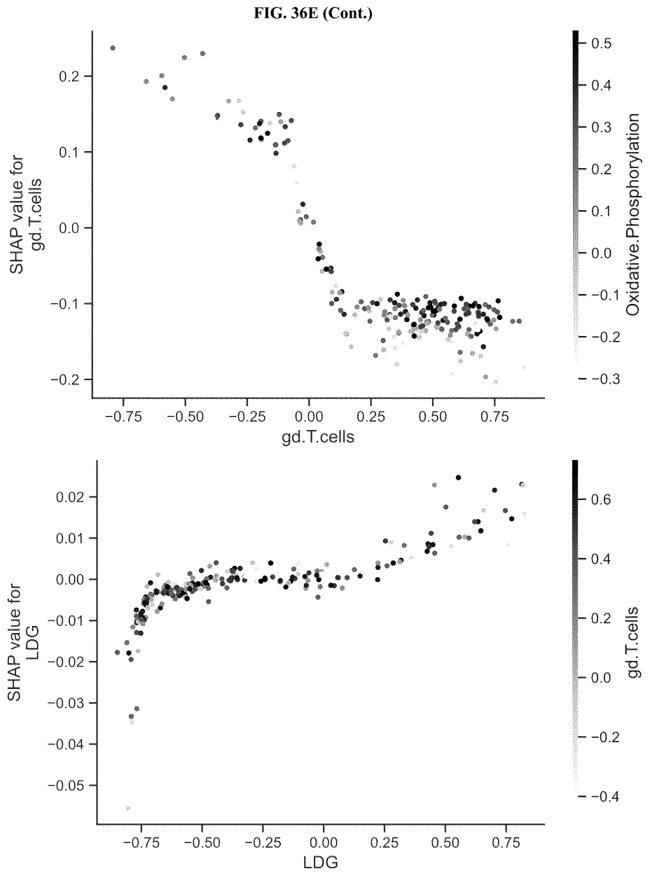

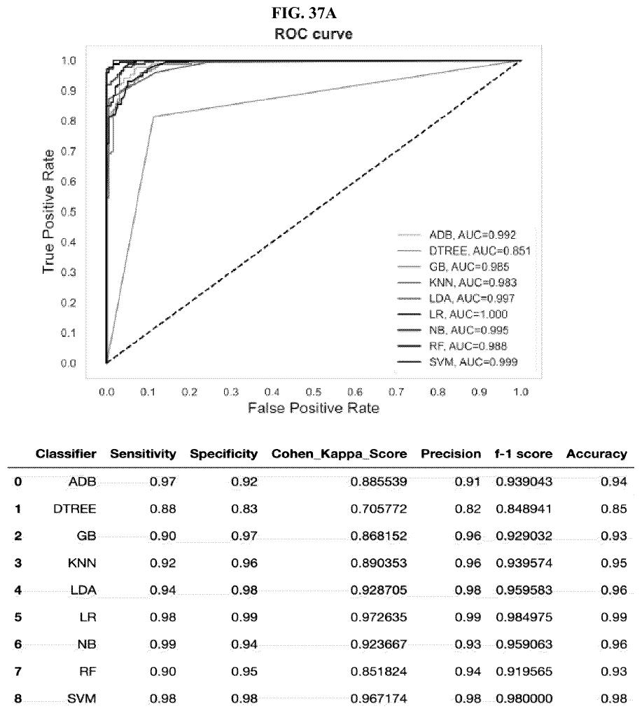

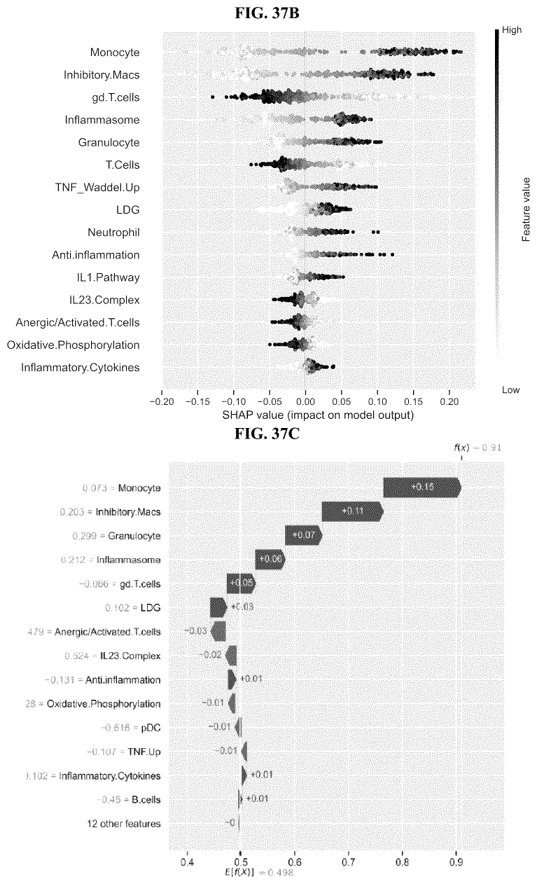

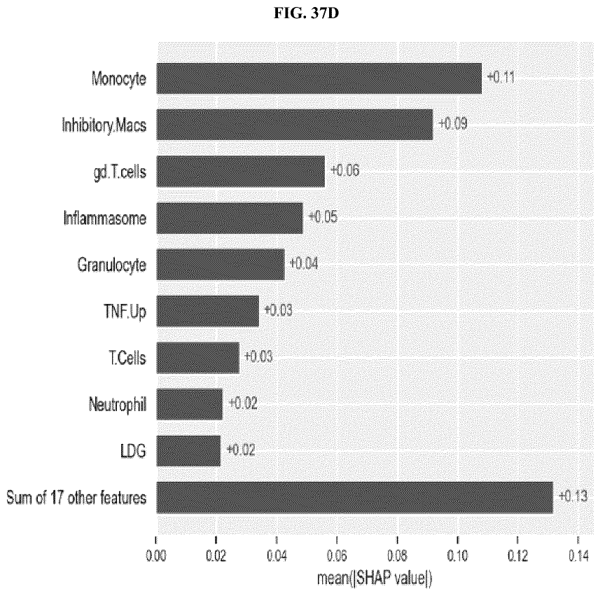

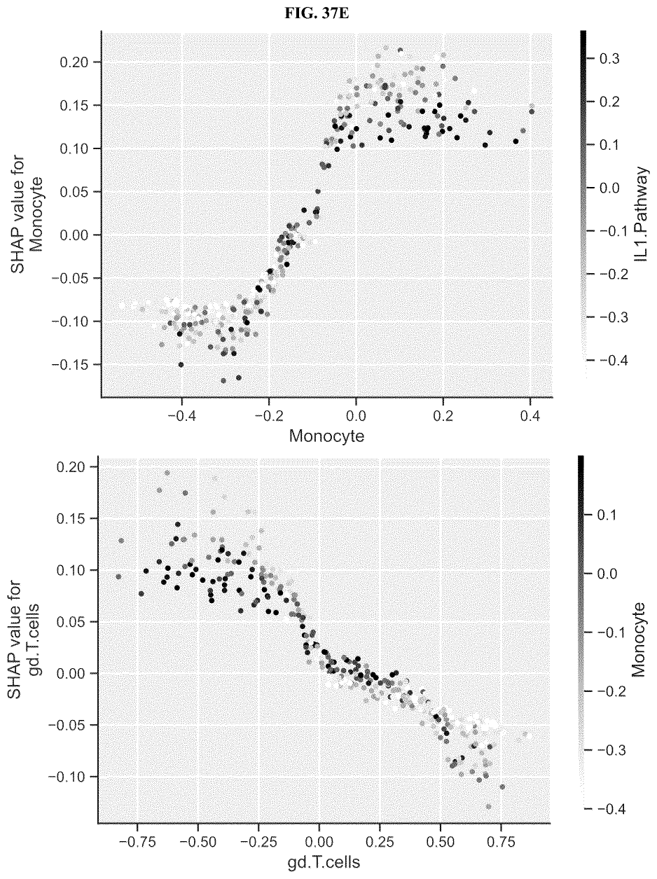

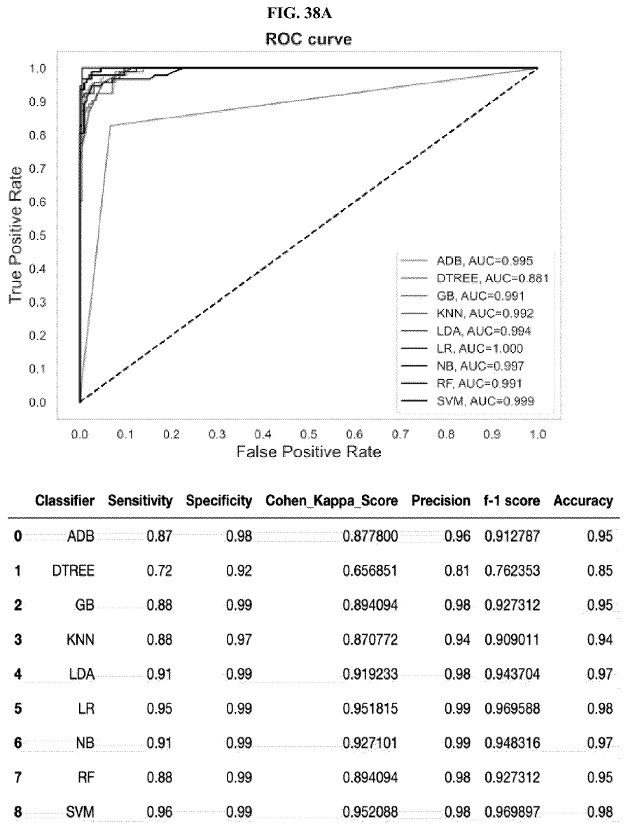

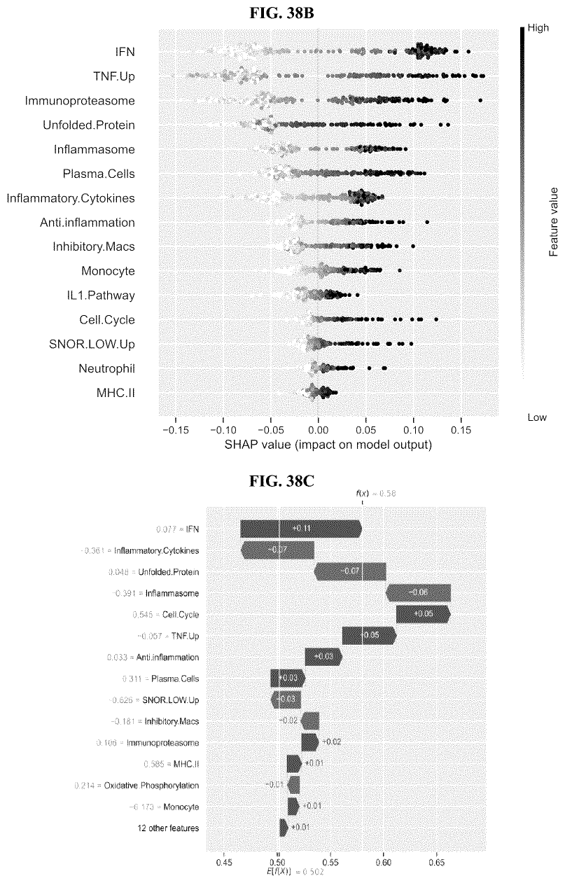

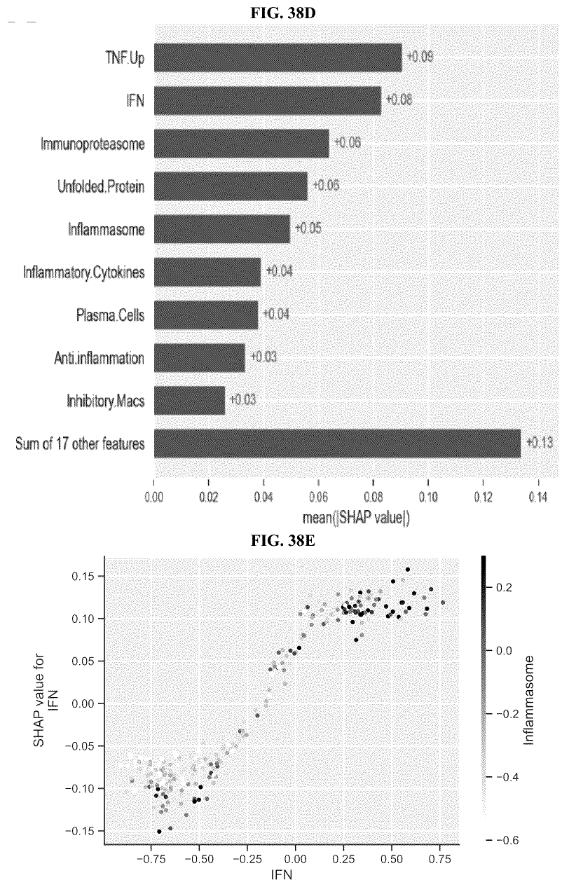

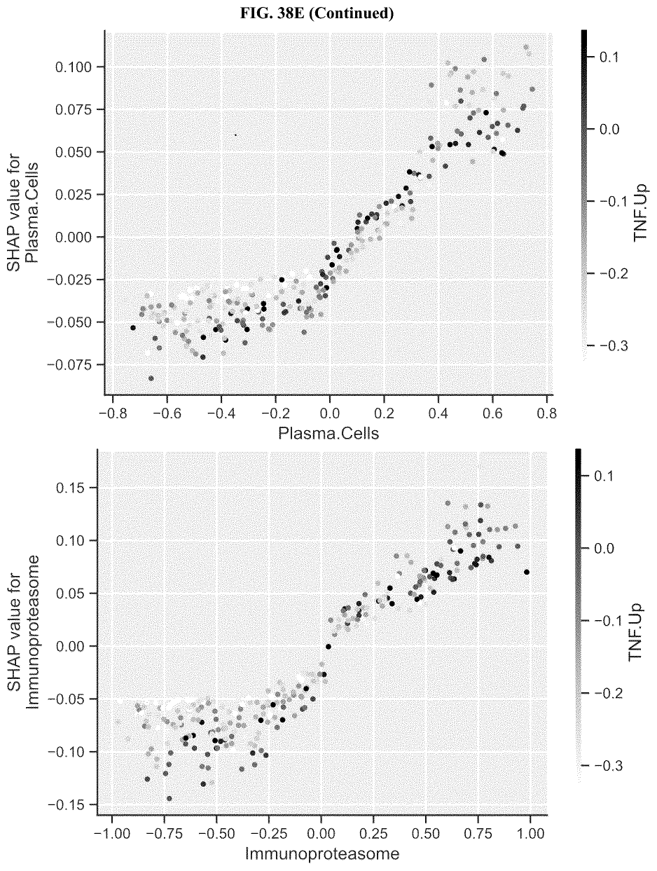

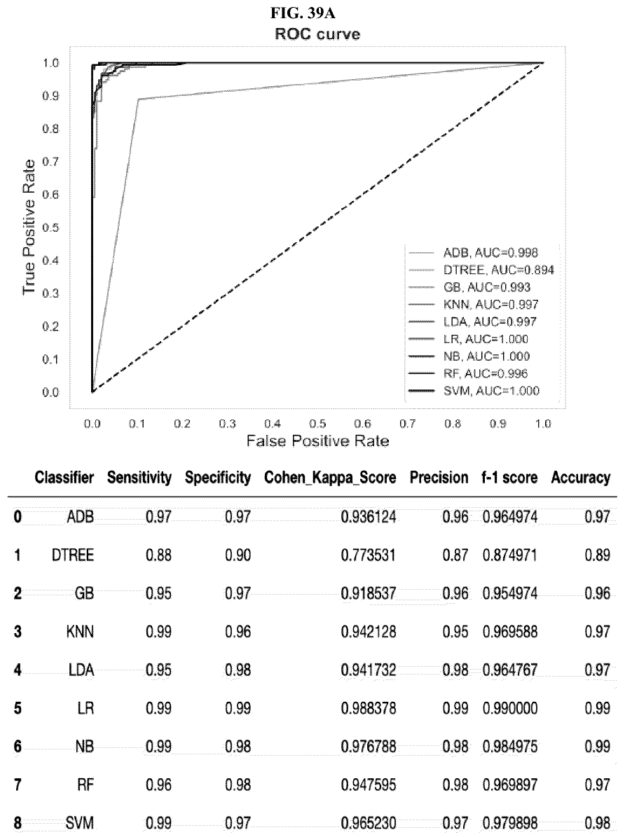

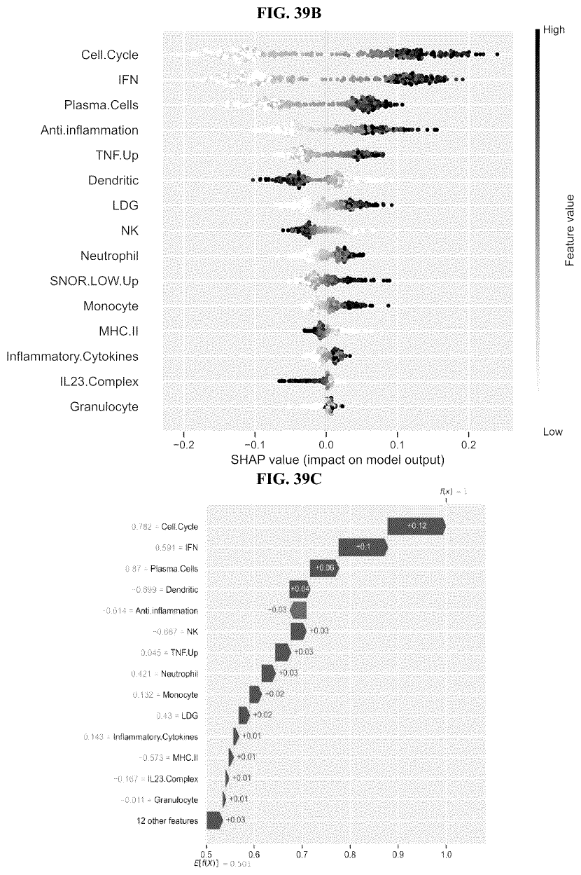

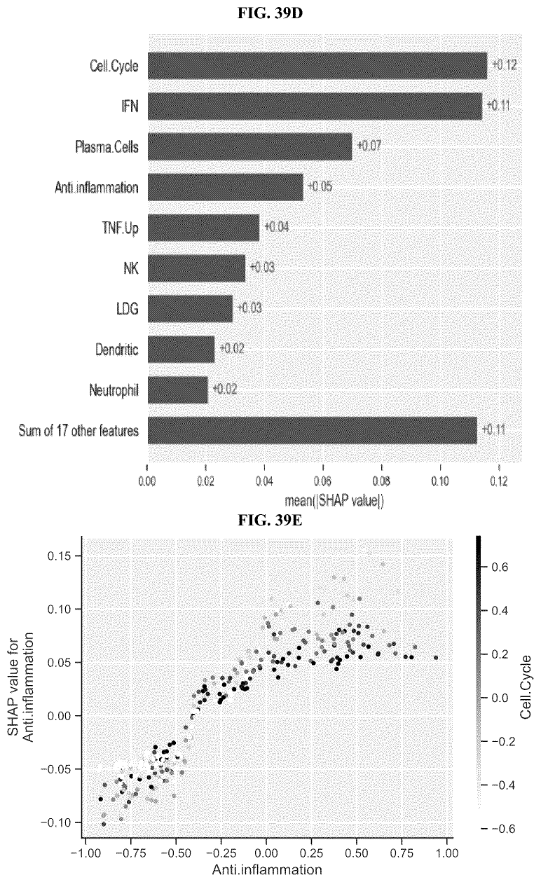

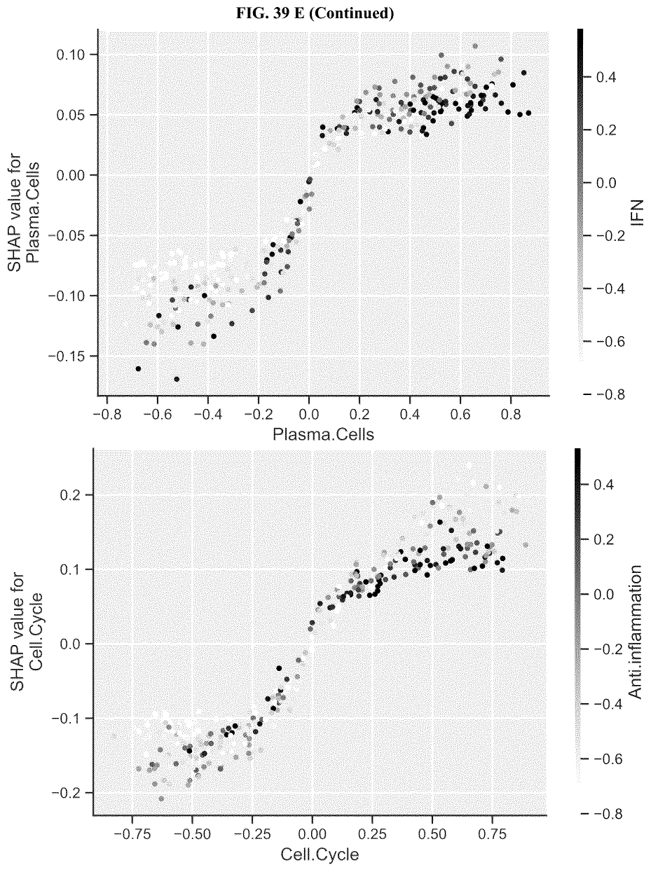

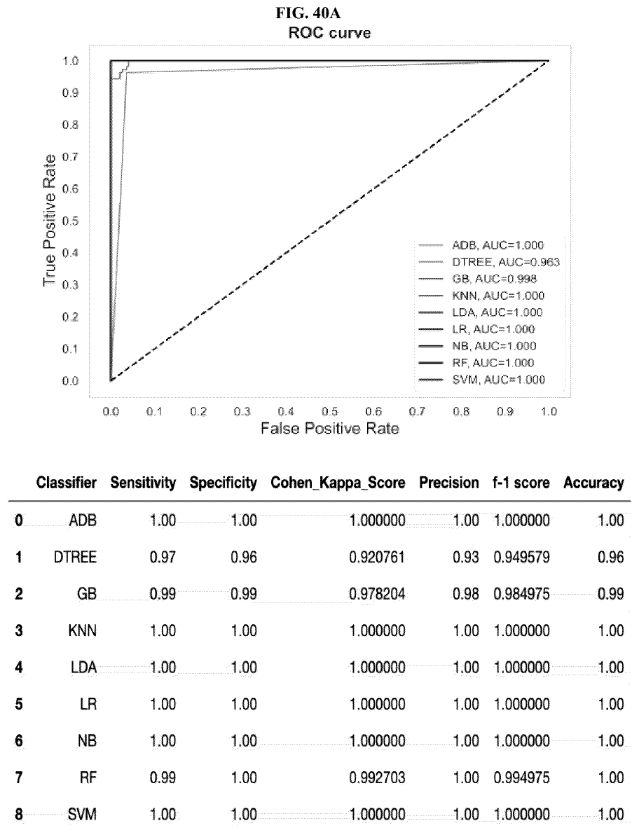

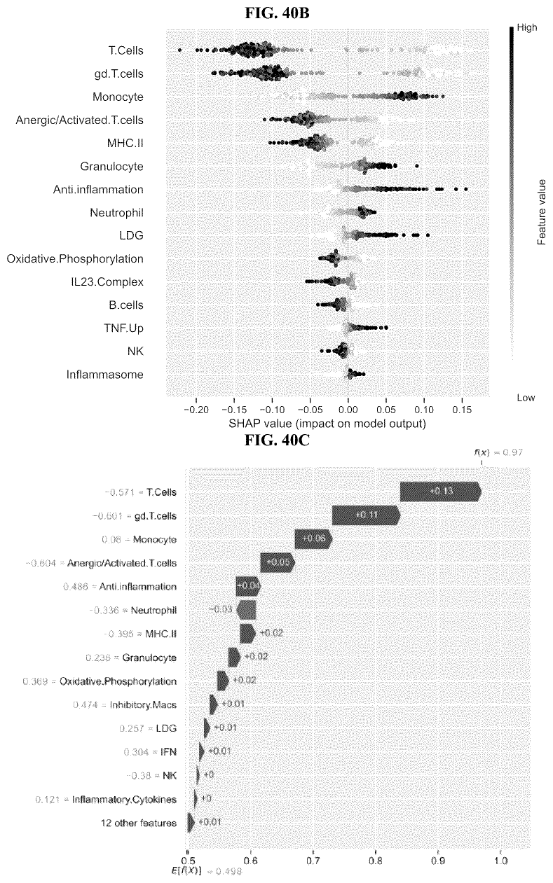

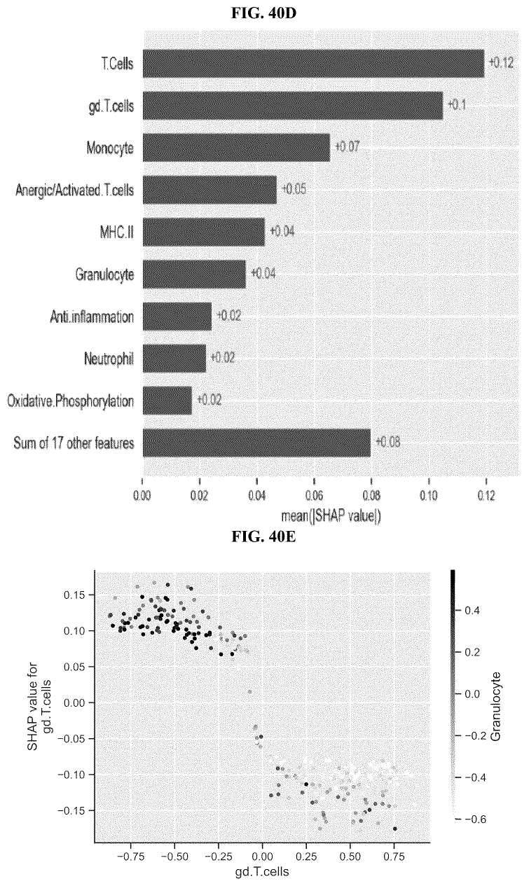

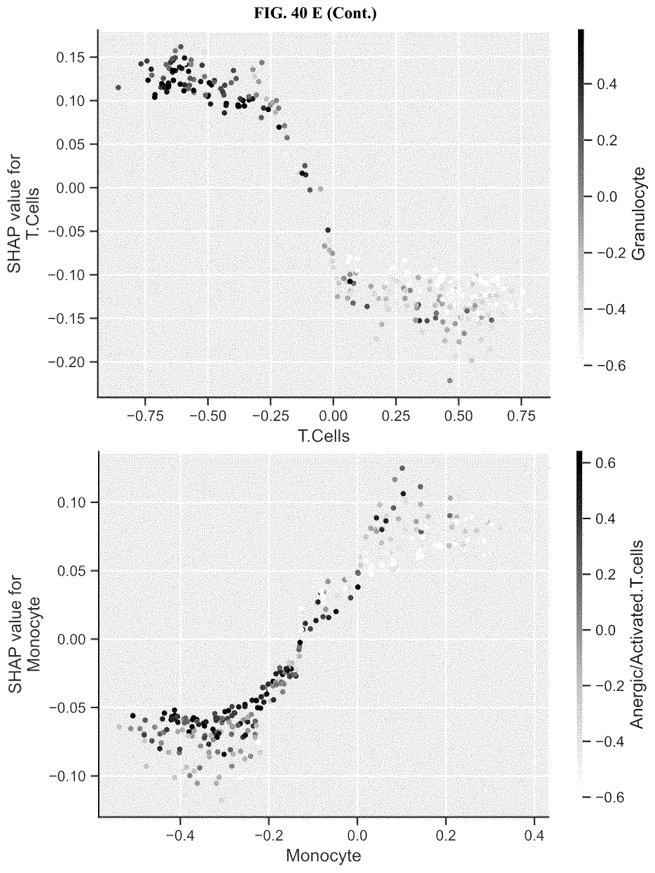

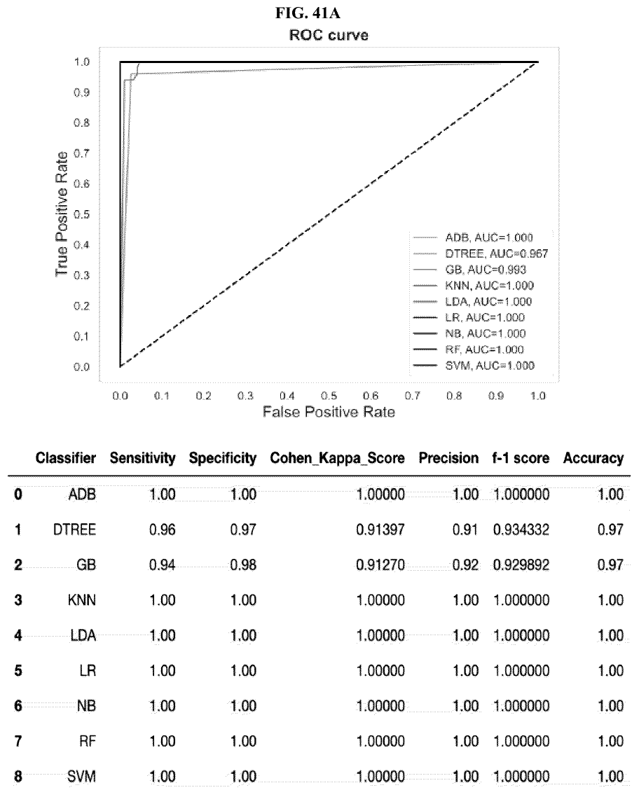

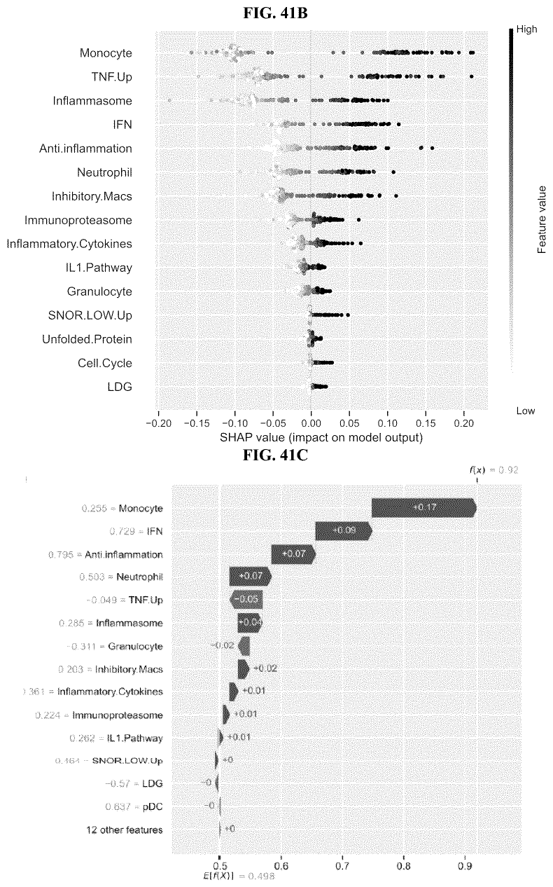

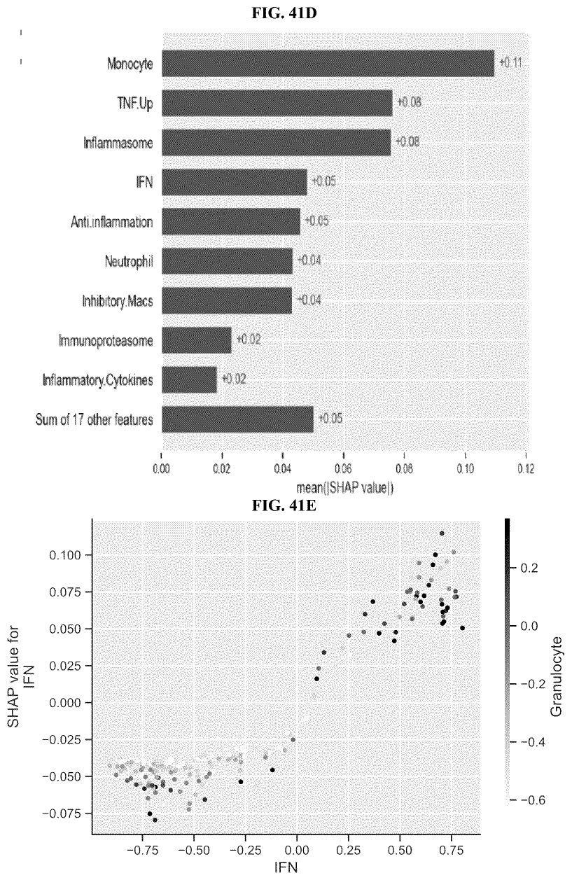

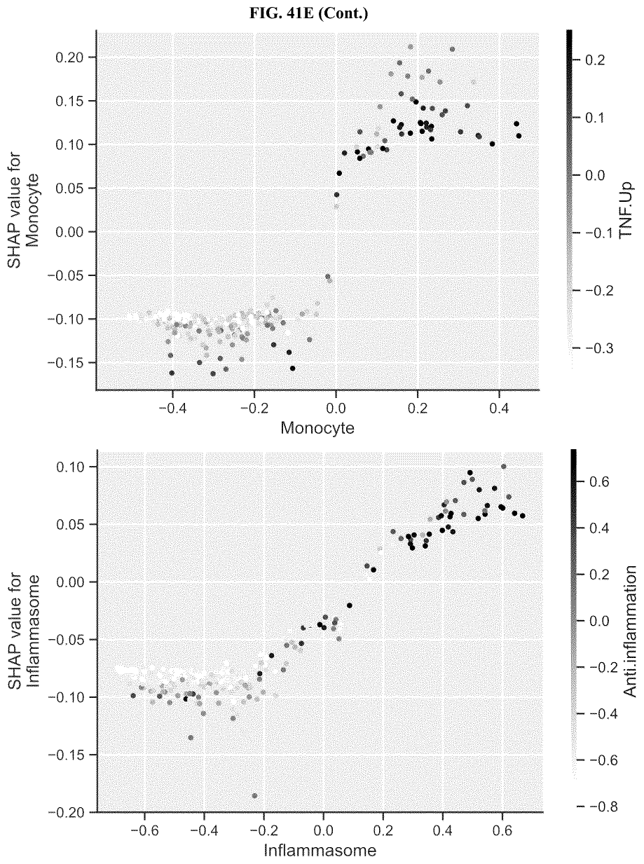

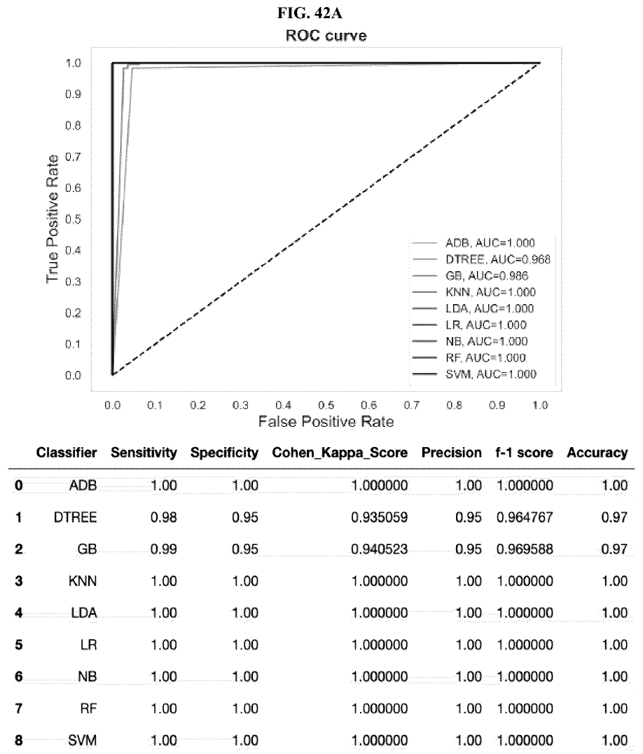

For further characterization, we additionally employed seven binary classifications comparing the seven most transcriptionally perturbed endotypes (B-H) to the least abnormal endotype (A). These classifiers (FIGS. 36-42) demonstrated excellent performance with an average positive predictive value of 0.97 for the RF classifier, but all classifiers performed well (Table 36).

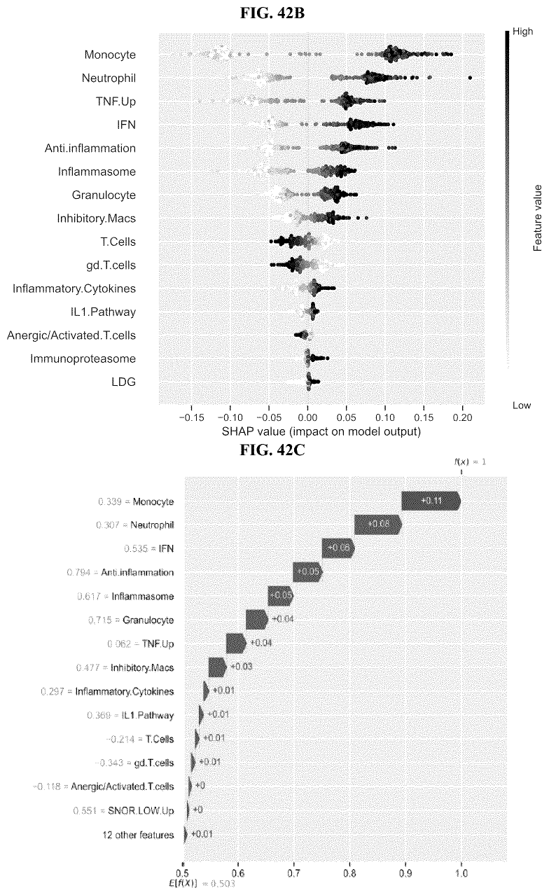

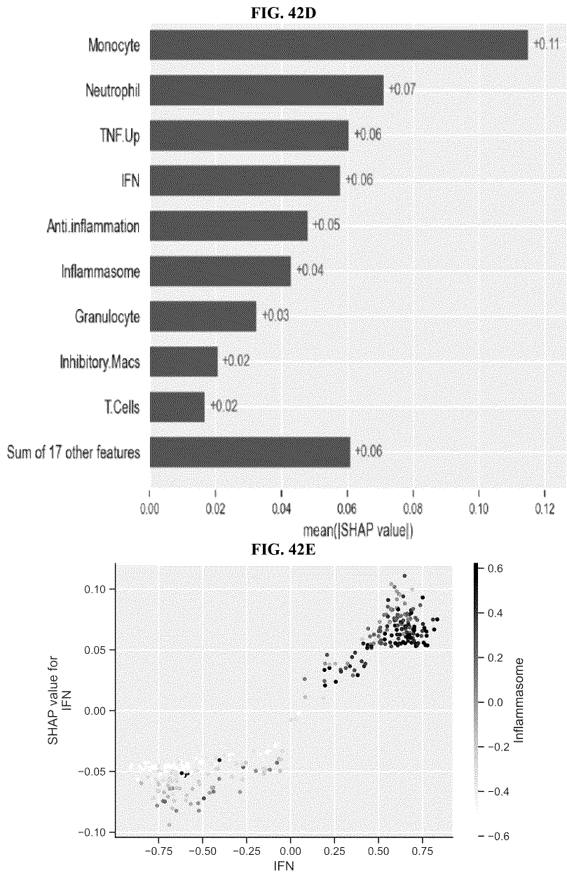

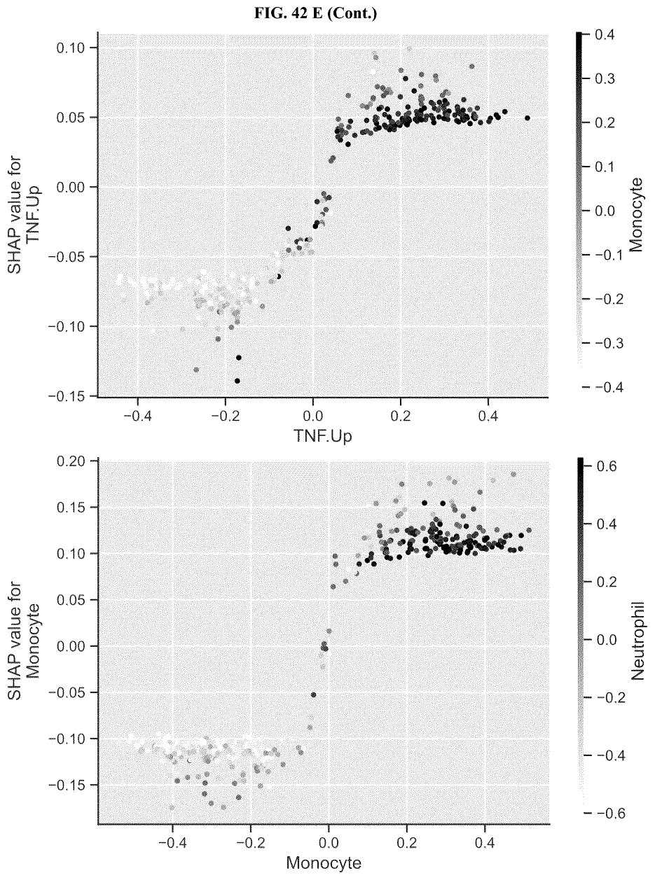

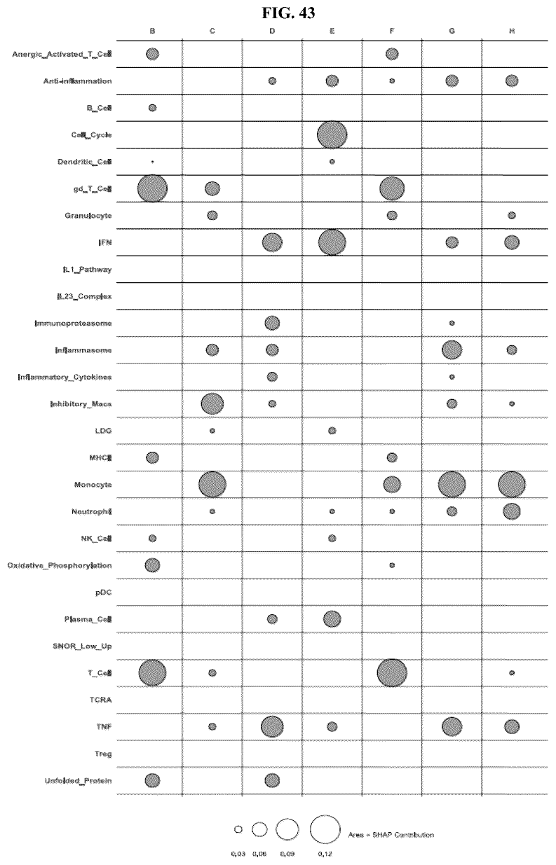

SHAP analysis of the RF classifiers enabled detailed insight into the features that characterized individual endotypes from that most resembling non-lupus controls (endotype A) (FIG. 43). The scatterplot enumerates the contribution of the mean GSVA enrichment score of each feature to the capacity of the ML model to distinguish a particular endotype from A; the impact of each feature at the individual patient levels is shown as SHAP waterfall plots (FIGS. 36-42). Different patterns of features distinguished most of the endotypes (FIG. 43). For example, the endotype with the most immunologic abnormalities (H) demonstrated high contributions from the monocyte, neutrophil, TNF, and IFN signatures (FIG. 43, FIG. 42), whereas T cell signatures were most contributory to the least perturbed endotype (FIG. 36).

SHAP dependence plots detailed the various effects of the individual features on predictions made by the model with, for example, IFN, TNF, and monocytes having an impact at the extremes of GSVA scores to distinguish endotype H (FIG. 42E). These analyses helped to demystify the ML and characterize in great detail the molecular endotypes in SLE.

Feature culling, starting with 26 molecular feature that were present in all 17 datasets, was done in sequential fashion by filtering out the gene modules with smaller ridge coefficients until k-Means clustering algorithm fails to determine the 8 lupus endotypes. The k-Means clustering of 26 features (Tables: 1; 2; 3; 4; 5; 6; 7; 8; 10; 11; 12; 13; 14; 15; 16; 17; 18; 19; 20; 21; 22; 23; 24; 25; 31; and 32) identifies all lupus endotypes. The k-Means clustering of 23 features after removing Anergic/Activated T cells (Table 1), B cells (Table 3), and Oxidative phosphorylation (Table 21) identifies all lupus endotypes except C. The 23 features are reduced to 21 by removing IL1 pathway and Unfolded Protein which yield same results as 23 features. The 21 features are then culled to 18, and 18 to 16, which yields the same results as 23 features. The resulting k-Means clustering on 14 features, 12 features, and 10 features reproduce only 2 out of 8 lupus endotypes.

Determining Module Z-Scores and Deviations from Endotype a, for Endotypes B-H (FIG. 32A).

To determine whether an endotype's (B-H's) gene expression for a particular module is abnormal we employed a Z-score method. This determines whether the endotype's gene expression deviates from that seen in the “normal” endotype (endotype A). If the endotype's gene expression is outside of normal (there is a less than 5% chance of the value occurring randomly), an abnormality value is reported. That is, the deviation of the endotype's value from that of endotype A is reported. This deviation is calculated as: endotype mean −endotype A mean for a module.

In this method, to calculate the z-score the module mean from endotype A is subtracted from the module mean for the endotype of interest and then divided by the standard deviation of endotype A:

[Z-score=(endotype module mean GSVA score−endotype A module mean GSVA score)/endotype A module standard deviation GSVA Score].

If the subset Z-score falls between −2 and 2, the endotype's gene expression for that module is considered within the normal range. If the subset's Z-score is <−2 or >2, the endotype's module gene expression is considered abnormal. The value reported for the abnormality is the difference between the endotype's module gene expression score and the endotype A module mean gene expression score. Table 37 shows the determining whether there are significant differences based on Z-score normalization. Table 38 shows the value of the difference relative to Subset A—if significant (only significantly changed modules are shown). Table 39 shows the abnormal modules/features in the endotypes B-H. Up arrows show the modules that are significantly enriched compared to endotype A, and down arrows show the modules that are significantly de-enriched compared to endotype A.

Determining effective number of genes per module/Table. Determination of effective number of genes per module/Table was done by performing k-Means clustering on randomly selected gene subsets by standard interval based on the total number of genes of each module/Table. Similarity between two clustering is measured by adjusted rand index (ARI). For example, the adjusted rand index (ARI) is calculated between K-Means cluster memberships from each randomly selected gene subset to the cluster memberships obtained using total number of genes of each module/Table. The higher the ARI, the similar the cluster memberships and lower the ARI the weaker the cluster memberships suggesting more genes are required. The ARI is calculated to determine the appropriate number of genes for each module. The adjusted rand indices are visualized using the line plot. FIGS. 46 A-D show ARI Line plots for 4 LuGene modules A) Monocyte (Table 18), B) IG Chains (Table 9), C) IFN (Table 8), D) LDG (Table 16). The * in FIGS. 46A-D show required number of genes. Monocytes require 110 genes, IG chains require 70 genes, whereas IFN and LDG require all the genes in the module. FIGS. 46E-H show the confusion matrixes showing the cluster memberships of various subsets to the reference population. FIG. 46E shows similarity of the kmeans cluster memberships from random monocyte subset to the reference Monocyte module (all genes). FIG. 46F shows similarity of the kmeans cluster memberships from random IFN subset to the reference IFN module (all genes). FIG. 46G shows similarity of the kmeans cluster memberships from random LDG subset to the reference LDG module (all genes). FIG. 46H shows similarity of the kmeans cluster memberships from random IG chain subset to the reference IG chain module (all genes). The above analysis suggests that modules with more number of genes may require more 60% of genes whereas smaller modules may require all the genes.

### Discussion

The autoimmune process in lupus is generally characterized by a loss of self-tolerance and production of pathogenic autoantibodies that can deposit in tissues and incite inflammation and damage, but the specific disease course in individual patients varies greatly and is difficult to predict. In addition, efforts to stratify lupus patients into clinically informative and actionable groups have been largely unsuccessful and often reduce to differences in serologic features, systemic features, patient demographics, and the presence (or absence) of an interferon signature [9, 29-31]. Herein, we describe a novel characterization of lupus patients based on identifiable endotypes using inflammatory and immunologic transcriptomic features and k-means clustering.

In a proof-of-concept cohort, the endotype with the least abnormal transcriptional profile manifested the lowest mean SLEDAI, lowest ANA titers, highest serum complement levels, and lowest incidence of lymphopenia, whereas the subsets with more abnormal transcriptional profiles showed significantly more abnormal clinical features. The endotype with the least abnormal transcriptional profile also had a significantly lower frequency of severe flares over the subsequent 52 weeks while receiving SoC and exhibited no significant clinical response to the investigational product, tabalumab. Moreover, we identified significant clinical responses to tabalumab in three endotypes with more perturbed gene expression profiles. Thus, we were able to identify clinically meaningful phenotypes based on the gene expression-based endotypes.

Because gene expression can be influenced by the use of corticosteroids, immunosuppressive agents or patient ancestry, we repeated our analyses in cohorts restricted to patients of single ancestries and separate cohorts of patients taking glucocorticoids and/or mycophenolate, which we have shown to significantly affect plasma cells and other gene expression [13]. Most endotypes were identified across ancestries, but we found different distributions of patients by ancestry among the endotypes; in particular, very few AA patients comprised the endotype with the least perturbed transcriptional profile, and, likewise, few EA patients were found in the subset with the greatest number of immunologic perturbations when all ancestries were considered. Additionally, anew endotype in patients of NAA/Hispanic ancestry emerged that was almost cytopenic but enriched for TCR gene signatures. These findings further suggest that these identified endotypes may be reflective of immunologic and systemic disease activity that presents clinically, as SLE is more severe among AA cohorts compared to EA cohorts [13, 32]. That is, the transcriptional profiling and subsequent endotyping presented herein may serve as a proxy for lupus immune activity.

We were able to demonstrate the utility of transcriptomic profiling over current, standard biomarkers and patient demographics. Sub-setting lupus patients based on clinical data alone stratified patients primarily by ancestry and was not robust to methodology, as employment of k-means clustering and clustering by a deep learning variational autoencoder produced significantly different results from each other and from the molecular subsets, which we confirmed by ML. Thus, the endotypes could not be identified based on clinical metadata. The molecular subsets were also associated with differences in responsiveness to therapy.

Extension of our methods to external datasets that, importantly, contained diverse patients and data from different NGS platforms, led to the identification of eight robust endotypes representative of the apparent universe of molecularly-defined lupus subsets. With these data we were able to overcome dataset-specific heterogeneity and develop two independent, informative tools: a multi-class ML classifier predicting endotype membership of individual lupus samples, and a transcriptomic-based composite value, LuCIS, estimating the level and severity of lupus-related immunologic activity. The ML classifiers showed high predictive capabilities (up to 100%) after cross-validation in unrelated datasets, demonstrating the robustness of the endotypes, and further interrogation of features contributing to the ML models demonstrated specific gene signatures involved in the classification of patients into endotypes, which could be further probed for druggable targets. The LuCIS calculations demonstrated that the endotypes are gradations of immunologic disease activity and that LuCIS values could serve as a new clinical metric to estimate lupus activity not captured by current approaches.

In keeping with the shift of clinical medicine to greater emphasize individualization and precision, we identified eight endotypes that map the molecular heterogeneity of individual lupus patients whilst also representing an entire disease population. Our body of work and the development of LuCIS based on these eight endotypes demonstrates how an individual patient can be classified using his or her blood gene expression profile. With the power of big data, bioinformatics, and machine learning, we anticipate this new method of clinical classification of lupus will aid physicians' overall care, including therapeutic courses of action and disease management strategies.

Dimensionality Reduction from Modules to Genes

GSVA scores of 26/32 cellular and processes modules (Tables: 1; 2; 3; 4; 5; 6; 7; 8; 10; 11; 12; 13; 14; 15; 16; 17; 18; 19; 20; 21; 22; 23; 24; 25; 31; and 32) were used as features to identify/derive the eight endotypes [A-H] in 3166 lupus blood cohort. 28 pairwise binary classification models were built from 8 lupus endotypes using gene modules and SHAP was applied to identify the top three predictors (e.g., modules) that classify the classes with high performance metrics. The 28 binary classification pairs are A vs. B (group A/endotype A vs group/endotype B); A vs. C; A vs. D; A vs. E; A vs F; A vs. G; A vs. H; B vs. C; B vs. D; B vs. E; B vs F; B vs. G; B vs. H; C vs. D; C vs. E; C vs F; C vs. G; C vs. H; D vs. E; D vs F; D vs. G; D vs. H; E vs F; E vs. G; E vs. H; F vs. G; F vs. H; and G vs. H.

Table 40 shows top 3 SHAP predictors (e.g., modules) for each of the 28 binary classifications. In Table 40, for each binary classification, the most important module is marked as 1, the 2nd most important module is marked as 2, and the 3rd most important module is marked as 3. Binary classifications are listed across the top row, for example, A_B denotes group A vs. group B classification, i.e., classification of group A from group B (and vice versa).

Mean SHAP scores of each module within each binary classification are calculated by calculating the mean of the positive SHAP scores and visualized using bubble plot

In order to reduce the modules to genes, the log 2 expression of genes from top3 gene modules identified by SHAP after culling the collinear genes (r>0.8) were used as features and 28 pairwise predictive models were built and top10 gene predictors were identified in each pairwise combination by random forest Gini scores.

A subset of genes from the gdT cell, T cell, and Oxidative Phosphorylation modules was formed and duplicate genes were removed. Collinear genes (r>0.8) were removed. 24 out of 145 genes were identified as collinear genes and were removed from machine learning analysis.121 genes (145-24) are used as features, supervised machine learning algorithms were run and performance metrics and ROC curves and confusion matrices were determined.

The 28 pairwise predictive models were further built using top10 genes from random forest and performance metrics were calculated to evaluate the performance of the model on validation set. Table 41 shows the top 10 gene predictors for each of the 28 binary classifications.

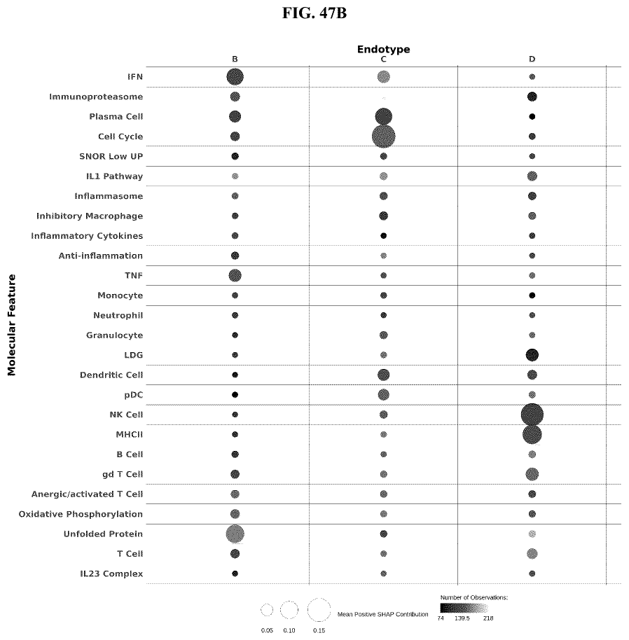

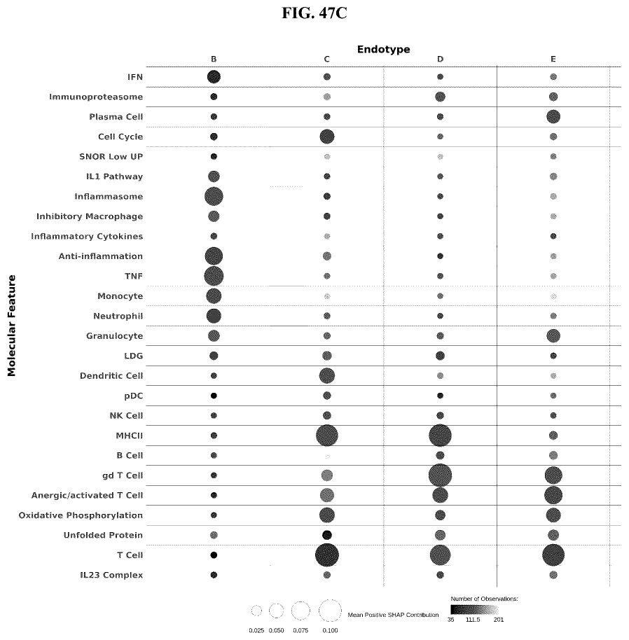

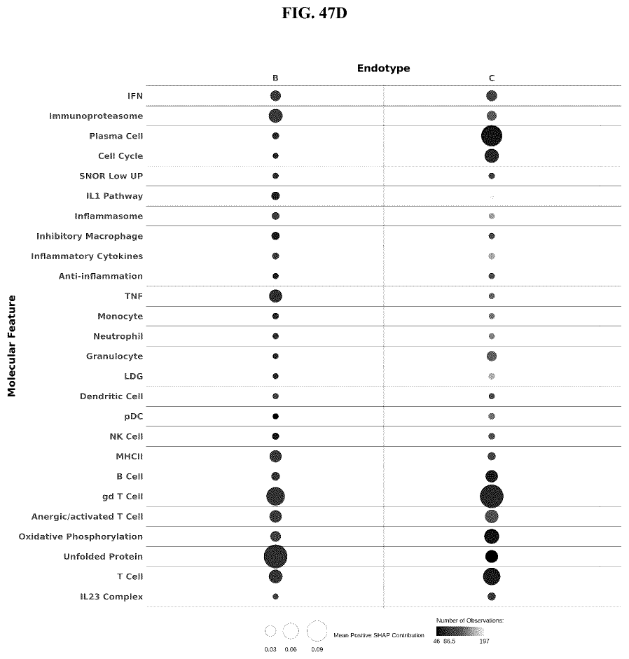

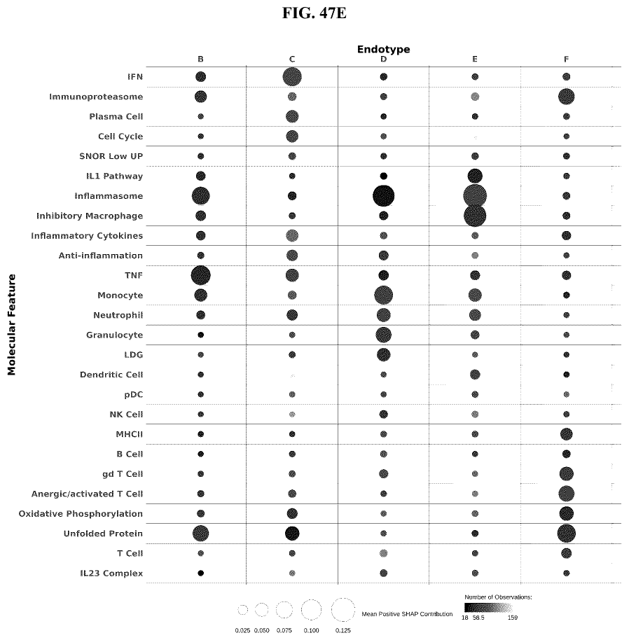

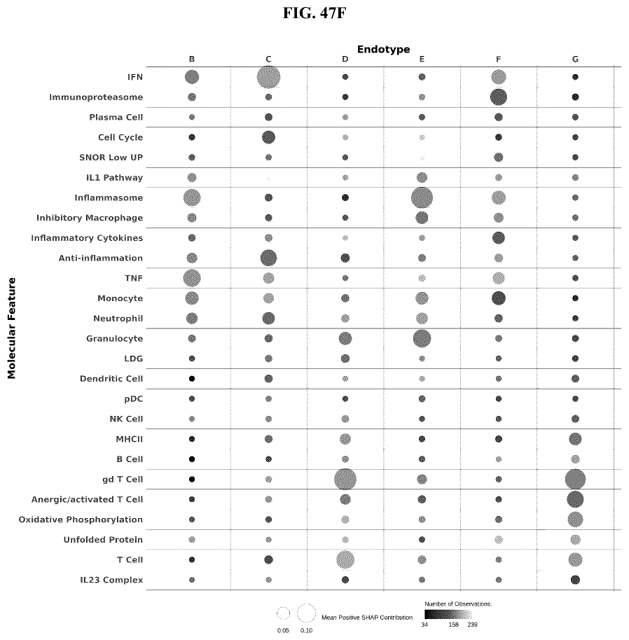

FIGS. 47A-F show SHAP analysis reveals features most distinctive of transcriptional perturbations in the endotypes. FIG. 47A shows SHAP analysis for binary classification of endotype A from endotypes B, C, D, E, F, G and H. FIG. 47B shows SHAP analysis for binary classification of endotype E from endotypes B, C, and D. FIG. 47C shows SHAP analysis for binary classification of endotype F from endotypes B, C, D and E. FIG. 47D shows SHAP analysis for binary classification of endotype D from endotypes B, and C. FIG. 47E shows SHAP analysis for binary classification of endotype G from endotypes B, C, D, E and F. FIG. 47F shows SHAP analysis for binary classification of endotype H from endotypes B, C, D, E, F and G.

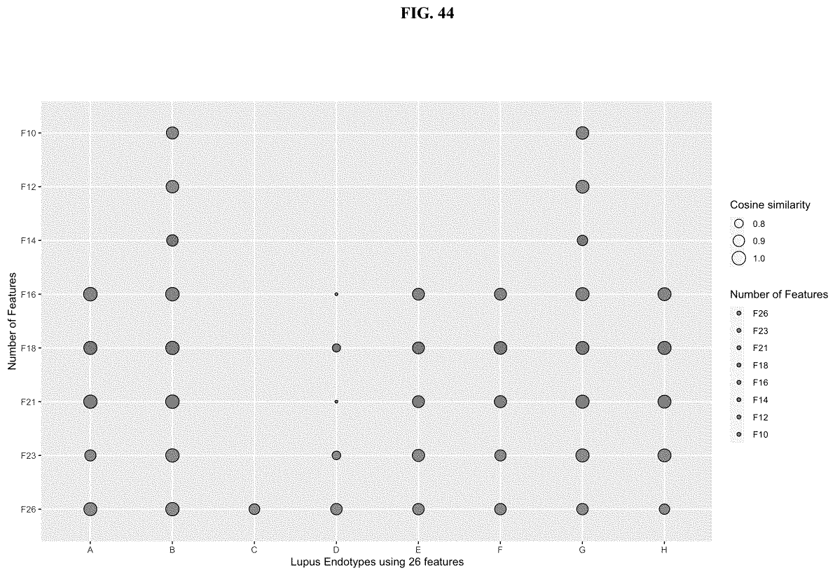

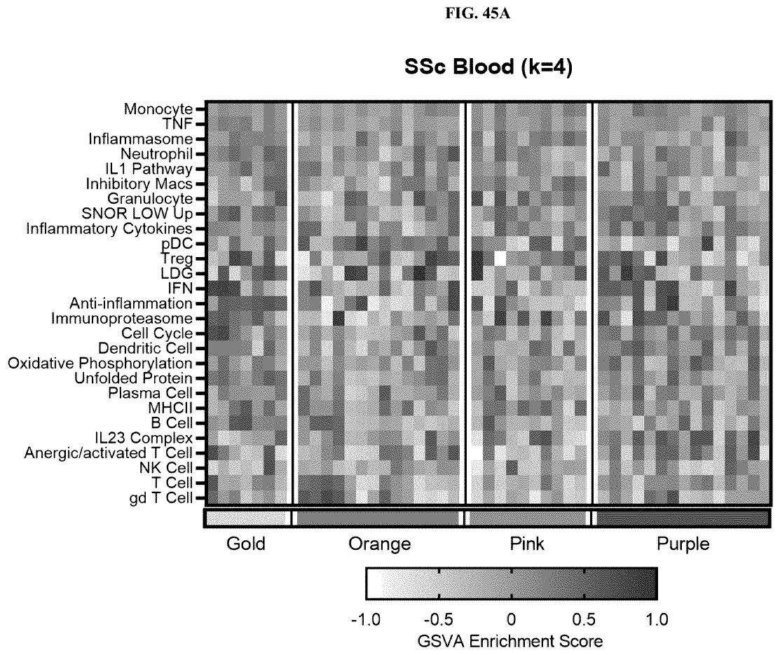

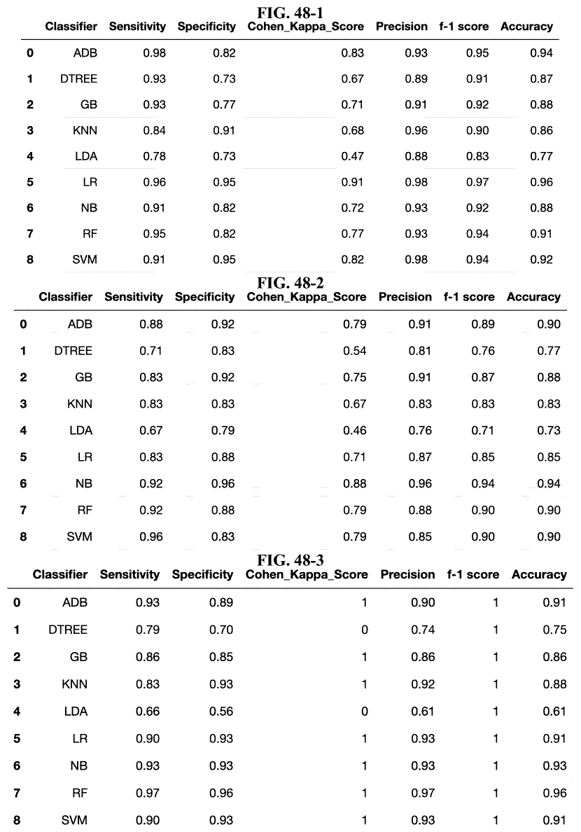

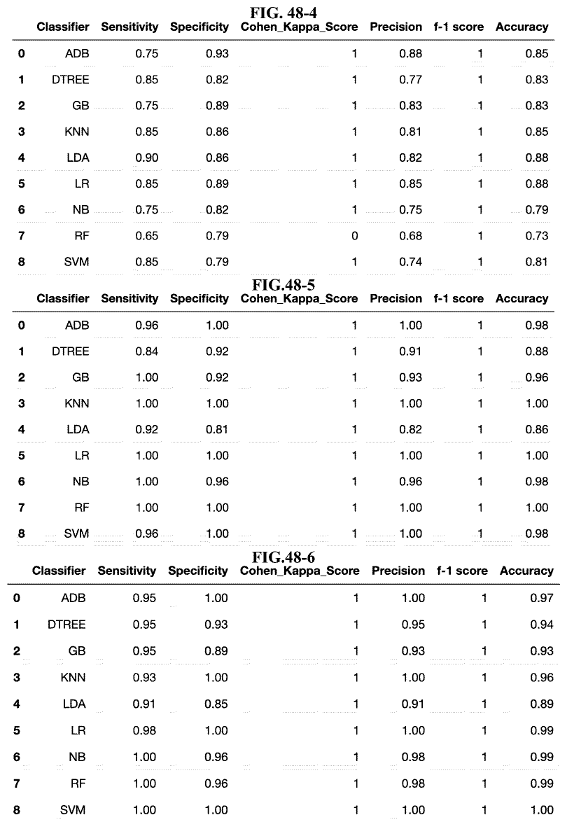

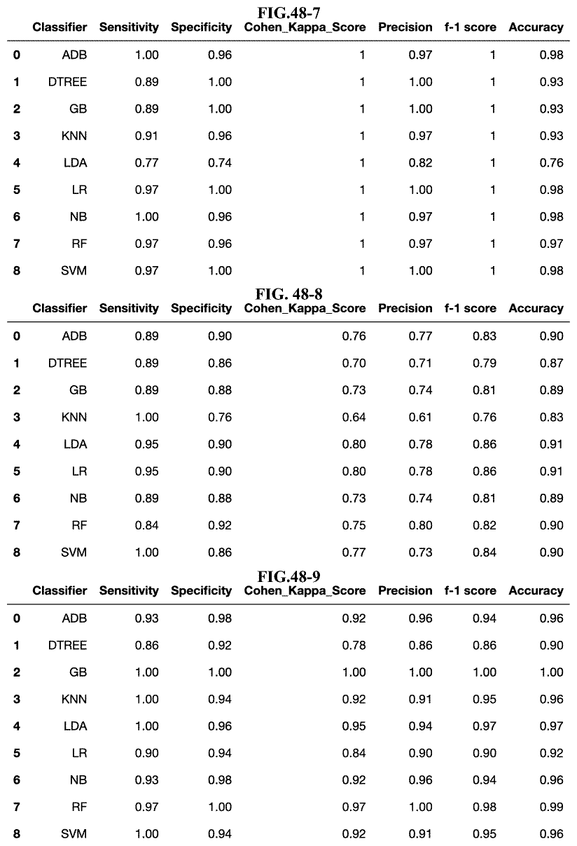

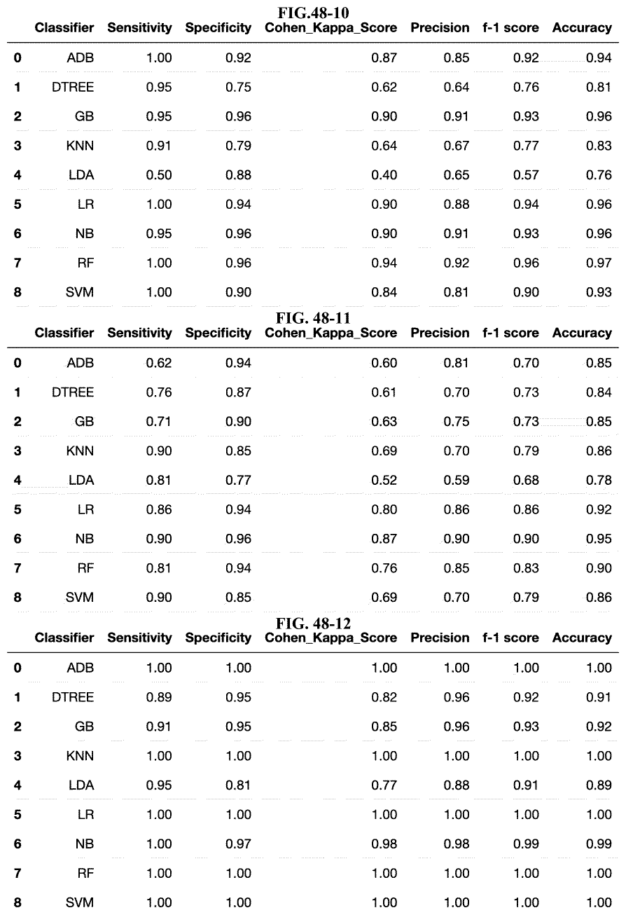

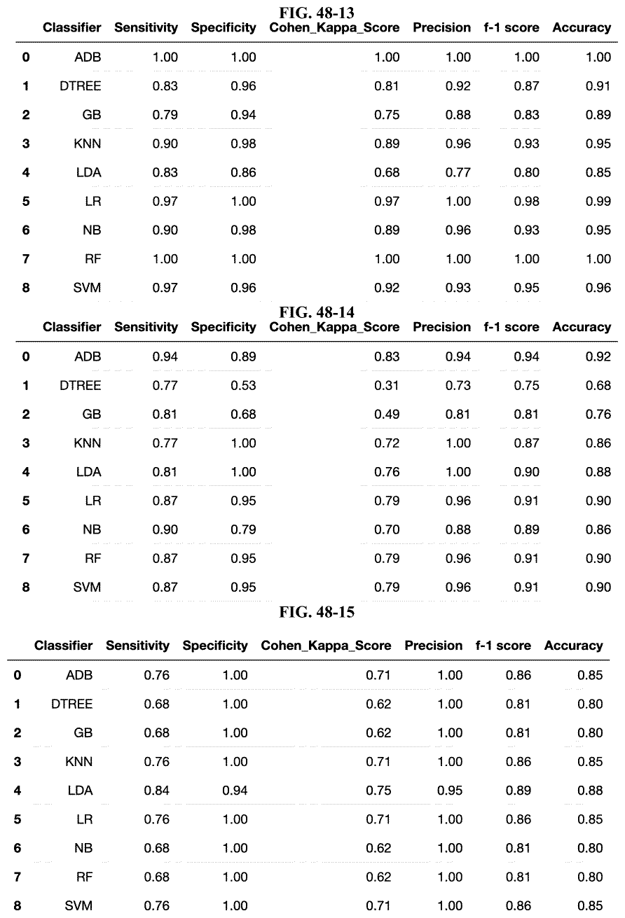

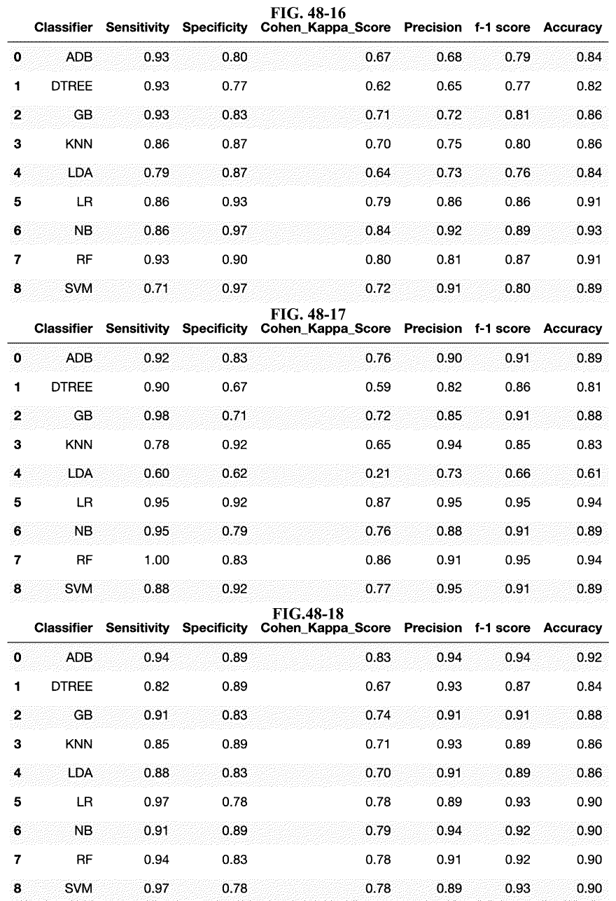

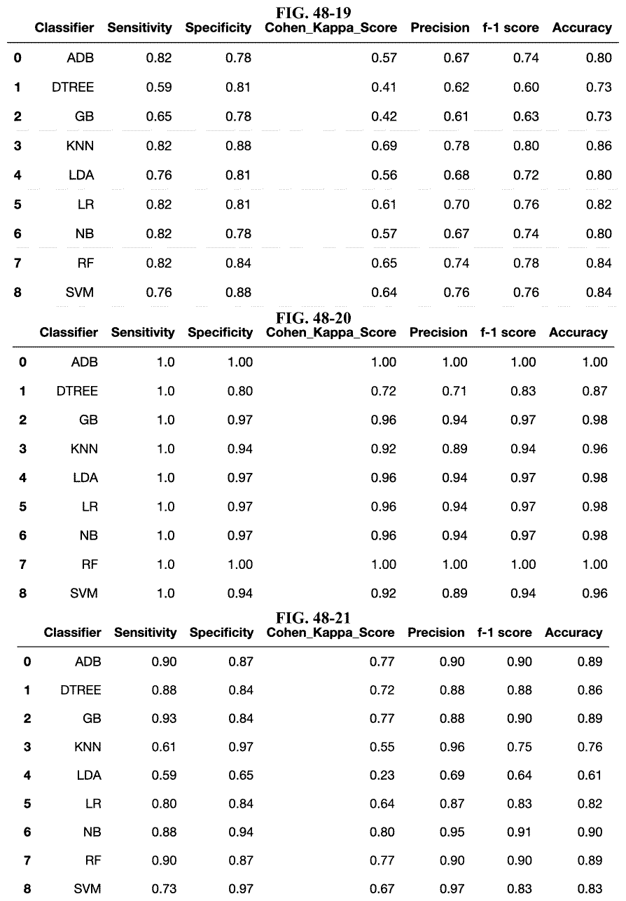

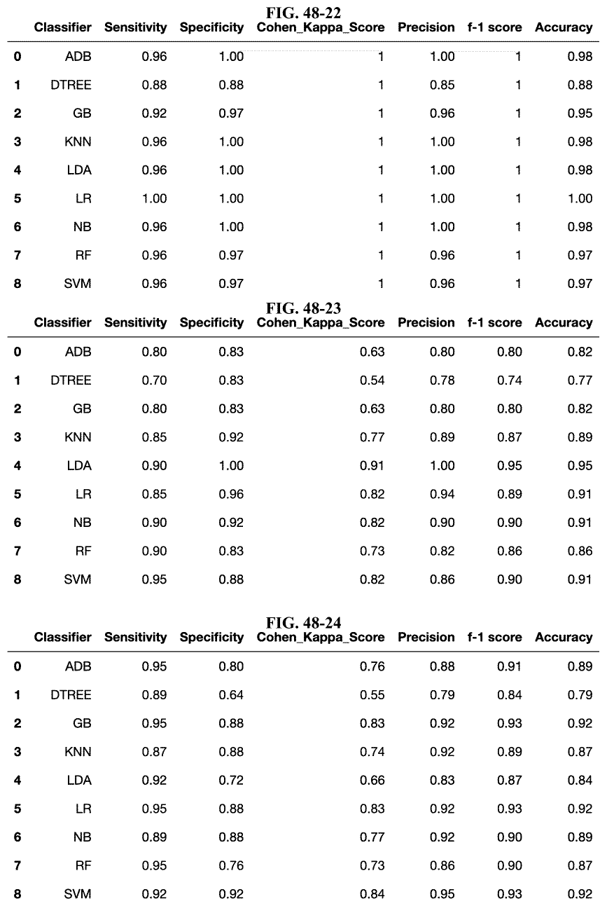

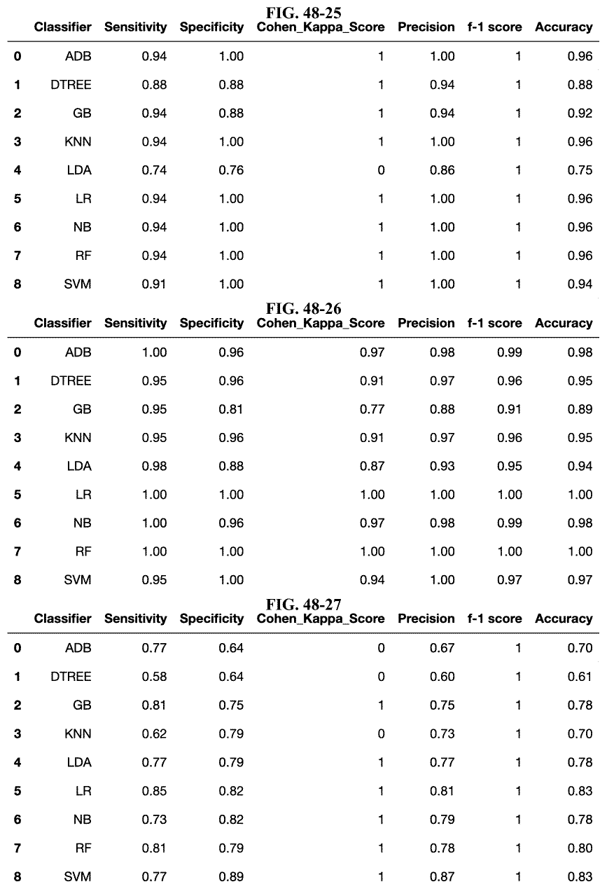

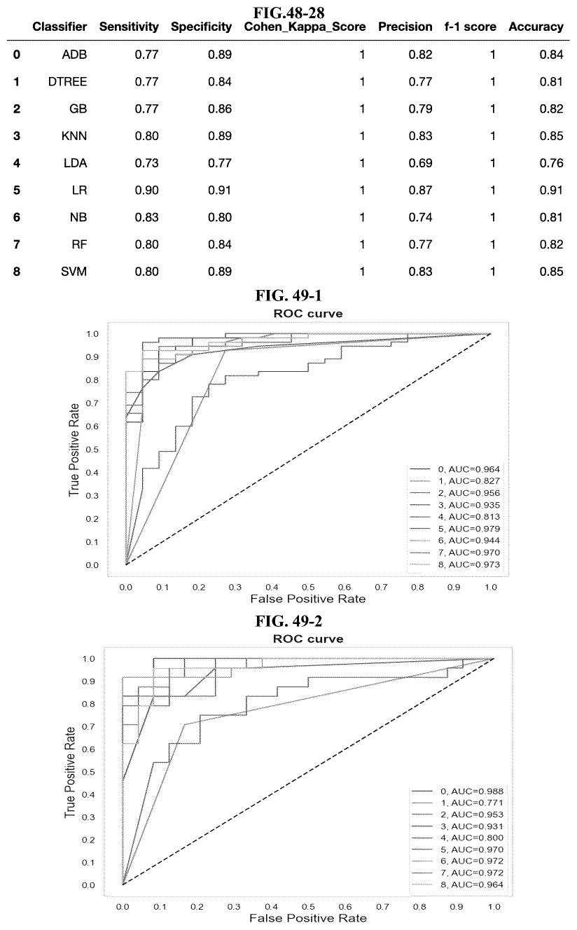

FIGS. 48-1 to 48-28 show performance metrics for 28 pairwise binary classifications (FIG. 48-1: group A vs. group B; FIG. 48-2: group A vs. group C; FIG. 48-3:group A vs. group D; FIG. 48-4: group A vs. group E; FIG. 48-5: group A vs. group F; FIG. 48-6: group A vs. group G; FIG. 48-7: group A vs. group H; FIG. 48-8: group B vs. group C; FIG. 48-9: group B vs. group D; FIG. 48-10: group B vs. group E; FIG. 48-11: group B vs. group F; FIG. 48-12: group B vs. group G; FIG. 48-13: group B vs. group H; FIG. 48-14: group C vs. group D; FIG. 48-15: group C vs. group E; FIG. 48-16: group C vs. group F; FIG. 48-17: group C vs. group G;

FIG. 48-18: group C vs. group H; 48-19: group D vs. group E; FIG. 48-20: group D vs. group F; FIG. 48-21: group D vs. group G; FIG. 48-22: group D vs. group H; 48-23: group E vs. group F; FIG. 48-24: group E vs. group G; FIG. 48-25: group E vs. group H; FIG. 48-26: group F vs. group G; FIG. 48-27: group F vs. group H; FIG. 48-28: group G vs. group H;) using the genes from top 3 SHAP predictors (e.g., modules) of each classification. The top 3 SHAP predictors (marked as 1, 2 and 3) of each classification is shown in Table 40.

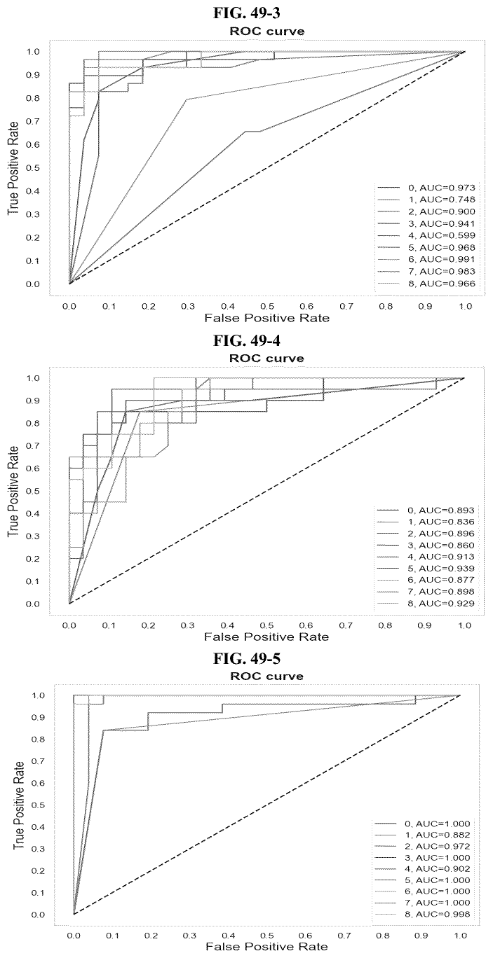

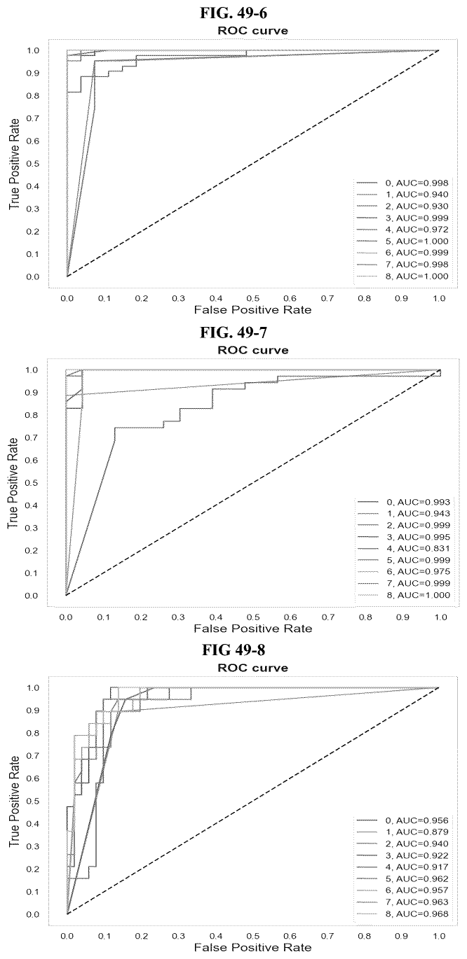

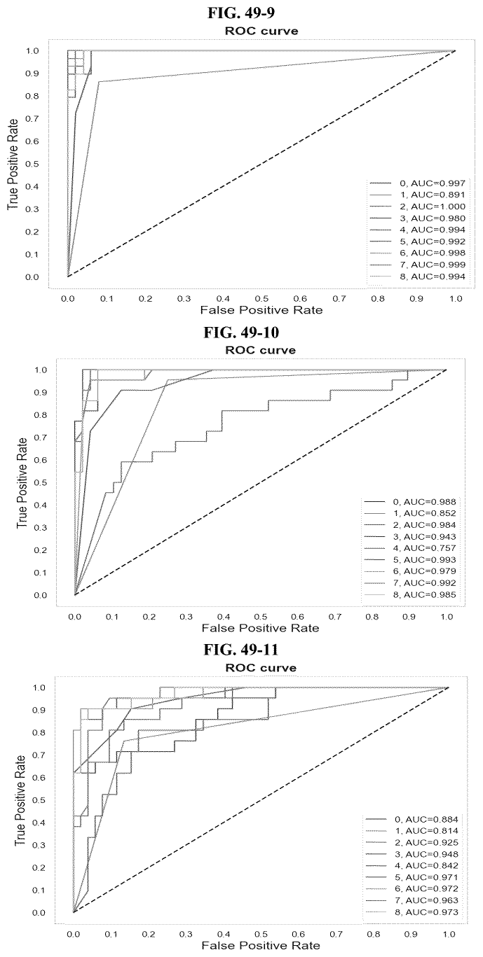

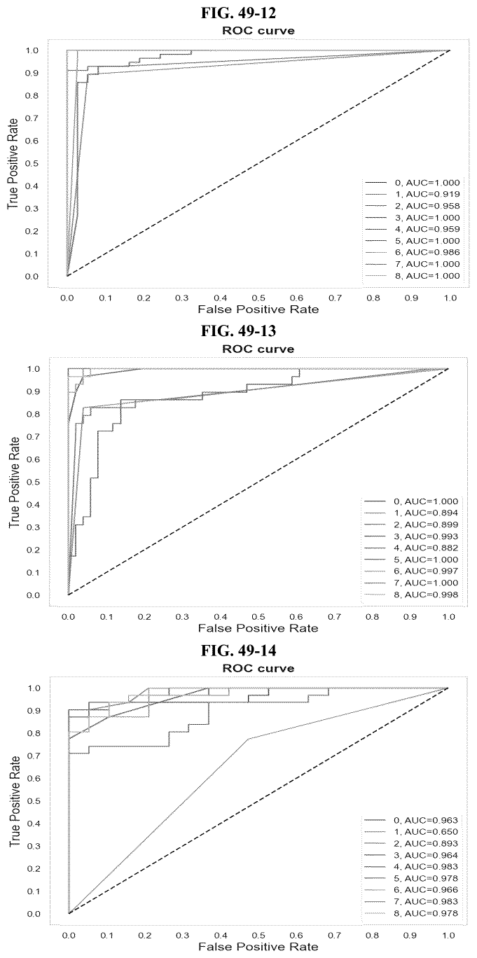

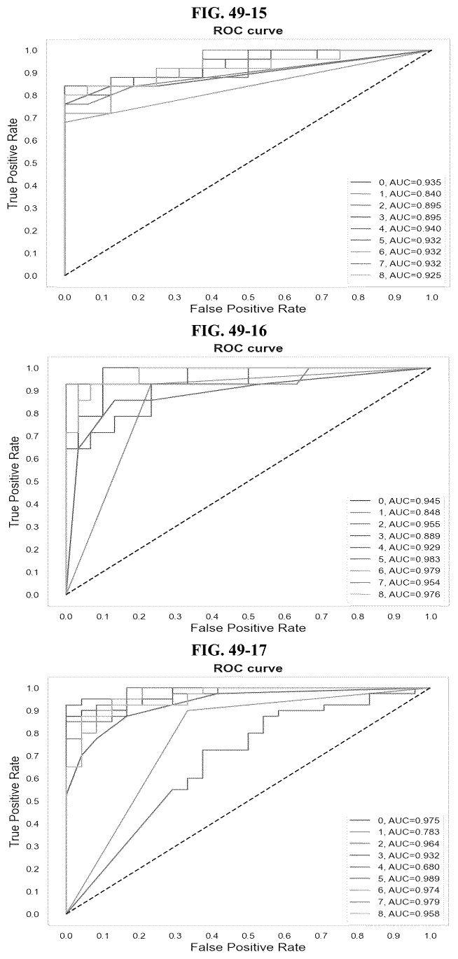

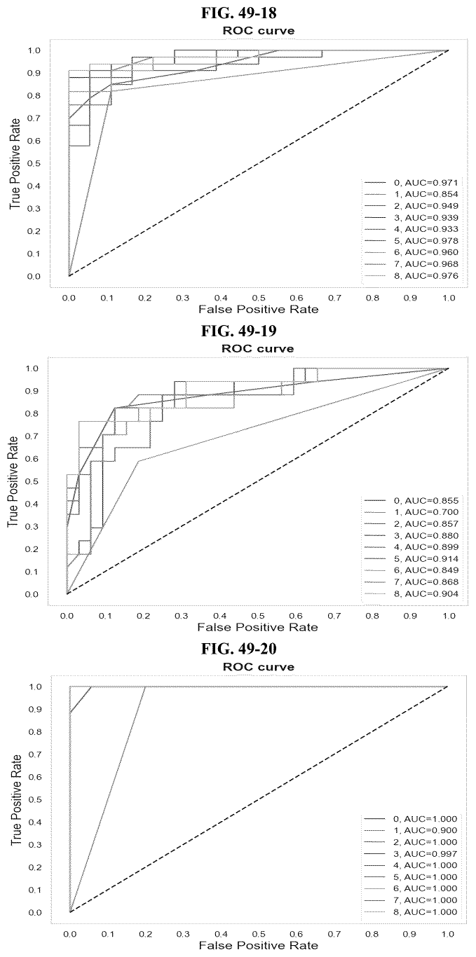

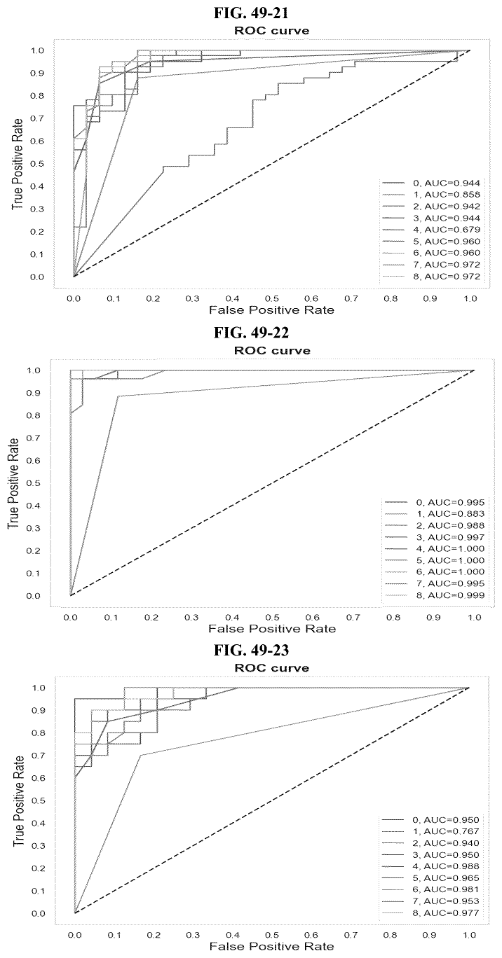

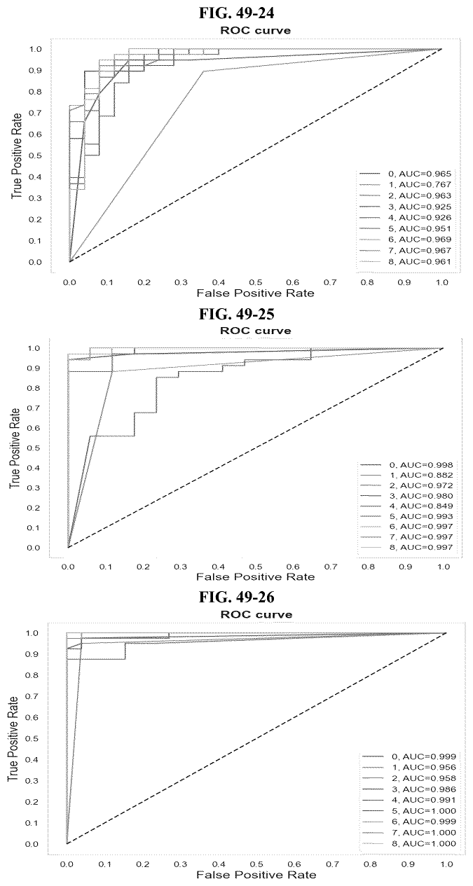

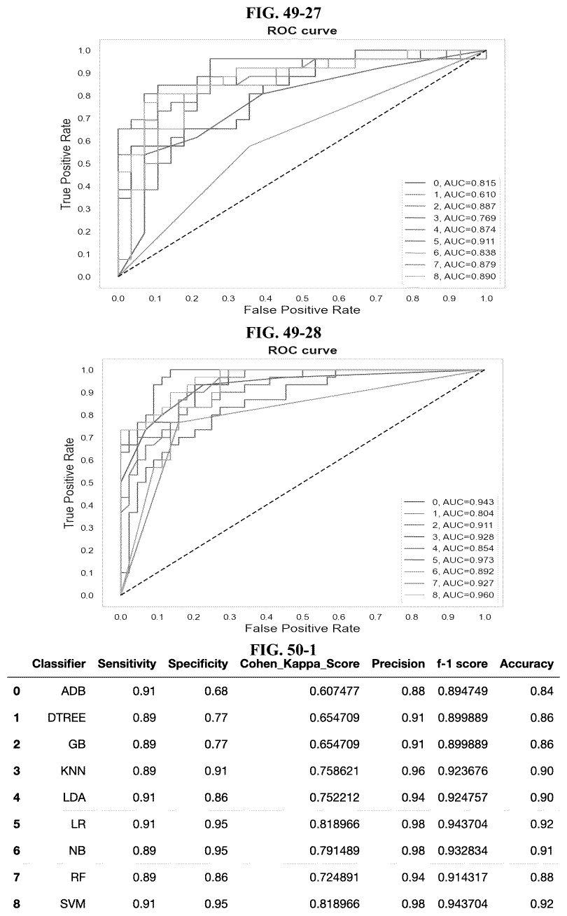

FIGS. 49-1 to 49-28 show ROC curves for 28 pairwise binary classifications (FIG. 49-1: group A vs. group B; FIG. 49-2: group A vs. group C; FIG. 49-3:group A vs. group D; FIG. 49-4: group A vs. group E; FIG. 49-5: group A vs. group F; FIG. 49-6: group A vs. group G;

FIG. 49-7: group A vs. group H; FIG. 49-8: group B vs. group C; FIG. 49-9: group B vs. group D; FIG. 49-10: group B vs. group E; FIG. 49-11: group B vs. group F; FIG. 49-12: group B vs. group G; FIG. 49-13: group B vs. group H; FIG. 49-14: group C vs. group D; FIG. 49-15: group C vs. group E; FIG. 49-16: group C vs. group F; FIG. 49-17: group C vs. group G; FIG. 49-18: group C vs. group H; 49-19: group D vs. group E; FIG. 49-20: group D vs. group F;

FIG. 49-21: group D vs. group G; FIG. 49-22: group D vs. group H; 49-23: group E vs. group F; FIG. 49-24: group E vs. group G; FIG. 49-25: group E vs. group H; FIG. 49-26: group F vs. group G; FIG. 49-27: group F vs. group H; FIG. 49-28: group G vs. group H;) using the genes from top 3 SHAP predictors (e.g., modules) of each classification. The top 3 SHAP predictors (marked as 1, 2 and 3) of each classification is shown in Table 40.

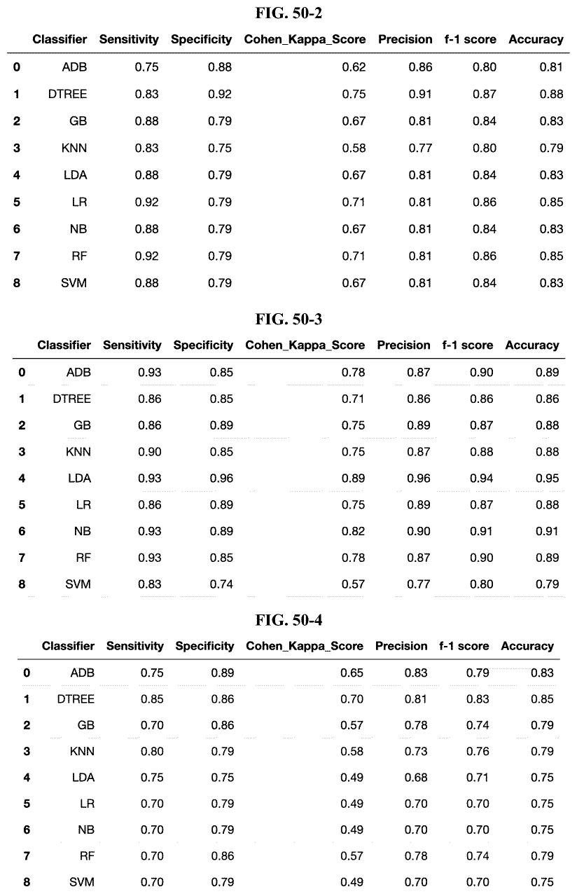

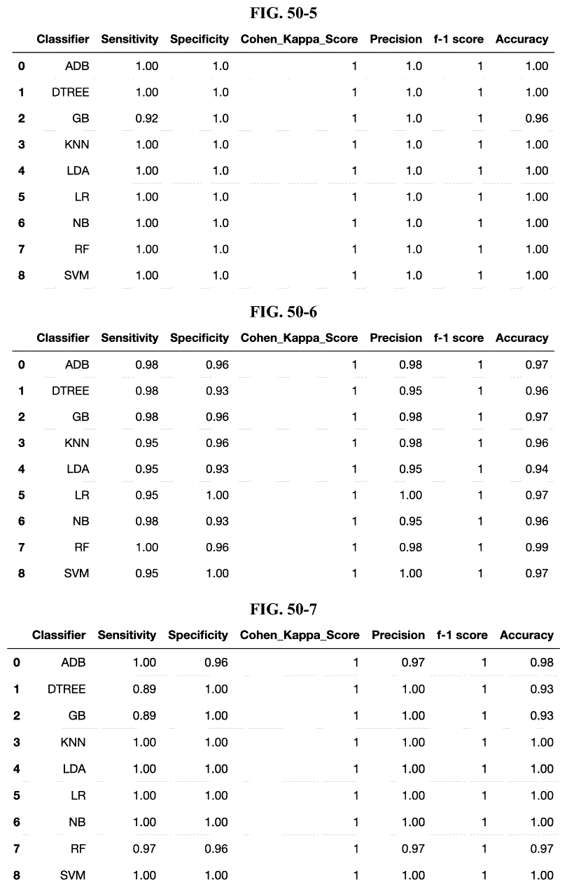

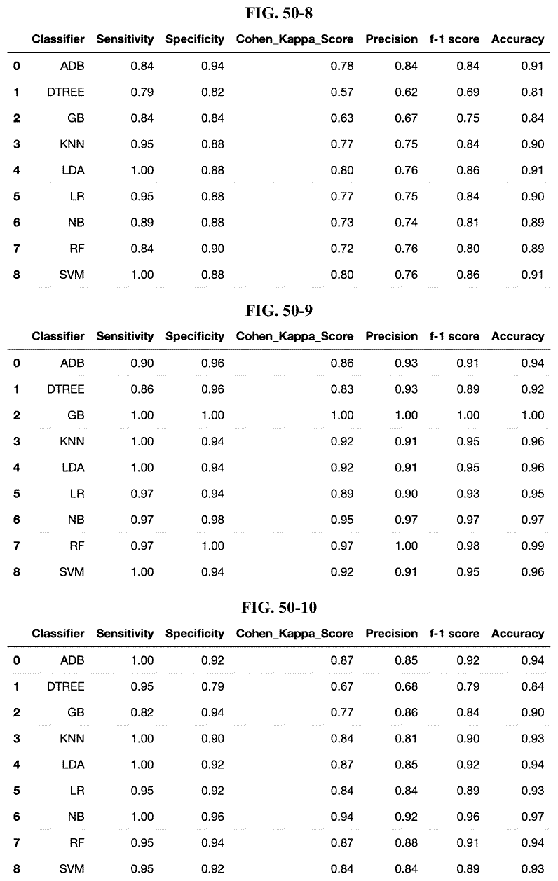

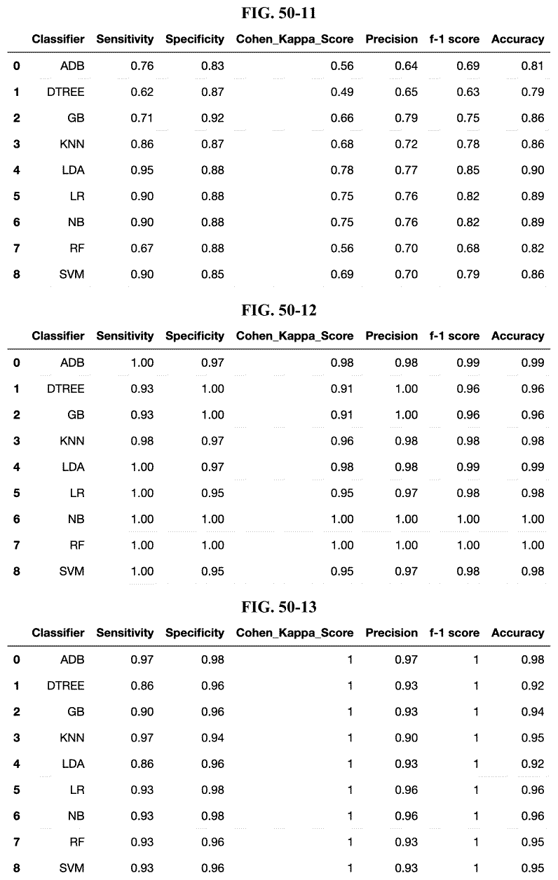

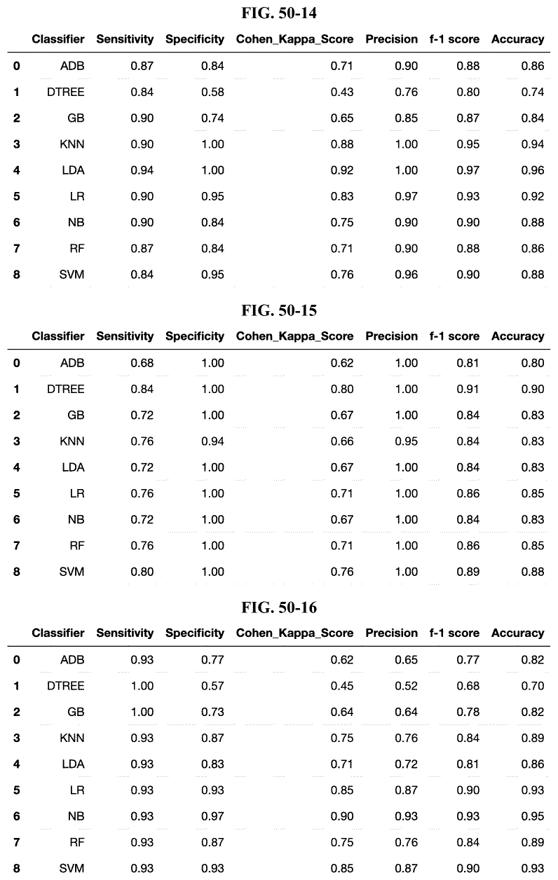

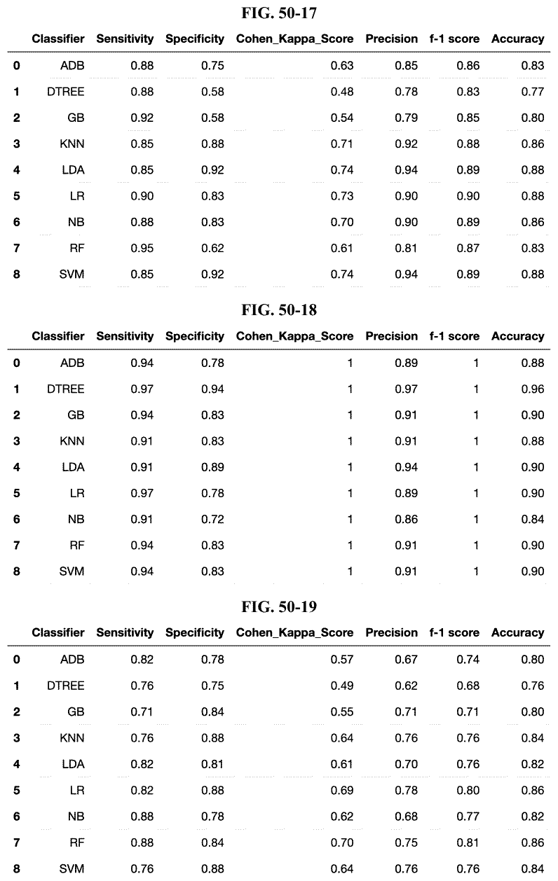

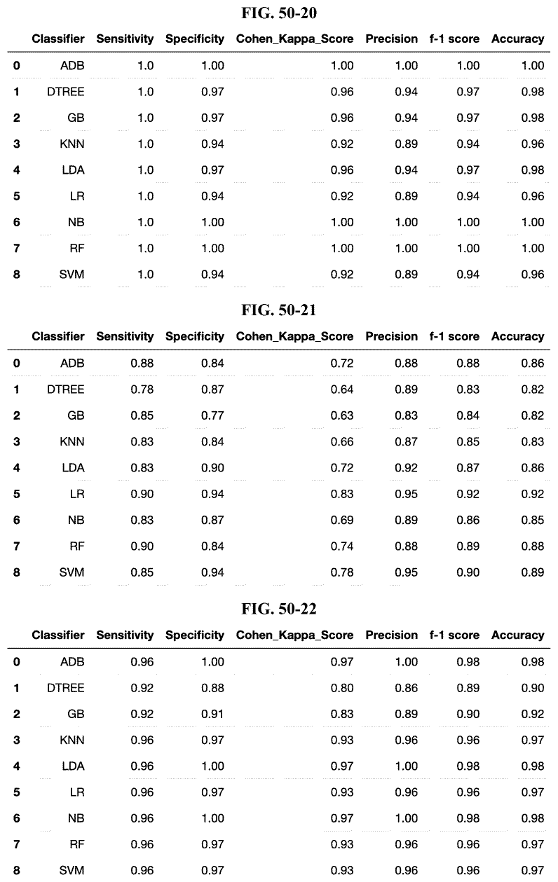

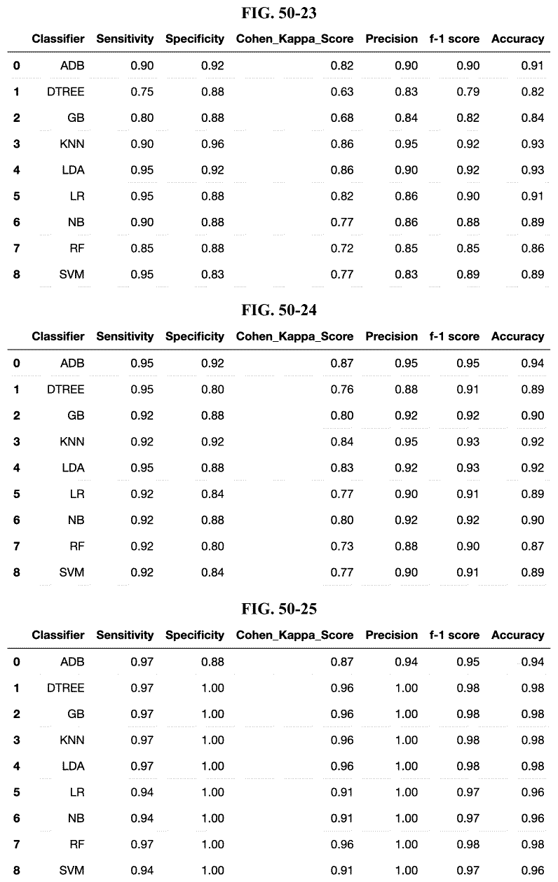

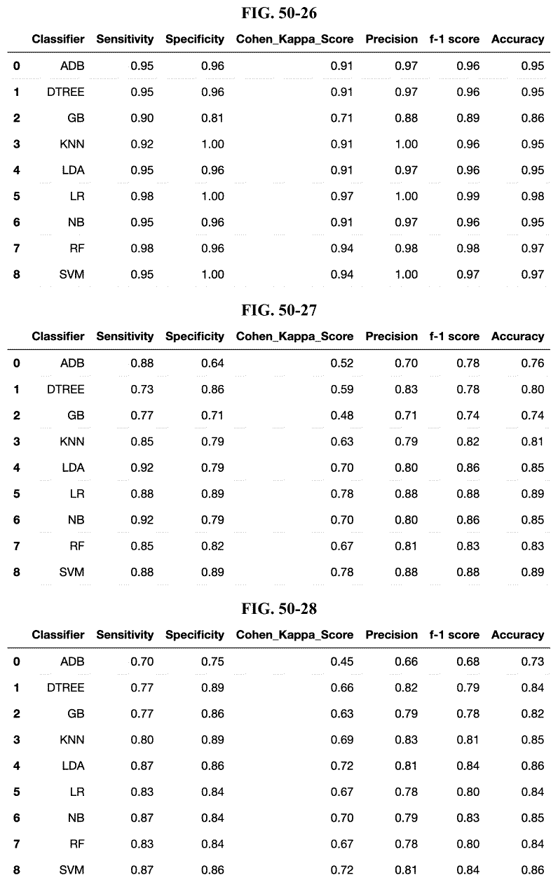

FIGS. 50-1 to 50-28 show performance metrics for 28 pairwise binary classifications (FIG. 50-1: group A vs. group B; FIG. 50-2: group A vs. group C; FIG. 50-3:group A vs. group D; FIG. 50-4: group A vs. group E; FIG. 50-5: group A vs. group F; FIG. 50-6: group A vs. group G; FIG. 50-7: group A vs. group H; FIG. 50-8: group B vs. group C; FIG. 50-9: group B vs. group D; FIG. 50-10: group B vs. group E; FIG. 50-11: group B vs. group F; FIG. 50-12: group B vs. group G; FIG. 50-13: group B vs. group H; FIG. 50-14: group C vs. group D; FIG. 50-15: group C vs. group E; FIG. 50-16: group C vs. group F; FIG. 50-17: group C vs. group G; FIG. 50-18: group C vs. group H; 50-19: group D vs. group E; FIG. 50-20: group D vs. group F; FIG. 50-21: group D vs. group G; FIG. 50-22: group D vs. group H; 50-23: group E vs. group F; FIG. 50-24: group E vs. group G; FIG. 50-25: group E vs. group H; FIG. 50-26: group F vs. group G; FIG. 50-27: group F vs. group H; FIG. 50-28: group G vs. group H;) using the top 10 gene predictors of each binary classification. The top 10 gene predictors of each classification is shown in Table 41.

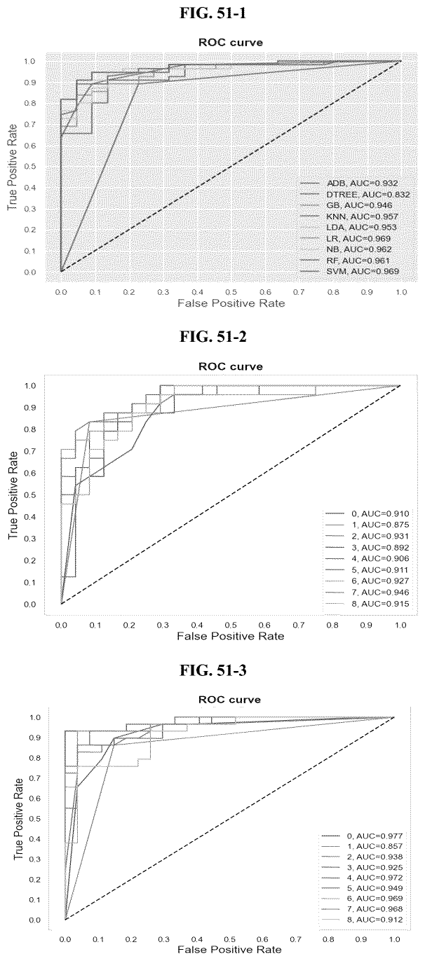

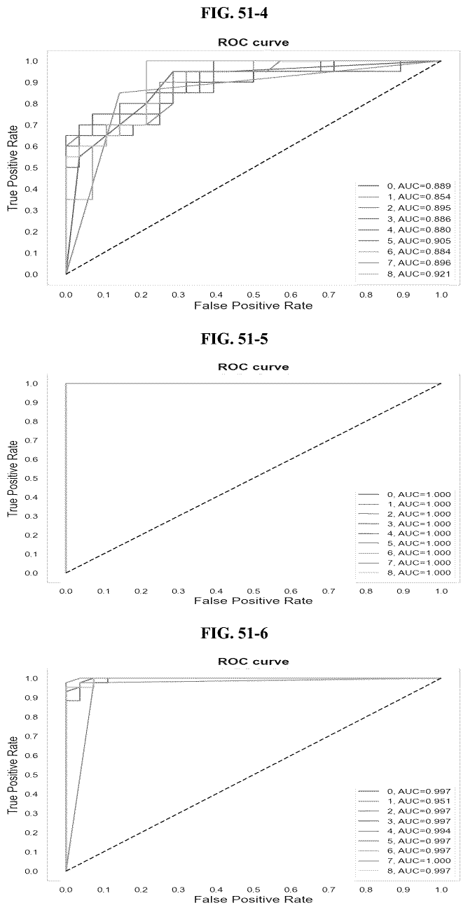

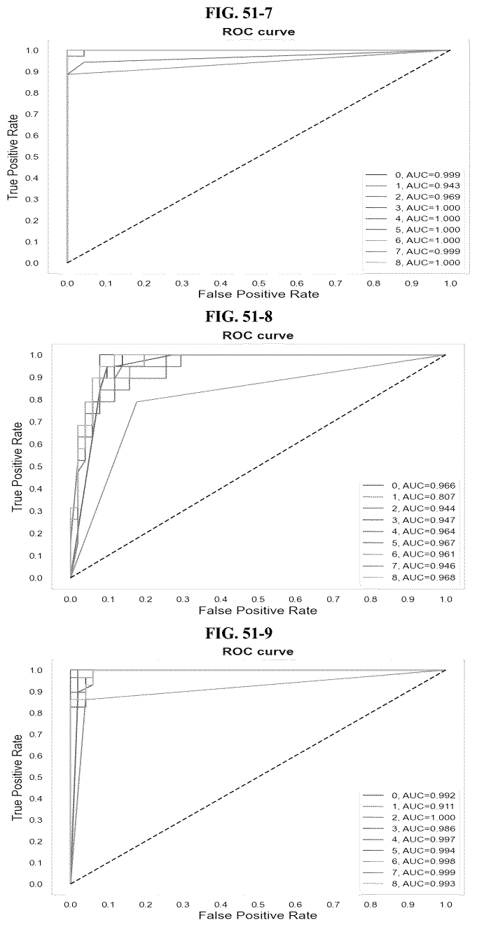

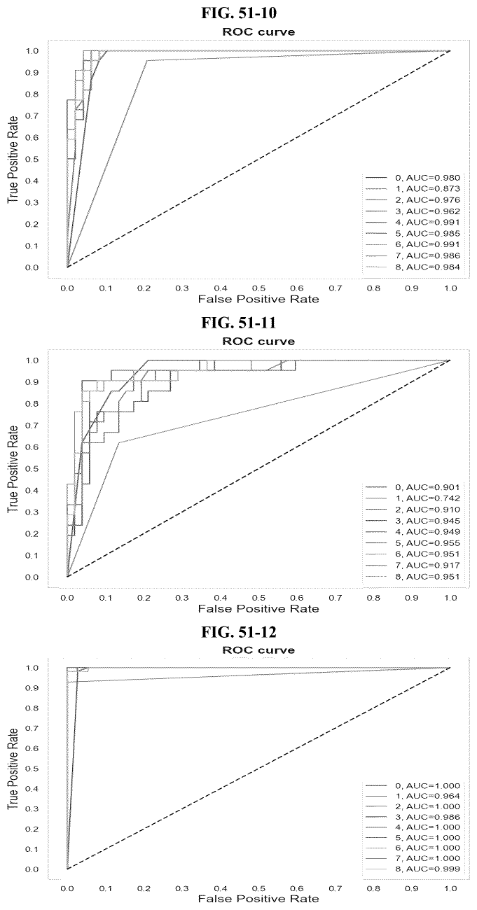

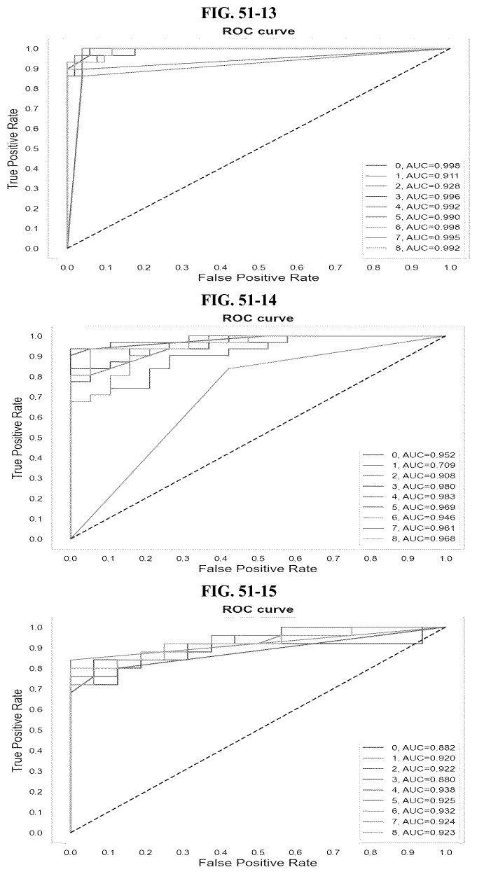

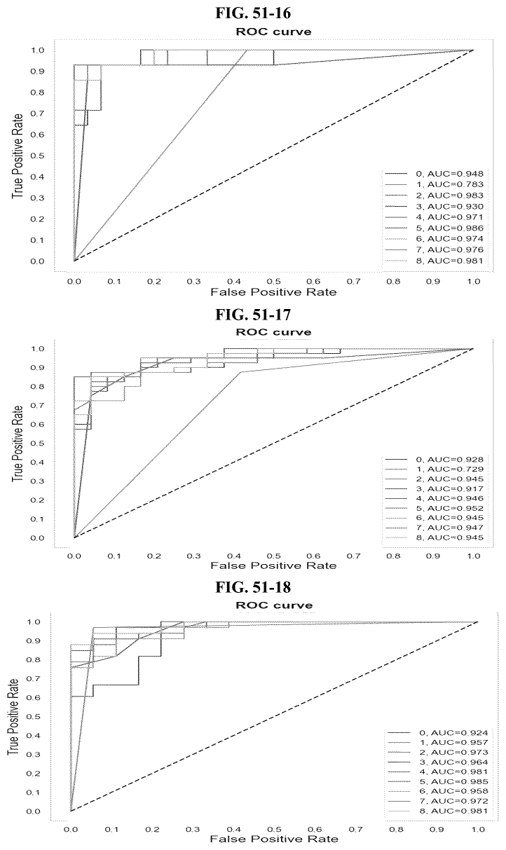

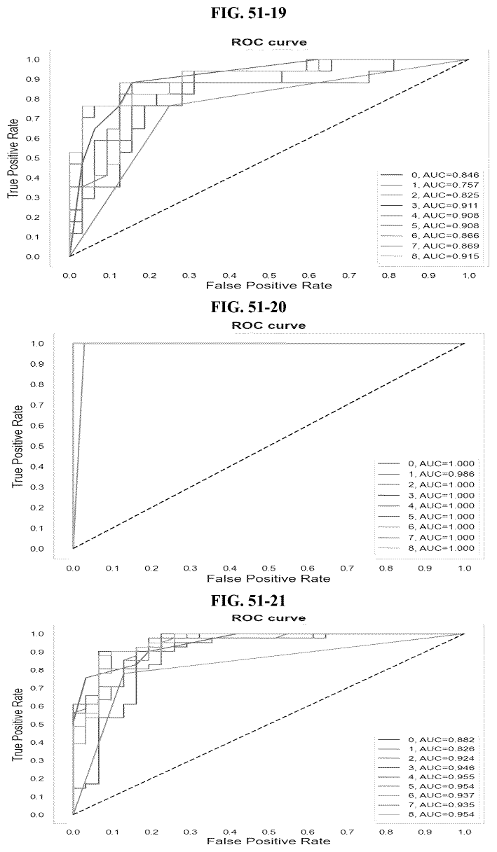

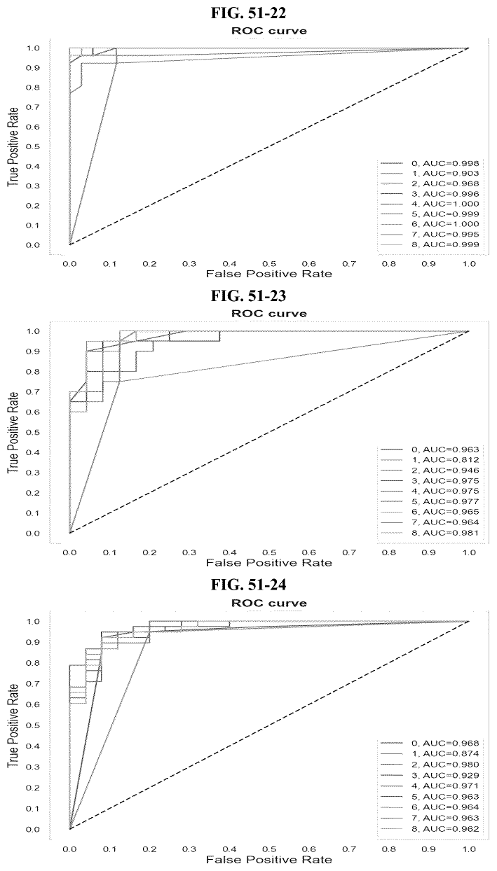

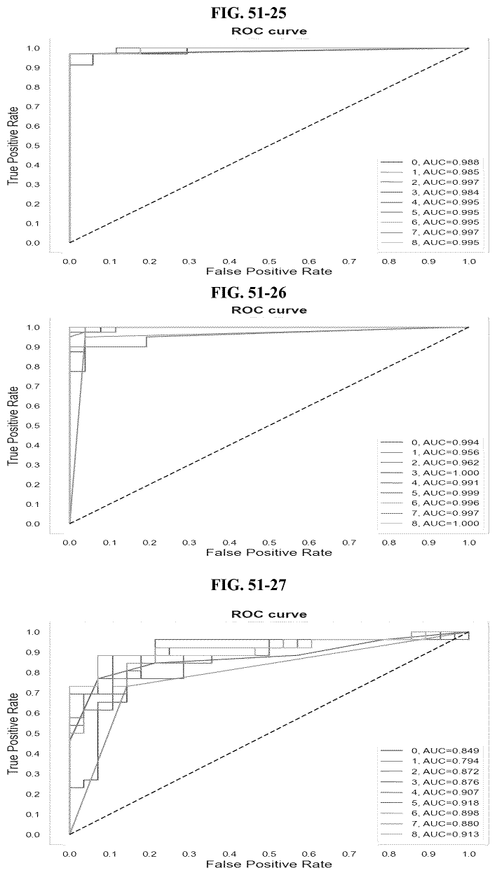

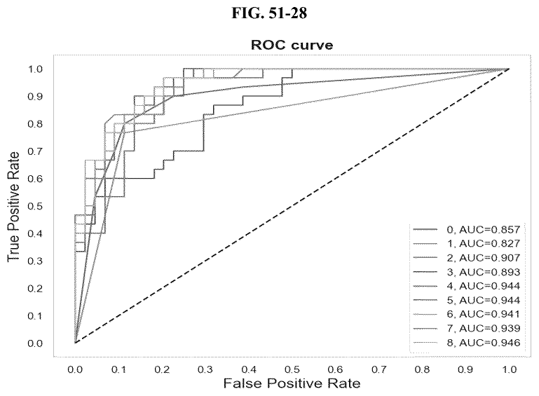

FIGS. 51-1 to 51-28 show ROC curves for 28 pairwise binary classifications (FIG. 51-1: group A vs. group B; FIG. 51-2: group A vs. group C; FIG. 51-3:group A vs. group D; FIG. 51-4: group A vs. group E; FIG. 51-5: group A vs. group F; FIG. 51-6: group A vs. group G;

FIG. 51-7: group A vs. group H; FIG. 51-8: group B vs. group C; FIG. 51-9: group B vs. group D; FIG. 51-10: group B vs. group E; FIG. 51-11: group B vs. group F; FIG. 51-12: group B vs. group G; FIG. 51-13: group B vs. group H; FIG. 51-14: group C vs. group D; FIG. 51-15: group C vs. group E; FIG. 51-16: group C vs. group F; FIG. 51-17: group C vs. group G; FIG. 51-18: group C vs. group H; 51-19: group D vs. group E; FIG. 51-20: group D vs. group F; FIG. 51-21: group D vs. group G; FIG. 51-22: group D vs. group H; 51-23: group E vs. group F; FIG. 51-24: group E vs. group G; FIG. 51-25: group E vs. group H; FIG. 51-26: group F vs. group G; FIG. 51-27: group F vs. group H; FIG. 51-28: group G vs. group H;) using the top 10 gene predictors of each binary classification. The top 10 gene predictors of each classification is shown in Table 41.

TABLE 33
Transcriptomic lupus datasets were utilized in the Example 2

| | | | | | | | | | No. |
|---|---|---|---|---|---|---|---|---|---|
| | | | | | Location | | | | Lupus |
| | Accession | | | | (collected | | Clinical | Clinical | Samples |
| Dataset | Number | Technology | Platform | Source | samples) | Tissue | Metadata | Trial | Analyzed |
| 1 | GSE22098 | microarray | Illumina | Baylor | Dallas, | whole | age, gender, | N/A | 28 |
| | | | HumanHT- | Institute | TX, USA | blood | ancestry | | adult |
| | | | 12 V3.0 | for | | | | | |
| | | | | Immunology | | | | | |
| | | | | Research | | | | | |
| 2 | GSE29536 | microarray | Illumina | Baylor | Mt Sinai | whole | none | N/A | 27 |
| | | | HumanHT- | Institute | Medical | blood | | | SLE |
| | | | 12 V3.0 | for | Center, | | | | |
| | | | and V4.0 | Immunology | New York, | | | | |
| | | | | Research | USA | | | | |
| 3 | GSE39088 | microarray | Affymetrix | Université | multicenter, | whole | age, gender, | N/A | 21 |
| | | | Human Genome | catholique | Europe | blood | ancestry | | SLE |
| | | | U133 Plus | de Louvain | | | | | |
| | | | 2.0 | | | | | | |
| 4 | GSE49454 | microarray | Illumina | Baylor | international | whole | extensive | NCT | 49 |
| | | | Human HT-12 | Institute | | blood | | 00920114 | SLE |
| | | | V4.0 | for | | | | | |
| | | | | Immunology | | | | | |
| | | | | Research | | | | | |
| 5 | GSE61635 | microarray | Affymetrix | Eli Lilly | unknown | whole | none | N/A | 64 |
| | | | Human Genome | & Co. | | blood | | | SLE |
| | | | U133 Plus | | | | | | |
| | | | 2.0 | | | | | | |
| 6 | GSE72747 | microarray | Affymetrix | Université | Belgium, | whole | age, gender, | NCT | 10 |
| | | | Human Genome | catholique | Bulgaria, | blood | SLEDAI, | 01058343 | baseline |
| | | | U133 Plus | de Louvain | Croatia, | | GFR, | | |
| | | | 2.0 | | France, | | dsDNA titer | | |
| | | | | | Germany, | | | | |
| | | | | | Switzerland | | | | |
| 7 | GSE88884 | microarray | Affymetrix | Eli Lilly | international | whole | extensive | NCT | 813 |
| | (ILL-1) | | Human | & Co. | | blood | | 01196091 | baseline |
| | | | Transcriptome | | | | | | active |
| | | | Array 2.0 | | | | | | female |
| 8 | GSE88884 | microarray | Affymetrix | Eli Lilly | international | whole | extensive | NCT | 807 |
| | (ILL-2) | | Human | & Co. | | blood | | 01205438 | baseline |
| | | | Transcriptome | | | | | | active |
| | | | Array 2.0 | | | | | | female |
| 9 | GSE110174 | microarray | Affymetrix | Bristol- | international | whole | neutrophils | NCT | 144 |
| | | | HT HG-U133 + | Myers | | blood | (absolute), | 00119678 | SLE |
| | | | PM Array Plate | Squibb | | | steroid use | | |
| 10 | GSE138458 | microarray | Illumina | Oklahoma | Oklahoma | whole | high/low | N/A | 307 |
| | | | Human HT-12 | Medical | City, OK, USA | blood | SLEDAI | | SLE |
| | | | V4.0 | Research | | | | | |
| | | | | Foundation | | | | | |
| 11 | GSE185047 | microarray | Affymetrix | Université | international | whole | none | NCT | 177 |
| | | | Human Genome | catholique | | blood | | 02665364 | baseline |
| | | | U133 Plus 2.0 | de Louvain | | | | | |
| 12 | GSE72509 | RNA-seq | Illumina | Genentech | UCSF Medical | whole | gender, | N/A | 99 |
| | | | HiSeq 2500 | | Center, San | blood | ancestry, | | SLE |
| | | | | | Francisco, CA, | | Anti-Ro60 | | |
| | | | | | USA | | status, | | |
| | | | | | | | interferon | | |
| | | | | | | | signature | | |
| | | | | | | | metric (ISM) | | |
| | | | | | | | status | | |
| 13 | GSE112087 | RNA-seq | Illumina | CSL | UCSF; | whole | age, gender, | N/A | 29 |
| | | | HiSeq 2500 | Limited, | Melbourne, | blood | ancestry | | female |
| | | | | Bio21 | Australia; | | | | |
| | | | | Institute | Yale-New Haven, | | | | |
| | | | | | CT, USA; | | | | |
| | | | | | Banaras Hindu | | | | |
| | | | | | University, | | | | |
| | | | | | India | | | | |
| 14 | GSE116006 | RNA-seq | Illumina | Wellcome | international | whole | age, gender, | NCT | 160 |
| | | | HiSeq 2000 | Sanger | | blood | IFN status, | 01405196 | SLE |
| | | | | Institute | | | drug exposure | | |
| 15 | SRX10438277 | RNA-seq | Illumina | Temple | Temple | whole | extensive | N/A | 28 |
| | PRJNA717024)¹ | | HiSeq 2000 | University | University | blood | | | SLE |
| | | | | | Hospital, | | | | |
| | | | | | Philadelphia, | | | | |
| | | | | | PA, USA | | | | |
| 16 | GSE65391 | microarray | Illumina | Baylor | Dallas, | whole | extensive | N/A | 137 |
| | | | Human HT-12 | Institute | TX, USA | blood | | | baseline |
| | | | V4.0 | for | | | | | |
| | | | | Immunology | | | | | |
| | | | | Research | | | | | |
| 17 | GSE45291 | microarray | Affymetrix | Johns | Baltimore, | whole | extensive | N/A | 266 |
| | | | HT HG-U133 + | Hopkins | MD, USA | blood | | | SLE |
| | | | PM Array Plate | Lupus | | | | | |
| | | | | Center | | | | | |

¹Stearrett N, Dawson T, Rahnavard A, Bachali P, Bendall M L, Zeng C, et al. Expression of Human Endogenous Retroviruses in Systemic Lupus Erythematosus: Multiomic Integration With Gene Expression. Front Immunol. 2021 Apr. 27; 12: 1485.

TABLE 34

| | K- | | | | |
|---|---|---|---|---|---|
| | means | Hierarchical | Spectral | GMM | SOM |
| Rand index | | | | | |
| K-means | 1 | | | | |
| Hierarchical | 0.84 | 1 | | | |
| Spectral | 0.88 | 0.84 | 1 | | |
| GMM | 0.76 | 0.73 | 0.73 | 1 | |
| SOM | 0.85 | 0.81 | 0.82 | 0.75 | 1 |
| Adjusted Rand Index | | | | | |
| K-means | 1 | | | | |
| Hierarchical | 0.48 | 1 | | | |
| Spectral | 0.6 | 0.51 | 1 | | |
| GMM | 0.23 | 0.17 | 0.16 | 1 | |
| SOM | 0.51 | 0.39 | 0.43 | 0.2 | 1 |
| Silhouette Score | | | | | |
| K-means | 0.141 | | | | |
| Hierarchical | 0.1168 | | | | |
| Spectral | 0.127 | | | | |
| GMM | 0.031 | | | | |
| SOM | 0.1206 | | | | |

TABLE 35

| | | |
|---|---|---|
| | DG4, LG1, LS3, | |
| IR2 Subset | L, and SB3 | |
| Patients | Subset Patients | Total |

Observed Frequency

| | | | |
|---|---|---|---|
| Severe Flare+ | 2 | 81 | 83 |
| Severe Flare− | 82 | 385 | 467 |
| Total | 84 | 466 | 550 |

Expected Frequency

| | | | |
|---|---|---|---|
| Severe Flare+ | 12.68 | 70.32 | 83 |
| Severe Flare− | 71.32 | 395.68 | 467 |
| Total | 84 | 466 | 550 |
| | | p= | 0.00041 |
| | | OR= | 0.116 |

TABLE 36
Seven binary classifications comparing the seven most transcriptionally
perturbed endotypes (B-H) to the least abnormal endotype (A).

| | LR | RF | SVM | DTREE | ADB | NB | LDA | KNN | GB |
|---|---|---|---|---|---|---|---|---|---|
| | Precision | | | | | | | | |
| C1 vs C0 | 1 | 0.91 | 0.97 | 0.67 | 0.89 | 0.93 | 0.97 | 0.78 | 0.85 |
| C2 vs C0 | 0.99 | 0.94 | 0.98 | 0.82 | 0.91 | 0.93 | 0.98 | 0.96 | 0.96 |
| C3 vs C0 | 0.99 | 0.98 | 0.98 | 0.81 | 0.96 | 0.99 | 0.98 | 0.94 | 0.98 |
| C4 vs C0 | 0.99 | 0.98 | 0.97 | 0.87 | 0.96 | 0.98 | 0.98 | 0.95 | 0.96 |
| C5 vs C0 | 1 | 1 | 1 | 0.93 | 1 | 1 | 1 | 1 | 0.98 |
| C6 vs C0 | 1 | 1 | 1 | 0.91 | 1 | 1 | 1 | 1 | 0.92 |
| C7 vs C0 | 1 | 1 | 1 | 0.95 | 1 | 1 | 1 | 1 | 0.95 |
| | AUC | | | | | | | | |
| C1 vs C0 | 0.999 | 0.984 | 0.999 | 0.88 | 0.991 | 0.988 | 0.989 | 0.957 | 0.978 |
| C2 vs C0 | 1 | 0.987 | 0.999 | 0.854 | 0.977 | 0.995 | 0.996 | 0.986 | 0.986 |
| C3 vs C0 | 1 | 0.99 | 0.999 | 0.819 | 0.989 | 0.996 | 0.994 | 0.99 | 0.991 |
| C4 vs C0 | 1 | 0.997 | 1 | 0.887 | 0.997 | 1 | 0.997 | 0.998 | 0.993 |
| C5 vs C0 | 1 | 1 | 1 | 0.965 | 1 | 1 | 1 | 1 | 1 |
| C6 vs C0 | 1 | 1 | 1 | 0.967 | 1 | 1 | 1 | 1 | 0.993 |
| C7 vs C0 | 1 | 1 | 1 | 0.968 | 1 | 1 | 1 | 1 | 0.986 |
| | Sensitivity | | | | | | | | |
| C1 vs C0 | 0.89 | 0.79 | 0.87 | 0.84 | 0.87 | 0.74 | 0.79 | 0.66 | 0.76 |
| C2 vs C0 | 0.98 | 0.9 | 0.98 | 0.88 | 0.97 | 0.99 | 0.94 | 0.92 | 0.9 |
| C3 vs C0 | 0.95 | 0.88 | 0.96 | 0.72 | 0.87 | 0.91 | 0.91 | 0.88 | 0.88 |
| C4 vs C0 | 0.99 | 0.96 | 0.99 | 0.88 | 0.97 | 0.99 | 0.95 | 0.99 | 0.95 |
| C5 vs C0 | 1 | 0.99 | 1 | 0.97 | 1 | 1 | 1 | 1 | 0.99 |
| C6 vs C0 | 1 | 1 | 1 | 0.96 | 1 | 1 | 1 | 1 | 0.94 |
| C7 vs C0 | 1 | 1 | 1 | 0.98 | 1 | 1 | 1 | 1 | 0.99 |
| | Specificity | | | | | | | | |
| C1 vs C0 | 1 | 0.98 | 0.99 | 0.92 | 0.98 | 0.99 | 0.99 | 0.96 | 0.97 |
| C2 vs C0 | 0.99 | 0.95 | 0.98 | 0.83 | 0.92 | 0.94 | 0.98 | 0.96 | 0.97 |
| C3 vs C0 | 0.99 | 0.99 | 0.99 | 0.92 | 0.98 | 0.99 | 0.99 | 0.97 | 0.99 |
| C4 vs C0 | 0.99 | 0.98 | 0.97 | 0.9 | 0.97 | 0.98 | 0.98 | 0.96 | 0.97 |
| C5 vs C0 | 1 | 1 | 1 | 0.96 | 1 | 1 | 1 | 1 | 0.99 |
| C6 vs C0 | 1 | 1 | 1 | 0.97 | 1 | 1 | 1 | 1 | 0.98 |
| C7 vs C0 | 1 | 1 | 1 | 0.95 | 1 | 1 | 1 | 1 | 0.95 |

TABLE 37
Determining whether there are significant differences based on Z-score normalization. Z-scores for each module
in each subset. Cutoff of −2 or 2 was used. GSVA scores for the modules shown in bold were imputed.

| | Z-score: | Z-score: | Z-score: | Z-score: | Z-score: | Z-score: | Z-score: |
|---|---|---|---|---|---|---|---|
| | endotype | endotype | endotype | endotype | endotype | endotype | endotype |
| | B | C | D | E | F | G | H |
| IFN | −0.13 | 0.98 | 2.79 | 2.78 | 1.56 | 3.84 | 3.89 |
| Immunoproteasome | −0.71 | 0.50 | 1.65 | 0.92 | −0.15 | 2.17 | 1.56 |
| Plasma Cell | −0.43 | −0.18 | 1.31 | 1.56 | −0.08 | 1.40 | 0.77 |
| IG Chains | −0.43 | −0.18 | 1.31 | 1.56 | −0.08 | 1.40 | 0.77 |
| Cell Cycle | 0.20 | −0.05 | 1.26 | 2.26 | 1.09 | 1.80 | 1.66 |
| SNOR Low UP | −0.24 | 0.64 | 1.30 | 1.10 | 0.32 | 1.78 | 1.61 |
| IL1 Pathway | −0.79 | 1.07 | 1.05 | 0.14 | 1.01 | 2.19 | 1.82 |
| Inflammasome | −0.47 | 1.73 | 1.74 | 0.89 | 1.52 | 3.45 | 3.12 |
| Inhibitory Macs | −0.31 | 1.80 | 1.79 | 0.87 | 1.61 | 3.25 | 2.95 |
| Inflammatory Cytokines | −0.17 | 0.89 | 1.69 | 1.16 | 0.76 | 2.53 | 2.25 |
| Anti-inflammation | −0.03 | 1.29 | 1.70 | 1.83 | 2.42 | 3.53 | 3.86 |
| TNF | −0.55 | 1.51 | 1.93 | 1.51 | 1.53 | 3.47 | 3.25 |
| Monocyte | 0.52 | 2.16 | 1.82 | 1.50 | 2.59 | 3.78 | 4.09 |
| Neutrophil | 0.03 | 1.30 | 1.30 | 1.39 | 2.07 | 3.19 | 3.44 |
| Granulocyte | 0.59 | 1.81 | 0.42 | 0.75 | 2.59 | 2.45 | 3.24 |
| LDG | 0.51 | 0.80 | −0.02 | 1.23 | 1.76 | 1.32 | 1.89 |
| Dendritic Cell | −1.04 | 0.33 | 0.16 | −0.99 | −0.94 | 0.13 | −0.69 |
| pDC | −0.18 | 0.53 | 0.12 | −0.55 | −0.12 | 0.24 | −0.04 |
| TCRD | −2.80 | −1.66 | 0.50 | −0.66 | −3.21 | −0.21 | −2.95 |
| NK Cell | −1.15 | −0.33 | 0.26 | −1.08 | −1.11 | −0.13 | −1.15 |
| MHCII | −1.65 | −0.19 | 0.74 | −0.74 | −1.97 | 0.30 | −1.43 |
| B Cell | −1.37 | −0.56 | 0.58 | −0.17 | −1.24 | 0.17 | −0.89 |
| gd T Cell | −2.80 | −1.66 | 0.50 | −0.66 | −3.21 | −0.21 | −2.95 |
| Anergic/activated T Cell | −1.66 | −0.76 | 0.58 | −0.10 | −2.04 | 0.36 | −1.78 |
| Oxidative Phosphorylation | −1.86 | −0.81 | 0.64 | −0.06 | −2.09 | 0.55 | −1.48 |
| Unfolded Protein | −1.58 | −0.06 | 1.28 | 0.61 | −0.89 | 1.61 | 0.07 |
| TCRAJ | −2.85 | −1.46 | 0.40 | −0.68 | −3.38 | −0.32 | −2.98 |
| T Cell | −2.85 | −1.46 | 0.40 | −0.68 | −3.38 | −0.32 | −2.98 |
| TCRA | −2.85 | −1.46 | 0.40 | −0.68 | −3.38 | −0.32 | −2.98 |
| TCRB | −2.85 | −1.46 | 0.40 | −0.68 | −3.38 | −0.32 | −2.98 |
| IL23 Complex | −0.89 | −0.87 | −0.01 | −0.59 | −1.50 | −0.47 | −1.30 |
| Treg | −2.85 | −1.46 | 0.40 | −0.68 | −3.38 | −0.32 | −2.98 |

TABLE 38
Value of the difference relative to endotype A - if significant (only significantly
changed modules are shown). Significant differences between endotype A and
endotypes B-H were determined using a Z-score approach. Modules in a subset
were considered significantly different in the mean module GSVA score in the
endotype was different from the same module in A by a Z-score of <−2
or a z-score >2. The value shown is the Difference between endotype mean
and endotype A mean. GSVA scores for the modules in bold were imputed.

| | B | C | D | E | F | G | H |
|---|---|---|---|---|---|---|---|
| IFN | | | 0.789 | 0.787 | | 1.836 | 1.188 |
| Immunoproteasome | | | | | | 0.928 | |
| Plasma Cell | | | | | | | |
| IG Chains | | | | | | | |
| Cell Cycle | | | | 0.733 | | | |
| SNOR Low UP | | | | | | | |
| IL1 Pathway | | | | | | 0.543 | |
| Inflammasome | | | | | | 0.814 | 0.735 |
| Inhibitory Macs | | | | | | 0.779 | 0.778 |
| Inflammatory Cytokines | | | | | | 0.736 | 0.656 |
| Anti-inflammation | | | | | 0.687 | 1.241 | 1.962 |
| TNF | | | | | | 0.422 | 0.394 |
| Monocyte | | 0.346 | | | 0.366 | 0.532 | 0.576 |
| Neutrophil | | | | | 0.477 | 0.736 | 0.793 |
| Granulocyte | | | | | 0.697 | 0.653 | 0.864 |
| LDG | | | | | | | |
| Dendritic Cell | | | | | | | |
| pDC | | | | | | | |
| TCRD | −0.885 | | | | −1.131 | | −0.932 |
| NK Cell | | | | | | | |
| MHCII | | | | | | | |
| B Cell | | | | | | | |
| gd T Cell | −0.797 | | | | −0.913 | | −0.839 |
| Anergic/activated T Cell | | | | | −0.640 | | |
| Oxidative Phosphorylation | | | | | −0.557 | | |
| Unfolded Protein | | | | | | | |
| TCRAJ | −0.815 | | | | −0.966 | | −0.852 |
| T Cell | −0.754 | | | | −0.894 | | −0.788 |
| TCRA | −0.793 | | | | −0.939 | | −0.829 |
| TCRB | −0.826 | | | | −0.978 | | −0.862 |
| IL23 Complex | | | | | | | |
| Treg | −0.857 | | | | −1.154 | | −0.896 |

TABLE 39
Summary of significant module/feature deviations from endotype A by z-score method (significantly enriched
modules are shown with upward arrow, and significantly de-enriched modules are shown with downward arrow).

| B | C | D | E | F | G | H |
|---|---|---|---|---|---|---|
| TCRD ↓ | Monocyte ↑ | IFN ↑ | IFN ↑ | Anti-inflammation ↑ | IFN ↑ | IFN ↑ |
| gd T Cell ↓ | | | Cell Cycle ↑ | Monocyte ↑ | Immunoproteasome ↑ | Inflammasome ↑ |
| TCRAJ ↓ | | | | Neutrophil↑ | IL1 Pathway ↑ | Inhibitory Macs ↑ |
| T Cell ↓ | | | | Granulocyte↑ | Inflammasome ↑ | Inflammatory |
| | | | | | | Cytokines ↑ |
| TCRA ↓ | | | | TCRD ↓ | Inhibitory Macs ↑ | Anti- |
| | | | | | | inflammation ↑ |
| TCRB ↓ | | | | gd T Cell ↓ | Inflammatory | TNF ↑ |
| | | | | | Cytokines ↑ | |
| Treg ↓ | | | | Anergic/activated | Anti-inflammation ↑ | Monocyte ↑ |
| | | | | T Cell ↓ | | |
| | | | | Oxidative | TNF ↑ | Neutrophil ↑ |
| | | | | Phosphorylation ↓ | | |
| | | | | TCRAJ ↓ | Monocyte ↑ | Granulocyte ↑ |
| | | | | T Cell ↓ | Neutrophil ↑ | TCRD ↓ |
| | | | | TCRA ↓ | Granulocyte ↑ | gd T Cell ↓ |
| | | | | TCRB ↓ | | TCRAJ ↓ |
| | | | | Treg ↓ | | T Cell ↓ |
| | | | | | | TCRA ↓ |
| | | | | | | TCRB ↓ |
| | | | | | | Treg ↓ |

-

TABLE 40
Top 3 SHAP predictors (e.g., modules) of each of the 28 binary classifications. For each binary classification,
the most important module is marked as 1, the 2nd most important module is marked as 2, and the 3rd
most important module is marked as 3. Binary classifications are listed across the top row.

| Modules | A_B | A_C | A_D | A_E | A_F | A_G | A_H | B_C | B_D | B_E | B_F | B_G | B_H |
|---|---|---|---|---|---|---|---|---|---|---|---|---|---|
| IFN (Table 8) | 0 | 0 | 1 | 2 | 0 | 3 | 3 | 0 | 0 | 2 | 0 | 0 | 3 |
| Immunoproteasome (Table 12) | 0 | 0 | 3 | 0 | 0 | 0 | 0 | 0 | 3 | 0 | 0 | 0 | 0 |
| Plasma Cell (Table 23) | 0 | 0 | 0 | 3 | 0 | 0 | 0 | 0 | 0 | 0 | 0 | 0 | 0 |
| Cell Cycle (Table 4) | 0 | 0 | 0 | 1 | 0 | 0 | 0 | 0 | 0 | 0 | 0 | 0 | 0 |
| SNOR Low Up (Table 24) | 0 | 0 | 0 | 0 | 0 | 0 | 0 | 0 | 0 | 0 | 0 | 0 | 0 |
| IL1 Pathway (Table 10) | 0 | 0 | 0 | 0 | 0 | 0 | 0 | 3 | 0 | 0 | 0 | 0 | 0 |
| Inflammasome (Table 13) | 0 | 0 | 0 | 0 | 0 | 0 | 0 | 1 | 0 | 0 | 2 | 2 | 2 |
| Inhibitory Macrophages | 0 | 2 | 0 | 0 | 0 | 0 | 0 | 0 | 0 | 0 | 0 | 0 | 0 |
| (Table 15) | | | | | | | | | | | | | |
| Inflammatory Cytokines | 0 | 0 | 0 | 0 | 0 | 0 | 0 | 0 | 0 | 0 | 0 | 0 | 0 |
| (Table 14) | | | | | | | | | | | | | |
| Anti-inflammation (Table 2) | 0 | 0 | 0 | 0 | 0 | 0 | 0 | 0 | 0 | 0 | 3 | 0 | 0 |
| TNF (Table 31) | 0 | 0 | 2 | 0 | 0 | 2 | 0 | 0 | 0 | 3 | 1 | 1 | 1 |
| Monocyte (Table 18) | 0 | 1 | 0 | 0 | 3 | 1 | 1 | 0 | 0 | 0 | 0 | 0 | 0 |
| Neutrophil (Table 19) | 0 | 0 | 0 | 0 | 0 | 0 | 2 | 0 | 0 | 0 | 0 | 0 | 0 |
| Granulocyte (Table 7) | 0 | 0 | 0 | 0 | 0 | 0 | 0 | 0 | 0 | 0 | 0 | 0 | 0 |
| LDG (Table 16) | 0 | 0 | 0 | 0 | 0 | 0 | 0 | 0 | 0 | 0 | 0 | 0 | 0 |
| Dendritic Cell (Table 5) | 0 | 0 | 0 | 0 | 0 | 0 | 0 | 0 | 0 | 0 | 0 | 0 | 0 |
| pDC (Table 22) | 0 | 0 | 0 | 0 | 0 | 0 | 0 | 0 | 0 | 0 | 0 | 0 | 0 |
| NK Cell (Table 20) | 0 | 0 | 0 | 0 | 0 | 0 | 0 | 0 | 0 | 0 | 0 | 0 | 0 |
| MHC II (Table 17) | 0 | 0 | 0 | 0 | 0 | 0 | 0 | 0 | 0 | 0 | 0 | 0 | 0 |
| B Cell (Table 3) | 0 | 0 | 0 | 0 | 0 | 0 | 0 | 0 | 0 | 0 | 0 | 0 | 0 |
| gd T Cell (Table 6) | 1 | 3 | 0 | 0 | 2 | 0 | 0 | 0 | 2 | 0 | 0 | 0 | 0 |
| Anergic/activated T cells | 0 | 0 | 0 | 0 | 0 | 0 | 0 | 0 | 0 | 0 | 0 | 0 | 0 |
| (Table 1) | | | | | | | | | | | | | |
| Oxidative Phosphorylation | 3 | 0 | 0 | 0 | 0 | 0 | 0 | 0 | 0 | 0 | 0 | 0 | 0 |
| (Table 21) | | | | | | | | | | | | | |
| Unfolded Protein (Table 32) | 0 | 0 | 0 | 0 | 0 | 0 | 0 | 2 | 1 | 1 | 0 | 3 | 0 |
| T Cell (Table 25) | 2 | 0 | 0 | 0 | 1 | 0 | 0 | 0 | 0 | 0 | 0 | 0 | 0 |
| IL23 Complex (Table 11) | 0 | 0 | 0 | 0 | 0 | 0 | 0 | 0 | 0 | 0 | 0 | 0 | 0 |

| Modules | C_D | C_E | C_F | C_G | C_H | D_E | D_F | D_G | D_H | E_F | E_G | E_H | F_G | F_H | G_H |
|---|---|---|---|---|---|---|---|---|---|---|---|---|---|---|---|
| IFN (Table 8) | 0 | 3 | 0 | 1 | 1 | 0 | 0 | 0 | 0 | 0 | 0 | 0 | 0 | 2 | 0 |
| Immunoproteasome (Table 12) | 0 | 0 | 0 | 0 | 0 | 0 | 0 | 0 | 0 | 0 | 0 | 0 | 2 | 1 | 0 |
| Plasma Cell (Table 23) | 2 | 2 | 0 | 0 | 0 | 0 | 0 | 0 | 0 | 0 | 0 | 0 | 0 | 0 | 0 |
| Cell Cycle (Table 4) | 0 | 1 | 0 | 0 | 3 | 0 | 0 | 0 | 0 | 0 | 0 | 0 | 0 | 0 | 0 |
| SNOR Low Up (Table 24) | 0 | 0 | 0 | 0 | 0 | 0 | 0 | 0 | 0 | 0 | 0 | 0 | 0 | 0 | 0 |
| IL1 Pathway (Table 10) | 0 | 0 | 0 | 0 | 0 | 0 | 0 | 0 | 0 | 0 | 3 | 0 | 0 | 0 | 0 |
| Inflammasome (Table 13) | 0 | 0 | 0 | 0 | 0 | 0 | 0 | 1 | 0 | 0 | 1 | 1 | 0 | 3 | 0 |
| Inhibitory Macrophages | 0 | 0 | 0 | 0 | 0 | 0 | 0 | 0 | 0 | 0 | 2 | 0 | 0 | 0 | 0 |
| (Table 15) | | | | | | | | | | | | | | | |
| Inflammatory Cytokines | 0 | 0 | 0 | 0 | 0 | 0 | 0 | 0 | 0 | 0 | 0 | 0 | 0 | 0 | 0 |
| (Table 14) | | | | | | | | | | | | | | | |
| Anti-inflammation (Table 2) | 0 | 0 | 0 | 0 | 2 | 0 | 0 | 0 | 0 | 0 | 0 | 0 | 0 | 0 | 0 |
| TNF (Table 31) | 0 | 0 | 0 | 3 | 0 | 0 | 0 | 0 | 0 | 0 | 0 | 0 | 0 | 0 | 0 |
| Monocyte (Table 18) | 0 | 0 | 0 | 0 | 0 | 0 | 0 | 2 | 0 | 0 | 0 | 3 | 0 | 0 | 0 |
| Neutrophil (Table 19) | 0 | 0 | 0 | 0 | 0 | 0 | 0 | 0 | 0 | 0 | 0 | 0 | 0 | 0 | 0 |
| Granulocyte (Table 7) | 0 | 0 | 0 | 0 | 0 | 0 | 0 | 3 | 3 | 0 | 0 | 2 | 0 | 0 | 0 |
| LDG (Table 16) | 0 | 0 | 0 | 0 | 0 | 0 | 0 | 0 | 0 | 0 | 0 | 0 | 0 | 0 | 0 |
| Dendritic Cell (Table 5) | 0 | 0 | 3 | 0 | 0 | 0 | 0 | 0 | 0 | 0 | 0 | 0 | 0 | 0 | 0 |
| pDC (Table 22) | 0 | 0 | 0 | 0 | 0 | 0 | 0 | 0 | 0 | 0 | 0 | 0 | 0 | 0 | 0 |
| NK Cell (Table 20) | 0 | 0 | 0 | 0 | 0 | 1 | 0 | 0 | 0 | 0 | 0 | 0 | 0 | 0 | 0 |
| MHC II (Table 17) | 0 | 0 | 2 | 0 | 0 | 2 | 2 | 0 | 0 | 0 | 0 | 0 | 0 | 0 | 0 |
| B Cell (Table 3) | 0 | 0 | 0 | 0 | 0 | 0 | 0 | 0 | 0 | 0 | 0 | 0 | 0 | 0 | 0 |
| gd T Cell (Table 6) | 1 | 0 | 0 | 0 | 0 | 3 | 1 | 0 | 1 | 3 | 0 | 0 | 0 | 0 | 1 |
| Anergic/activated T cells | 0 | 0 | 0 | 0 | 0 | 0 | 0 | 0 | 0 | 2 | 0 | 0 | 3 | 0 | 2 |
| (Table 1) | | | | | | | | | | | | | | | |
| Oxidative Phosphorylation | 0 | 0 | 0 | 0 | 0 | 0 | 0 | 0 | 0 | 0 | 0 | 0 | 0 | 0 | 3 |
| (Table 21) | | | | | | | | | | | | | | | |
| Unfolded Protein (Table 32) | 0 | 0 | 0 | 2 | 0 | 0 | 0 | 0 | 0 | 0 | 0 | 0 | 1 | 0 | 0 |
| T Cell (Table 25) | 3 | 0 | 1 | 0 | 0 | 0 | 3 | 0 | 2 | 1 | 0 | 0 | 0 | 0 | 0 |
| IL23 Complex (Table 11) | 0 | 0 | 0 | 0 | 0 | 0 | 0 | 0 | 0 | 0 | 0 | 0 | 0 | 0 | 0 |

TABLE 41
Top 10 gene predictors of each of the 28 binary classification.

A_B: ATP5A1, CD247, COX15, COX6B2, NDUFA9, NDUFB2-AS1, NDUFC2, NDUFS1,
NDUFS7, and SH2D1A
A_C: ADGRE2, AOAH, BACH1, CLEC4D, CLEC7A, FFAR2, LILRA6, LMNB1, TLR2,
and TNFRSF1B
A_D: ACLY, ARSE, CASP1, CASP10, CTNND2, EIF2AK2, GBP1, IFI30, IL1RN and
PSMB8
A_E: AURKB, CCNE1, EIF2AK2, GBP2, IFITM3, IGHG1, IGLV4-60, IGLV5-45, ISG20,
and PTTG1
A_F: CCL28, CD247, CHIT1, CXCL1, FFAR2, LILRB5, LMNB1, PYHIN1, SECTM1, and
SIGLEC5
A_G: APOBR, CASP1, CASP10, FFAR2, MS4A4A, MTF1, SECTMI, SEMA4A, TLR8,
and TNFRSF1B
A_H: ADAM8, APOBEC3B, CCL28, CD177, CXCL1, EIF2AK2, FCGR3B, IL10RA,
LILRA5, and OSCAR
B_C: CANX, CASP1, CHUK, DERL2, ERGIC2, HERPUD1, IRAK1, IRAK4, RIPK1, and
SEC24D
B_D: CALR, EDEM2, EMC9, ERAP1, KDELC1, MANF, NUCB2, PSMB8, SEC24D, and
TRDC
B_E: ACLY, ARSE, CD38, DERL1, DERL2, EDEM3, EIF2AK2, MANF, NFKB1 and
SEC24D
B_F: ACSL1, AIM2, ASAP1, CASP1, IL18, IL1B, IL1RN, MTF1, RIPK1, and SPI1
B_G: ACLY, ARSE, BHMT, CASP10, CD37, EDEM2, GLS, HOMER2, IL1A, and
TNFAIP3
B_H: ACSL1, AIM2, AKAP10, CASP10, CD38, CKB, IL18, NAIP, NFKB1, and TYROBP
C_D: BLK, CD247, CD3D, CD8A, IGHG1, IGHV3-20, SH2D1A, THEMIS2, TRDC, and
TRG-AS1
C_E: AURKB, CCNB1, CCNE1, EIF2AK2, IGHG1, IGHV3-20, IGLL1, IGLV4-3, IGLVI-
70, and PTTG1
C_F: CD226, CD247, CD28, CD4, CLEC10A, HLA-DRB1, HLA-DRB6, IGIP, LY75, and
TRDC
C_G: ACLY, CASP10, CD37, CD38, DERL1, DERL2, EDEM2, EIF2AK2, IFI30, and
MANF
C_H: AURKB, BRCA1, E2F3, EIF2AK2, IFITM3, MCM10, NDC80, PTTG1, SOCS3, and
TNFAIP3
D_E: CD3E, HLA-DMA, HLA-DPA1, HLA-DPB2, HLA-DQA2, HLA-DRB1, HLA-DRB6,
KLRF1, NCAM1, and TRDC
D_F: BLK, CD226, CD247, CD8A, HLA-DQA1, HLA-DQA2, HLA-DRB5, HLA-DRB6,
TARP, and TRG-AS1
D_G: CD14, CLEC5A, LILRA2, LILRA5, LILRA6, LMNB1, NLRC4, OLR1, OSCAR, and
SEMA4A
D_H: BLK, CD177, CD247, CXCR2, FUT7, LTB4R, OSM, TARP, TRDC, and TRG-AS1
E_F: BLK, CCR3, CD247, CD28, CD4, CD8A, CTLA4, HAVCR2, KLRG1, and TRG-AS1
E_G: CALR, CD244, CTLA4, DERL2, EMC9, ERAP1, GALNT2, HAVCR2, ICOS, and
PSMB8
E_H: CASP1, EIF2AK2, GBP1, IFI30, IFITM3, NEK7, NLRC4, PSMB10, PSMB8, and
RIPK1
F_G: CALR, CD244, CTLA4, DERL2, EMC9, ERAP1, GALNT2, HAVCR2, ICOS, and
PSMB8
F_H: CASP1, EIF2AK2, GBP1, IFI30, IFITM3, NEK7, NLRC4, PSMB10, PSMB8, and
RIPK1
G_H: ATP5A1, CD160, CD244, CD3E, COX16, COX18, NDUFAF4, NDUFS1, TIMMDC1,
and TTC19

### REFERENCES (EACH INCORPORATED BY REFERENCE HEREIN IN ITS ENTIRETY)

1. Fanouriakis A, Tziolos N, Bertsias G, et al., Update on the diagnosis and management of systemic lupus erythematosus. Ann Rheum Dis [Internet]. 2020 Jan. 1[cited 2022 Apr. 18]; 80(1):14-25. Available from: https://ard.bmj.com/content/80/1/14
2. van Vollenhoven R F, Askanase A D, Bomback A S, et al. Conceptual framework for defining disease modification in systemic lupus erythematosus: a call for formal criteria. Lupus Sci Med [Internet]. 2022 Mar. 1[cited 2022 Apr. 22]; 9(1):e000634. Available from: https://lupus.bmj.com/content/9/1/e000634
3. Agache I, Akdis C A. Precision medicine and phenotypes, endotypes, genotypes, regiotypes, and theratypes of allergic diseases. J Clin Invest. 2019 Apr. 1; 129(4):1493-503.
4. Petrelli A, Giovenzana A, Insalaco V, et al. Autoimmune Inflammation and Insulin Resistance: Hallmarks So Far and Yet So Close to Explain Diabetes Endotypes. Curr Diab Rep [Internet]. 2021 Dec. 1[cited 2022 Apr. 25]; 21(12):1-10. Available from: https://link.springer.com/article/10.1007/s11892-021-01430-3
5. Battaglia M, Ahmed S, Anderson M S, et al. Introducing the Endotype Concept to Address the Challenge of Disease Heterogeneity in Type 1 Diabetes. Diabetes Care [Internet]. 2020 Jan. 1[cited 2022 Apr. 25]; 43(1):5-12. Available from: https://diabetesjournals.org/care/article/43/1/5/35967/Introducing-the-Endotype-Concept-to-Address-the
6. Tarn J R, Lendrem D W, Isaacs J D. In search of pathobiological endotypes: a systems approach to early rheumatoid arthritis. Expert Rev Clin Immunol [Internet]. 2020 Jun. 2[cited 2022 Apr. 25]; 16(6):621-30. Available from: https://pubmed.ncbi.nlm.nih.gov/32456483/
7. Neumann M, Bastian L, Hänzelmann S, et al. Molecular Subgroups of T Cell Acute Lymphoblastic Leukemia in Adults Treated According to GMALL Protocols. Blood [Internet]. 2020 Nov. 5 [cited 2022 Jun. 22]; 136(Supplement 1):37-8. Available from: https://ashpublications.org/blood/article/136/Supplement 1/37/469979/Molecular-Subgroups-of-T-Cell-Acute-Lymphoblastic
8. Bastian L, Hänzelmann S, Neumann M, et al. Molecular Subtypes with Distinct Clinical Phenotypes and Actionable Targets in Adult B Cell Precursor ALL Treatment According to GMALL Protocols. Blood [Internet]. 2020 Nov. 5[cited 2022 Jun. 22]; 136(Supplement 1):11-2. Available from: https://ashpublications.org/blood/article/136/Supplement 1/11/472511/Molecular-Subtypes-with-Distinct-Clinical
9. Banchereau R, Hong S, Cantarel B, et al. Personalized Immunomonitoring Uncovers Molecular Networks That Stratify Lupus Patients. Cell [Internet]. 2016 Apr. 4[cited 2022 Jun. 22]; 165(3):551. Available from: https://www.ncbi.nlm.nih.gov//pmc/articles/PMC5426482/
10. Toro-Dominguez D, Martorell-Marugin J, Goldman D, et al. Stratification of Systemic Lupus Erythematosus Patients Into Three Groups of Disease Activity Progression According to Longitudinal Gene Expression. Arthritis Rheumatol [Internet]. 2018 Dec. 1[cited 2021 Jun. 4]; 70(12):2025-35. Available from: https://onlinelibrary.wiley.com/doi/full/10.1002/art.40653
11. Kegerreis B, Catalina M D, Bachali P, et al. Machine learning approaches to predict lupus disease activity from gene expression data. Sci Rep [Internet]. 2019 Dec. 1[cited 2021 Jun. 4]; 9(1):1-12. Available from: https://www.nature.com/articles/s41598-019-45989-0
12. Nehar-Belaid D, Hong S, Marches R, et al. Mapping systemic lupus erythematosus heterogeneity at the single-cell level. Nat Immunol [Internet]. 2020 Aug. 3[cited 2022 Jun. 23]; 21(9):1094-106. Available from: https://www.nature.com/articles/s41590-020-0743-0
13. Catalina M D, Bachali P, Yeo A E, et al. Patient ancestry significantly contributes to molecular heterogeneity of systemic lupus erythematosus. JCI Insight. 2020 Aug. 6; 5(15). Available from: https://insight.jci.org/articles/view/140380
14. Chiche L, Jourde-Chiche N, Whalen E, et al. Modular transcriptional repertoire analyses of adults with systemic lupus erythematosus reveal distinct type I and type I I interferon signatures. Arthritis Rheumatol. 2014; 66(6):1583-95.
15. Guthridge J M, Lu R, Tran L T H, et al. Adults with systemic lupus exhibit distinct molecular phenotypes in a cross-sectional study. EClinicalMedicine [Internet]. 2020 Mar. 1 [cited 2021 Jun. 4]; 20. Available from: http://www.thelancet.com/article/S2589537020300353/fulltext
16. Andreoletti G, Lanata C M, Trupin L, Paranjpe I, Jain T S, Nititham J, et al. Transcriptomic analysis of immune cells in a multi-ethnic cohort of systemic lupus erythematosus patients identifies ethnicity- and disease-specific expression signatures. Commun Biol [Internet]. 2021 Apr. 21[cited 2022 Jun. 29]; 4(1):1-13. Available from: https://www.nature.com/articles/s42003-021-02000-9
17. Lopez-Dominguez R, Toro-Dominguez D, Martorell-Marugan J, et al. Transcription Factor Activity Inference in Systemic Lupus Erythematosus. Life (Basel) [Internet]. 2021 Apr. 1[cited 2022 Jun. 29]; 11(4). Available from: https://pubmed.ncbi.nlm.nih.gov/33915751/
18. Garantziotis P, Nikolakis D, Doumas S, et al. Molecular Taxonomy of Systemic Lupus Erythematosus Through Data-Driven Patient Stratification: Molecular Endotypes and Cluster-Tailored Drugs. Front Immunol. 2022 May 9; 0:1862.
19. Ding Y, Li H, He X, et al. Identification of a gene-expression predictor for diagnosis and personalized stratification of lupus patients. PLoS One [Internet]. 2018 Jul. 1[cited 2022 Jul. 8]; 13(7):e0198325. Available from: https://journals.plos.org/plosone/article?id=10.1371/journal.pone.0198325
20. Figgett W A, Monaghan K, Ng M, et al. Machine learning applied to whole-blood RNA-sequencing data uncovers distinct subsets of patients with systemic lupus erythematosus. Clin Transl Immunology [Internet]. 2019 Jan. 1[cited 2022 Jul. 8]; 8(12):e01093. Available from: https://onlinelibrary.wiley.com/doi/full/10.1002/cti2.1093
21. Yones S A, Annett A, Stoll P, et al. Interpretable machine learning identifies paediatric Systemic Lupus Erythematosus subtypes based on gene expression data. Sci Rep [Internet]. 2022 May 6[cited 2022 Jul. 19]; 12(1):1-10. Available from: https://www.nature.com/articles/s41598-022-10853-1
22. Hubbard E L, Grammer A C, Lipsky P E. Transcriptomics data: pointing the way to subclassification and personalized medicine in systemic lupus erythematosus. Curr Opin Rheumatol [Internet]. 2021 Nov. 1[cited 2022 Apr. 22]; 33(6):579-85. Available from: https://journals.lww.com/co-rheumatology/Fulltext/2021/11000/Transcriptomics_data_pointing_the_way_to.17.aspx
23. Merrill J T, van Vollenhoven R F, Buyon J P, et al. Efficacy and safety of subcutaneous tabalumab, a monoclonal antibody to B-cell activating factor, in patients with systemic lupus erythematosus: Results from ILLUMINATE-2, a 52-week, phase III, multicentre, randomised, double-blind, placebo-controlled study. Ann Rheum Dis [Internet]. 2016 Feb. 1[cited 2022 Jun. 13]; 75(2):332-40. Available from: https://ard.bmj.com/content/75/2/332
24. Hothorn T, van de Wiel M A, Hornik K, et al. Implementing a Class of Permutation Tests: The coin Package. J Stat Softw [Internet]. 2008 Nov. 13[cited 2022 Jun. 23]; 28(8):1-23. Available from: https://www.jstatsoft.org/index.php/jss/article/view/v028i08
25. Petri M, Buyon J, Kim M. Classification and definition of major flares in SLE clinical trials. Lupus [Internet]. 1999 Jul. 2[cited 2022 Jul. 19]; 8(8):685-91. Available from: https://journals.sagepub.com/doi/10.1191/096120399680411281?url_ver=Z39.88-2003&rfr_id=ori %3Arid %3Acrossref.org&rfr_dat=cr_pub++Opubmed
26. Lundberg S M, Lee S I. A Unified Approach to Interpreting Model Predictions. Adv Neural Inf Process Syst [Internet]. 2017 May 22[cited 2022 Jul. 11]; 2017-December:4766-75. Available from: https://arxiv.org/abs/1705.07874v2
27. Hoffman R W, Merrill J T, Alarcón-Riquelme M E, et al. Gene Expression and Pharmacodynamic Changes in 1,760 Systemic Lupus Erythematosus Patients From Two Phase III Trials of BAFF Blockade With Tabalumab. Arthritis Rheumatol [Internet]. 2017 Mar. 1[cited 2022 Jun. 13]; 69(3):643-54. Available from: https://onlinelibrary.wiley.com/doi/full/10.1002/art.39950
28. Furie R A, Petri M A, Wallace D J, et al. Novel Evidence-Based Systemic Lupus Erythematosus Responder Index. Arthritis Rheum [Internet]. 2009 Sep. 9[cited 2022 Jun. 30]; 61(9):1143. Available from: https://ncbi.nlm.nih.gov/pmc/articles/PMC2748175/
29. Petri M, Watts S D, Higgs R E, et al. Sub-setting systemic lupus erythematosus by combined molecular phenotypes defines divergent populations in two phase III randomized trials. Rheumatology (Oxford) [Internet]. 2021 Nov. 3[cited 2022 Jun. 22]; 60(11):5390-6. Available from: https://academic.oup.com/rheumatology/article/60/11/5390/6134338
30. Shobha V, Mohan A, Malini A V, et al. Identification and stratification of systemic lupus erythematosus patients into two transcriptionally distinct clusters based on IFN-I signature. Lupus [Internet]. 2021 Apr. 1[cited 2021 Jun. 4]; 30(5):762-74. Available from: https://journals.sagepub.com/doi/abs/10.1177/0961203321990107
31. Diaz-Gallo L, Oke V, Lundstrom E, et al. Four Systemic Lupus Erythematosus Subgroups, Defined by Autoantibodies Status, Differ Regarding HLA-DRB1 Genotype Associations and Immunological and Clinical Manifestations. ACR Open Rheumatol [Internet]. 2022 January [cited 2022 Jul. 8]; 4(1):27. Available from: https://ncbi.nlm.nih.gov/pmc/articles/PMC8754019/
32. Lewis M J, Jawad A S. The effect of ethnicity and genetic ancestry on the epidemiology, clinical features and outcome of systemic lupus erythematosus. Rheumatology (Oxford) [Internet]. 2017 Apr. 1[cited 2022 Jun. 23]; 56(suppl_1):i67-77. Available from: https://academic.oup.com/rheumatology/article/56/suppl_1/i67/2629213
33. Hanzelmann S, Castelo R, Guinney J. GSVA: Gene set variation analysis for microarray and RNA-Seq data. BMC Bioinformatics [Internet]. 2013 Jan. 16[cited 2022 May 16]; 14(1):1-15. Available from: https://bmcbioinformatics.biomedcentral.com/articles/10.1186/1471-2105-14-7
34. Hubbard E L, Catalina M D, Heuer S, et al. Analysis of gene expression from systemic lupus erythematosus synovium reveals myeloid cell-driven pathogenesis of lupus arthritis. Sci Rep [Internet]. 2020 Oct. 15[cited 2021 May 1]; 10(1). Available from: https://ncbi.nlm.nih.gov/pmc/articles/PMC7562741/
35. Kingsmore K M, Bachali P, Catalina M D, et al. Altered expression of genes controlling metabolism characterizes the tissue response to immune injury in lupus. Sci Rep [Internet]. 2021 Dec. 1[cited 2022 Jun. 23]; 11(1). Available from: https://pubmed.ncbi.nlm.nih.gov/34285256/
36. Daamen A R, Bachali P, Owen K A, et al. Comprehensive transcriptomic analysis of COVID-19 blood, lung, and airway. Sci Rep [Internet]. 2021 Mar. 29[cited 2021 Oct. 4]; 11(1). Available from: https://ncbi.nlm.nih.gov/pmc/articles/PMC8007747/
37. Martinez B A, Shrotri S, Kingsmore K M, et al. Machine learning reveals distinct gene signature profiles in lesional and nonlesional regions of inflammatory skin diseases. Sci Adv [Internet]. 2022 Apr. 1[cited 2022 Jun. 29]; 8(17). Available from: https://pubmed.ncbi.nlm.nih.gov/35486723/
38. Pedregosa F, Michel V, Grisel O, et al. Scikit-leam: Machine Learning in Python. J Mach Learn Res [Internet]. 2011 October [cited 2022 Jun. 23]; 12:2825-30. Available from: http://scikit-learn.sourceforge.net.
39. Dörner T, Farner N L, Lipsky P E. Ig λ and Heavy Chain Gene Usage in Early Untreated Systemic Lupus Erythematosus Suggests Intensive B Cell Stimulation. J Immunol. 1999; 163(2).
40. R Core Team (2021). R: A language and environment for statistical computing. R Foundation for Statistical Computing, Vienna, Austria. URL https://www.R-project.org/.
41. Hunter J D. Matplotlib: A 2D graphics environment. Comput Sci Eng. 2007; 9(3):90-5.
42. Wild F (2022). lsa: Latent Semantic Analysis. R package version 0.73.3. https://CRAN.R-project.org/package=lsa
43. Dilokthanakul N, Mediano P A M, Garnelo M, et al. Deep Unsupervised Clustering with Gaussian Mixture Variational Autoencoders. 2016 Nov. 8[cited 2022 Jul. 19]. Available from: https://arxiv.org/abs/1611.02648v2
44. Chawla N V, Bowyer K W, Hall L O, et al. SMOTE: Synthetic Minority Over-sampling Technique. J Artif Intell Res [Internet]. 2002 Jun. 1[cited 2022 Jul. 11]; 16:321-57. Available from: https://www.jair.org/index.php/jair/article/view/10302
45. Kuhn M (2022). caret: Classification and Regression Training. R package version 6.0-92. https://CRAN.R-project.org/package=caret

### Example 3: Analysis of the Transcriptomic Profiles of Rheumatic Skin Diseases Reveals Disease-Specific Endotypes

Background: Patients with rheumatic skin diseases such as systemic sclerosis (SSc) can be classified from individuals with healthy skin using the enrichment of the 32 molecular signatures (Tables 1 to 32) [1]. However, as patient heterogeneity is a well-known feature of these diseases, it is important to ascertain whether there are distinct patient molecular phenotypes (endotypes). Moreover, identifying specific disease endotypes from analysis of peripheral blood might increase the capacity to recognize patient endotypes.

Methods: Gene expression data derived from publicly available lesional skin biopsies of SSc patients was analyzed using gene set variation analysis (GSVA) of informative gene modules. Paired blood samples were also analyzed when available. K-means clustering was then applied to the GSVA enrichment scores to identify molecular endotypes in skin diseases.

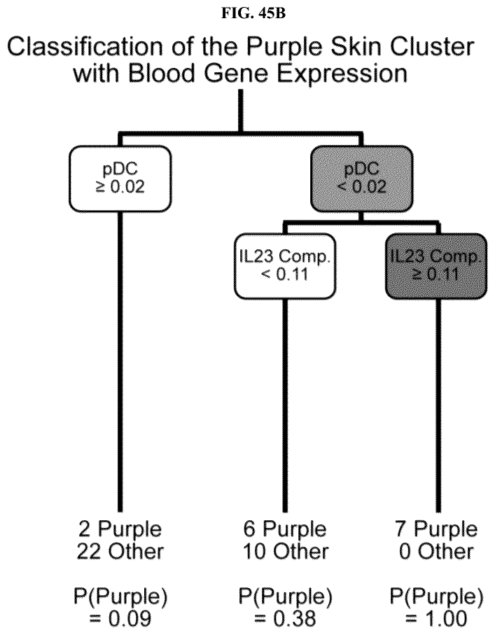

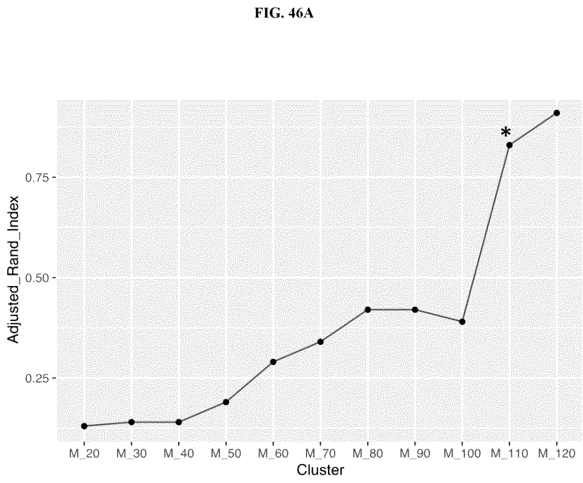

Results: We examined the ability to predict SSc skin endotypes from the blood of paired samples. Blood was analyzed for enrichment of blood-specific gene signatures and then the blood samples were grouped into 4 clusters, gold (group 1), orange (group 2), pink (group 3) and purple (group 4) cluster. (FIG. 45A). The first cluster (gold) was the least abnormal, and the purple cluster was most severe SSc endotype (purple). The most severe SSc skin endotype could be predicted by Classification and Regression Tree (CART) analysis of blood, a negative plasmacytoid dendritic cell (pDC) GSVA score in the blood predicted the purple cluster (FIG. 45B).

Conclusions: SSc skin exhibit distinct subsets based upon their molecular profile (endotype). The most severe SSc skin subset can be identified from blood gene expression.

Identifying specific molecular endotypes of patients with inflammatory skin disease may facilitate matching individual patients with effective therapies.

### REFERENCES

[1]Martinez B A, Shrotri S, Kingsmore K M, Bachali P, Grammer A C, Lipsky P E. Sci Adv. 2022; 8(17): eabn4776.

### INCLUDED EMBODIMENTS

1. A method for assessing a SSc disease state of a patient, the method comprising: analyzing a data set comprising or derived from gene expression measurements data of at least 2 genes selected from the genes listed in Tables 1 to 32, in a biological sample obtained or derived from the patient, to classify the SSc disease state of the patient.
2. The method of embodiment 1, wherein the SSc disease state of the patient is classified as group 1, group 2, group 3 or group 4 SSc disease state.
3. The method of embodiment 1 or embodiment 2, wherein the data set comprises or is derived from gene expression measurements data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, or 2000 genes, selected from the genes listed in Tables 1-32, in the biological sample obtained or derived from the patient.
4. The method of any one of embodiments 1 to 4, wherein the data set comprises or is derived from gene expression measurements data of at least 2 to all, or any value or range there between, genes selected from the genes listed in each of one or more Tables selected from Tables 1 to 32, in the biological sample obtained or derived from the patient, wherein number of genes selected from the genes in each selected table may be different or same.
5. The method of embodiment 4, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, Tables selected from Tables 1 to 32.
6. The method of embodiments 4 or 5, wherein the selected Tables are Tables 1 to 32.
7. The method of any one of embodiments 1 to 6, wherein the SSc disease state of the patient is classified with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
8. The method of any one of embodiments 1 to 7, wherein the SSc disease state of the patient is classified with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
9. The method of any one of embodiments 1 to 8, wherein the SSc disease state of the patient is classified with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
10. The method of any one of embodiments 1 to 9, wherein the SSc disease state of the patient is classified with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
11. The method of any one of embodiments 1 to 10, wherein the SSc disease state of the patient is classified with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
12. The method of any one of embodiments 1 to 11, wherein the SSc disease state of the patient is classified with a Receiver operating characteristic (ROC) curve having an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.
13. The method of any one of embodiments 1 to 12, wherein the data set is derived from the gene expression measurements data using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.
14. The method of any one of embodiments 1 to 12, wherein the data set is derived from the gene expression measurements data using GSVA.
15. The method of embodiment 14, wherein the data set comprises one or more GSVA scores of the patient, wherein the one or more GSVA scores are generated based on one or more Tables selected from Tables 1 to 32, wherein for each selected Table, at least one GSVA score of the patient is generated based on enrichment of expression of at least 2 genes thereof listed in the selected Table in the biological sample, and wherein the one or more GSVA scores comprise each generated GSVA score.
16. The method of embodiment 15, wherein for each selected Table, the at least one GSVA score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 genes listed in the respective Table, wherein number of genes selected from different selected Tables can be same or different.
17. The method of any one of embodiments 1 to 16, wherein the analyzing the data set comprises providing the data set as an input to a trained machine-learning model to classify the SSc disease state of the patient, wherein the trained machine-learning model generate an inference indicative of the SSc disease state of the patient based at least on the data set.
18. The method of embodiment 17, wherein the data set comprises the one or more GSVA scores of the patient, and the trained machine-learning model generate the inference based at least on the one or more GSVA scores.
19. The method of embodiments 17 or 18, wherein the method further comprises receiving, as an output of the trained machine-learning model, the inference; and/or electronically outputting a report indicating the SSc disease state of the patient.
20. The method of any one of embodiments 17 to 19, wherein the machine-learning model is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.
21. The method of any one of embodiments 1 to 20, the patient is at elevated risk of having, is suspected of having, is asymptomatic for, and/or has SSc.
22. The method of any one of embodiments 1 to 21, further comprising selecting, recommending and/or administering a treatment to the patient based at least in part on the classification of the SSc disease state of the patient.
23. The method of embodiment 22, wherein the treatment is configured to treat, reduce a severity of lupus nephritis, and/or reduce a risk of having SSc.
24. The method of any one of embodiments 1 to 23, wherein the treatment comprises a pharmaceutical composition.
25. The method of any one of embodiments 1 to 24, wherein the treatment for SSc comprises an agent that targets TGFB fibroblasts (e.g., nintedanib, pirfenidone), and/or dendritic cells (e.g., BIIB059, Daxdilmab).
26. The method of any one of embodiments 1 to 25, wherein the biological sample comprises a blood sample, isolated peripheral blood mononuclear cells (PBMCs), skin biopsy sample, or any derivative thereof.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It may be understood that various alternatives to the embodiments described herein may be employed in practice. Numerous different combinations of embodiments described herein are possible, and such combinations are considered part of the present disclosure. In addition, all features discussed in connection with any one embodiment herein may be readily adapted for use in other embodiments herein.

It is intended that the following claims define the scope of the disclosure and that methods and structures within the scope of these claims and their equivalents be covered thereby.

1. A method for classifying a lupus disease state of a patient, the method comprising:
analyzing a data set comprising or derived from gene expression measurements of at least 2 genes selected from genes listed in each of one or more Tables selected from Tables: 1 to 32, to classify the lupus disease state of the patient,
wherein the gene expression measurements are obtained from a biological sample obtained or derived from the patient.

2. The method of claim 1, wherein the lupus disease state of the patient is classified as group A lupus disease state, group B lupus disease state, group C lupus disease state, group D lupus disease state, group E lupus disease state, group F lupus disease state, group G lupus disease state, or group H lupus disease state.

3. The method of claim 1, wherein the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, or all genes selected from genes listed in each of the one or more Tables selected from Tables: 1 to 32, wherein the number of genes selected from different selected Tables may be the same or different.

4. (canceled)

5. The method of claim 1, wherein at least 23 Tables are selected from Tables: 1 to 32.

6. (canceled)

7. (canceled)

8. The method of claim 1, wherein the data set comprises or is derived from gene expression measurements of all the genes listed in the Tables selected.

9. The method of claim 1, wherein the method for classifying the lupus disease state of the patient with; (i) an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (ii) a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (iii) a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (iv) a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (v) a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

10. (canceled)

11. (canceled)

12. (canceled)

13. (canceled)

14. The method of claim 1, wherein the data set; (i) is derived from the gene expression measurements using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof; (ii) is derived from the gene expression measurements using GSVA; or (iii) comprises one or more GSVA scores of the patient, wherein the one or more GSVA scores are generated based on the one or more Tables selected from Tables 1 to 32, wherein for each selected Table the at least 2 genes selected from the selected Table forms an input gene set for generating a GSVA score based on the selected Table using GSVA, and wherein the one or more GSVA scores comprise each generated GSVA score.

15. (canceled)

16. (canceled)

17. (canceled)

18. The method of claim 1, wherein analyzing the data set comprises providing the data set as an input to a trained machine-learning model trained to generate an inference of whether the data set is indicative of the patient having group A lupus disease state, group B lupus disease state, group C lupus disease state, group D lupus disease state, group E lupus disease state, group F lupus disease state, group G lupus disease state, or group H lupus disease state, wherein the method classify the lupus disease state of the patient based on the inference of the trained machine-learning model.

19. The method of claim 18, further comprising:
a) receiving, as an output of the trained machine-learning model, the inference; and
b) electronically outputting a report classifying the lupus disease state of a patient.

20. The method of claim 18, wherein the trained machine-learning model is trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), Classification and Regression Tree (CART), hierarchical clustering, or any combination thereof.

21. The method of claim 18, wherein the inference comprises a confidence value between 0 and 1 that the patient has the group A lupus disease state, the group B lupus disease state, the group C lupus disease state, the group D lupus disease state, the group E lupus disease state, the group F lupus disease state, group G lupus disease state, or the group H lupus disease state.

22. The method of claim 18, wherein the trained machine-learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

23. The method of claim 1, wherein analyzing the data set comprises generating a risk score of the patient based on the data set, wherein the method classify the lupus disease state of the patient based on the risk score, optionally wherein the risk score of the patient is based on the one or more GSVA scores of the patient.

24. (canceled)

25. The method of claim 1, wherein method further comprises performing Shapley Additive Explanations (SHAP) on the data set to determine contribution of one or more gene features to the lupus disease state classification of the patient.

26. The method of claim 1, wherein the biological sample comprises a blood sample, isolated peripheral blood mononuclear cells (PBMCs), a tissue biopsy sample, or any derivative thereof.

27. The method of claim 1, wherein the patient; (i) has lupus: (ii) is at elevated risk of having lupus: or (iii) patient is asymptomatic for lupus.

28. (canceled)

29. (canceled)

30. The method of claim 27, further comprising selecting, recommending and/or administering a treatment to the patient based on the classification of the lupus disease state of the patient.

31. The method of claim 30, wherein the treatment is: (i) configured to treat lupus: (ii) configured to treat reduce severity of lupus: or (iii) configured to reduce risk of having lupus.

32. (canceled)

33. (canceled)

34. The method of claim 30, wherein the treatment: (i) comprises one or more pharmaceutical compositions: (ii) is based on the contribution of the one or more gene features to the lupus disease state classification of the patient; (iii) targets one or more gene features significantly enriched in the biological sample: (iv) targets one or more gene features significantly enriched in the biological sample: (v) comprises a neutrophil function inhibitor, a TNF inhibitor, an IL1 inhibitor, a Plasma cell inhibitor, a NK cell inhibitor, a B Cell Inhibitor, IFN inhibitor, or any combination thereof; or (vi) comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof.

35. (canceled)

36. (canceled)

37. (canceled)

38. (canceled)

39. The method of claim 30, wherein the treatment for: group B lupus disease state comprises a neutrophil function inhibitor; group C lupus disease state comprises a neutrophil function inhibitor, a TNF inhibitor, an IL1 inhibitor, an IFN inhibitor or any combination thereof; group D lupus disease state comprises a B cell inhibitor, an IFN inhibitor, a NK cell inhibitor, a neutrophil function inhibitor, a TNF inhibitor, an IL1 inhibitor, a Plasma cell inhibitor or any combination thereof, group E lupus disease state comprises an IFN inhibitor, a neutrophil function inhibitor, a TNF inhibitor, a Plasma cell inhibitor or any combination thereof; group F lupus disease state comprises an IFN inhibitor, a neutrophil function inhibitor, a TNF inhibitor, an IL1 inhibitor, or any combination thereof; group G lupus disease state comprises a B cell inhibitor, an IFN inhibitor, a neutrophil function inhibitor, a TNF inhibitor, an IL1 inhibitor, a Plasma cell inhibitor or any combination thereof, and/or group H lupus disease state comprises an IFN inhibitor, a neutrophil function inhibitor, a TNF inhibitor, an IL1 inhibitor, a Plasma cell inhibitor or any combination thereof.

40. The method of claim 30, wherein the treatment for: group B lupus disease state comprises Belimumab, Dasatinib, and/or Apremilast; group C lupus disease state comprises Anifrolumab, Anakinra, Adalimumab, Certolizumab pegol, Etanercept, Golimumab, Infiximab, Dasatinib, Apremilast, or any combination thereof, group D lupus disease state comprises Belimumab, Anifrolumab, Mycophenolate, AZA, Bortezomib, Isatuximab, Elotuzumab, Anakinra, Adalimumab, Certolizumab pegol, Etanercept, Golimumab, Infiximab, Dasatinib, Apremilast or any combination thereof, group E lupus disease state comprises Anifrolumab, Mycophenolate, Bortezomib, Isatuximab, Elotuzumab, Adalimumab, Certolizumab pegol, Etanercept, Golimumab, Infiximab, Dasatinib, Apremilast or any combination thereof; group F lupus disease state comprises Anifrolumab, Anakinra, Adalimumab, Certolizumab pegol, Etanercept, Golimumab, Infiximab, Dasatinib, Apremilast, Belimumab, or any combination thereof; group G lupus disease state comprises Belimumab, Anifrolumab, Mycophenolate, Bortezomib, Isatuximab, Elotuzumab, Anakinra, Adalimumab, Certolizumab pegol, Etanercept, Golimumab, Infiximab, Dasatinib, Apremilast or any combination thereof; and group H lupus disease state comprises Anifrolumab, Mycophenolate, Bortezomib, Isatuximab, Elotuzumab, Anakinra, Adalimumab, Certolizumab pegol, Etanercept, Golimumab, Infiximab, Dasatinib, Apremilast, Belimumab, or any combination thereof.
